(19) United States
(12) Patent Application Publication
(10) Pub. No.: US 2026/0070893 A1
(43) Pub. Date: Mar. 12, 2026

(54) NOVEL COMPOUND AND ORGANIC LIGHT EMITTING DEVICE COMPRISING THE SAME

(71) Applicant: LG CHEM, LTD., Seoul (KR)

(72) Inventors: Donghee KIM, Daejeon (KR); Sang Duk SUH, Daejeon (KR); Min Woo JUNG, Daejeon (KR); Sunmi LEE, Daejeon (KR); Dongyeun JEONG, Daejeon (KR)

(21) Appl. No.: 19/320,838

(22) Filed: Sep. 5, 2025

(30) Foreign Application Priority Data
Sep. 10, 2024 (KR) 10-2024-0123096
Aug. 25, 2025 (KR) 10-2025-0118495

(51) Int. Cl. C07D 403/10; C07B 59/00; C07D 209/86; C07D 405/14; C07D 409/14; C09K 11/06; H10K 50/13; H10K 85/60

(52) U.S. Cl. CPC C07D 403/10; C07B 59/002; C07D 209/86; C07D 405/14; C07D 409/14; C09K 11/06; H10K 50/13; H10K 85/6572; H10K 85/6574; H10K 85/6576; C07B 2200/05

(57) ABSTRACT

A compound of Chemical Formula 1:

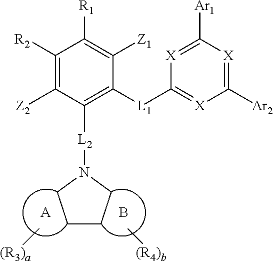

where: A is a naphthalene ring, B is a C₆₋₂₀ aromatic ring, and A and B are fused with a neighboring pentagonal ring; X are each independently N or CR, and at least one X is N; R₁ is hydrogen, deuterium, tritium, substituted or unsubstituted C₆₋₆₀ aryl, or substituted or unsubstituted C₂₋₆₀ heteroaryl including O or S, and R₂ is hydrogen, deuterium, tritium, or substituted or unsubstituted C₂₋₆₀ heteroaryl including O or S, but neither R₁ or R₂ is 4-dibenzofuranyl or 4-dibenzothiophenyl, and R₁ and R₂ are not simultaneously hydrogen, deuterium, or tritium; Ar₁ and Ar₂ are each independently substituted or unsubstituted C₁₋₆₀ alkyl, substituted or unsubstituted C₆₋₆₀ aryl, or substituted or unsubstituted C₂₋₆₀ heteroaryl including one or more of N, O and S; and the other substituents are defined in the specification; and an organic light emitting device including the same.

### CROSS-REFERENCE TO RELATED APPLICATIONS

This application claims priority to and the benefit of Korean Patent Application No. 10-2024-0123096 filed on Sep. 10, 2024 in the Korean Intellectual Property Office, and Korean Patent Application No. 10-2025-0118495 filed on Aug. 25, 2025 in the Korean Intellectual Property Office the disclosure of each of which is incorporated herein by reference in its entirety.

### TECHNICAL FIELD

The present disclosure relates to a novel compound and an organic light emitting device comprising the same.

### BACKGROUND ART

In general, an organic light emitting phenomenon refers to a phenomenon where electric energy is converted into light energy by using an organic material. The organic light emitting device using the organic light emitting phenomenon has characteristics such as a wide viewing angle, an excellent contrast, a fast response time, an excellent luminance, driving voltage and response speed, and thus many studies have proceeded.

The organic light emitting device generally has a structure which comprises an anode, a cathode, and an organic material layer interposed between the anode and the cathode. The organic material layer frequently has a multilayered structure that comprises different materials in order to enhance efficiency and stability of the organic light emitting device, and for example, the organic material layer may be formed of a hole injection layer, a hole transport layer, a light emitting layer, an electron transport layer, an electron injection layer and the like. In the structure of the organic light emitting device, if a voltage is applied between two electrodes, the holes are injected from an anode into the organic material layer and the electrons are injected from the cathode into the organic material layer, and when the injected holes and electrons meet each other, an exciton is formed, and light is emitted when the exciton falls to a ground state again.

There is a continuous need to develop a new material for the organic material used in the organic light emitting device as described above.

### PRIOR ART LITERATURE

### Patent Literature

(Patent Literature 0001) Korean Unexamined Patent Publication No. 10-2000-0051826

### DETAILED DESCRIPTION OF THE INVENTION

### Technical Problem

It is an object of the present disclosure to provide a novel compound and an organic light emitting device comprising the same.

### Technical Solution

According to the present disclosure, there is provided a compound represented by the following Chemical Formula 1:

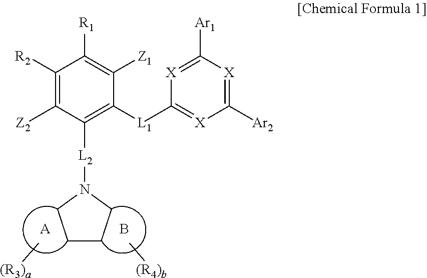

wherein in Chemical Formula 1,
A is a naphthalene ring fused with a neighboring pentagonal ring,
B is a C₆₋₂₀ aromatic ring fused with a neighboring pentagonal ring,
X are each independently N or CR, provided that at least one of X is N,
R is hydrogen, deuterium, or tritium,
R₁ is hydrogen, deuterium, tritium, substituted or unsubstituted C₆₋₆₀ aryl, or substituted or unsubstituted C₂₋₆₀ heteroaryl comprising O or S, but is not 4-dibenzofuranyl and 4-dibenzothiophenyl,
R₂ is hydrogen, deuterium, tritium, or substituted or unsubstituted C₂₋₆₀ heteroaryl comprising O or S, but is not 4-dibenzofuranyl and 4-dibenzothiophenyl,
provided that the case where R₁ and R₂ are simultaneously hydrogen, deuterium, or tritium is excluded,
Z₁ and Z₂ are each independently hydrogen, deuterium, or tritium,
L₁ and L₂ are each independently a single bond, substituted or unsubstituted C₁₋₆₀ alkylene, substituted or unsubstituted C₆₋₆₀ arylene, or substituted or unsubstituted C₂₋₆₀ heteroarylene comprising one or more heteroatoms among N, O and S,
Ar₁ and Ar₂ are each independently substituted or unsubstituted C₁₋₆₀ alkyl, substituted or unsubstituted C₆₋₆₀ aryl, or substituted or unsubstituted C₂₋₆₀ heteroaryl comprising one or more heteroatoms among N, O and S,
R₃ and R₄ are each independently hydrogen, deuterium, tritium, substituted or unsubstituted C₁₋₆₀ alkyl, or substituted or unsubstituted C₆₋₆₀ aryl,
a is an integer from 0 to 6,
b is an integer from 0 to 10, and
the “substituted or unsubstituted” means being unsubstituted or substituted with one or more substituents selected from the group consisting of deuterium, tritium, halogen, nitro, amino, alkyl, and aryl, or being unsubstituted or substituted with a substituent to which two or more substituents of the above-exemplified substituents are connected.

According to another aspect of the present disclosure, there is provided an organic light emitting device comprising: a first electrode; a second electrode that is provided opposite to the first electrode; and one or more organic material layers that are provided between the first electrode and the second electrode, wherein one or more layers of the organic material layers comprises the compound represented by Chemical Formula 1.

### Advantageous Effects

The above-mentioned compound represented by Chemical Formula 1 can be used as a material of an organic material layer in an organic light emitting device, and can improve the efficiency, achieve low driving voltage and/or improve lifetime characteristics in the organic light emitting device.

### BRIEF DESCRIPTION OF THE DRAWINGS

FIG. 1 shows an example of an organic light emitting device comprising a substrate 1, an anode 2, a hole transport layer 3, a light emitting layer 4, an electron injection and transport layer 5, and a cathode 6.

FIG. 2 shows an example of an organic light emitting device comprising a substrate 1, an anode 2, a hole injection layer 7, a hole transport layer 3, an electron blocking layer 8, a light emitting layer 4, a hole blocking layer 9, an electron injection and transport layer 5, and a cathode 6.

### DETAILED DESCRIPTION OF THE EMBODIMENTS

Hereinafter, embodiments of the present disclosure will be described in more detail to help understanding of the invention.

### DEFINITION OF TERMS

In the present disclosure, the notation [symbol] or [symbol] means a bond linked to another substituent group, and “D” means deuterium.

In the present disclosure, the term “substituted or unsubstituted” means being unsubstituted or substituted with one or more substituents selected from the group consisting of deuterium; a halogen group; a cyano group; a nitro group; a hydroxy group; a carbonyl group; an ester group; an imide group; an amino group; a phosphine oxide group; an alkoxy group; an aryloxy group; an alkylthioxy group; an arylthioxy group; an alkylsulfoxy group; an arylsulfoxy group; a silyl group; a boron group; an alkyl group; a cycloalkyl group; an alkenyl group; an aryl group; an aralkyl group; an aralkenyl group; an alkylaryl group; an alkylamine group; an aralkylamine group; a heteroarylamine group; an arylamine group; an arylphosphine group; and a heterocyclic group containing at least one of N, O and S atoms, or being unsubstituted or substituted with a substituent group to which two or more substituent groups of the above-exemplified substituent groups are linked. For example, “a substituent in which two or more substituents are linked” may be a biphenylyl group. Namely, a biphenylyl group may be an aryl group, or it may be interpreted as a substituent formed by linking two phenyl groups. In one example, the term “substituted or unsubstituted” may be understood as meaning “being unsubstituted or substituted with one or more substituents selected from the group consisting of deuterium, halogen, cyano, a C₁₋₁₀ alkyl, a C₁₋₁₀ alkoxy and a C₆₋₂₀ aryl”, or “being unsubstituted or substituted with one or more substituents selected from the group consisting of deuterium, halogen, cyano, methyl, ethyl, phenyl, biphenylyl, naphthyl, pyridinyl, and pyrimidinyl”. Further, the term “substituted or unsubstituted” may be understood as meaning “being unsubstituted or substituted with one or more substituents selected from the group consisting of deuterium, halogen, cyano, a C₁₋₁₀ alkyl, a C₁₋₁₀ alkoxy, a C₃₋₇ Cycloalkyl, a C₆₋₂₀ aryl, pyridinyl, pyrimidinyl, dibenzofuranyl, dibenzothiophenyl, benzoxazolyl, and benzothiazolyl, or being unsubstituted or substituted with a substituent to which two or more substituents of the above-exemplified substituents are connected”. Further, the term “substituted with one or more substituents” as used herein may be understood as meaning “being substituted with mono to the maximum number of substitutable hydrogens”. Alternatively, the term “substituted with one or more substituents” as used herein may be understood as meaning “being substituted with 1 to 5 substituents”, or “being substituted with one or two substituents”.

In the present disclosure, the carbon number of a carbonyl group is not particularly limited, but is preferably 1 to 40. Specifically, the carbonyl group may be a substituent having the following structural formulas, but is not limited thereto.

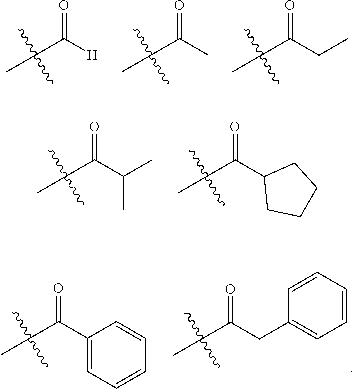

In the present disclosure, an ester group may have a structure in which oxygen of the ester group may be substituted by a straight-chain, branched-chain, or cyclic alkyl group having 1 to 25 carbon atoms, or an aryl group having 6 to 25 carbon atoms. Specifically, the ester group may be a substituent having the following structural formulas, but is not limited thereto.

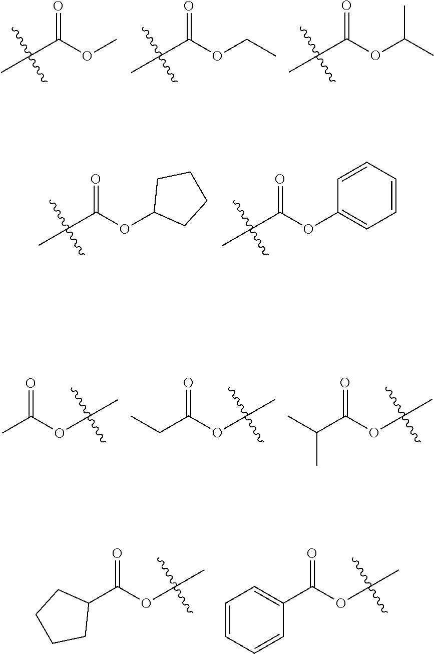

In the present disclosure, the carbon number of an imide group is not particularly limited, but is preferably 1 to 25. Specifically, the imide group may be a substituent group having the following structural formulas, but is not limited thereto.

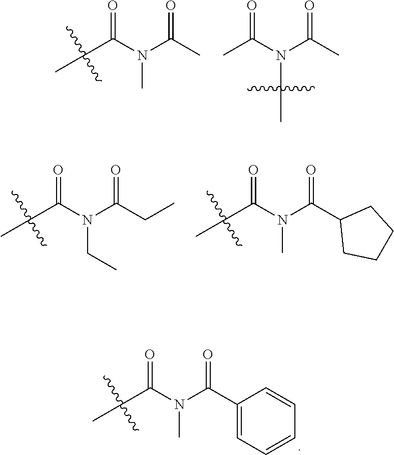

In the present disclosure, a substituted or unsubstituted silyl group means —Si(Z₁)(Z₂)(Z₃), wherein Z₁, Z₂ and Zs are each independently hydrogen, deuterium, a substituted or unsubstituted C₁₋₆₀ alkyl, a substituted or unsubstituted C₁₋₆₀ haloalkyl, a substituted or unsubstituted C₂₋₆₀ alkenyl, a substituted or unsubstituted C₂₋₆₀ haloalkenyl, or a substituted or unsubstituted C₆₋₆₀ aryl. According to one embodiment, Z₁, Z₂ and Zs may be each independently hydrogen, deuterium, a substituted or unsubstituted C₁₋₁₀ alkyl, a substituted or unsubstituted C₁₋₁₀ haloalkyl, or a substituted or unsubstituted C₆₋₂₀ aryl. Specific examples of the silyl group include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but are not limited thereto.

In the present disclosure, a boron group specifically includes a trimethylboron group, a triethylboron group, a t-butyldimethylboron group, a triphenylboron group, and a phenylboron group, but is not limited thereto.

In the present disclosure, examples of a halogen group include fluoro, chloro, bromo, or iodo.

In the present disclosure, the alkyl group may be straight-chain or branched-chain, and the carbon number thereof is not particularly limited, but is preferably 1 to 40. According to one embodiment, the carbon number of the alkyl group is 1 to 20. According to another embodiment, the carbon number of the alkyl group is 1 to 10. Specific examples of the alkyl group include methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, 1-ethyl-propyl, 1,1-dimethylpropyl, hexyl, n-hexyl, 1-methylpentyl, 2-methylpentyl, 4-methyl-2-pentyl, 3,3-dimethylbutyl, 2-ethylbutyl, heptyl, n-heptyl, isohexyl, 1-methylhexyl, 2-methylhexyl, 3-methylhexyl, 4-methylhexyl, 5-methylhexyl, cyclopentylmethyl, cyclohexylmethyl, octyl, n-octyl, tert-octyl, 1-methylheptyl, 2-ethylhexyl, 2,4,4-trimethyl-1-pentyl, 2,4,4-trimethyl-2-pentyl, 2-propylpentyl, n-nonyl, 2,2-dimethylheptyl, and the like, but are not limited thereto.

In the present disclosure, the alkenyl group may be straight-chain or branched-chain, and the carbon number thereof is not particularly limited, but is preferably 2 to 40. According to one embodiment, the carbon number of the alkenyl group is 2 to 20. According to another embodiment, the carbon number of the alkenyl group is 2 to 10. According to still another embodiment, the carbon number of the alkenyl group is 2 to 6. Specific examples thereof include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, a stilbenyl group, a styrenyl group, and the like, but are not limited thereto.

In the present disclosure, the alicyclic group means a monovalent substituent derived from a saturated or unsaturated hydrocarbon ring compound that contains only carbon as a ring-forming atom, but does not have aromaticity, which is understood to encompass both monocyclic and fused polycyclic compounds. According to one embodiment, the carbon number of the alicyclic group is 3 to 60. According to another embodiment, the carbon number of the alicyclic group is 3 to 30. According to another embodiment, the carbon number of the alicyclic group is 3 to 20. Examples of the alicyclic group include a monocyclic group such as a cycloalkyl group, a bridged hydrocarbon group, a spiro hydrocarbon group, a substituent derived from hydrogenated derivatives of aromatic hydrocarbon compound, and the like.

Specifically, examples of the cycloalkyl group include cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, cyclooctyl, and the like, but are not limited thereto.

Further, examples of the bridged hydrocarbon group include bicyclo[1.1.0]butyl, bicyclo[2.2.1]heptyl, bicyclo[4.2.0]octa-1,3,5-trienyl, adamantyl, decalinyl, and the like, but are not limited thereto.

Further, examples of the spiro hydrocarbon group include spiro[3.4]octyl, spiro[5.5]undecanyl, and the like, but are not limited thereto.

Further, a substituent derived from a hydrogenated derivative of the aromatic hydrocarbon compound means a substituent derived from a monocyclic or polycyclic aromatic hydrocarbon compound in which a part of the compound is hydrogenated. Examples of such a substituent include 1H-indenyl, 2H-indenyl, 4H-indenyl, 2,3-dihydro-1H-indenyl, 1,4-dihydronaphthalenyl, 1,2,3,4-tetrahydronaphthalenyl, 6,7,8,9-tetrahydro-5H-benzo[7]annulenyl, 6,7-dihydro-5H-benzocycloheptenyl, and the like, but are not limited thereto.

In the present disclosure, an aryl group is understood to mean a substituent derived from a monocyclic or fused polycyclic compound containing only carbon as a ring-forming atom and also having aromaticity, and the carbon number thereof is not particularly limited, but is preferably 6 to 60. According to one embodiment, the carbon number of the aryl group is 6 to 30. According to one embodiment, the carbon number of the aryl group is 6 to 20. The aryl group may be a phenyl group, a biphenylyl group, a terphenylyl group, quaterphenylyl or the like as the monocyclic aryl group, but is not limited thereto. The polycyclic aryl group includes a naphthyl group, an anthracenyl group, a phenanthryl group, a pyrenyl group, a perylenyl group, a chrysenyl group, a fluorenyl group, or the like, but is not limited thereto.

In the present disclosure, the fluorenyl group may be substituted, and two substituent groups may be linked with each other to form a spiro structure. In the case where the fluorenyl group is substituted,

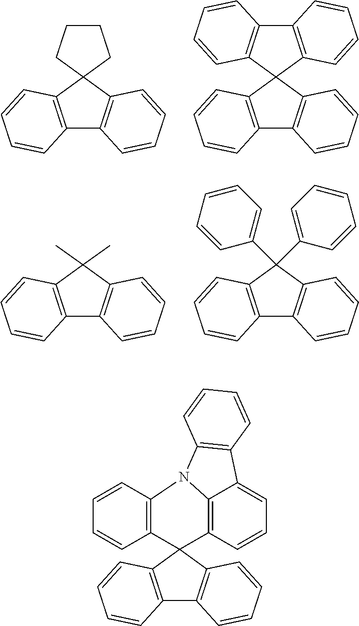

and the like can be formed. However, the structure is not limited thereto.

In the present disclosure, a heterocyclic group means a monovalent substituent derived from a monocyclic or fused polycyclic compound that further contains at least one heteroatom selected among O, N, Si, and S in addition to carbon as a ring-forming atom, and is understood to encompass both substituents with aromaticity and substituents without aromaticity. According to one embodiment, the carbon number of the heterocyclic group is 2 to 60 carbon atoms. According to another embodiment, the carbon number of the heterocyclic group is 2 to 30. According to another embodiment, the carbon number of the heterocyclic group is 2 to 20. Examples of such a heterocyclic group include a heteroaryl group, a substituent derived from a hydrogenated derivative of the heteroaromatic compound, and the like.

Specifically, the heteroaryl group means a substituent derived from a monocyclic or fused polycyclic compound which further contains at least one heteroatom selected among N, O and S in addition to carbon as a ring forming atom, and refers to a substituent having aromaticity. According to one embodiment, the carbon number of the heteroaryl group is 2 to 60. According to another embodiment, the carbon number of the heteroaryl group is 2 to 30. According to another embodiment, the carbon number of the heteroaryl group is 2 to 20. According to another embodiment, the carbon number of the heteroaryl group is 2 to 12. According to another embodiment, the carbon number of the heteroaryl group is 2 to 10. According to another embodiment, the carbon number of the heteroaryl group is 2 to 8. According to another embodiment, the carbon number of the heteroaryl group is 10 to 20. Examples of the heteroaryl group include a thiophenyl group, a furanyl group, a pyrrole group, an imidazolyl group, a thiazolyl group, an oxazolyl group, an oxadiazolyl group, a triazolyl group, a pyridinyl group, a bipyridinyl group, a pyrimidinyl group, a triazinyl group, an acridinyl group, a pyridazinyl group, a pyrazinyl group, a quinolinyl group, a quinazolinyl group, a quinoxalinyl group, a phthalazinyl group, a pyridopyrimidinyl group, a pyridopyrazinyl group, an isoquinolinyl group, an indolyl group, a carbazolyl group, a benzoxazolyl group, a benzoimidazolyl group, a benzothiazolyl group, a benzocarbazolyl group, a benzothiophenyl group, a dibenzothiophenyl group, a benzofuranyl group, a dibenzofuranyl group, a phenanthrolinyl group, an isoxazolyl group, a thiadiazolyl group, a phenothiazinyl group, and the like, but are not limited thereto.

Further, a substituent derived from a hydrogenated derivative of a heteroaromatic compound means a substituent derived from a monocyclic or polycyclic heteroaromatic compound in which a part of the unsaturated bond of the compound is hydrogenated. Examples of such substituents include 1,3-dihydroisobenzofuranyl, 2,3-dihydrobenzofuranyl, 1,3-dihydrobenzo[c]thiophenyl, 2,3-dihydro[b]thiophenyl, and the like, but are not limited thereto.

In the present disclosure, the aryl group in the aralkyl group, the aralkenyl group, the alkylaryl group, the arylamine group and the arylsilyl group is the same as the examples of the aryl group as defined above. In the present disclosure, the alkyl group in the aralkyl group, the alkylaryl group and the alkylamine group is the same as the examples of the alkyl group as defined above. In the present disclosure, the heteroaryl in the heteroarylamine can be applied to the description of the heteroaryl as defined above. In the present disclosure, the alkenyl group in the aralkenyl group is the same as the examples of the alkenyl group as defined above. In the present disclosure, the description of the aryl group as defined above may be applied except that the arylene is a divalent group. In the present disclosure, the description of the heteroaryl as defined above can be applied except that the heteroarylene is a divalent group. In the present disclosure, the description of the aryl group or cycloalkyl group as defined above can be applied except that the hydrocarbon ring is not a monovalent group but formed by combining two substituent groups. In the present disclosure, the description of the heteroaryl as defined above can be applied, except that the heterocycle is not a monovalent group but formed by combining two substituent groups.

In the present disclosure, the term “deuterated or substituted with deuterium” means that at least one of the substitutable hydrogens in a compound, a divalent linking group, or a monovalent substituent has been substituted with deuterium.

Further, the term “unsubstituted or substituted with deuterium” or “unsubstituted or substituted with deuterium” means that “mono to the maximum number of unsubstituted or substitutable hydrogens have been substituted with deuterium.” In one example, the term “phenanthryl unsubstituted or substituted with deuterium” may be understood as meaning “phenanthryl unsubstituted or substituted with 1 to 9 deuterium atoms”, considering that the maximum number of hydrogens that can be substituted with deuterium in the phenanthryl structure is 9.

Further, “deuterated structure” means to include compounds, divalent linking groups, or monovalent substituents of all structures in which at least one hydrogen is substituted with deuterium. As an example, the deuterated structure of phenyl can be understood to refer to monovalent substituents of all structures in which at least one substitutable hydrogen in the phenyl group is substituted with deuterium, as follows.

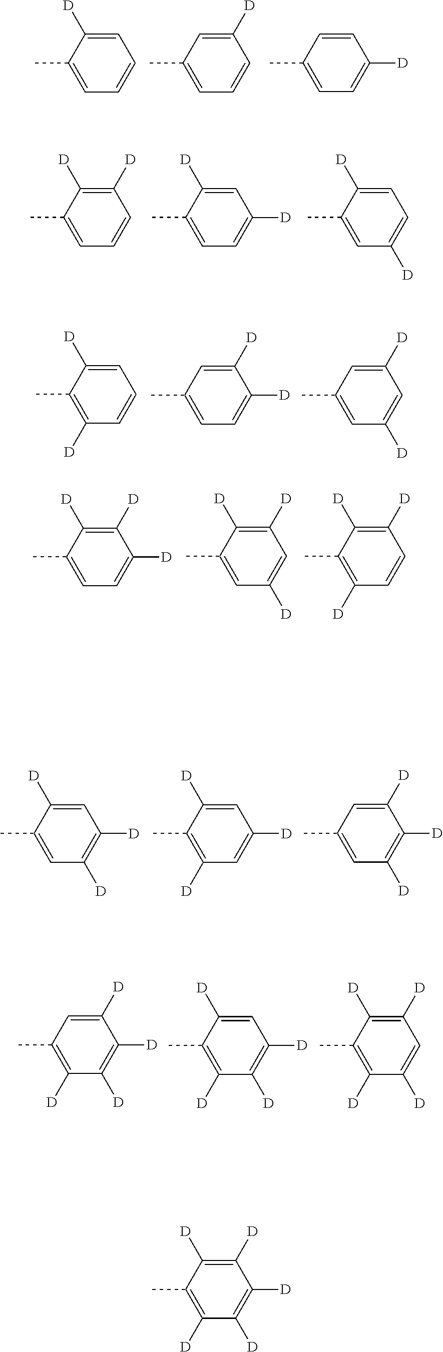

In addition, the “deuterium substitution rate” or “degree of deuteration” of a compound means that the ratio of the number of substituted deuterium atoms to the total number of hydrogen atoms (the sum of the number of hydrogen atoms substitutable with deuterium and the number of substituted deuterium atoms in a compound) that can exist in the compound is calculated as a percentage. Therefore, when the “deuterium substitution rate” or “degree of deuteration” of a compound is “K %”, it means that K % of the hydrogen atoms substitutable with deuterium in the compound are substituted with deuterium.

At this time, the “deuterium substitution rate” or “degree of deuteration” can be determined according to a commonly known method using MALDI-TOF MS (Matrix-Assisted Laser Desorption/Ionization Time-of-Flight Mass Spectrometer), a nuclear magnetic resonance spectroscopy (¹H NMR), TLC/MS (Thin-Layer Chromatography/Mass Spectrometry), GC/MS (Gas Chromatography/Mass Spectrometry), or the like. More specifically, when using MALDI-TOF MS, the “deuterium substitution rate” or “degree of deuteration” may be obtained by determining the number of substituted deuterium in the compound through MALDI-TOF MS analysis, and then calculating the ratio of the number of substituted deuterium to the total number of hydrogen atoms that can exist in the compound as a percentage.

### (Compound)

Meanwhile, according to the present disclosure, there is provided the compound represented by Chemical Formula 1.

Specifically, the compound represented by Chemical Formula 1 has a structure in which a (benzocarbazole substituent) and an (N-containing 6-membered heterocyclic ring) are linked at an ortho position, and at least one of a meta position and a para position of the (N-containing 6-membered heterocyclic ring) is substituted with a substituent other than hydrogen/deuterium/tritium. More specifically, in the compound, in a case in which a meta position of the (N-containing 6-membered heterocyclic ring) has a substituent other than hydrogen/deuterium/tritium, the substituent is an aryl or a heteroaryl including O or S, and in a case in which a para position of the (N-containing 6-membered heterocyclic ring) has a substituent other than hydrogen/deuterium/tritium, the substituent is a heteroaryl including O or S. In these cases, 4-dibenzofuranyl and 4-dibenzothiophenyl are excluded as the substituent at the meta and para positions of the (N-containing 6-membered heterocyclic ring). Wherein, 4-dibenzofuranyl means a structure of

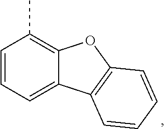

and 4-dibenzothiophenyl means a structure of

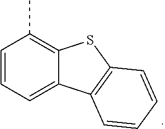

The compound having such a structure, compared to a compound having hydrogen/deuterium/tritium at both the meta and para positions of the (N-containing 6-membered heterocyclic ring), a compound substituted with a heteroaryl including N at least one of the meta and para positions of the (N-containing 6-membered heterocyclic ring), and a compound substituted with 4-dibenzofuranyl/4-dibenzothiophenyl at least one of the meta and para positions of the (N-containing 6-membered heterocyclic ring), is structurally stable and thus can contribute to improving the efficiency and lifetime characteristics of an organic light emitting device.

Meanwhile, B may be a benzene, naphthalene, phenanthrene, or anthracene ring fused with an adjacent pentagonal ring.

In this case, when B is a benzene ring, b is an integer of 0 to 4,
when B is a naphthalene ring, b is an integer of 0 to 6,
when B is a phenanthrene or anthracene ring, b is an integer of 0 to 8.

Meanwhile, the substituent

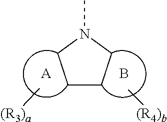

may be any one of the substituents represented by the following chemical formulas 1a to 1l:

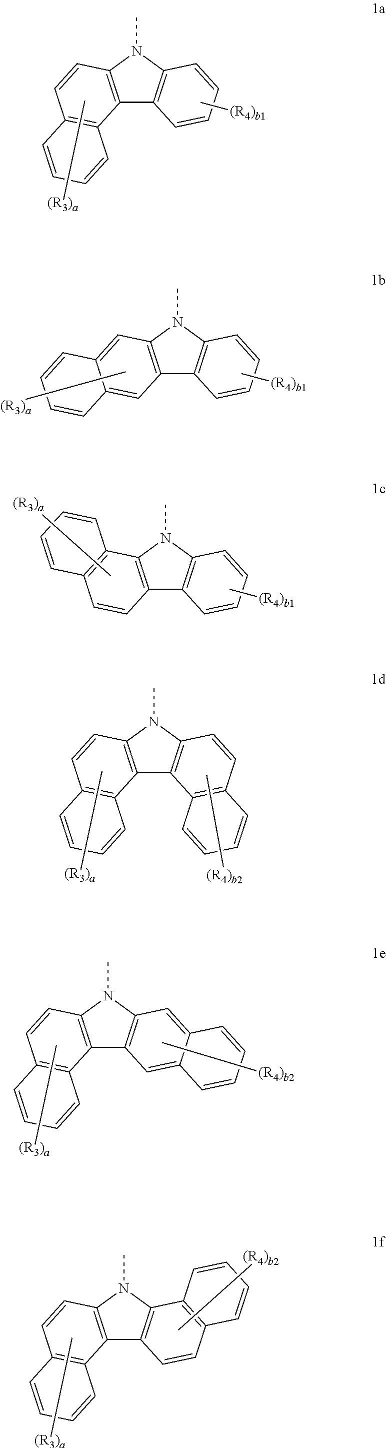

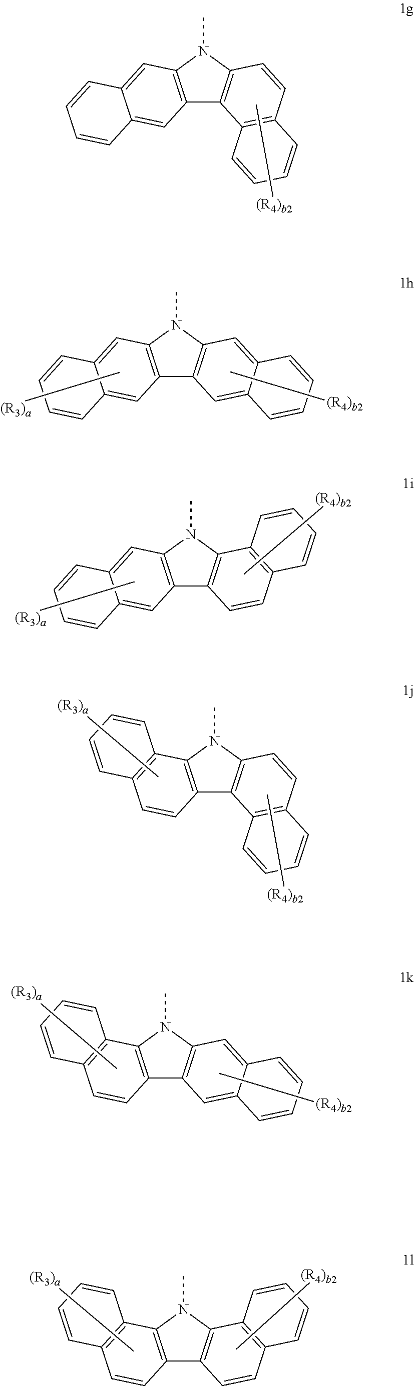

wherein in Chemical Formulas 1a to 1l,
b1 is an integer from 0 to 4,
b2 is an integer from 0 to 6, and
R₃, R₄ and a are as defined in Chemical Formula 1.

Preferably, the substituent

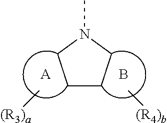

may be any one selected from the group consisting of the following and the deuterated structure thereof:

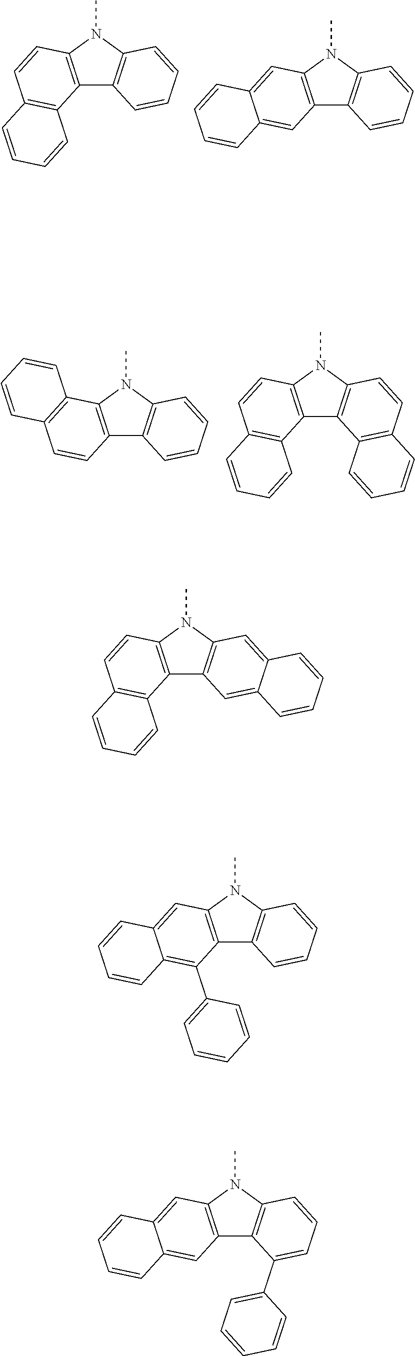

In one embodiment, one of X may be N.

In another embodiment, two of X may be N.

In yet another embodiment, all X may be N.

Further, in one embodiment, R₁ may be hydrogen, deuterium, tritium, a substituted or unsubstituted C₆₋₂₀ aryl, or a substituted or unsubstituted C₂₋₂₀ heteroaryl comprising one or two heteroatoms selected from O and S (excluding 4-dibenzofuranyl and 4-dibenzothiophenyl).

For example, R₁ may be hydrogen, deuterium, tritium, substituted or unsubstituted phenyl, substituted or unsubstituted biphenylyl, substituted or unsubstituted terphenylyl, substituted or unsubstituted quaterphenylyl, substituted or unsubstituted naphthyl, substituted or unsubstituted phenanthryl, substituted or unsubstituted 1-dibenzofuranyl, substituted or unsubstituted 2-dibenzofuranyl, substituted or unsubstituted 3-dibenzofuranyl, substituted or unsubstituted 1-dibenzothiophenyl, substituted or unsubstituted 2-dibenzothiophenyl, substituted or unsubstituted 3-dibenzothiophenyl, substituted or unsubstituted benzonaphthofuranyl, or substituted or unsubstituted benzonaphthothiophenyl, and
where the “substituted or unsubstituted” means being unsubstituted or substituted with one or more substituents selected from the group consisting of deuterium, tritium, C₁₋₁₀ alkyl and C₆₋₂₀ aryl, or being unsubstituted or substituted with a substituent to which two or more substituents of the above-exemplified substituents are connected.

Further, for example, R₁ may be C₆₋₂₄ monocyclic aryl unsubstituted or substituted with deuterium. Accordingly, R₁ may be substituted or unsubstituted phenyl, substituted or unsubstituted biphenylyl, substituted or unsubstituted terphenylyl, or substituted or unsubstituted quaterphenylyl.

Further, in one embodiment, R₂ may be hydrogen, deuterium, tritium, a substituted or unsubstituted C₂₋₂₀ heteroaryl comprising one or two heteroatoms selected from O and S (excluding 4-dibenzofuranyl and 4-dibenzothiophenyl).

For example, R₂ may be hydrogen, deuterium, tritium, substituted or unsubstituted 1-dibenzofuranyl, substituted or unsubstituted 2-dibenzofuranyl, substituted or unsubstituted 3-dibenzofuranyl, substituted or unsubstituted 1-dibenzothiophenyl, substituted or unsubstituted 2-dibenzothiophenyl, substituted or unsubstituted 3-dibenzothiophenyl, substituted or unsubstituted benzonaphthofuranyl, or substituted or unsubstituted benzonaphthothiophenyl, and
where the “substituted or unsubstituted” means being unsubstituted or substituted with one or more substituents selected from the group consisting of deuterium, tritium, C₁₋₁₀ alkyl and C₆₋₂₀ aryl, or being unsubstituted or substituted with a substituent to which two or more substituents of the above-exemplified substituents are connected.

Preferably, the “substituted or unsubstituted” in R₁ and R₂ means being unsubstituted or substituted with one or more substituents selected from the group consisting of deuterium, tritium, phenyl, biphenyl, and naphthyl, or being unsubstituted or substituted with a substituent to which two or more substituents of the above-exemplified substituents are connected.

More specifically, R₁ may be hydrogen, deuterium, tritium, or any one selected from the group consisting of the following and the deuterated structure thereof:

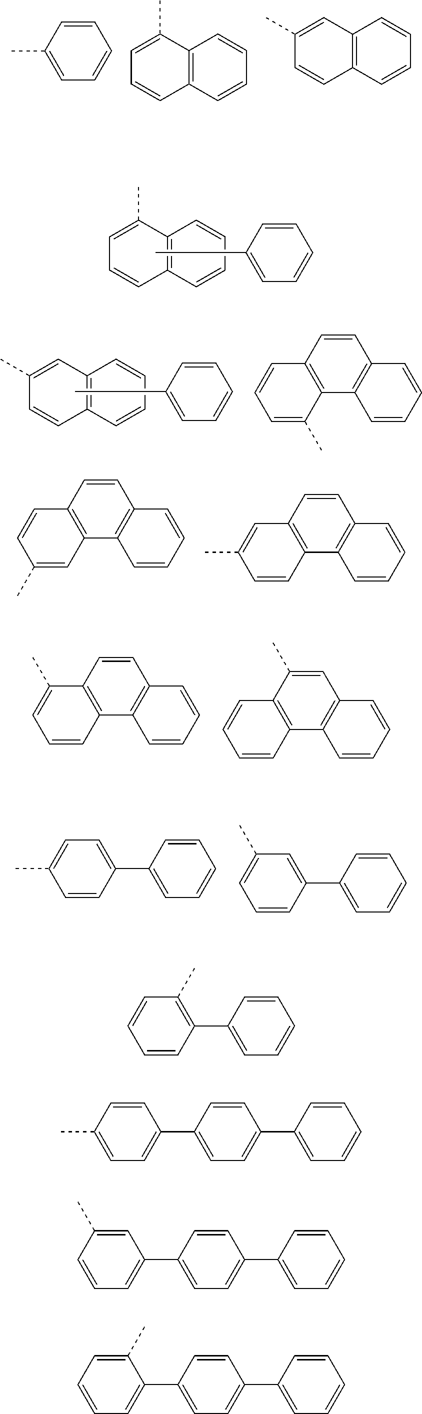

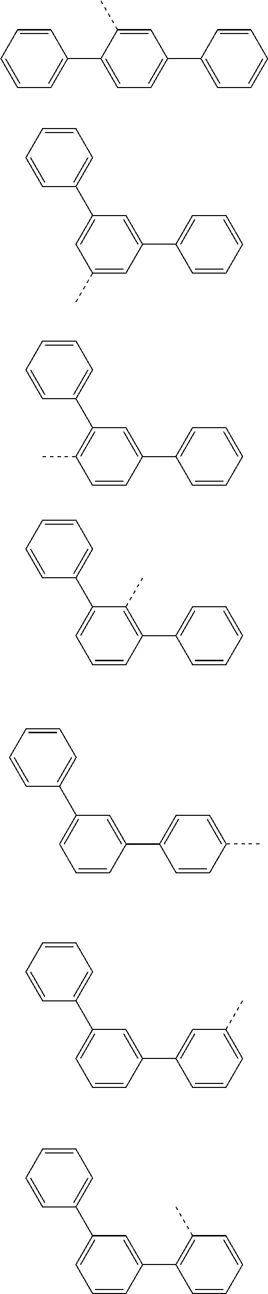

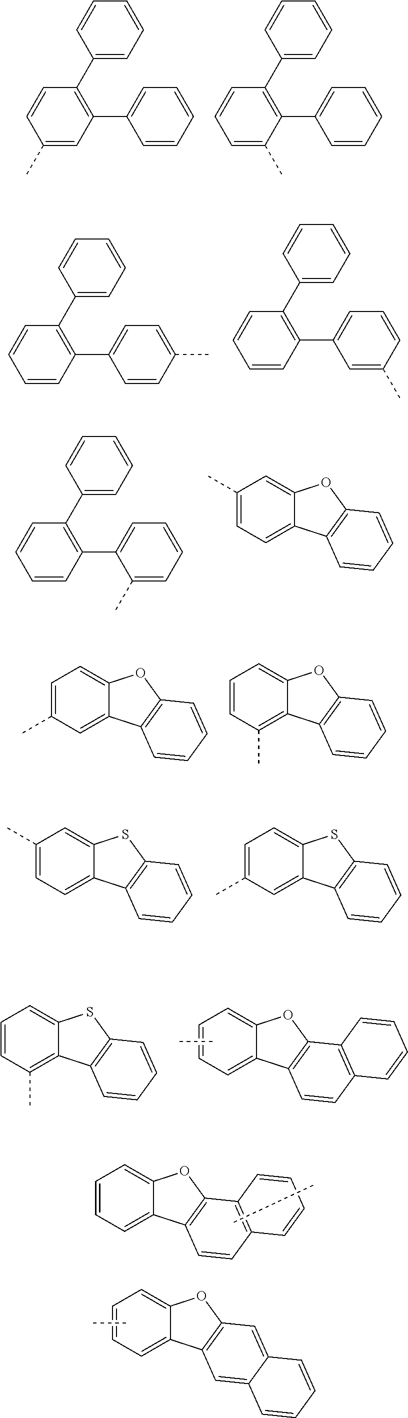

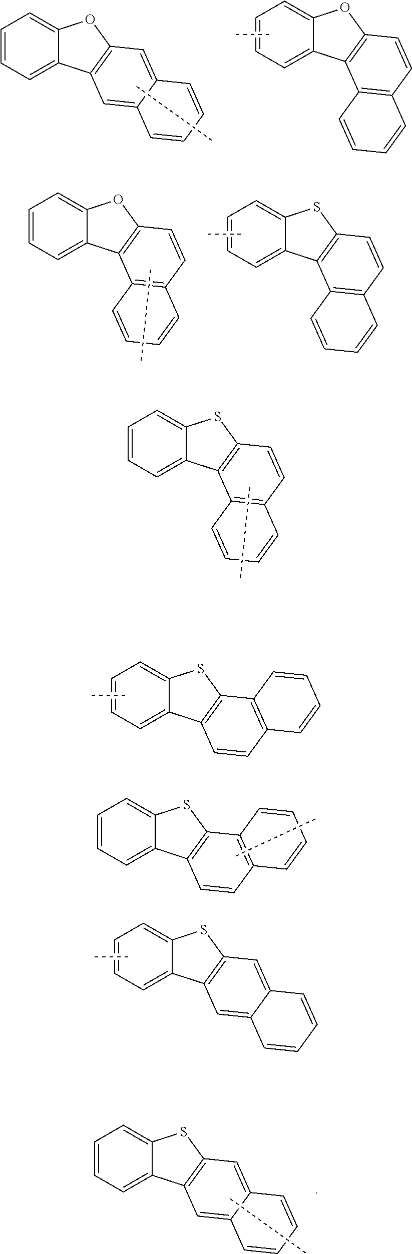

More specifically, R₂ may be hydrogen, deuterium, tritium, or may be any one selected from the group consisting of the following and the deuterated structure thereof:

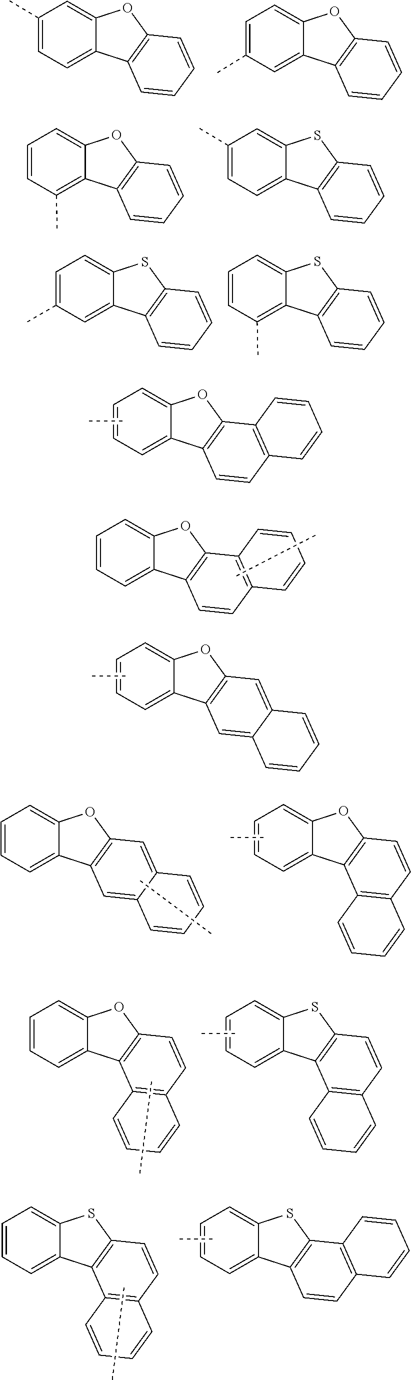

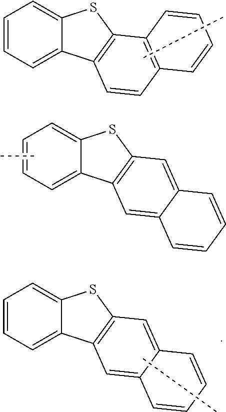

Further, in one embodiment, Z₁ and Z₂ may be each independently hydrogen or deuterium.

In another embodiment, Z₁ and Z₂ may be both hydrogens, or both deuteriums.

Further, in one embodiment, L₁ and L₂ may be each independently a single bond, or substituted or unsubstituted C₆₋₂₀ arylene.

In another embodiment, L₁ and L₂ may be each independently a single bond; or C₆₋₂₀ arylene which is unsubstituted, or substituted unsubstituted or substituted with deuterium, tritium, substituted or unsubstituted C₁₋₁₀ alkyl and substituted or unsubstituted C₆₋₂₀ aryl.

In yet another embodiment, L₁ and L₂ may be each independently a single bond, phenylene unsubstituted or substituted with deuterium, biphenyldiyl unsubstituted or substituted with deuterium, or naphthylene unsubstituted or substituted with deuterium.

Wherein, “substitution” in L₁ and L₂ means being unsubstituted or substituted with one or more substituents selected from the group consisting of deuterium, tritium, substituted or unsubstituted C₁₋₁₀ alkyl and substituted or unsubstituted C₆₋₂₀ aryl.

For example, L₁ and L₂ may be each independently a single bond, or any one selected from the group consisting of the following and the deuterated structure thereof:

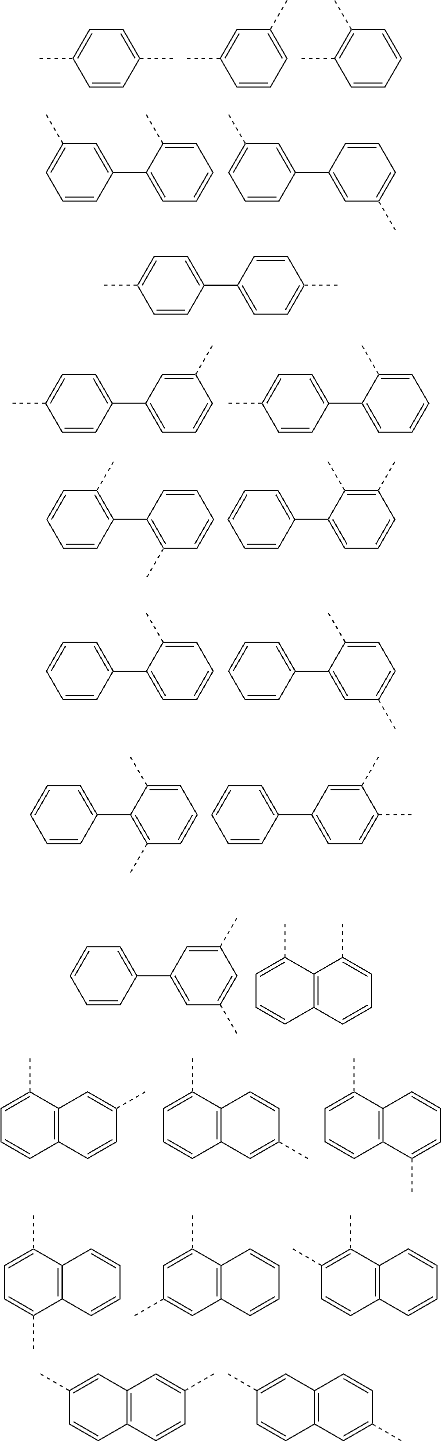

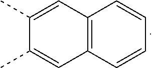

in one embodiment, L₁ and L₂ may be identical to each other.

Alternatively, L₁ and L₂ may be may be different from each other.

Further, in one embodiment, Ar₁ and Ar₂ may be each independently substituted or unsubstituted C₆₋₂₀ aryl, or substituted or unsubstituted C₂₋₆₀ heteroaryl comprising one or more heteroatoms among N, O and S.

In another embodiment, Ar₁ and Ar₂ may be each independently phenyl, biphenylyl, terphenylyl, 1-naphthyl, or 2-naphthyl,
wherein, Ar₁ and Ar₂ are unsubstituted or substituted with one or more substituents selected from the group consisting of deuterium, tritium, C₁₋₁₀ alkyl and C₆₋₂₀ aryl, or being unsubstituted or substituted with a substituent to which two or more substituents of the above-exemplified substituents are connected.

Preferably, Ar₁ and Ar₂ may be unsubstituted or substituted with one or more substituents selected from the group consisting of deuterium, tritium, phenyl, biphenyl, and naphthyl, or being unsubstituted or substituted with a substituent to which two or more substituents of the above-exemplified substituents are connected.

Further, Ar₁ and Ar₂ may be identical to each other.

Alternatively, Ar₁ and Ar₂ may be may be different from each other.

Further, in one embodiment, R₃ and R₄ may be each independently hydrogen, deuterium, tritium, substituted or unsubstituted C₁₋₁₀ alkyl, or substituted or unsubstituted C₆₋₂₀ aryl

In another embodiment, R₃ and R₄ may be each independently hydrogen, deuterium, tritium, or phenyl unsubstituted or substituted with deuterium.

Further, a denotes the number of R₃s, and when R₃ is 2 or more, two or more R₃s may be identical or different.

Further, b denotes the number of R₄s, and when R₄ is 2 or more, two or more R₄s may be identical or different.

in one embodiment, a and b may be each independently 0, 1, 4, or 6.

In another embodiment, a and b may be each independently 0 or 1.

In yet another embodiment, a+b may be 0, 1, 10, or 12.

In yet another embodiment, a+b may be 0; or
a+b is 1, and one of R₃ and R₄ may be phenyl unsubstituted or substituted with deuterium; or
a+b is 1 to 12, and one of R₃ and R₄ may be phenyl unsubstituted or substituted with deuterium, and the rest are hydrogen or deuterium.

Further, the compound may be represented by the following Chemical Formulas 1-1 or 1-2:

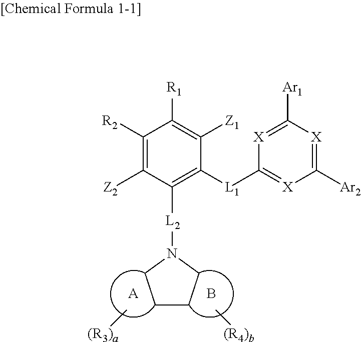

wherein in Chemical Formula 1-1,
R₁ is substituted or unsubstituted C₆₋₆₀ aryl, or substituted or unsubstituted C₂₋₆₀ heteroaryl comprising O or S, but is not 4-dibenzofuranyl and 4-dibenzothiophenyl,
Z₃ is hydrogen, deuterium, or tritium, and
A, B, X, Z₁, Z₂, L₁, L₂, Ar₁, Ar₂, R₃, R₄, a and b are as defined in Chemical Formula 1, and

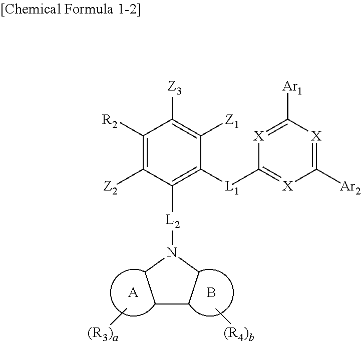

wherein in Chemical Formula 1-2,
R₂ is substituted or unsubstituted C₂₋₆₀ heteroaryl comprising O or S, but is not 4-dibenzofuranyl and 4-dibenzothiophenyl,
Z₃ is hydrogen, deuterium, or tritium, and
A, B, X, Z₁, Z₂, L₁, L₂, Ar₁, Ar₂, R₃, R₄, a and b are as defined in Chemical Formula 1.

Further, the compound may not contain deuterium, or may contain at least one deuterium.

In one embodiment, the compound represented by Chemical formula 1 the compound represented by Chemical formula 1 may not contain deuterium, or may contain 1 to 50 deuterium atoms. More specifically, the compound may not contain deuterium, or may contain 1 or more, 2 or more, 3 or more, 4 or more, 5 or more, 6 or more, 7 or more, 8 or more, 9 or more, 10 or more, 11 or more, 12 or more, 13 or more, 14 or more, or 15 or more, and 50 or less, 40 or less, 30 or less, 28 or less, 26 or less, 24 or less, 22 or less, or 20 or less deuterium atoms.

In this case, when the number of deuterium substitution of the compound represented by Chemical formula 1 is to be represented, it may be represented by the following Chemical Formula 1 D:

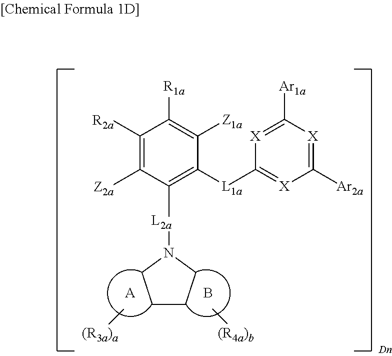

wherein in Chemical Formula 1D,
Dn means that n hydrogens have been replaced by deuterium,
wherein n is an integer of 0 or more,
X, A, B, a and b are as defined in Chemical Formula 1, and
Z₁ₐ, Z₂ₐ, L₁ₐ, L₂ₐ, Ar₁ₐ, Ar₂ₐ, R₁ₐ, R₂ₐ, R₃ₐ and R₄ₐ mean Z₁, Z₂, L₁, L₂, Ar₁, Ar₂, R₁, R₂, R₃ and R₄ substituents which are not substituted with deuterium, respectively.

Meanwhile, representative examples of the compound represented by Chemical Formula 1 are as follows:

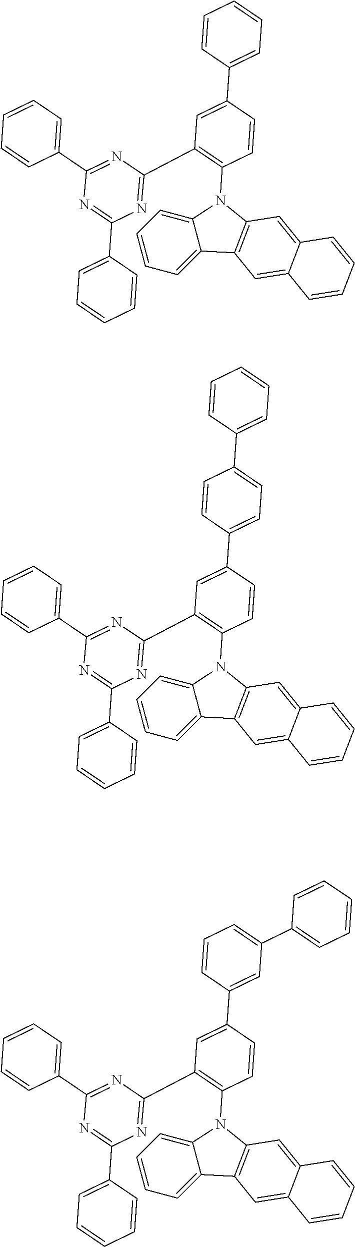

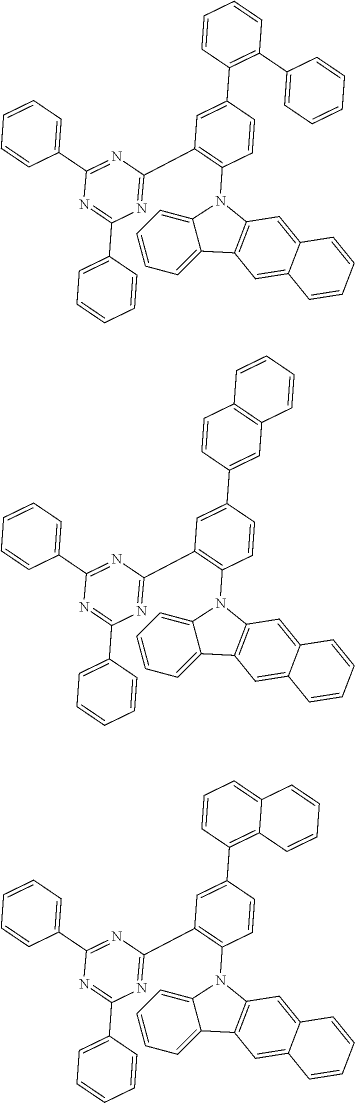

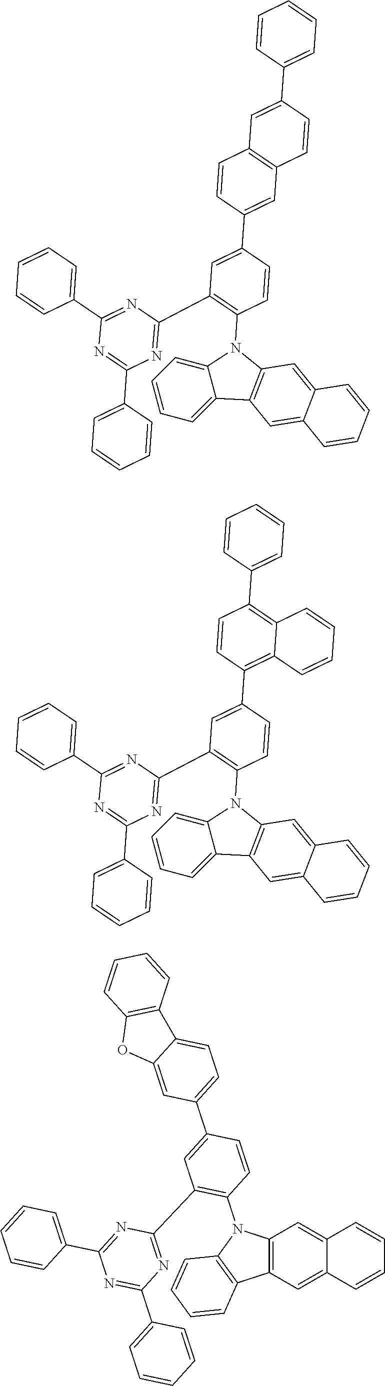

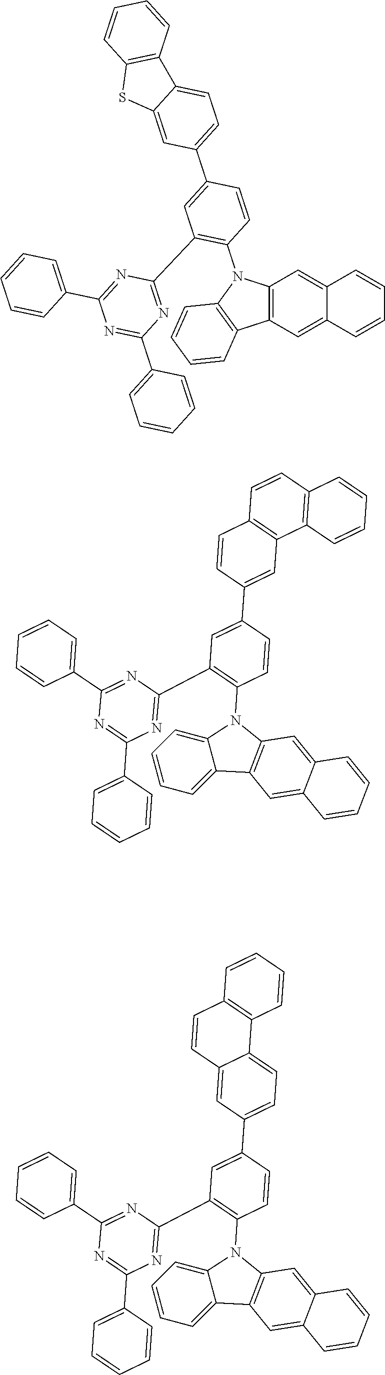

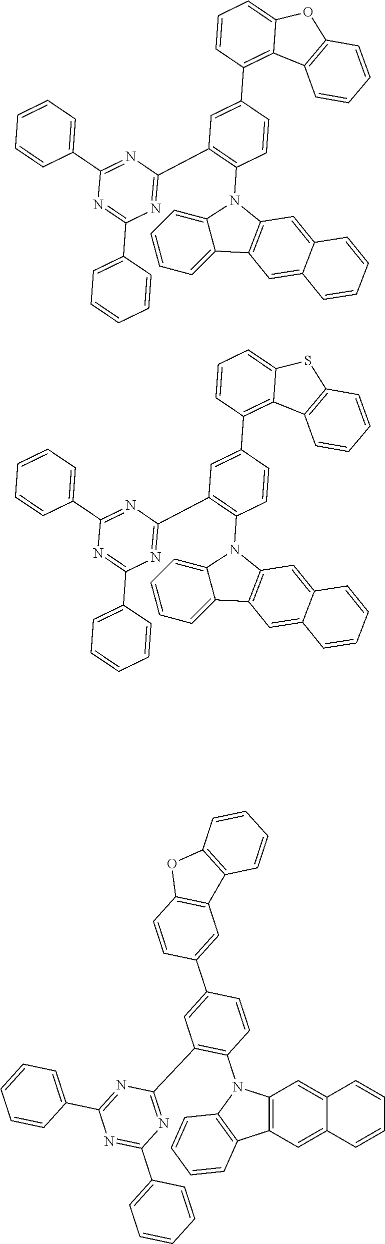

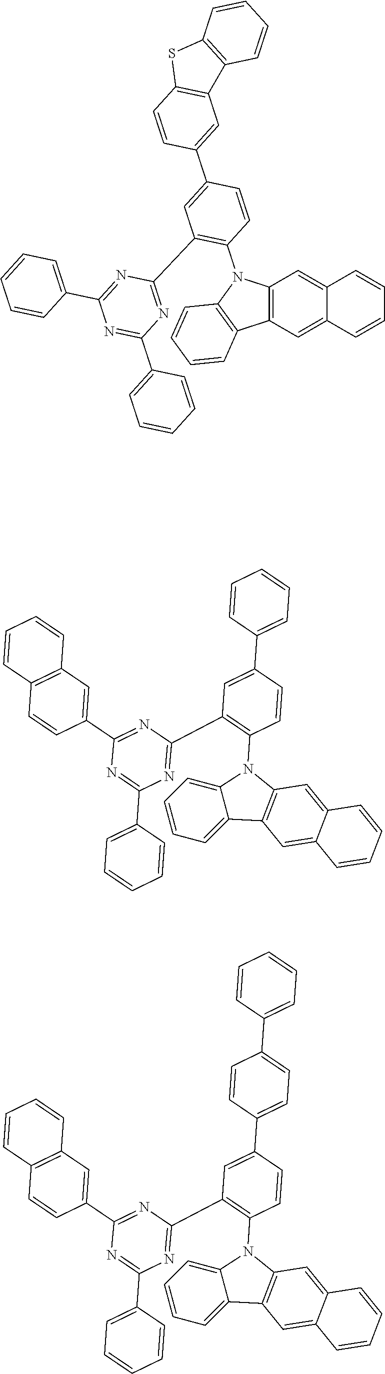

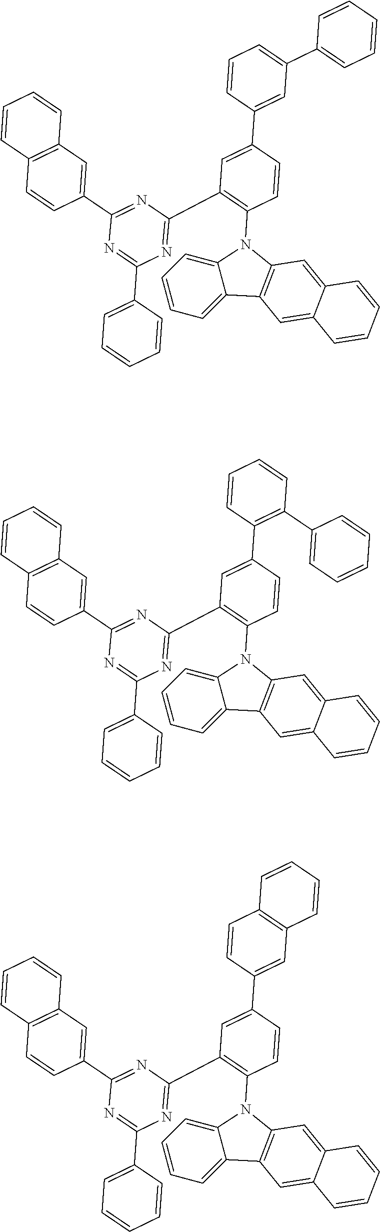

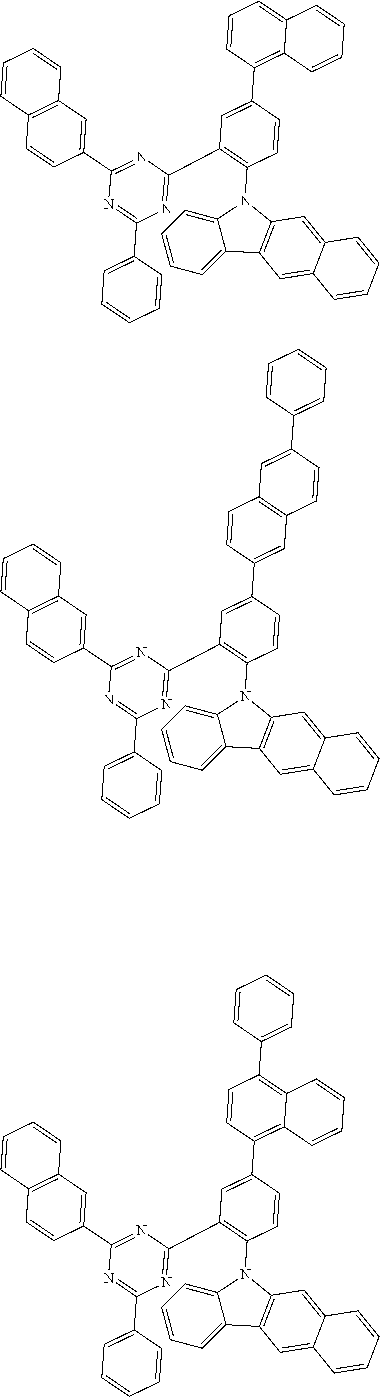

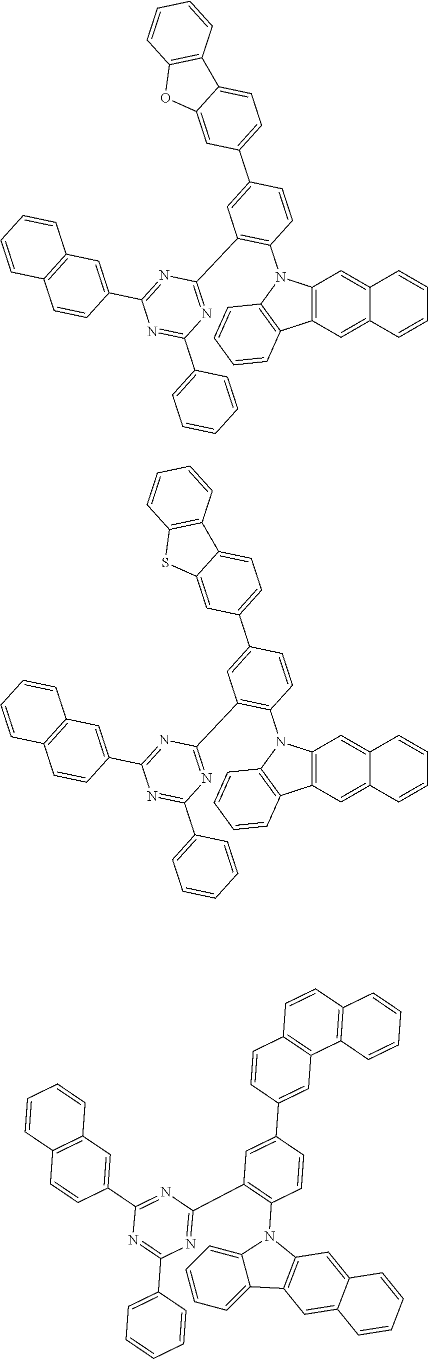

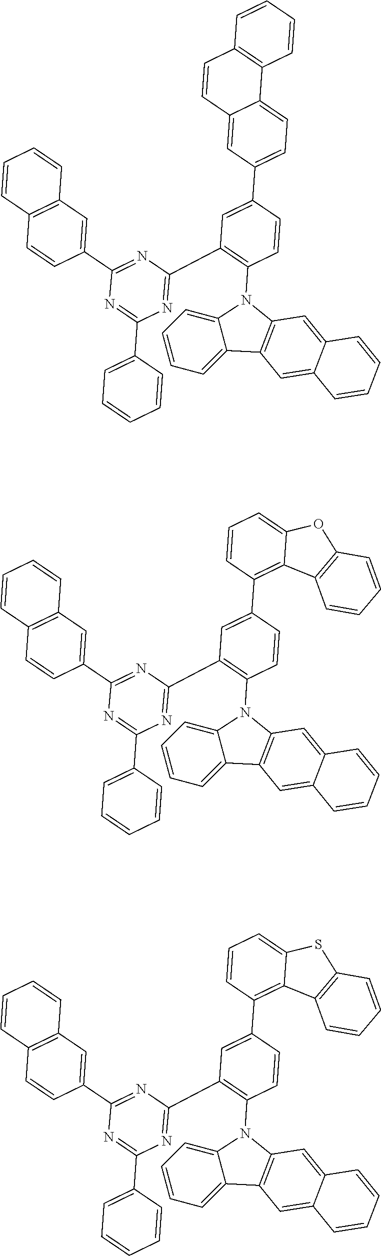

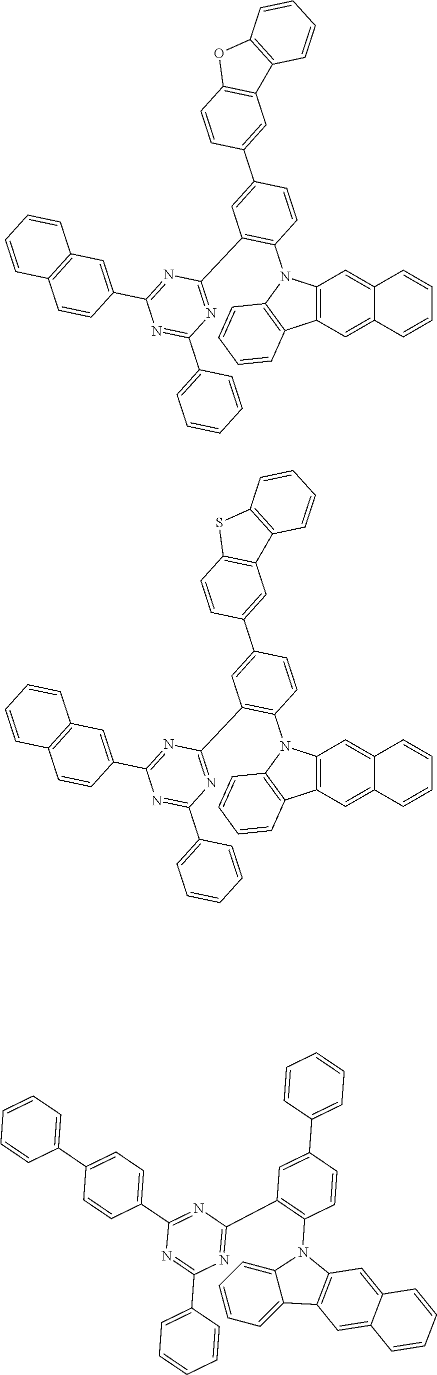

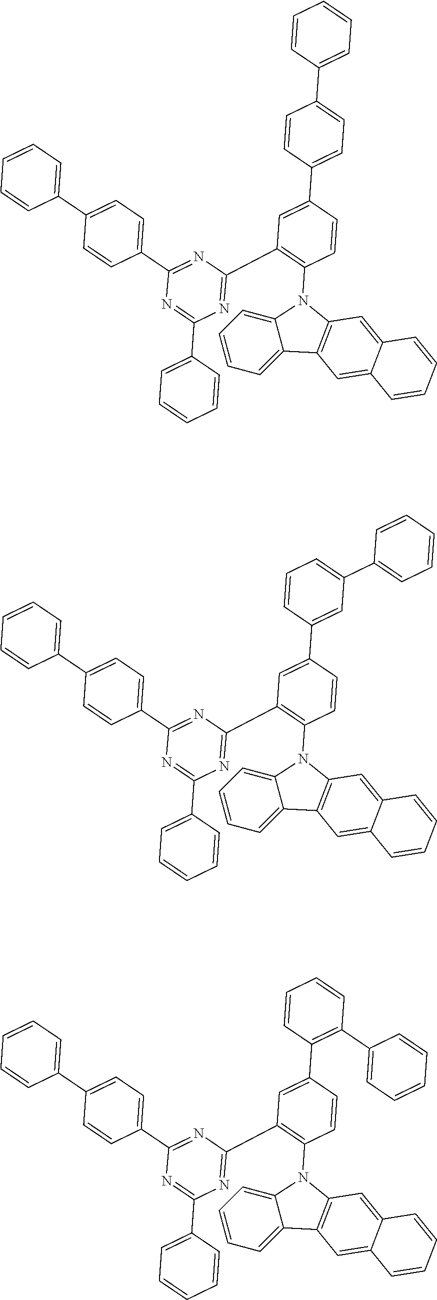

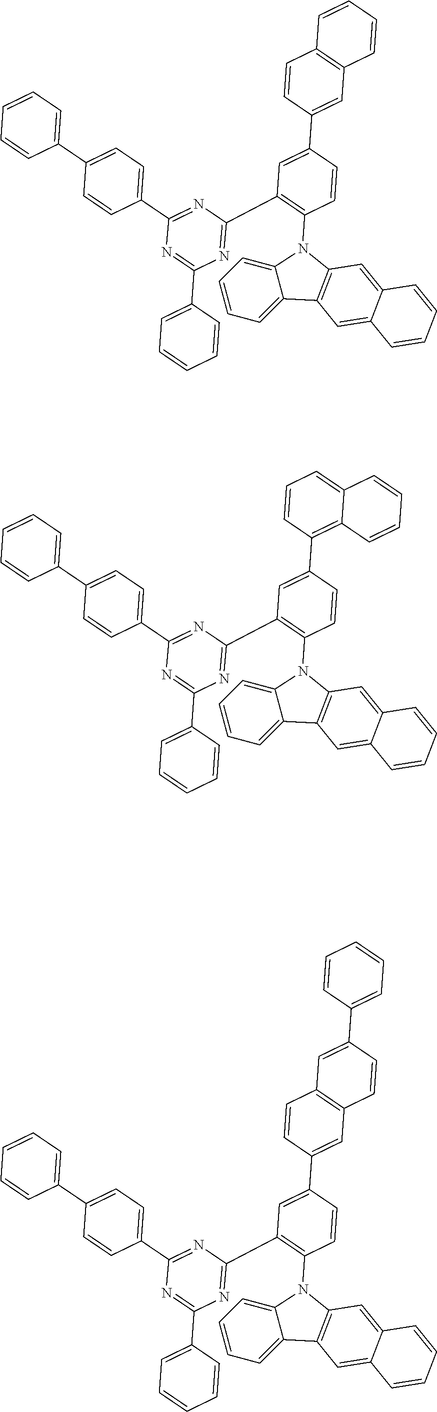

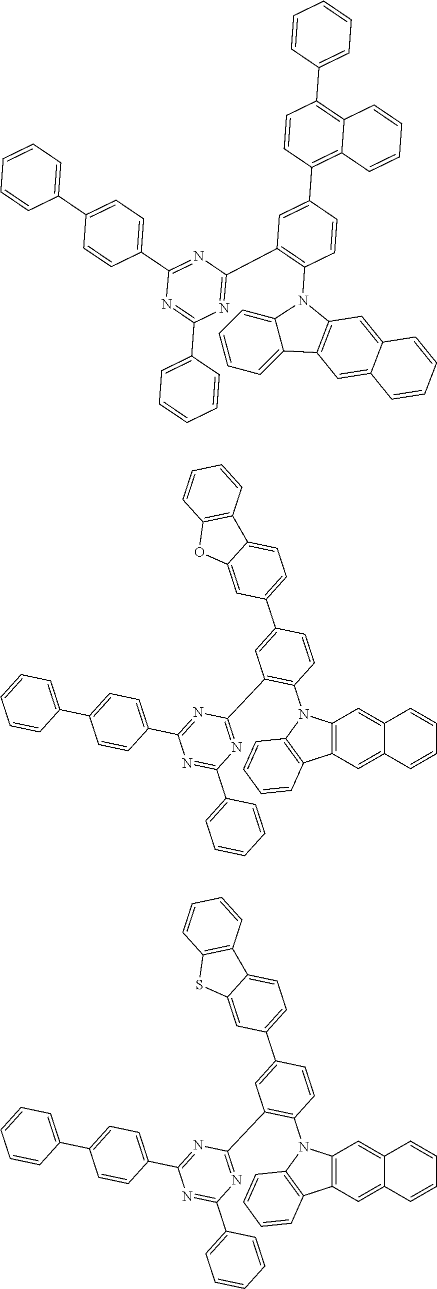

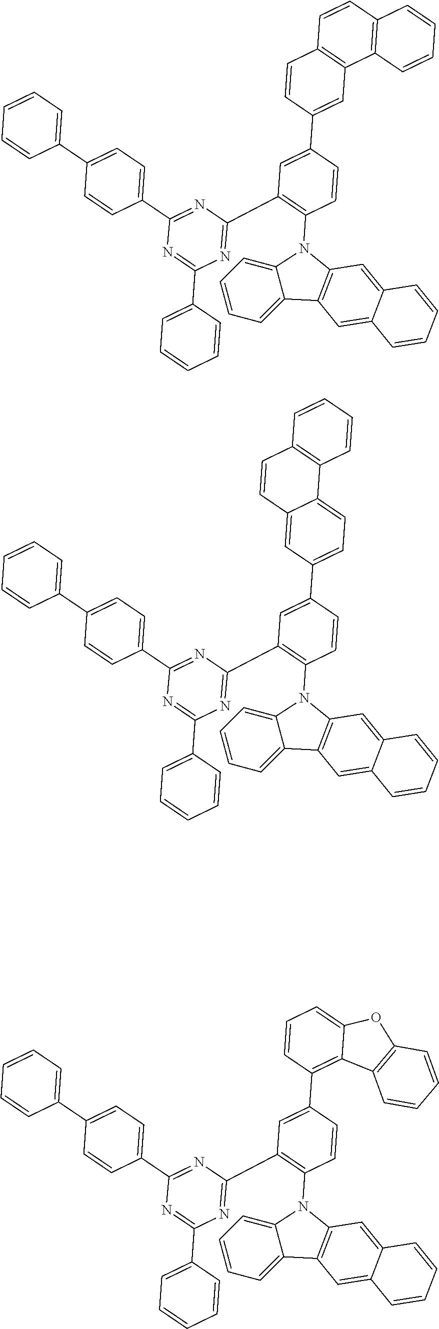

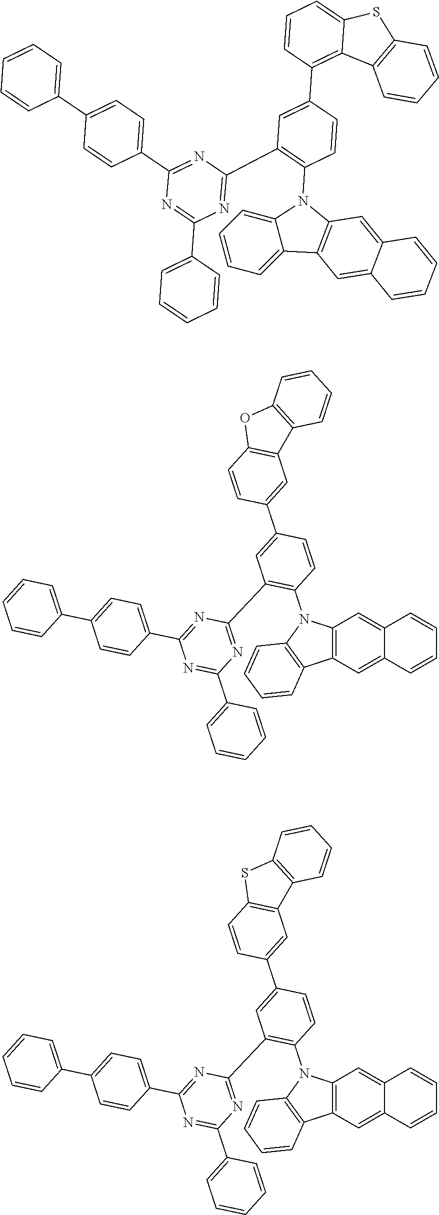

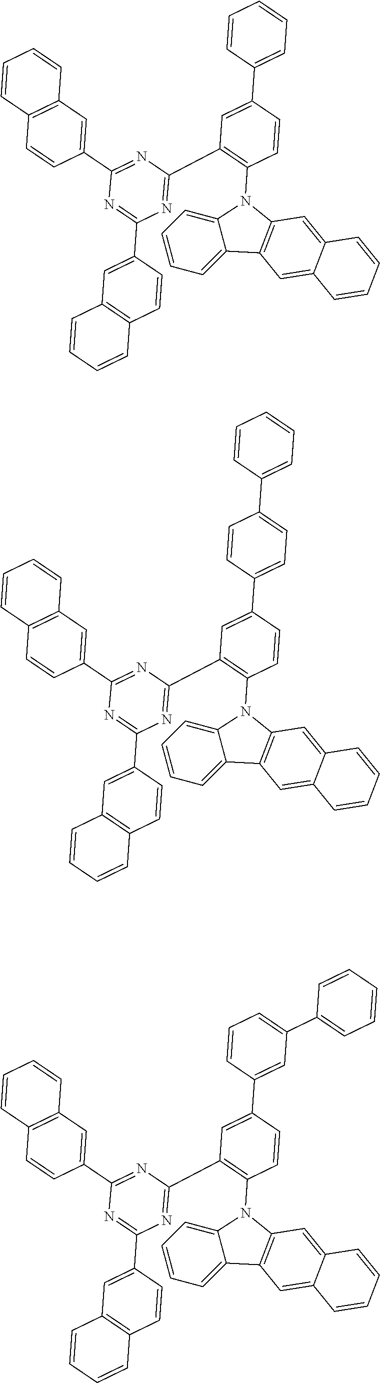

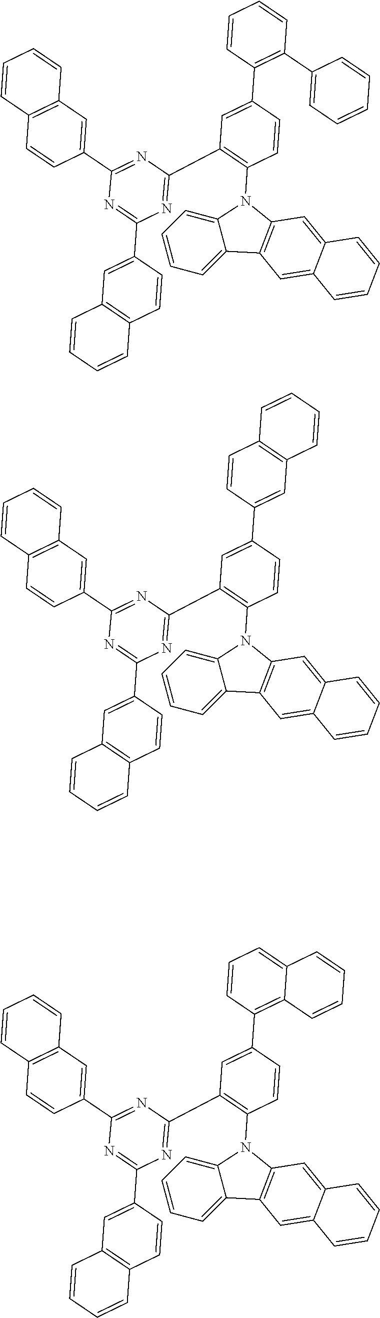

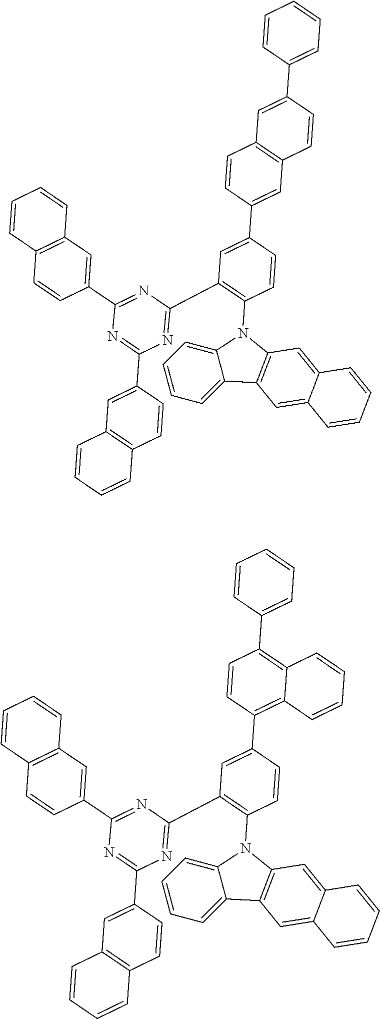

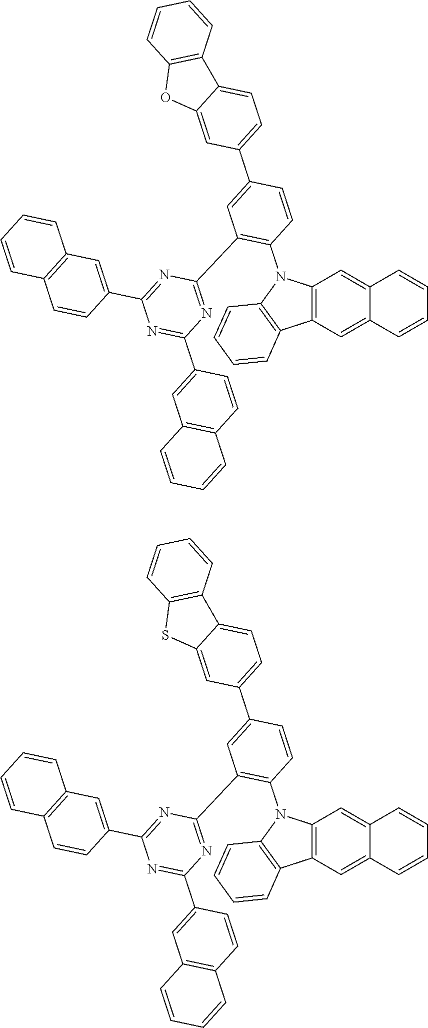

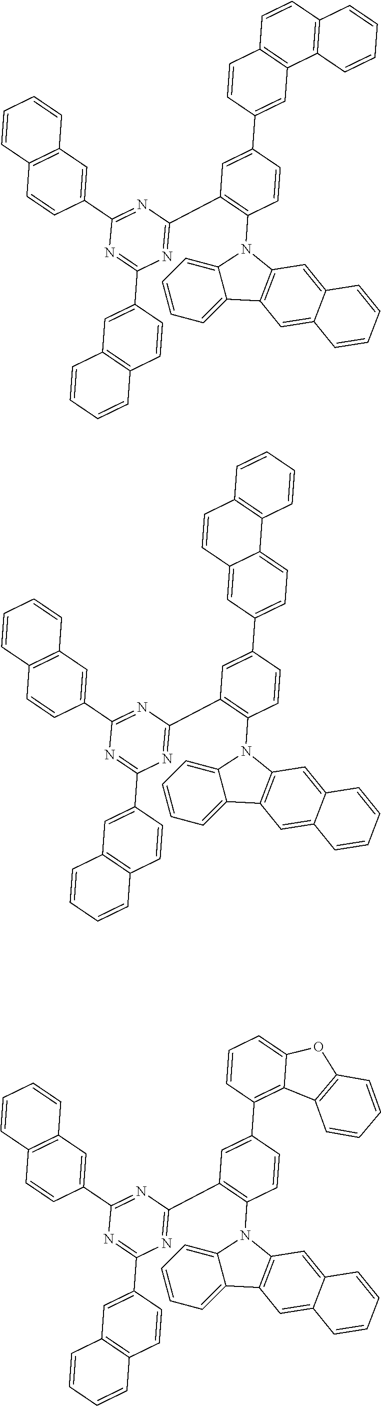

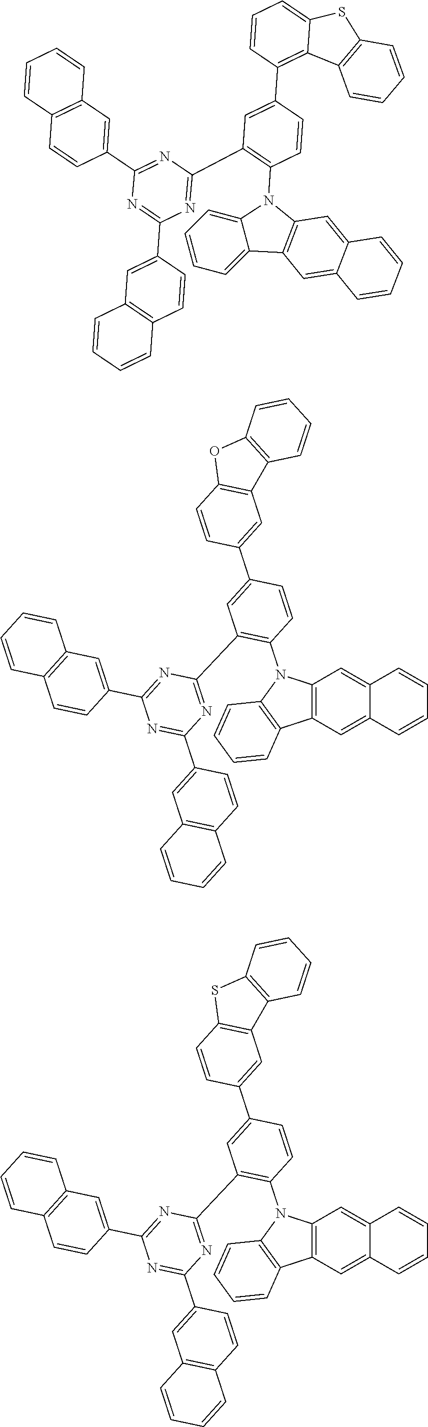

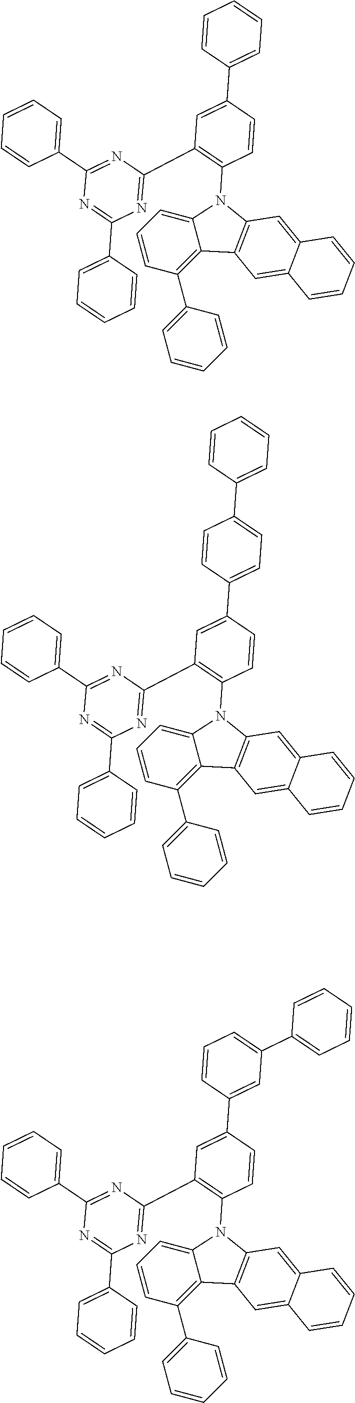

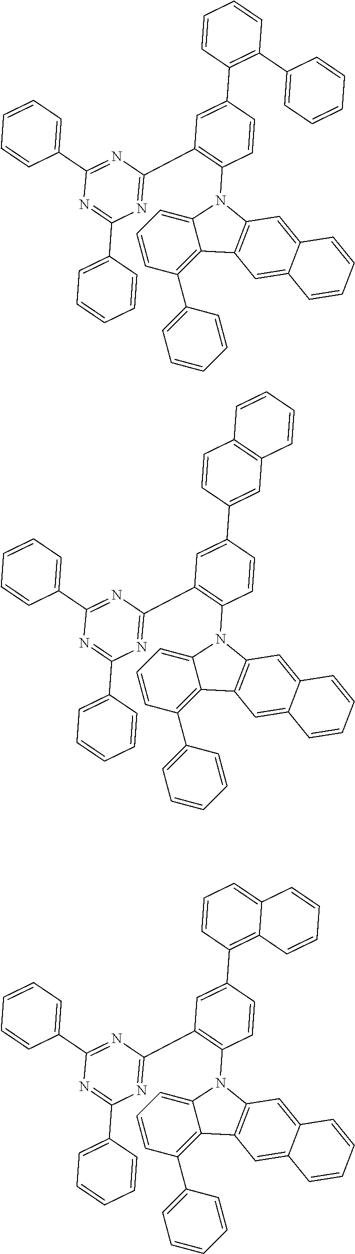

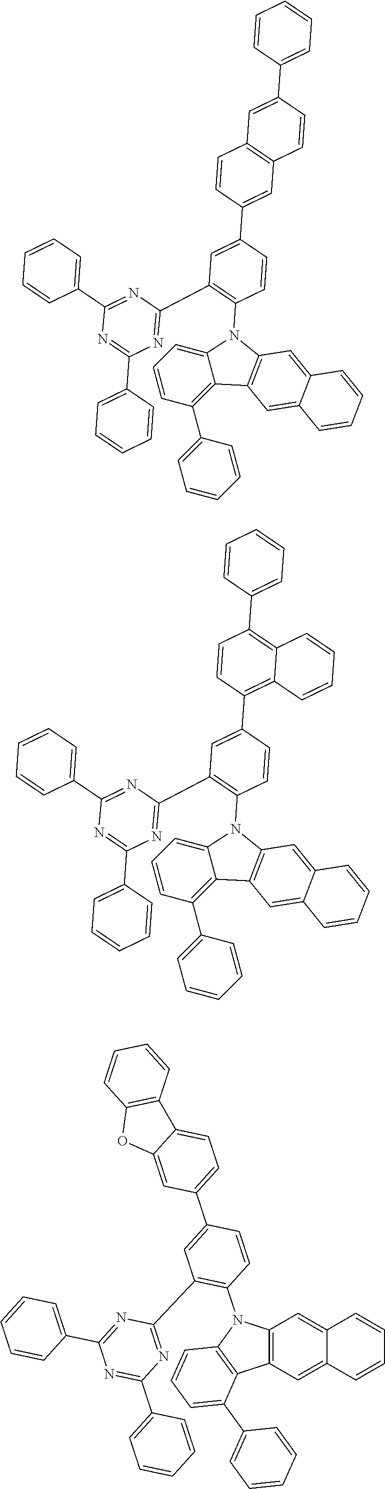

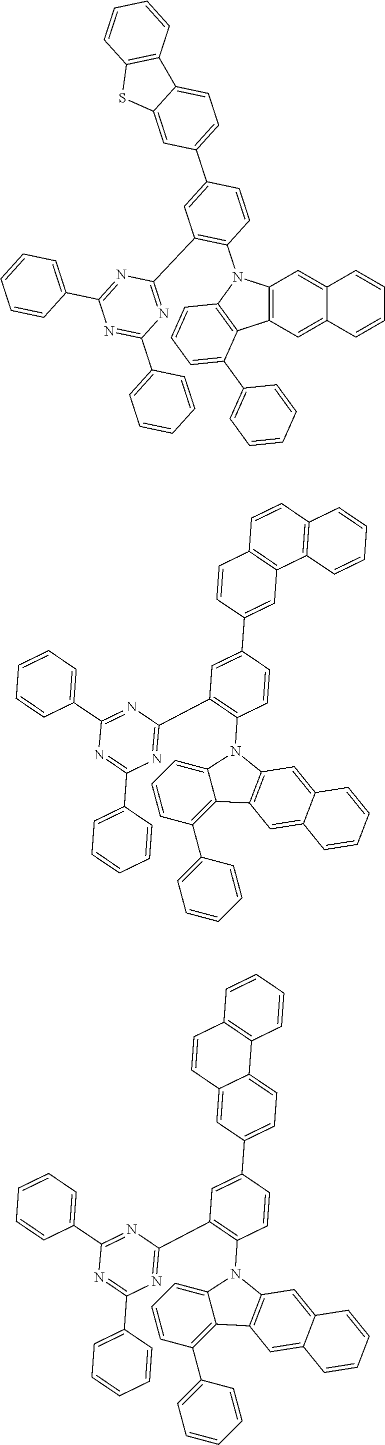

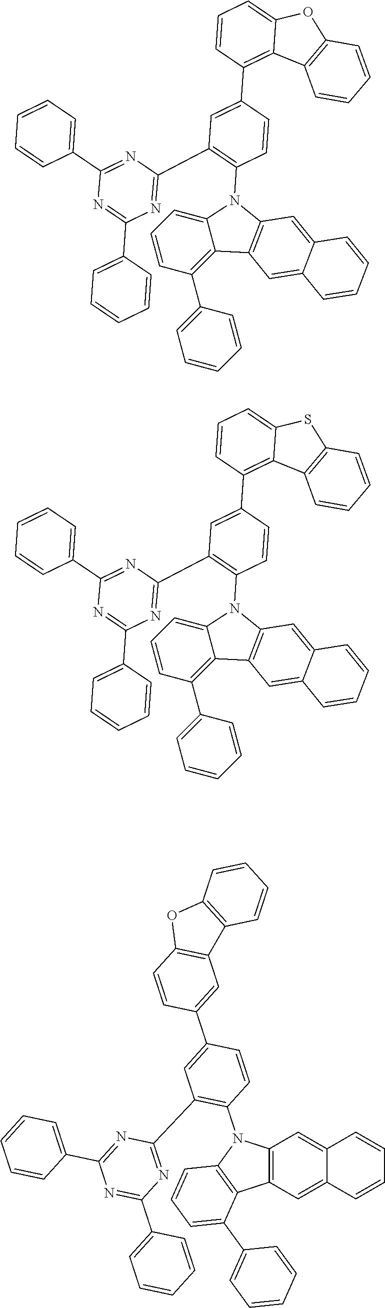

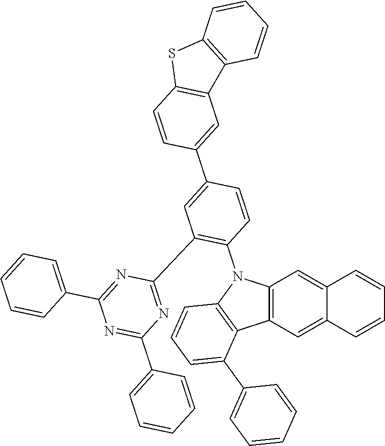

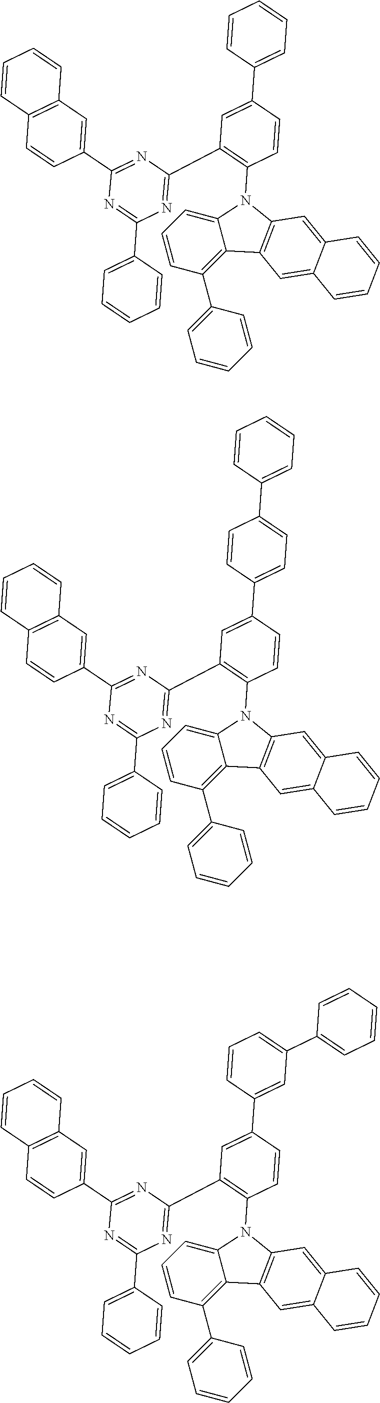

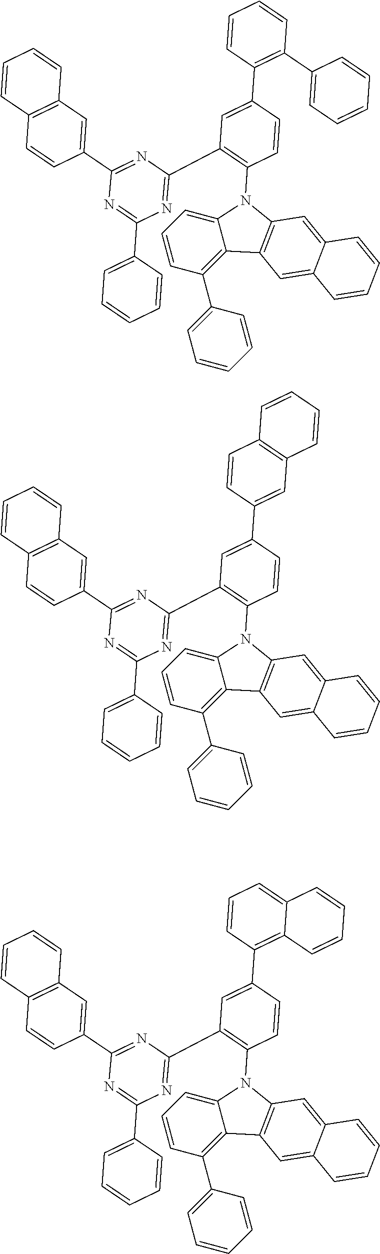

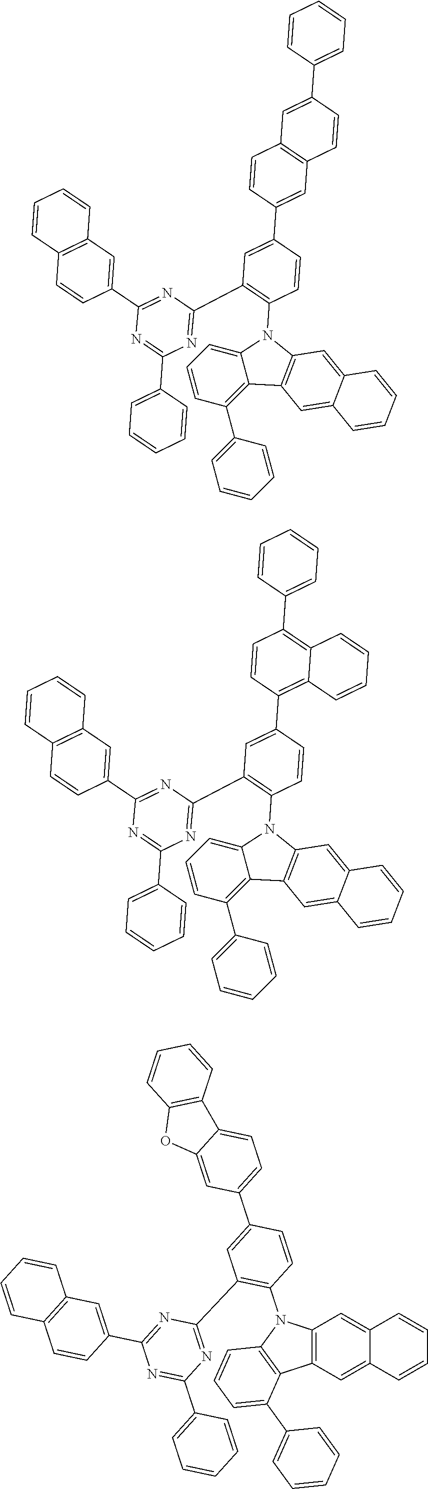

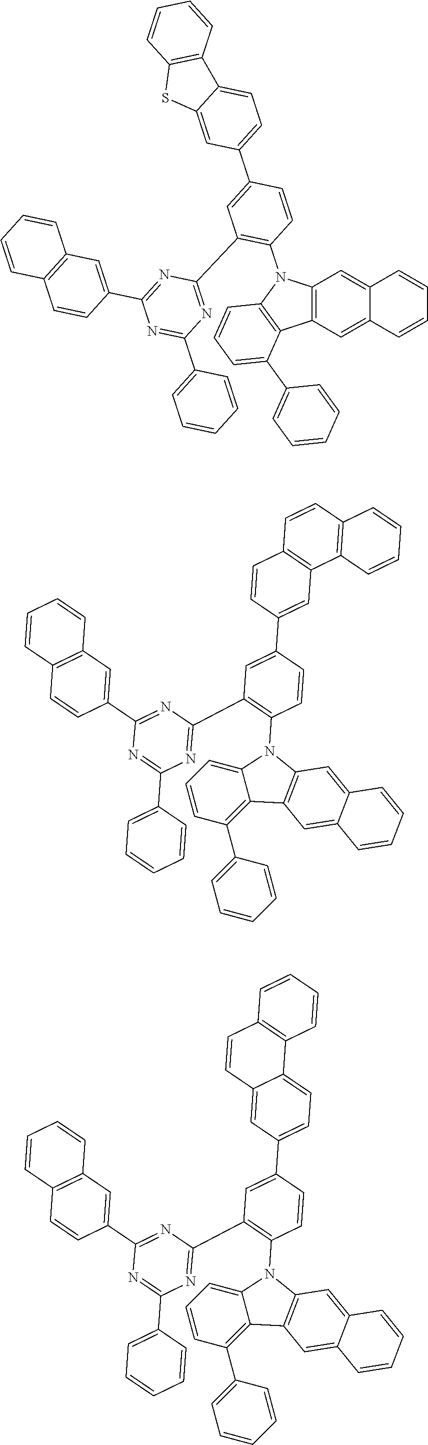

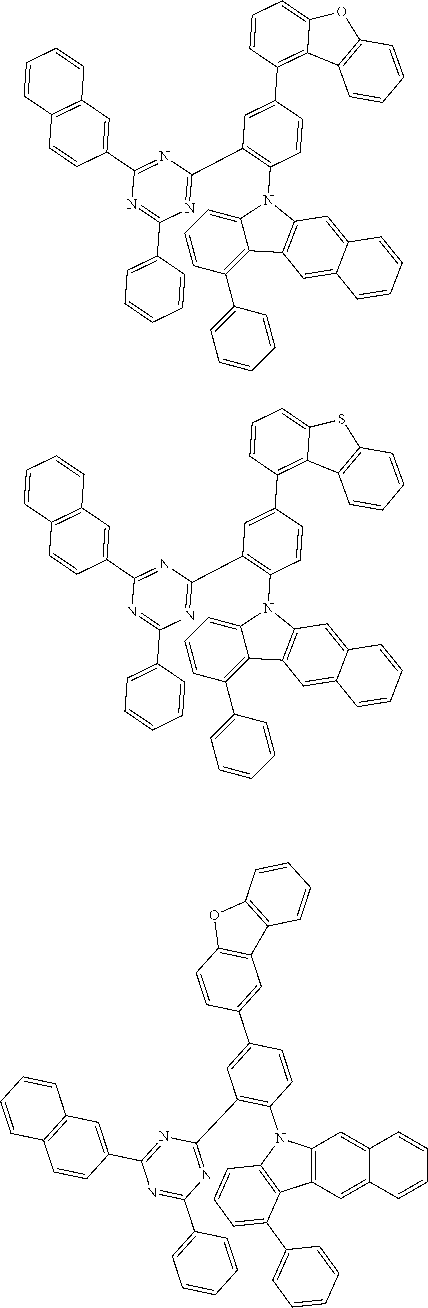

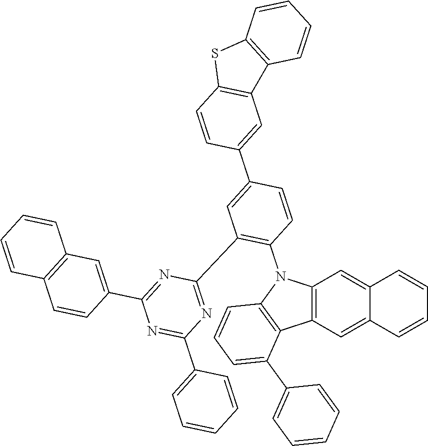

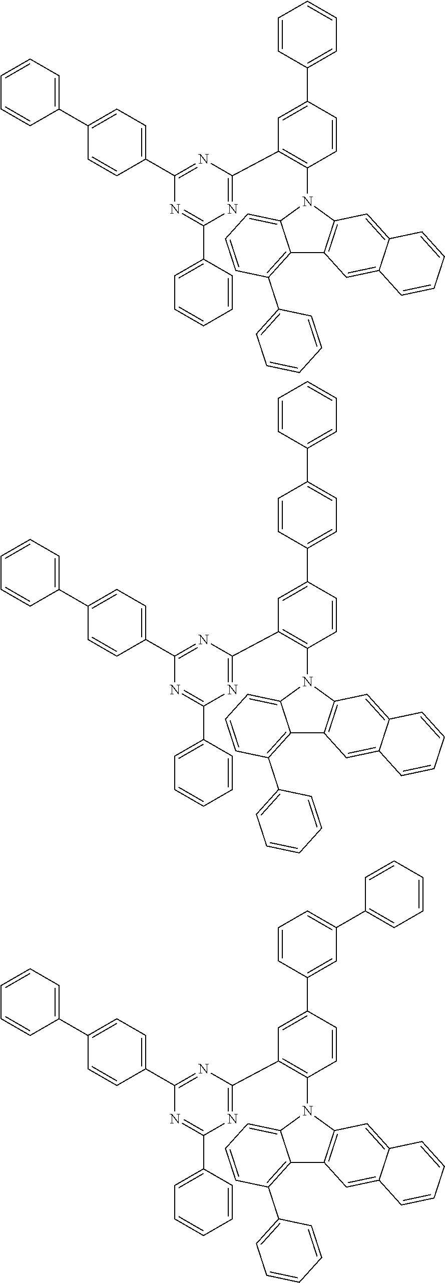

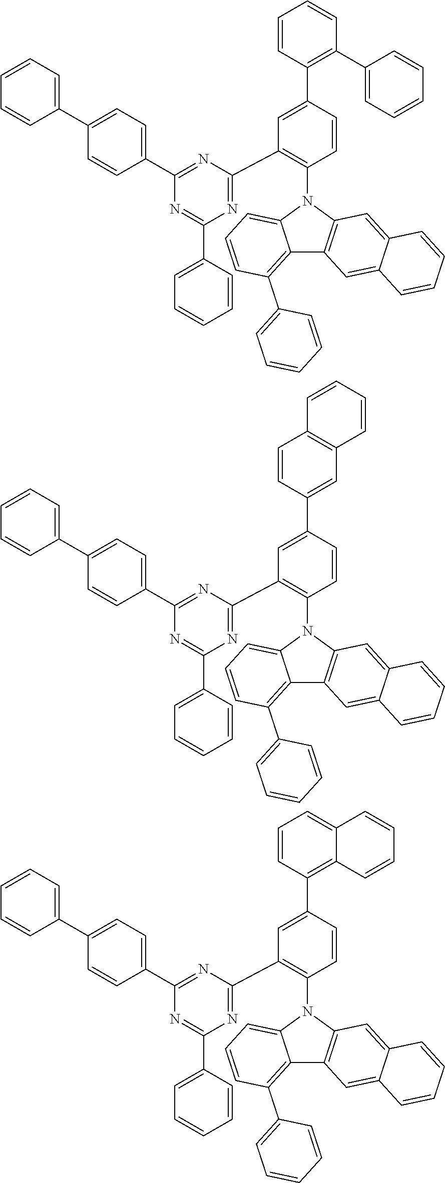

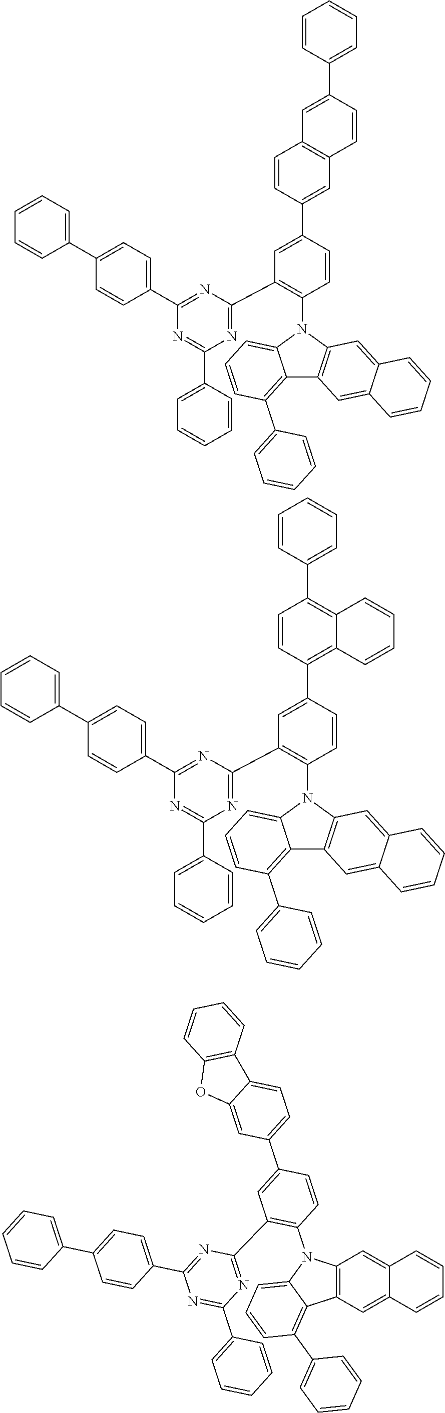

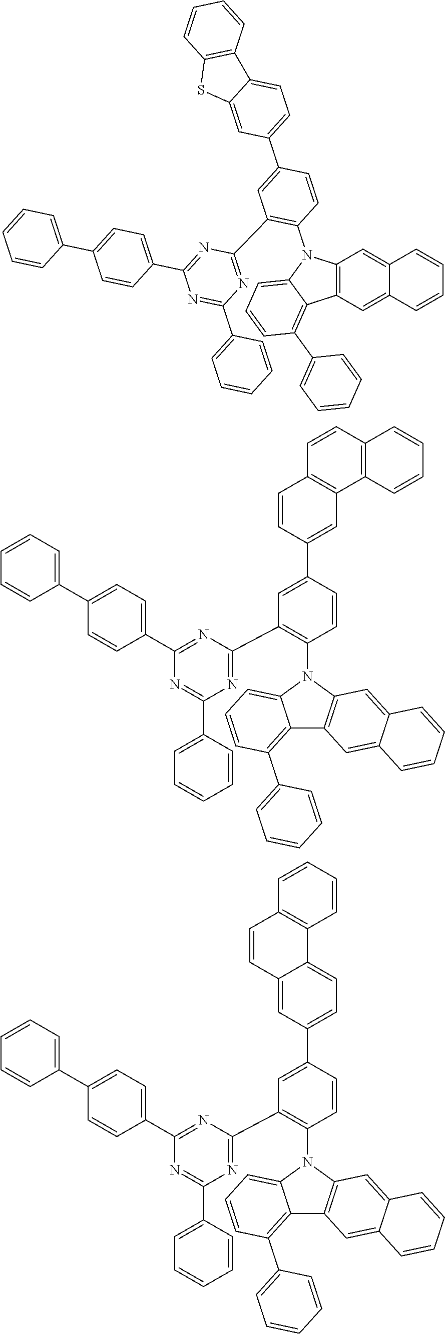

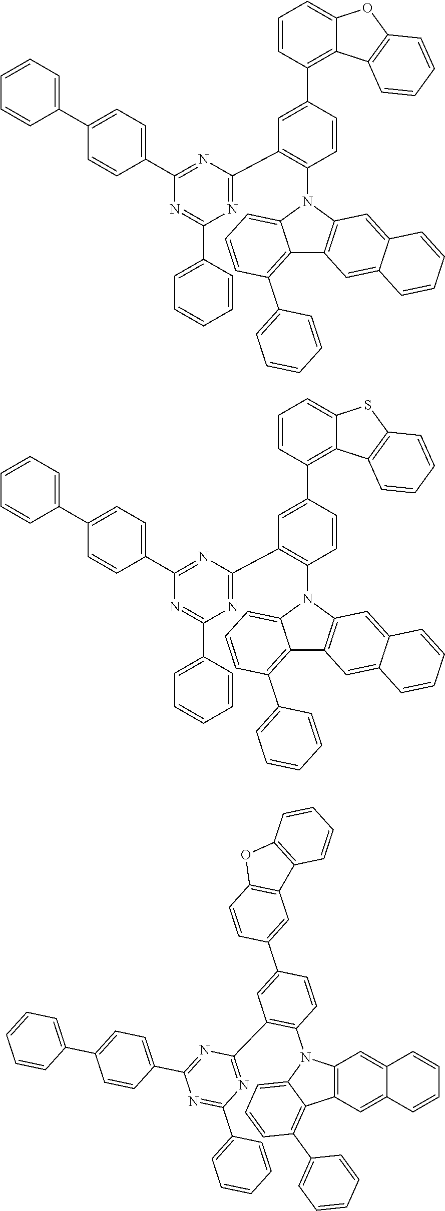

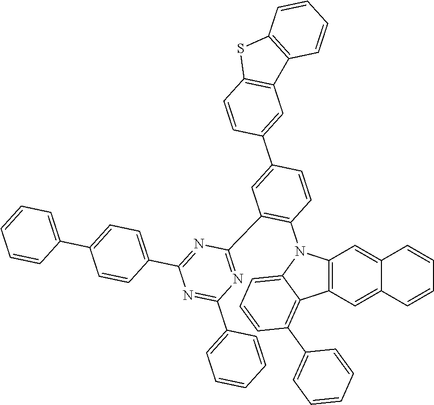

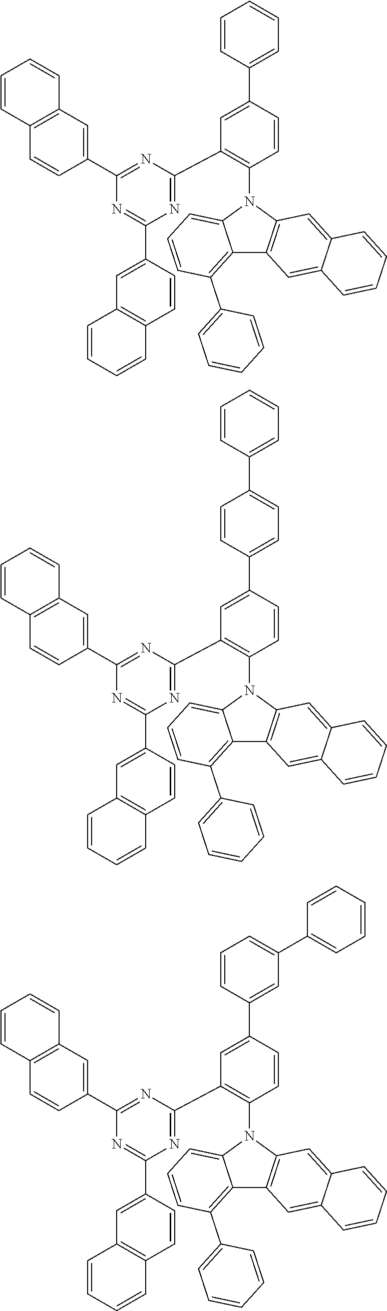

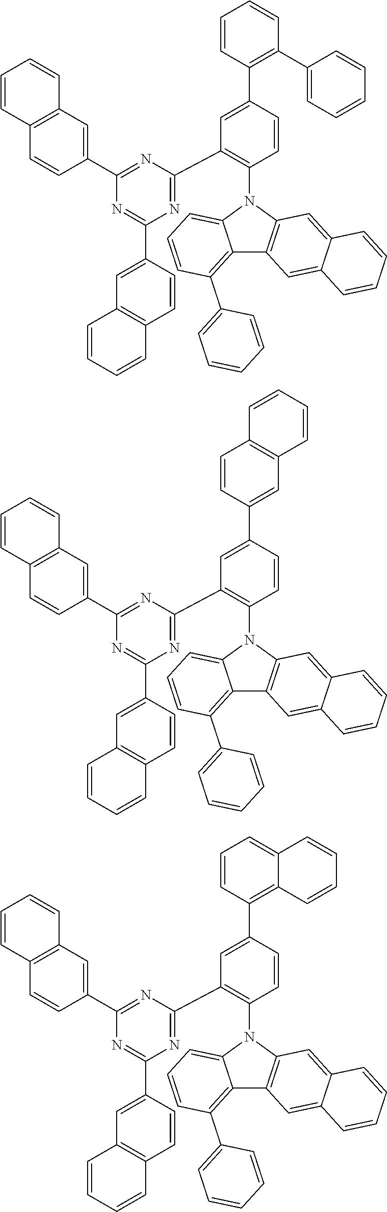

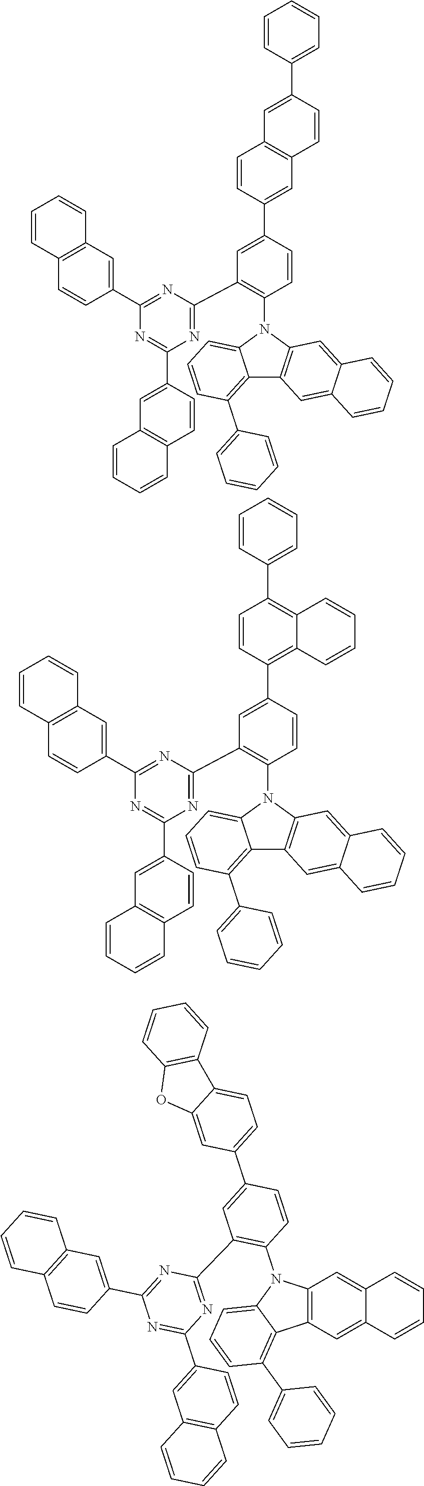

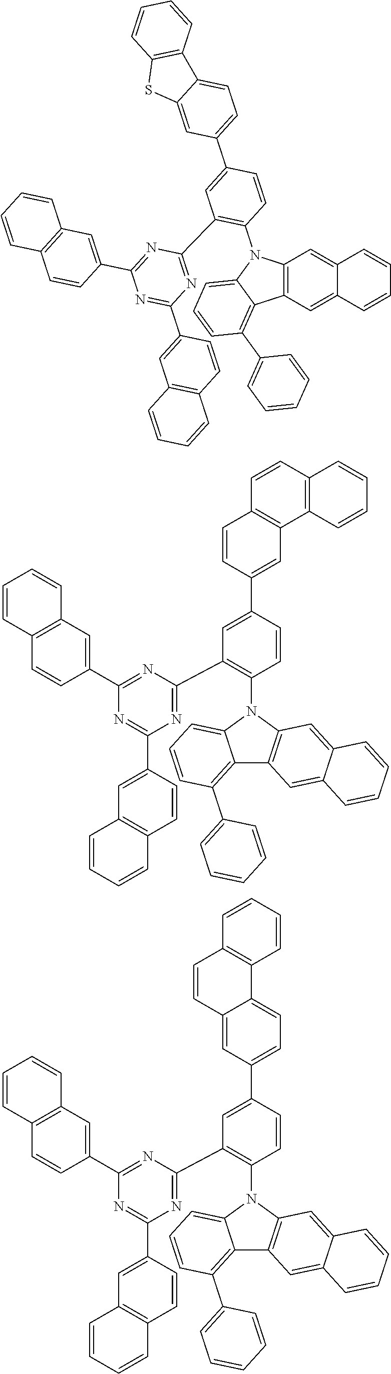

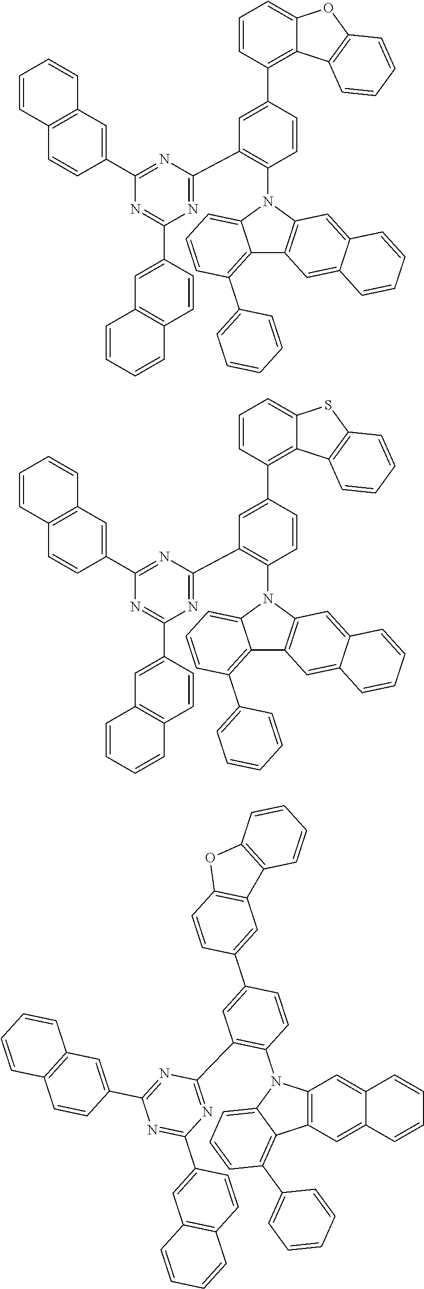

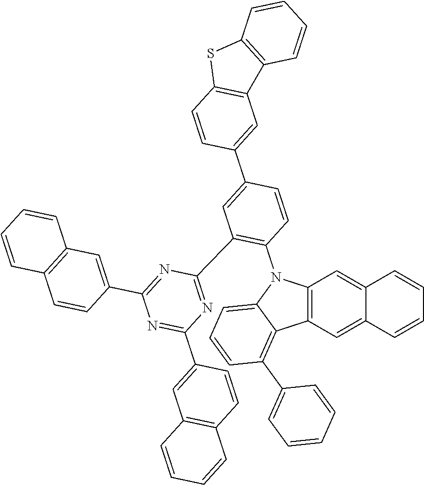

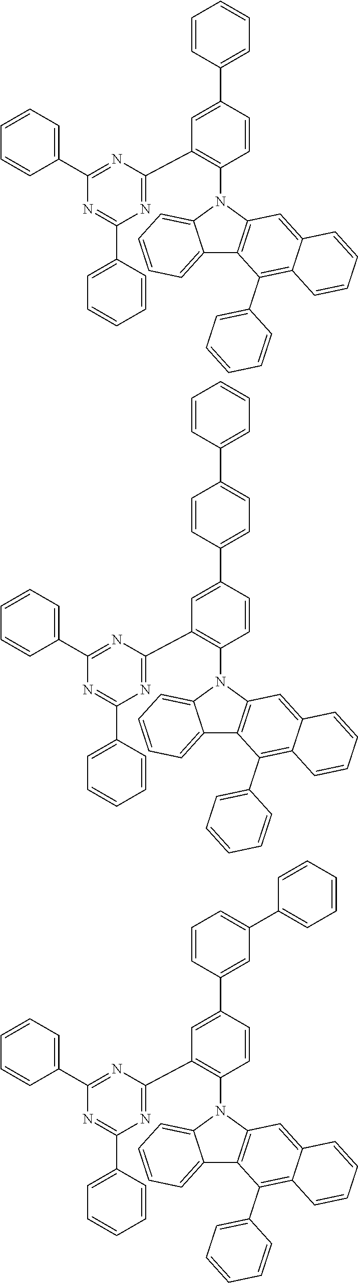

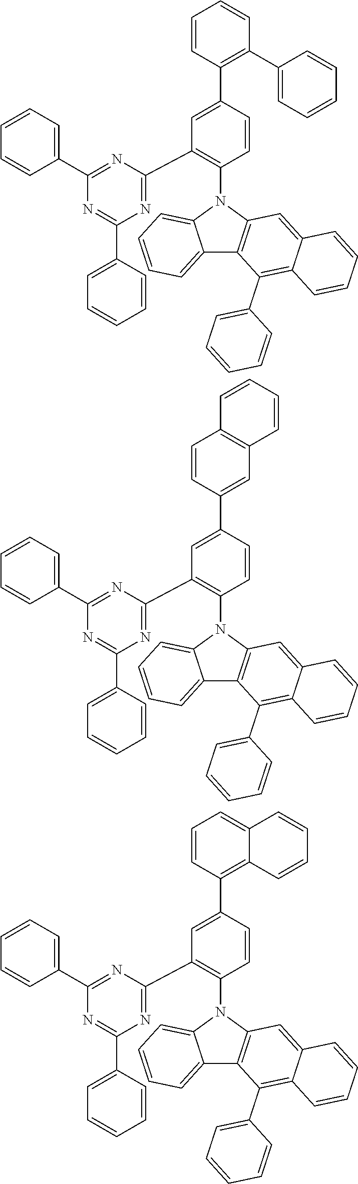

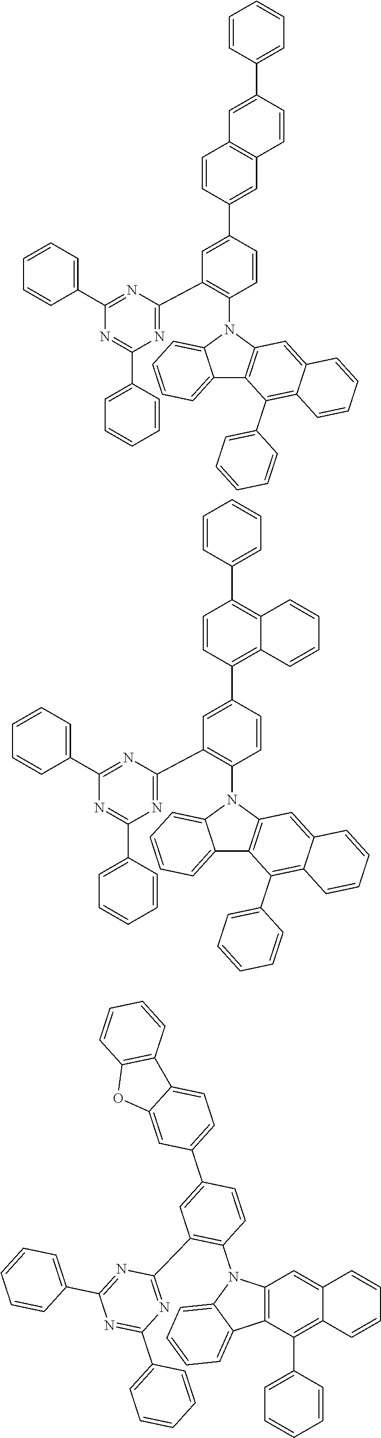

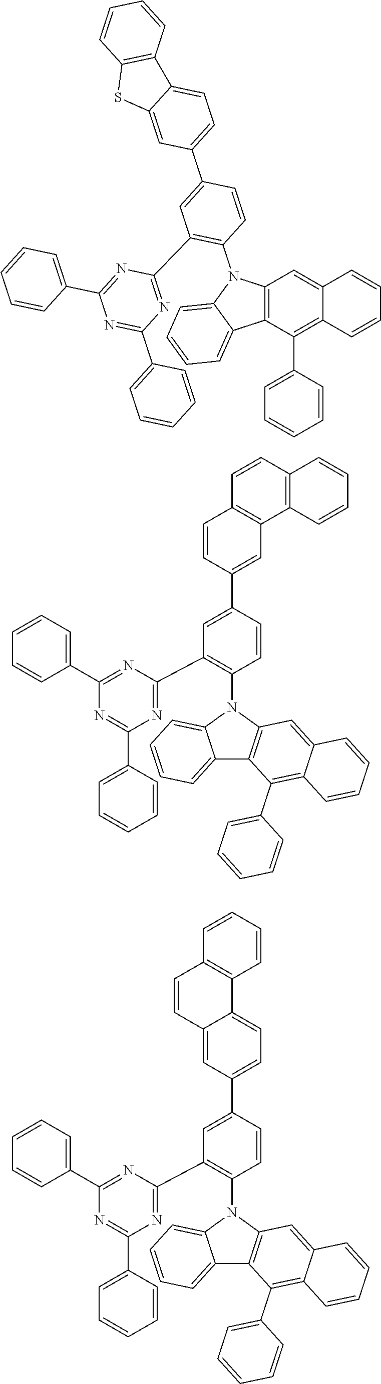

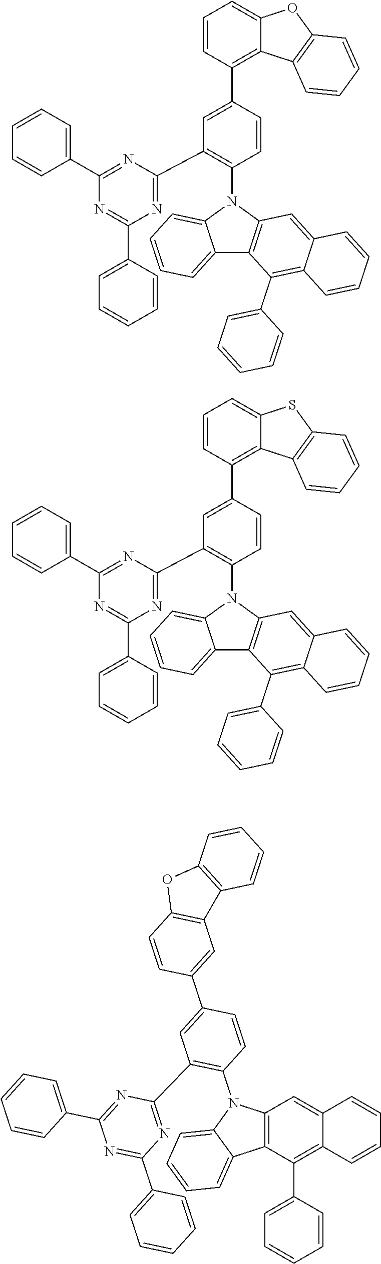

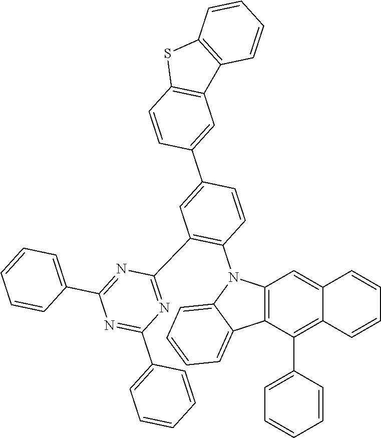

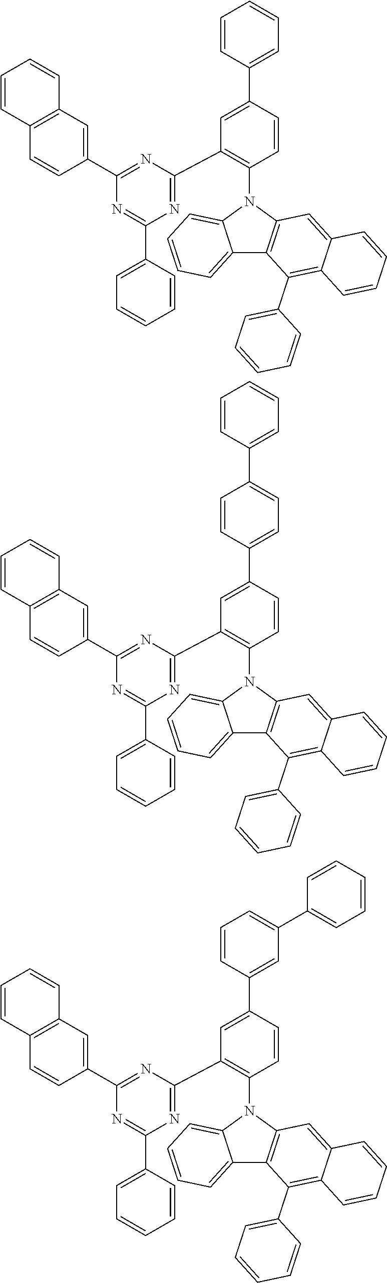

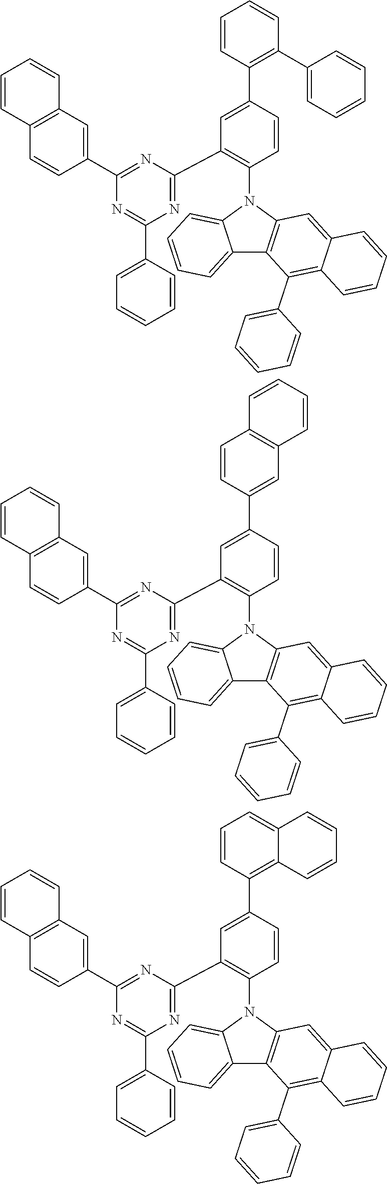

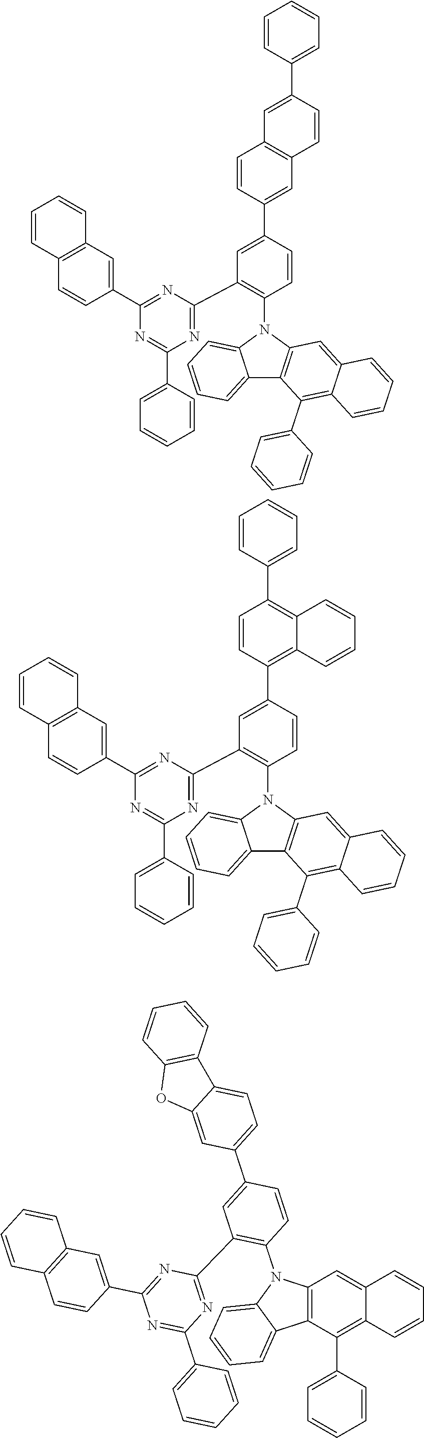

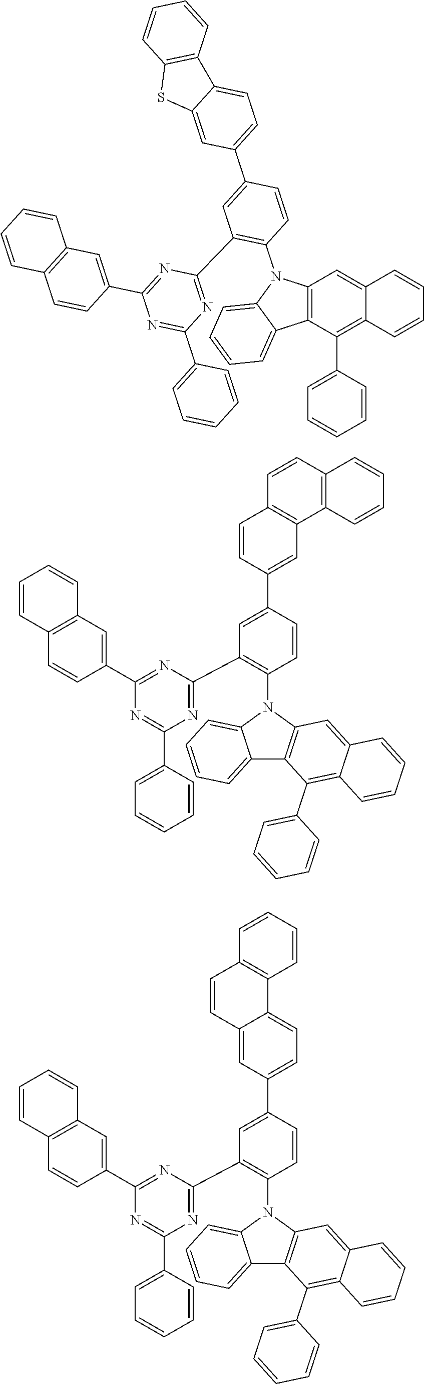

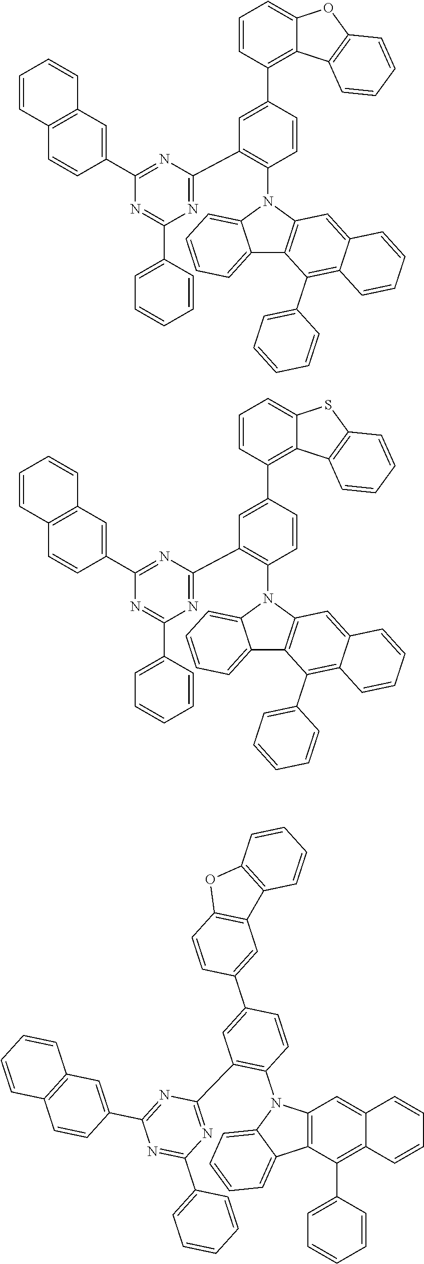

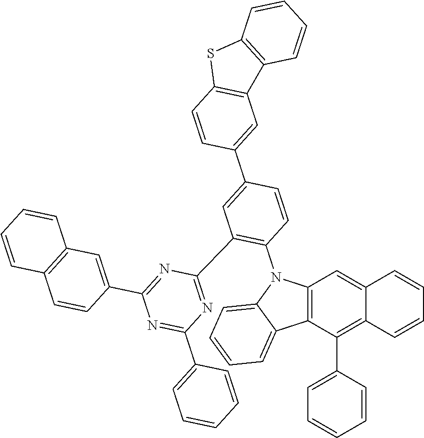

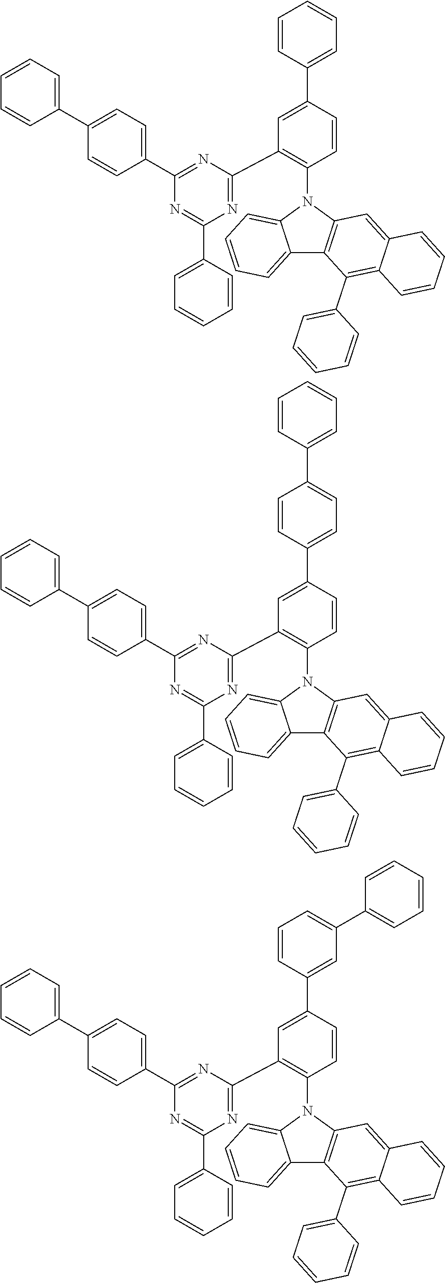

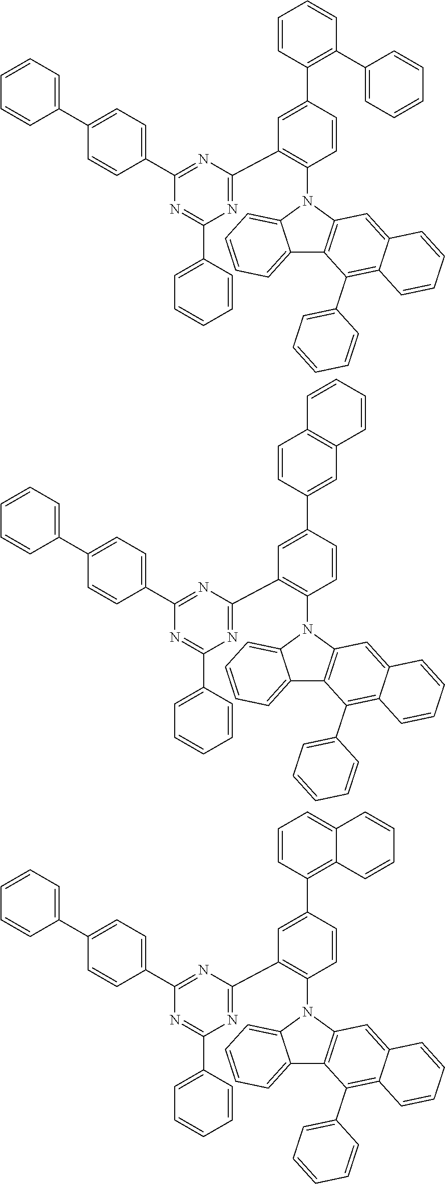

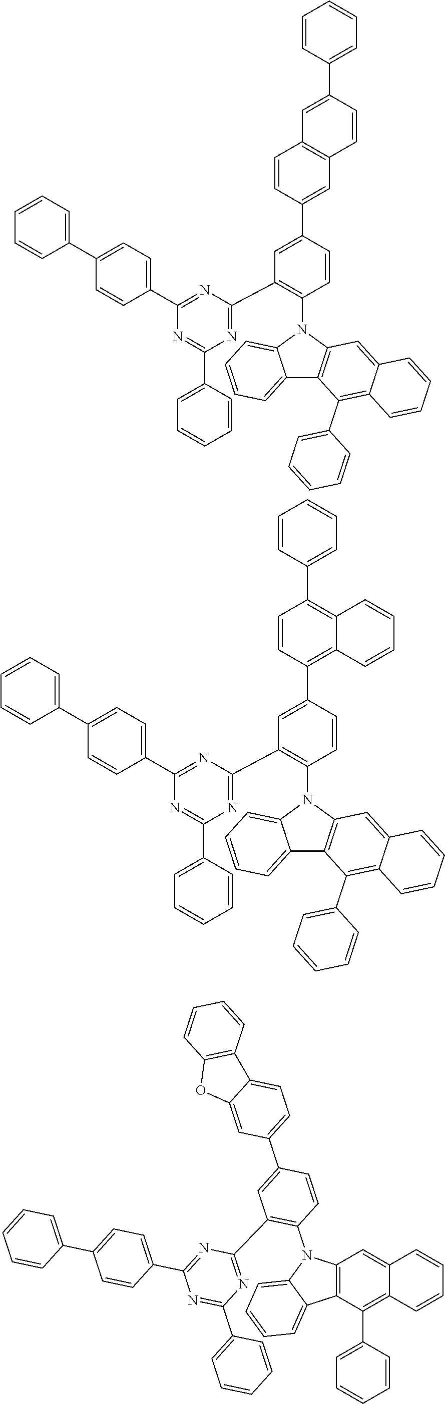

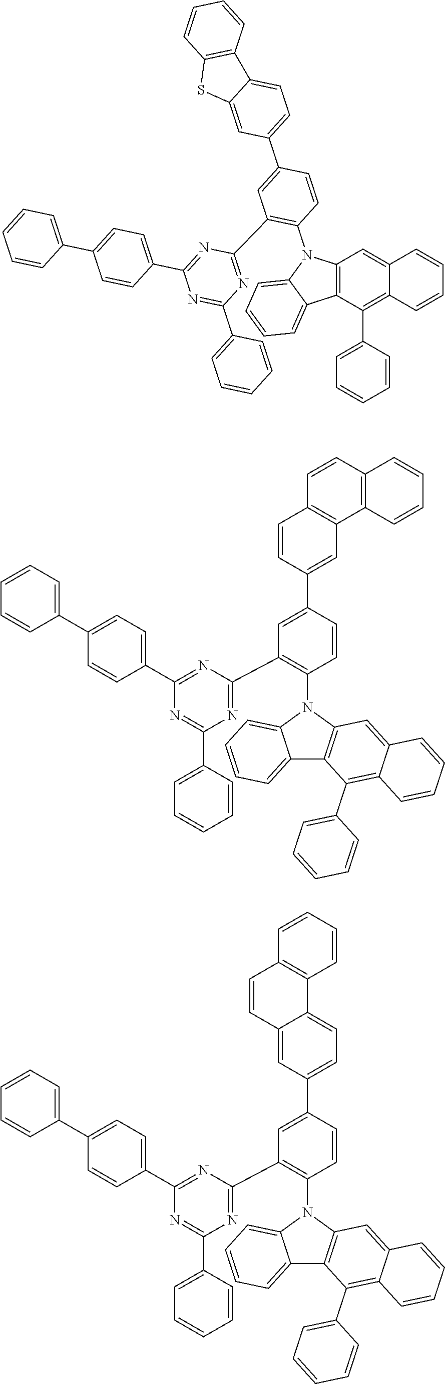

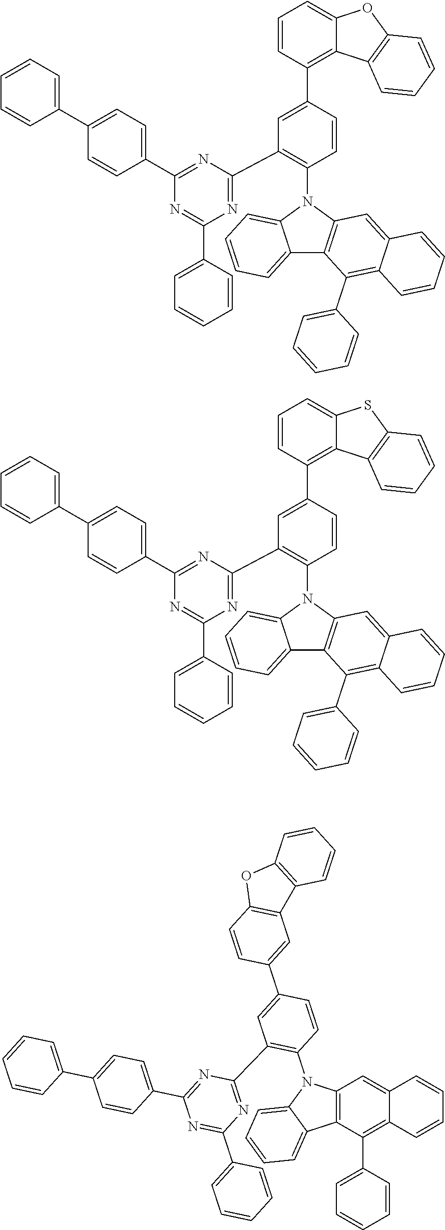

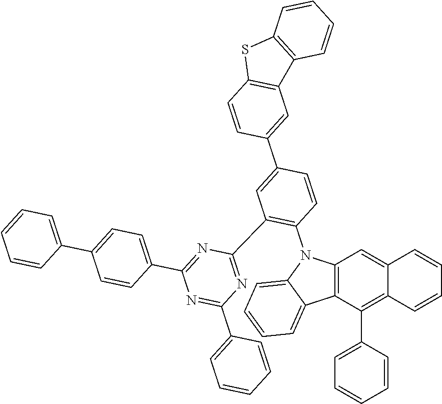

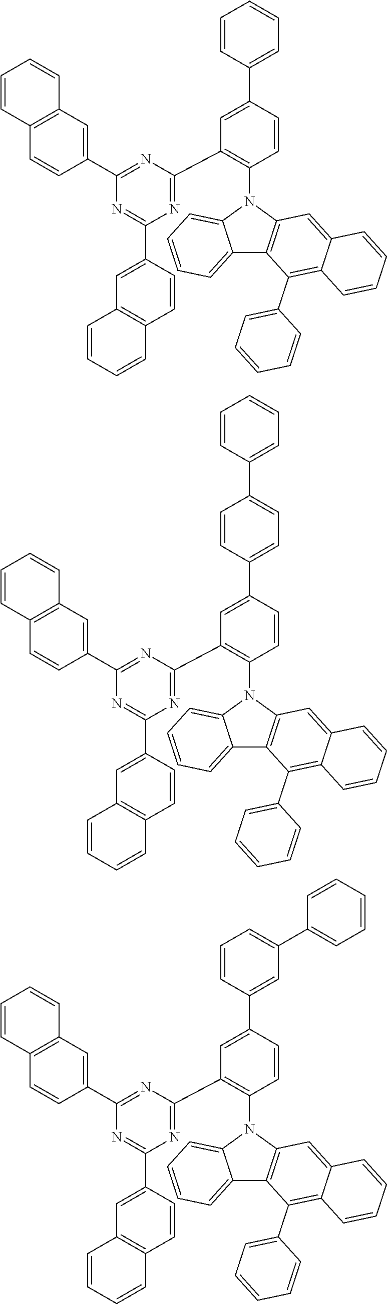

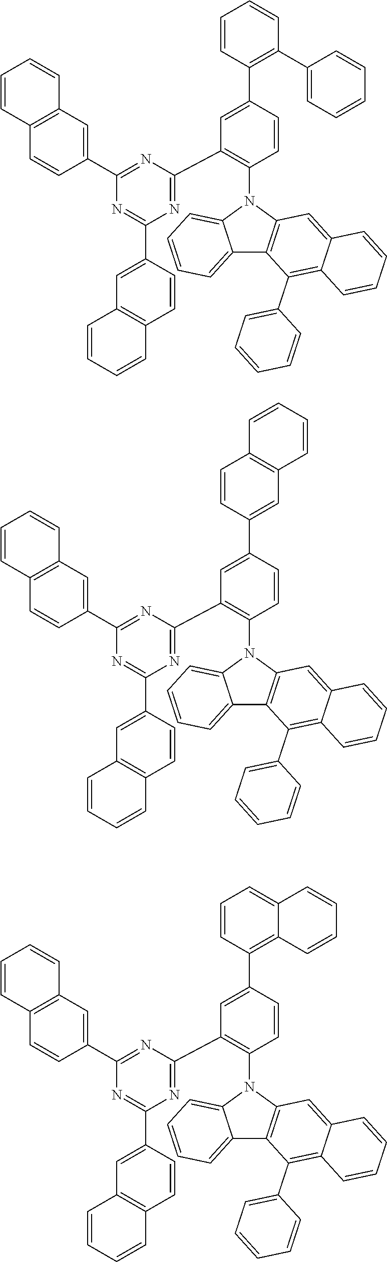

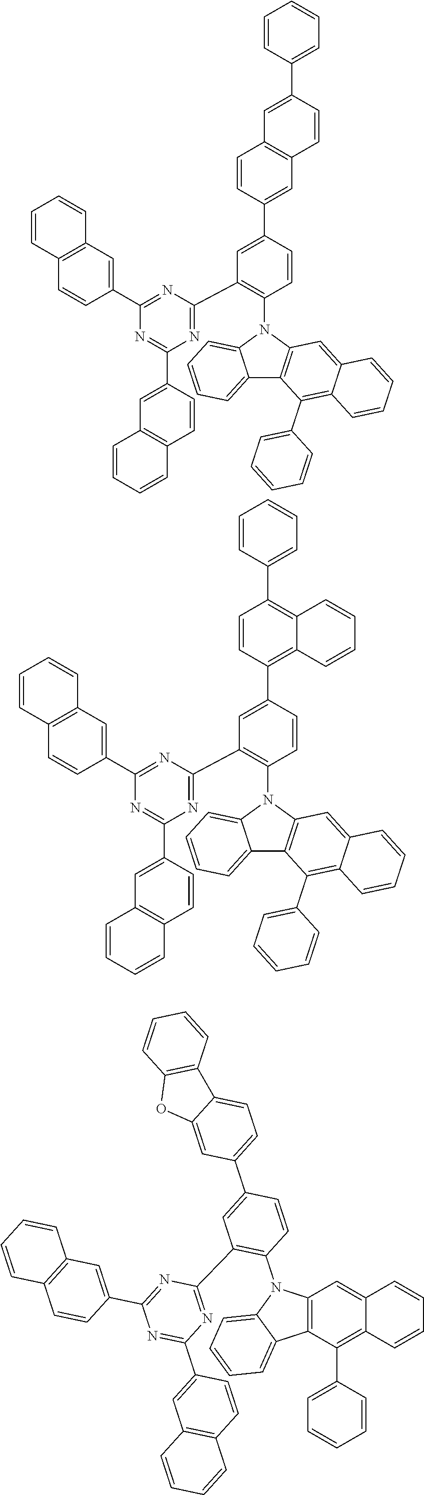

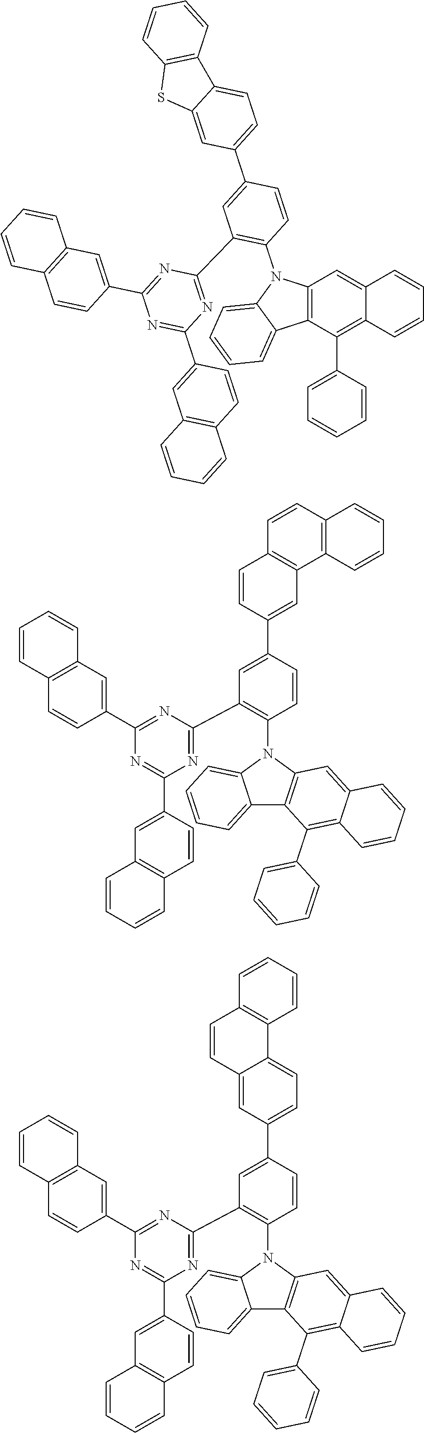

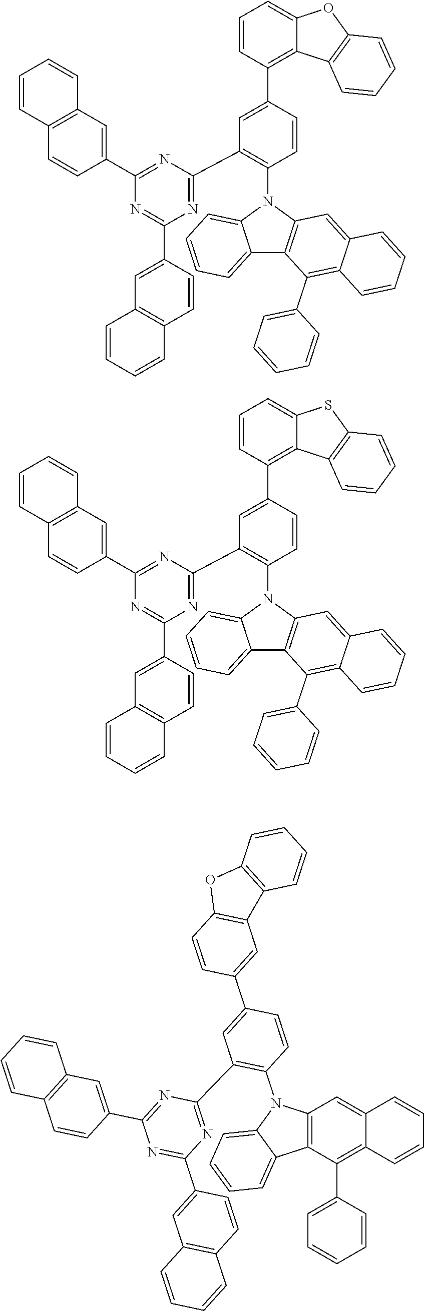

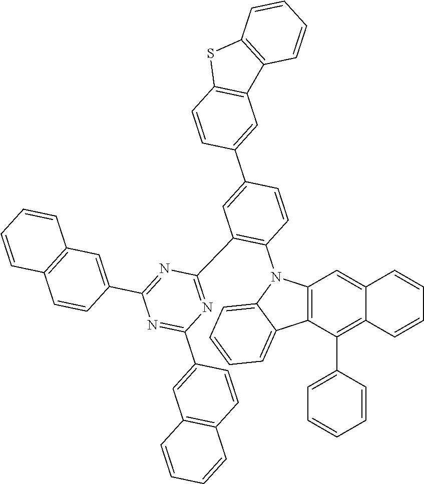

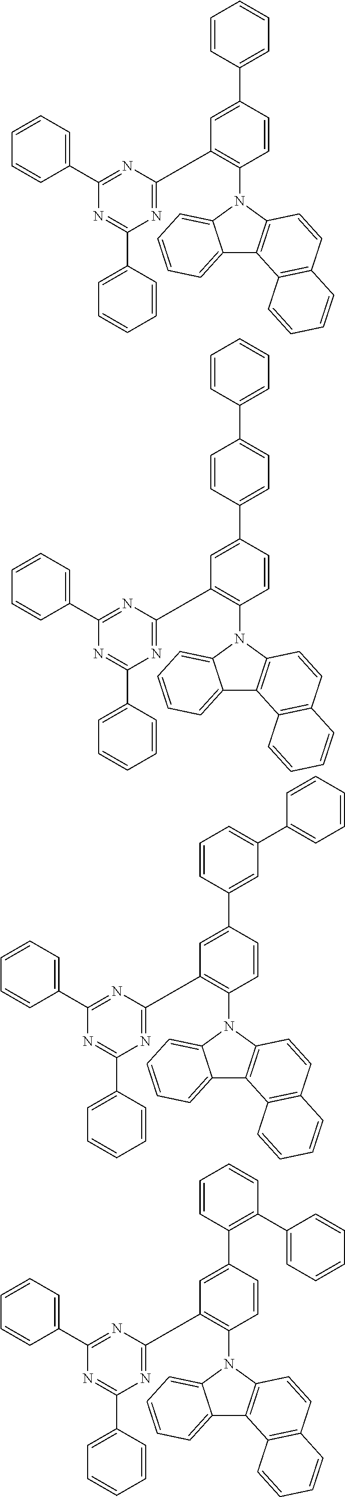

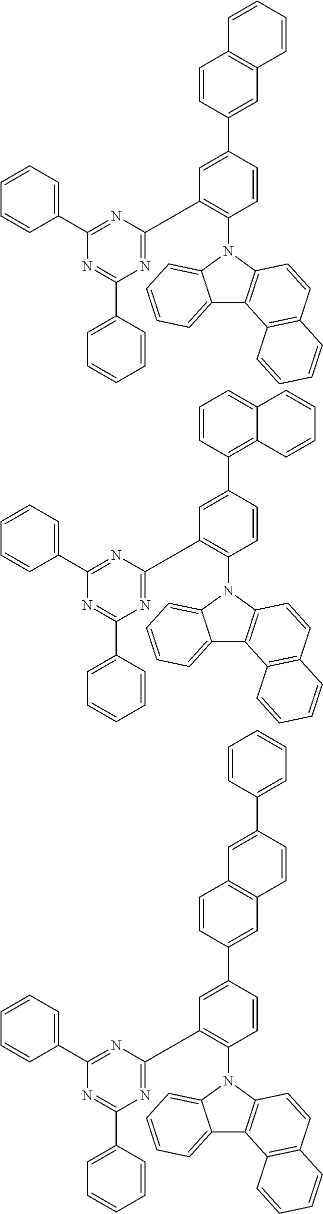

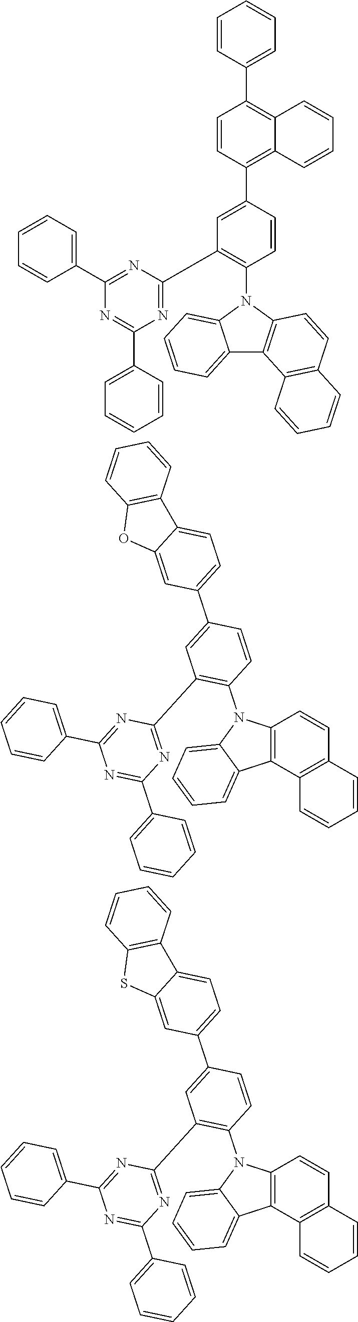

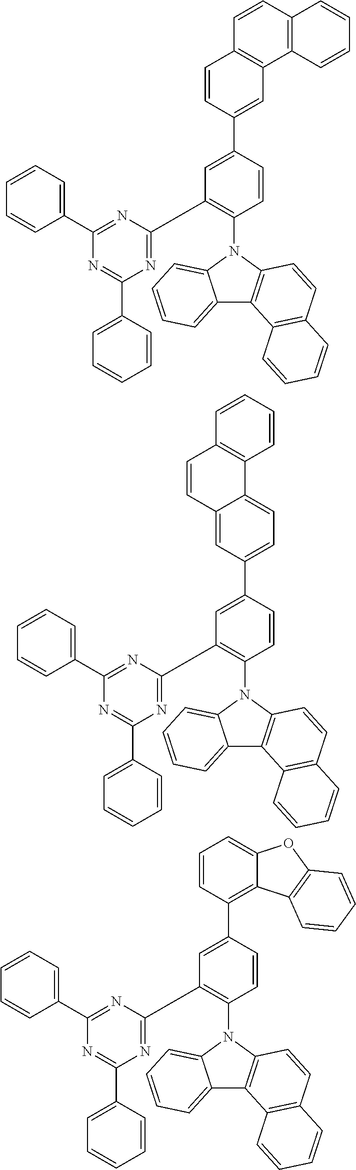

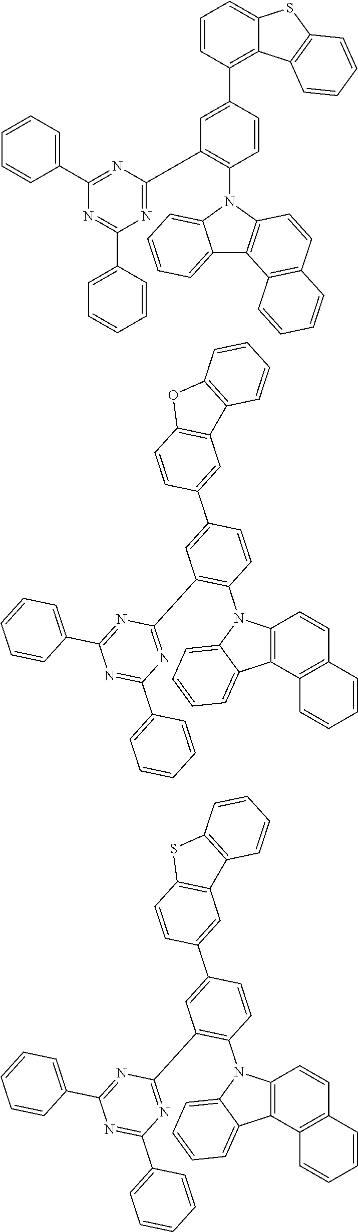

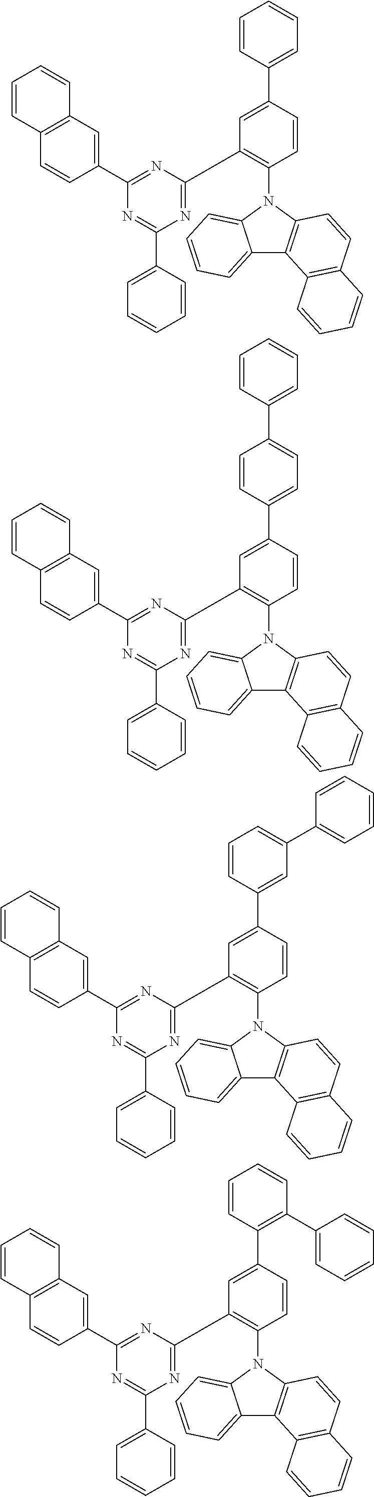

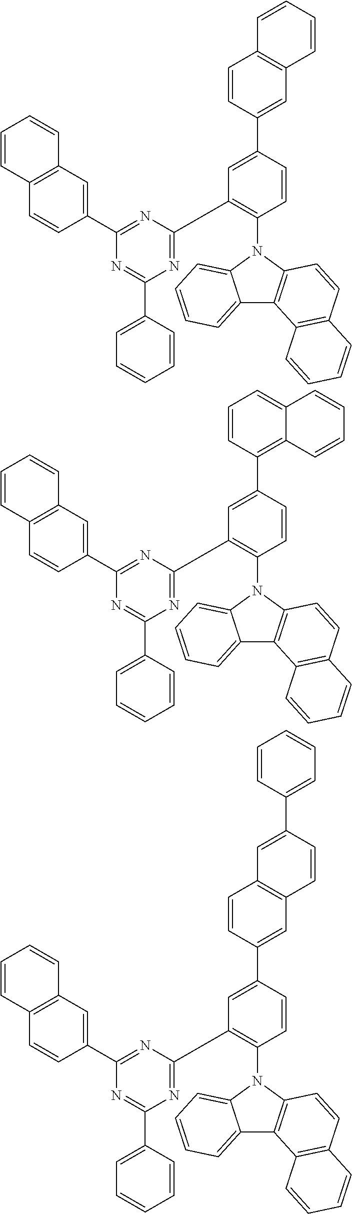

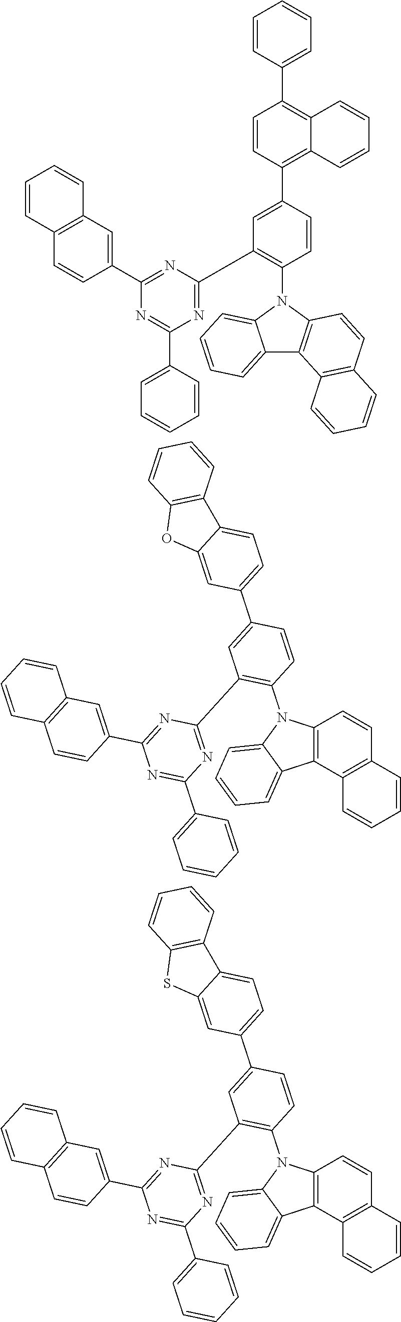

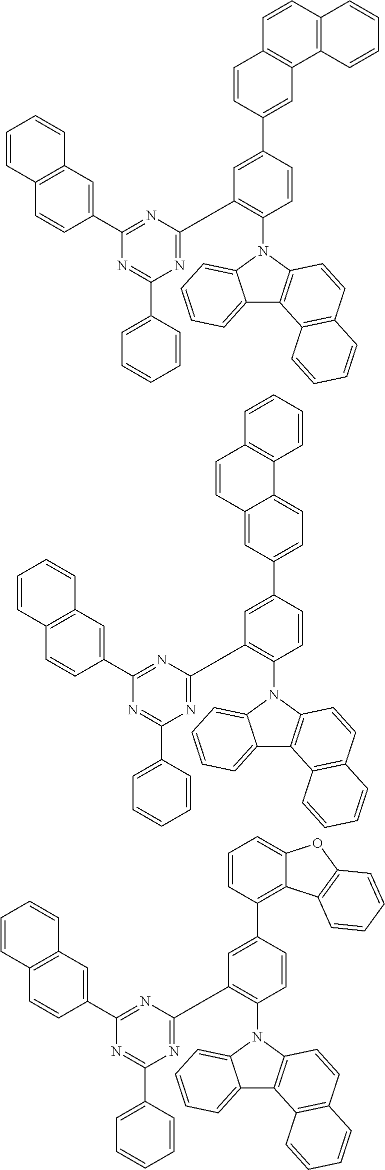

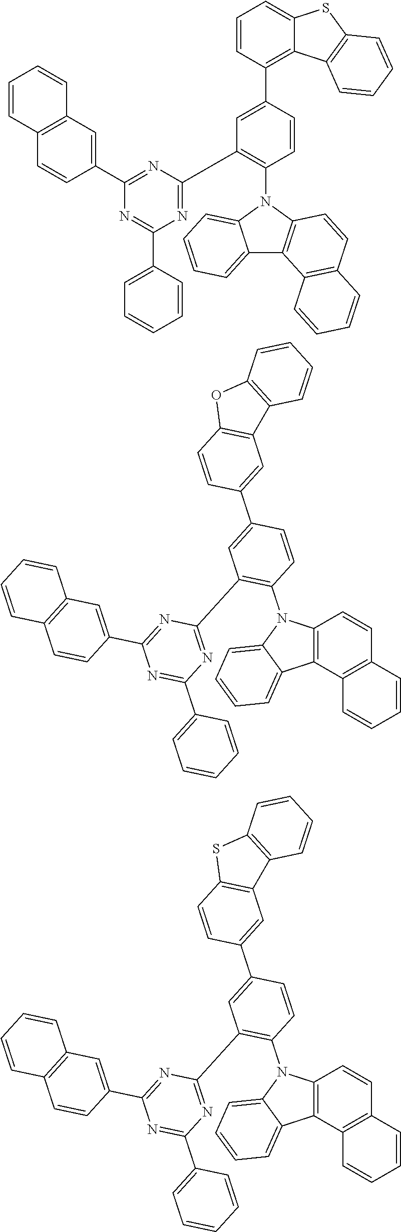

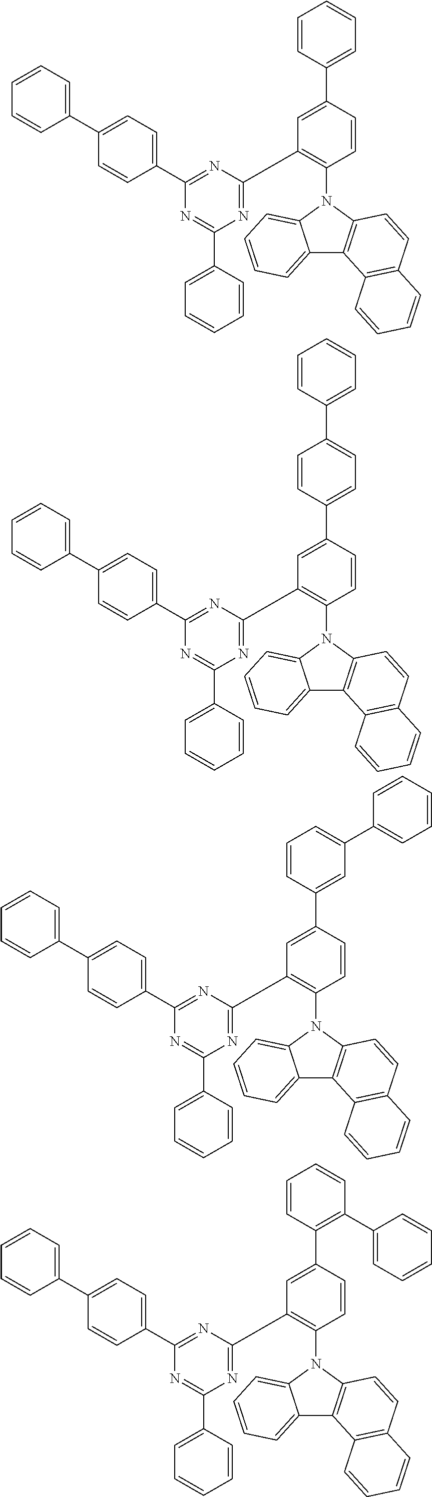

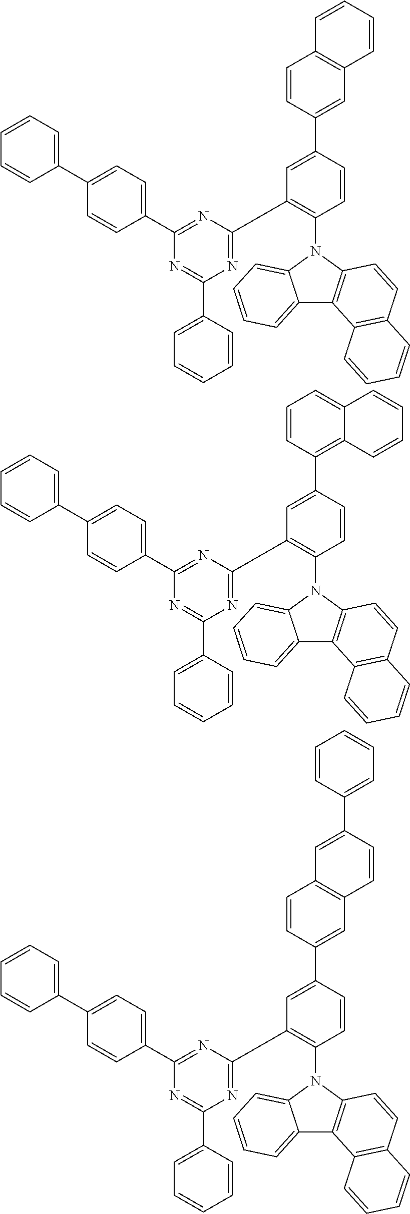

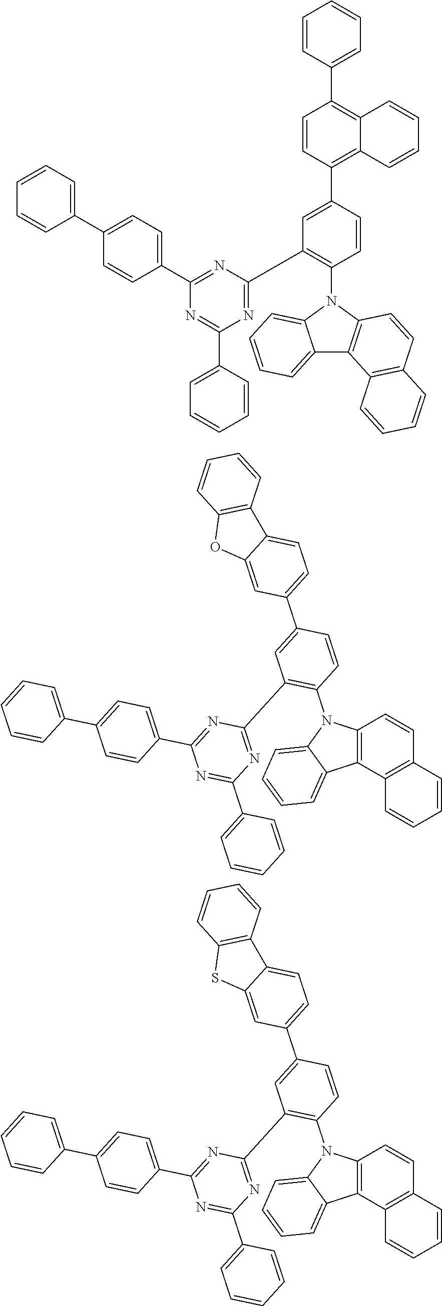

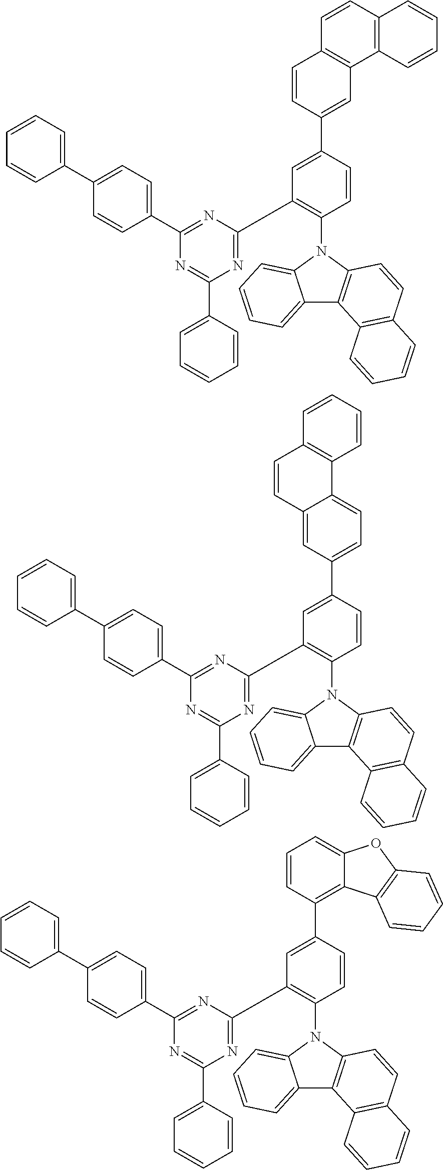

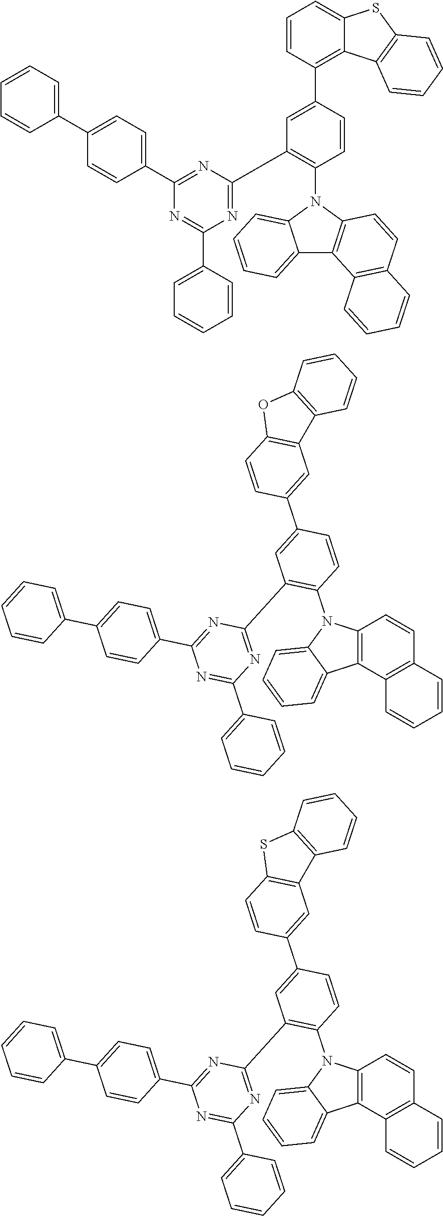

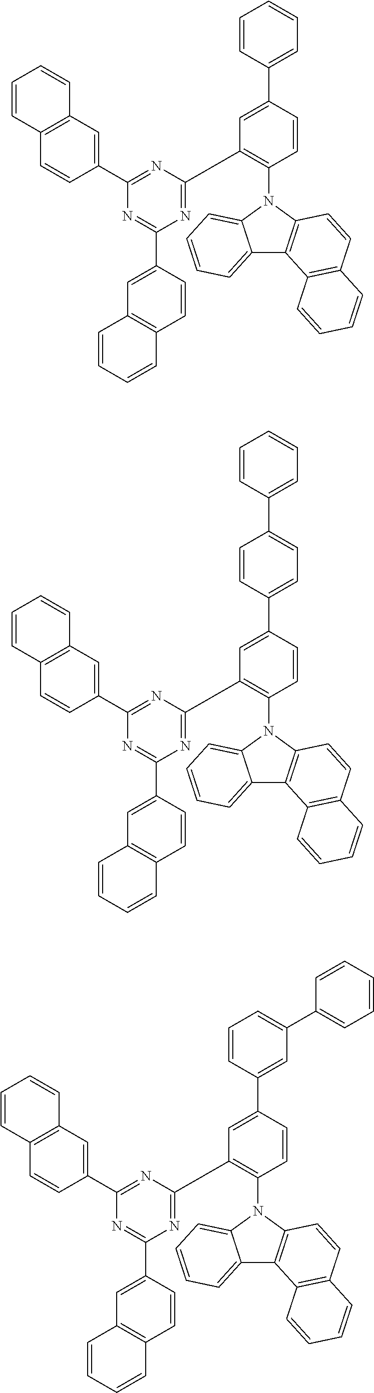

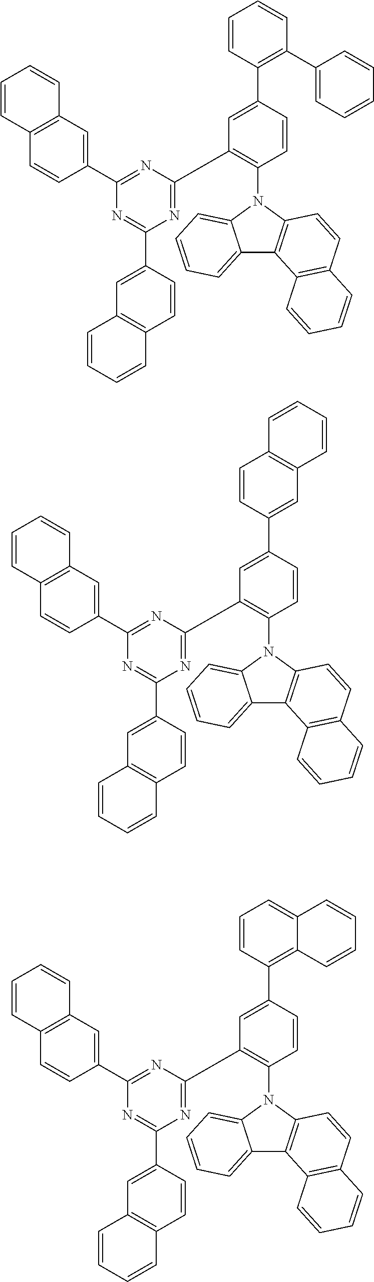

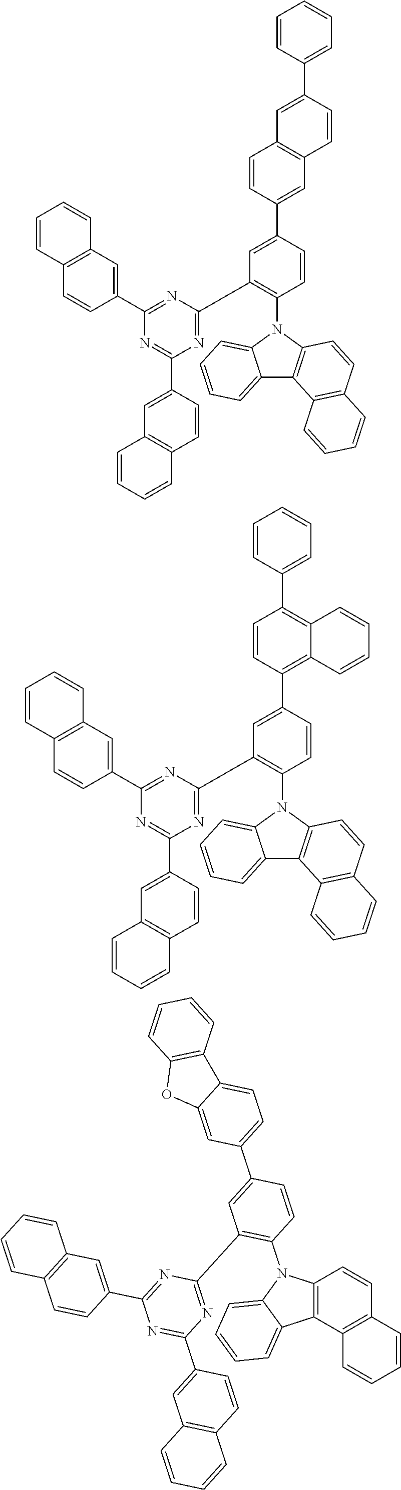

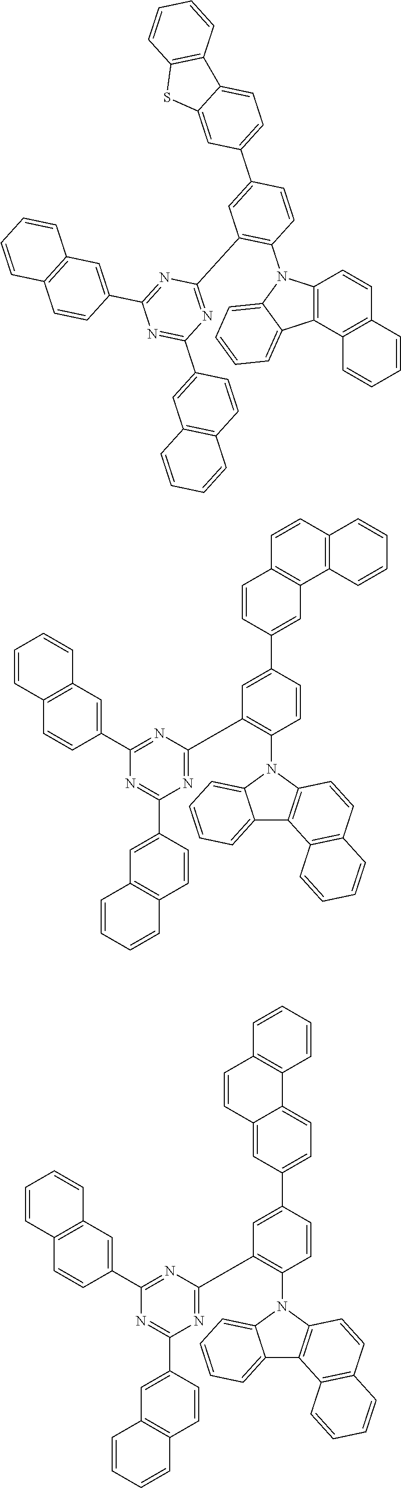

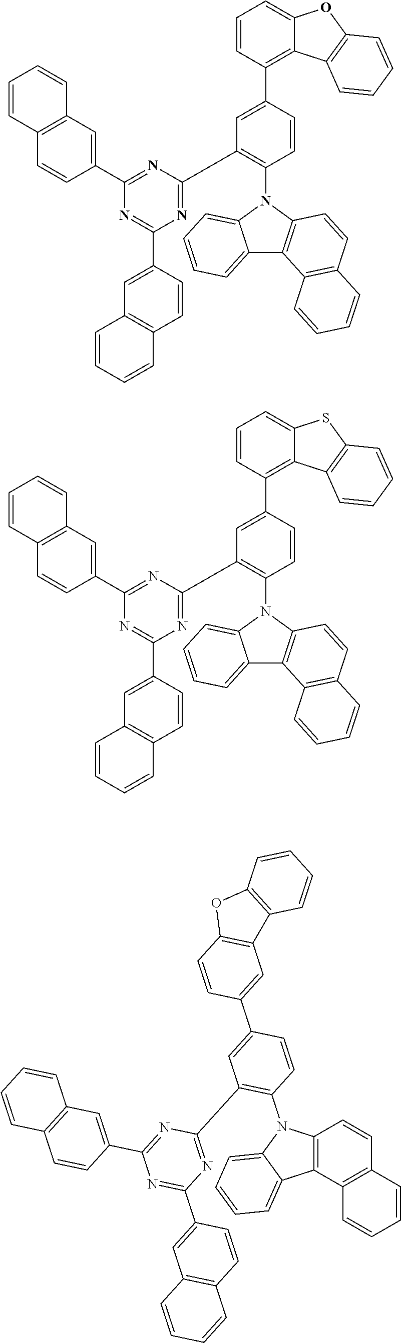

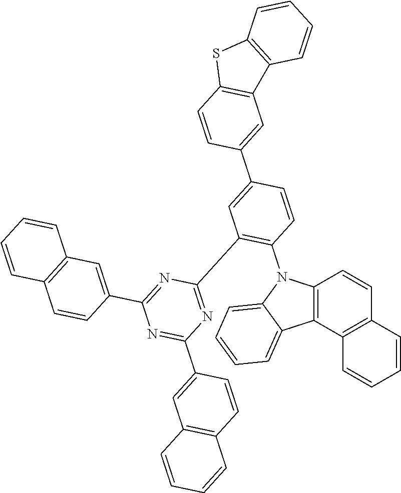

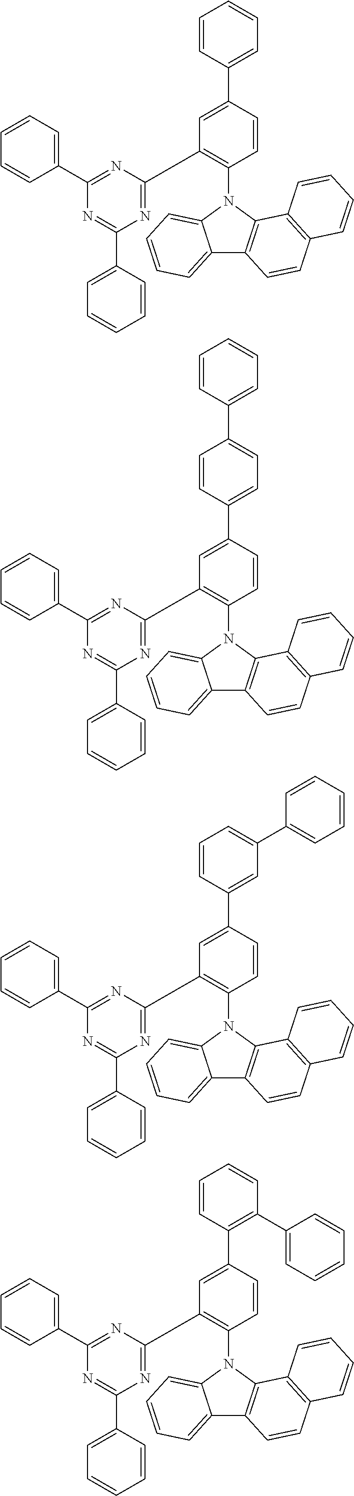

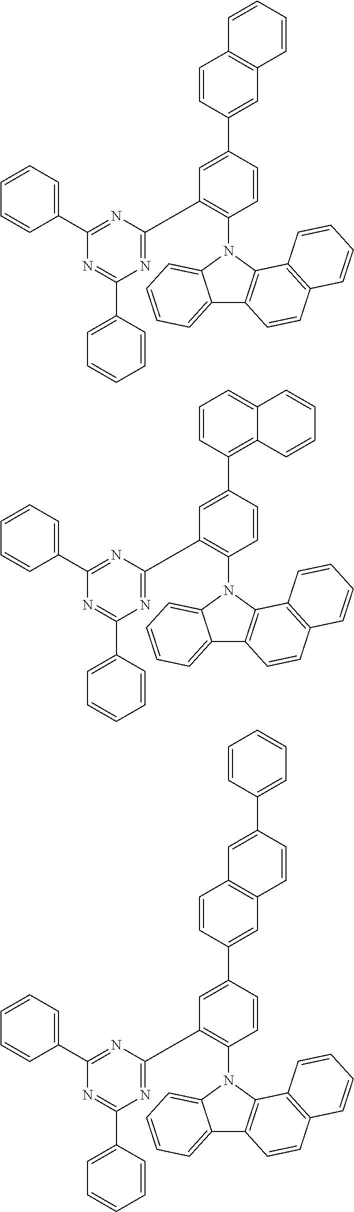

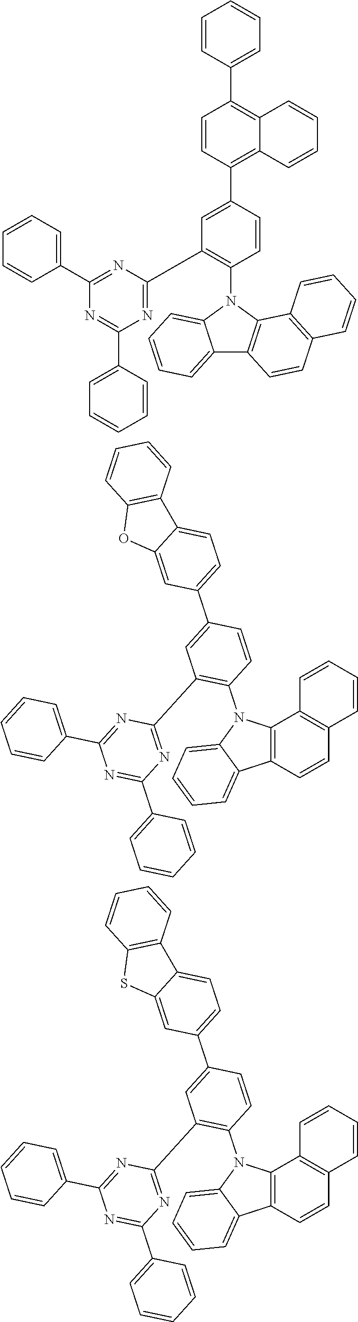

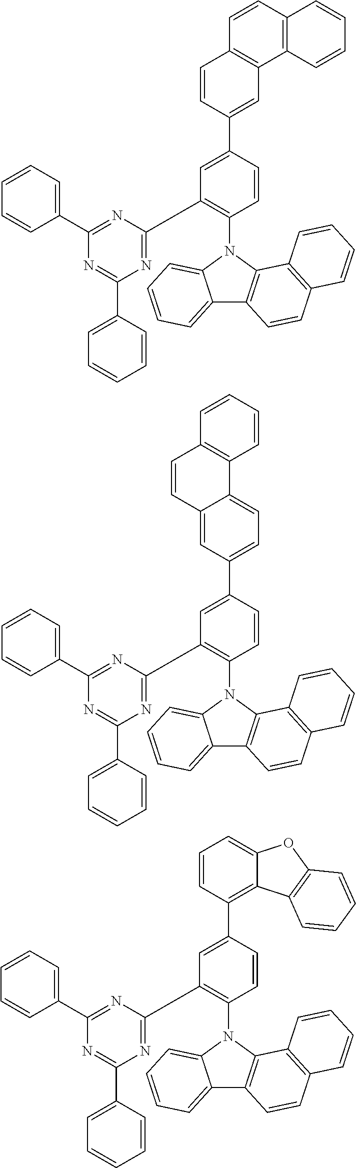

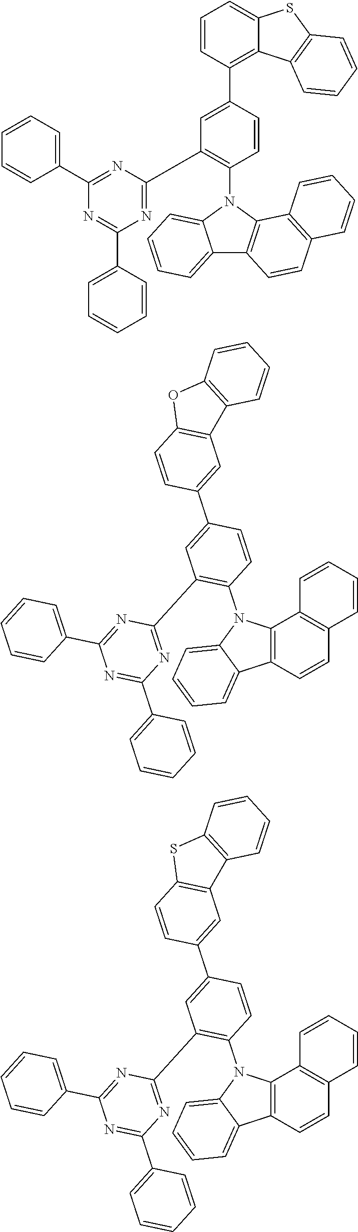

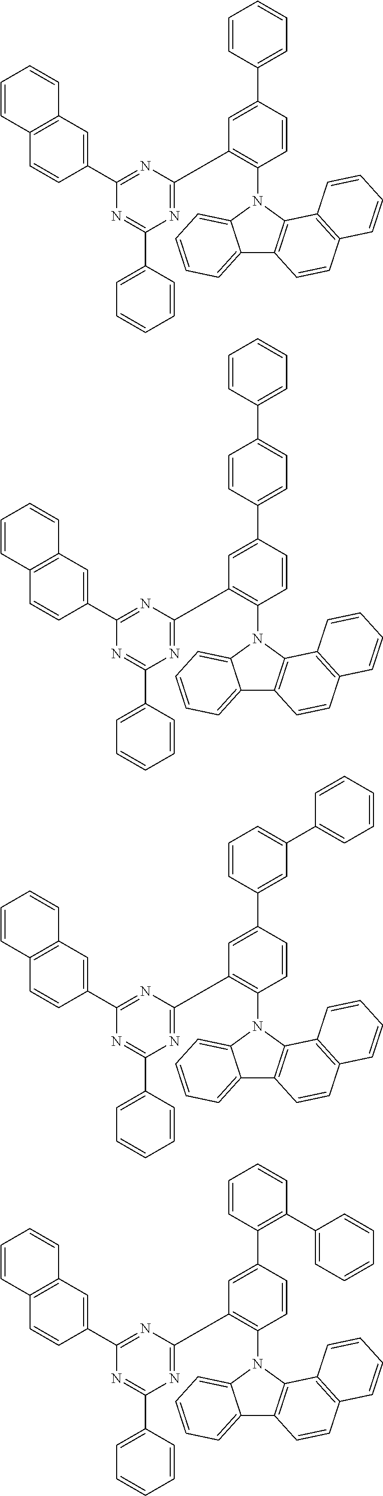

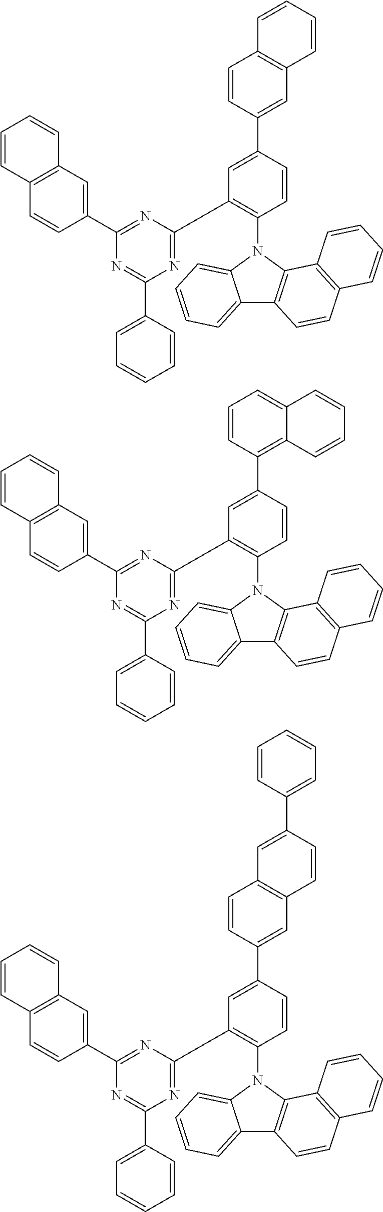

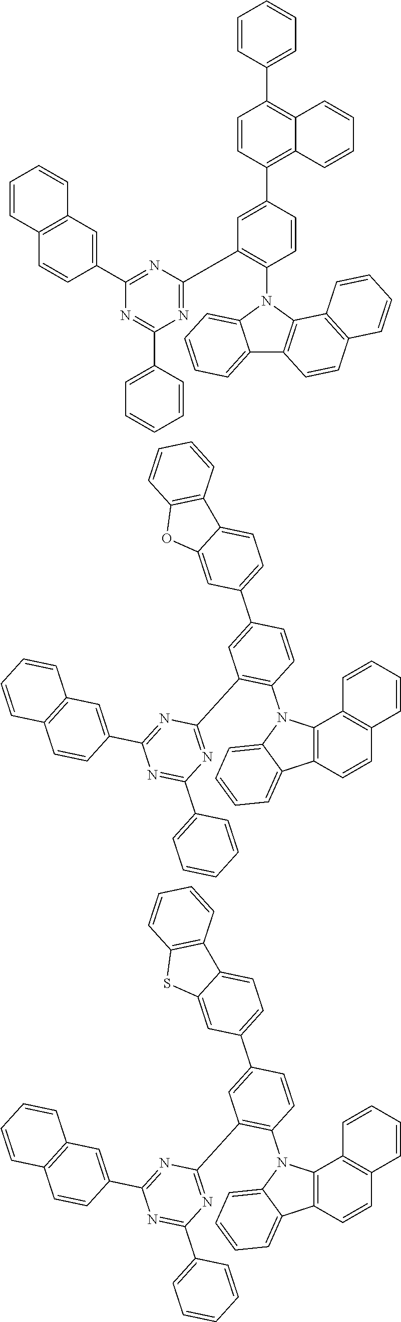

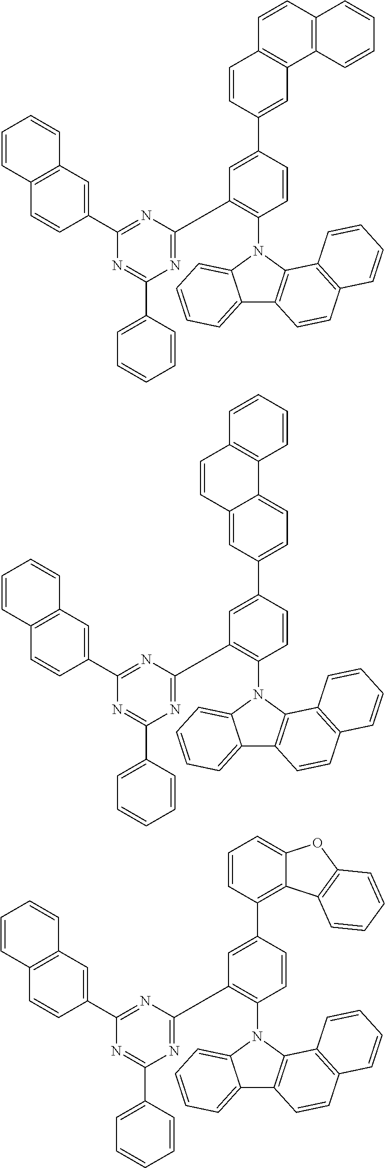

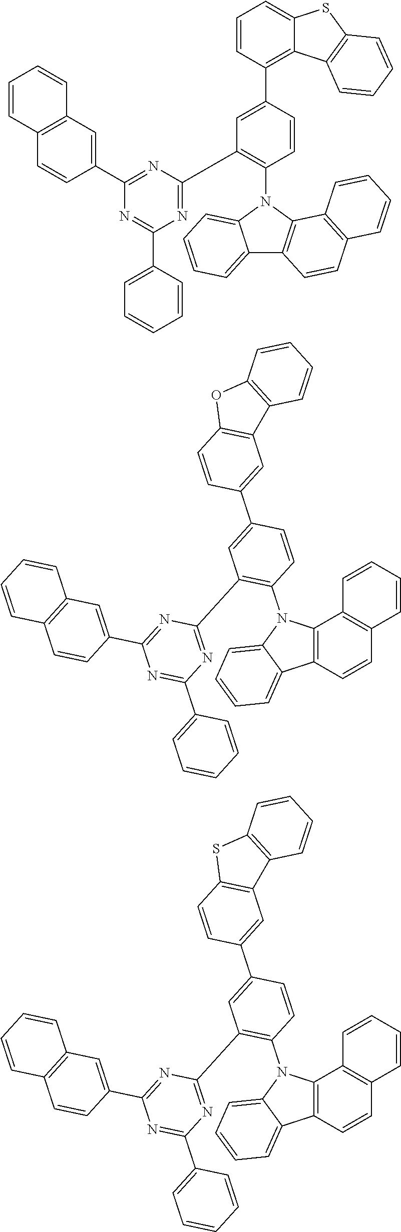

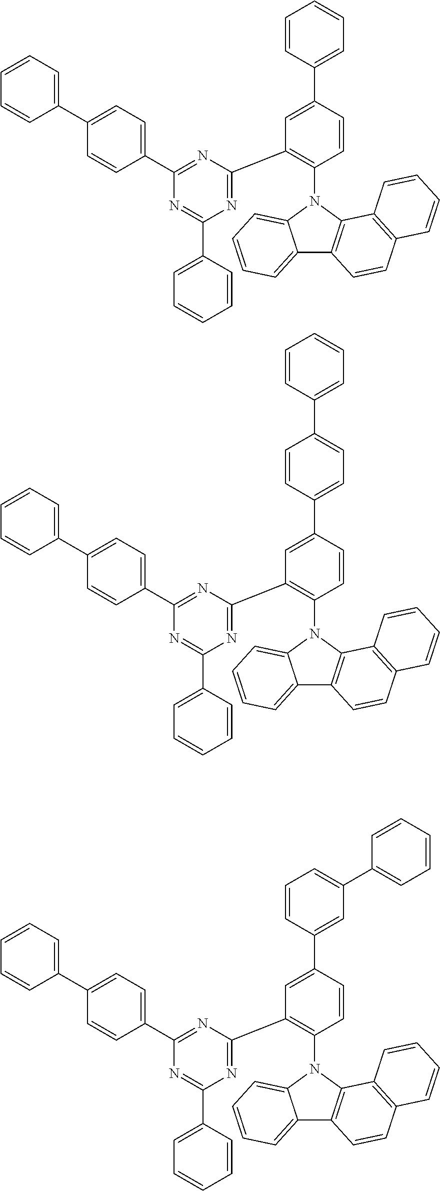

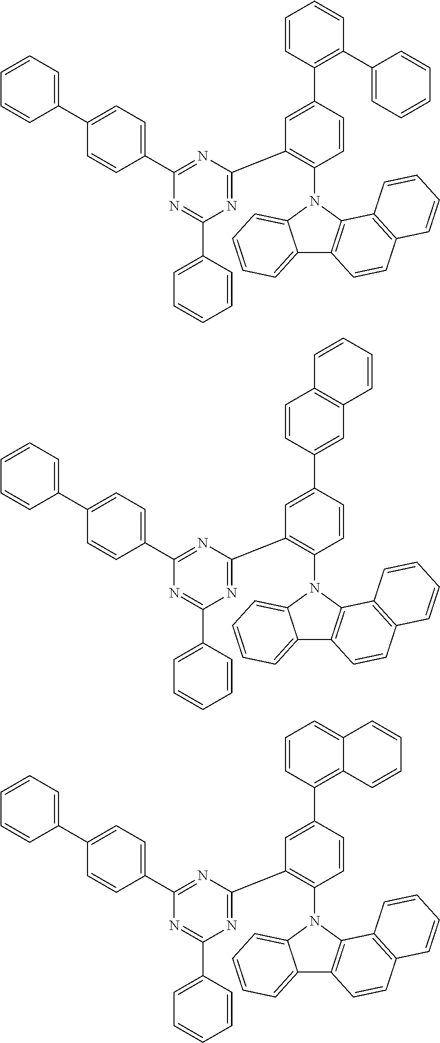

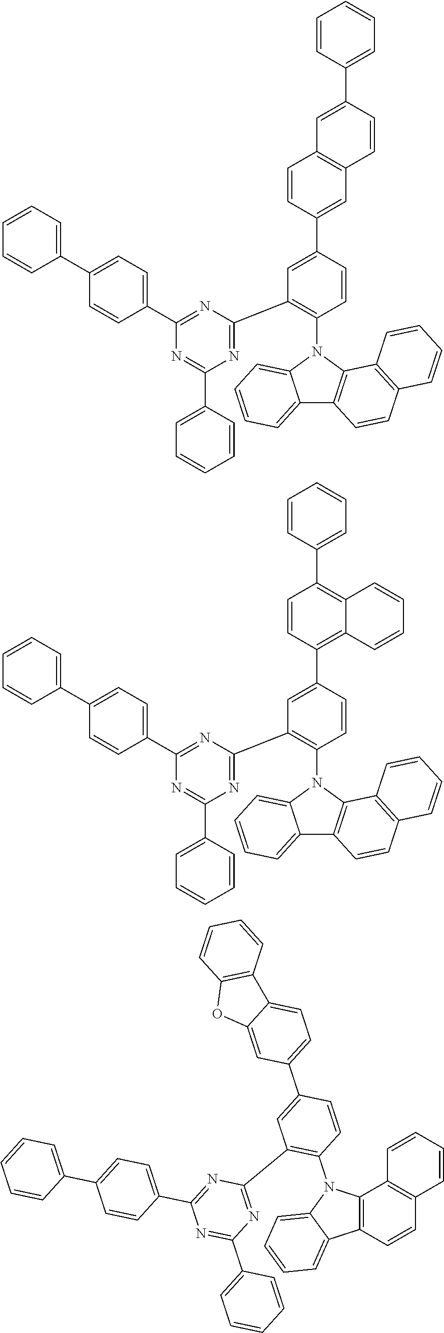

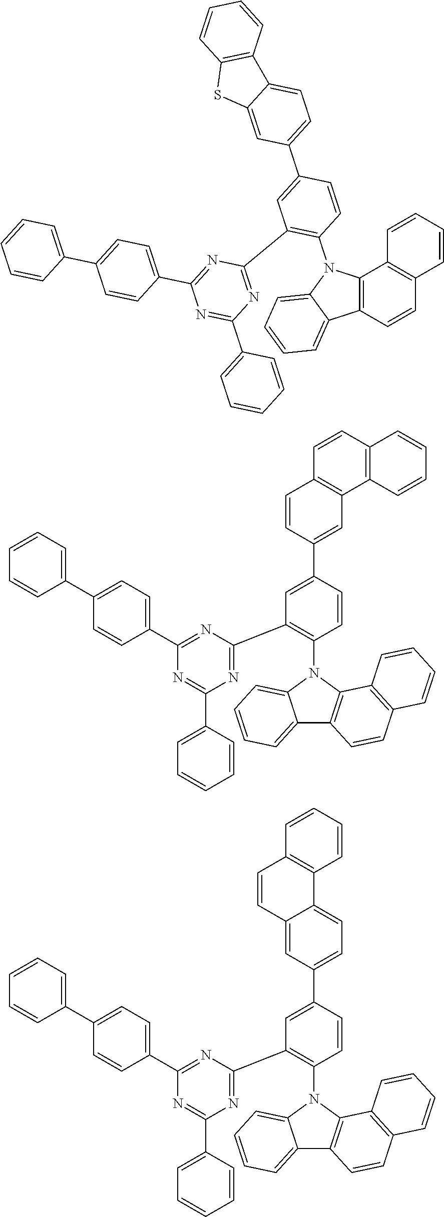

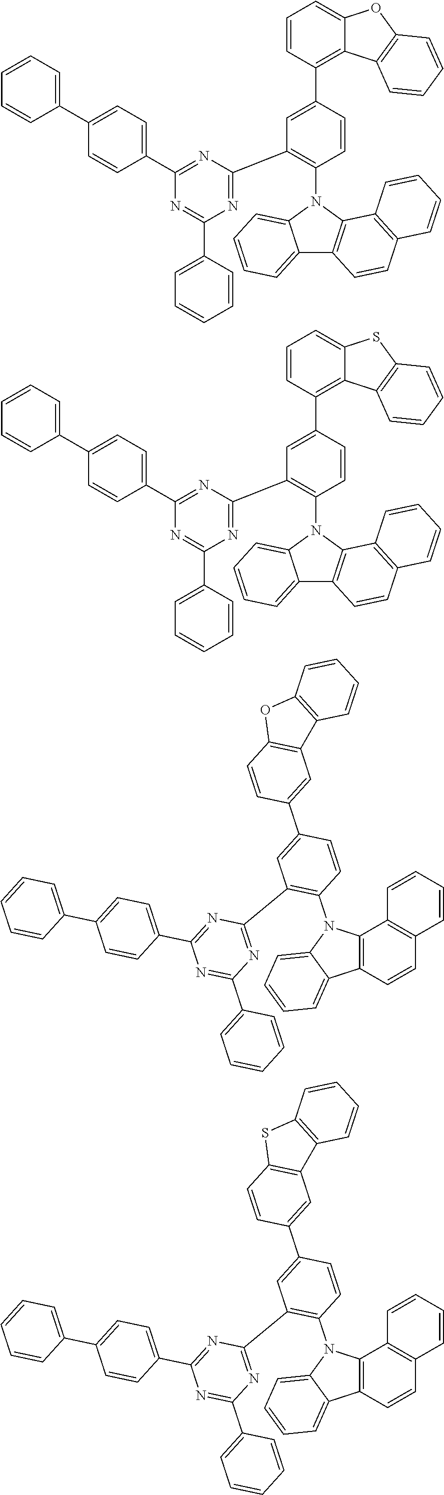

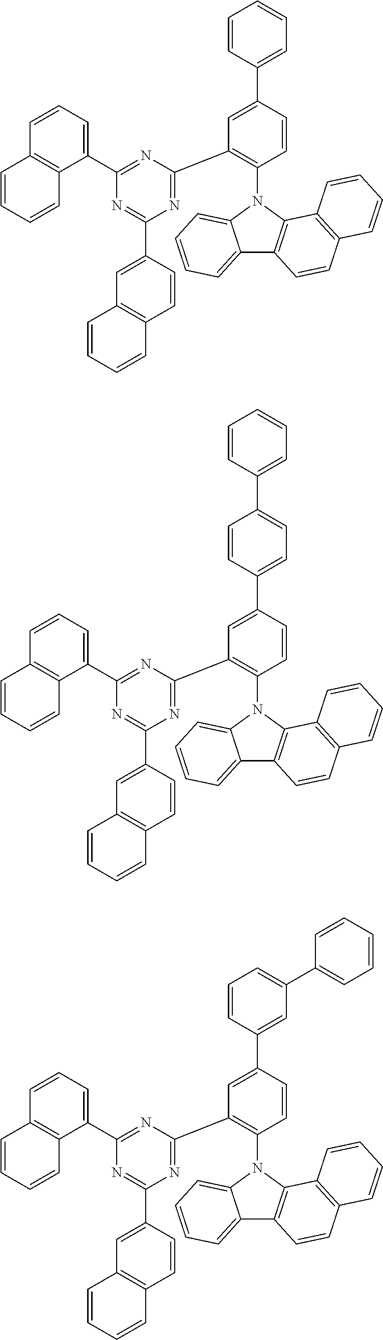

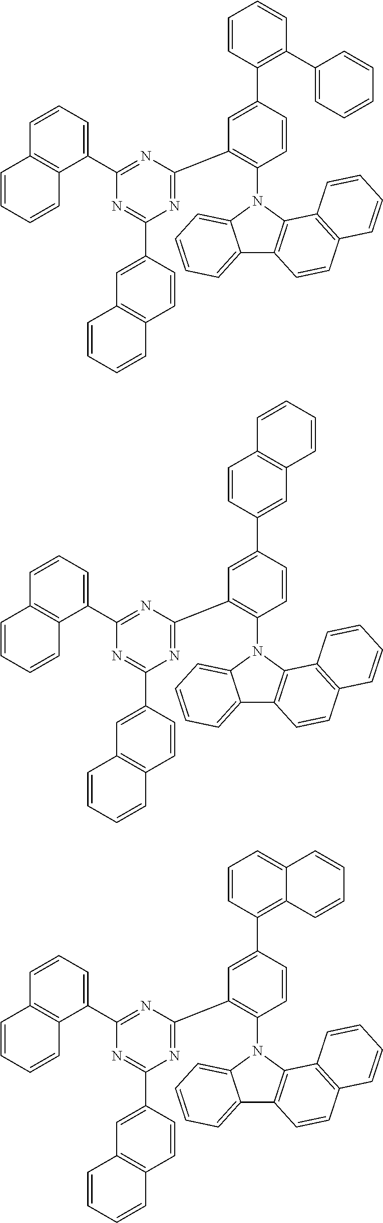

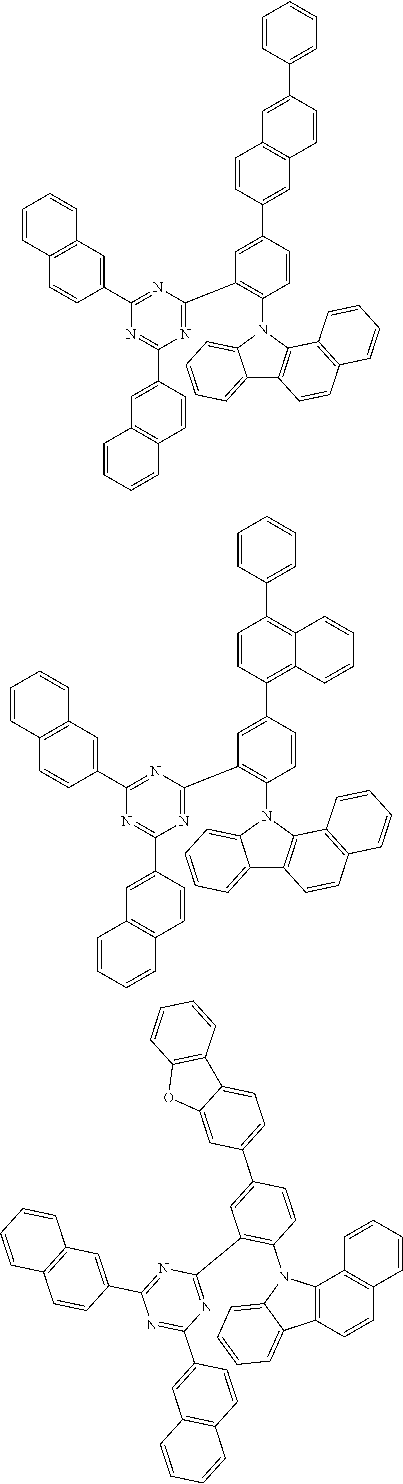

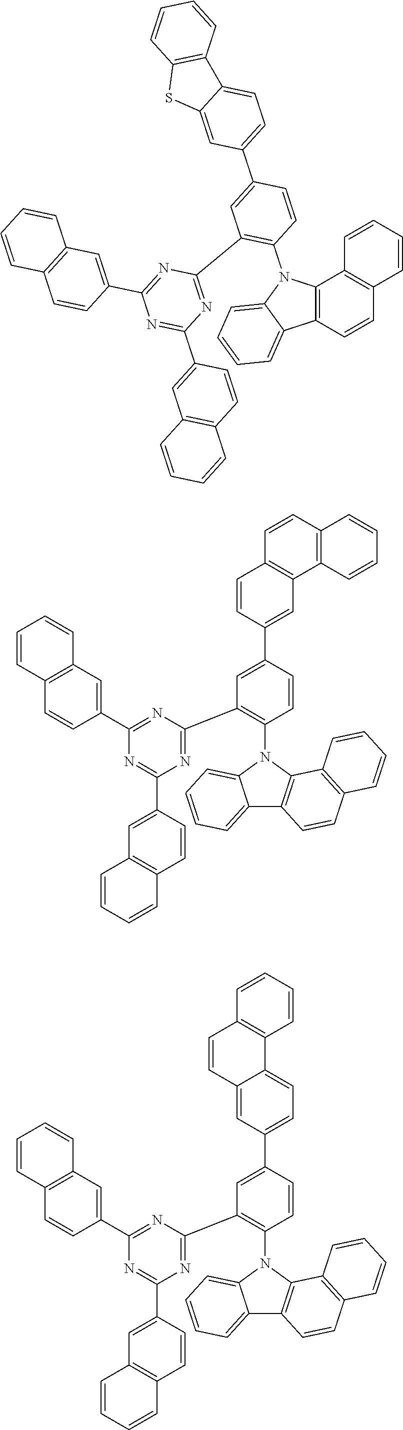

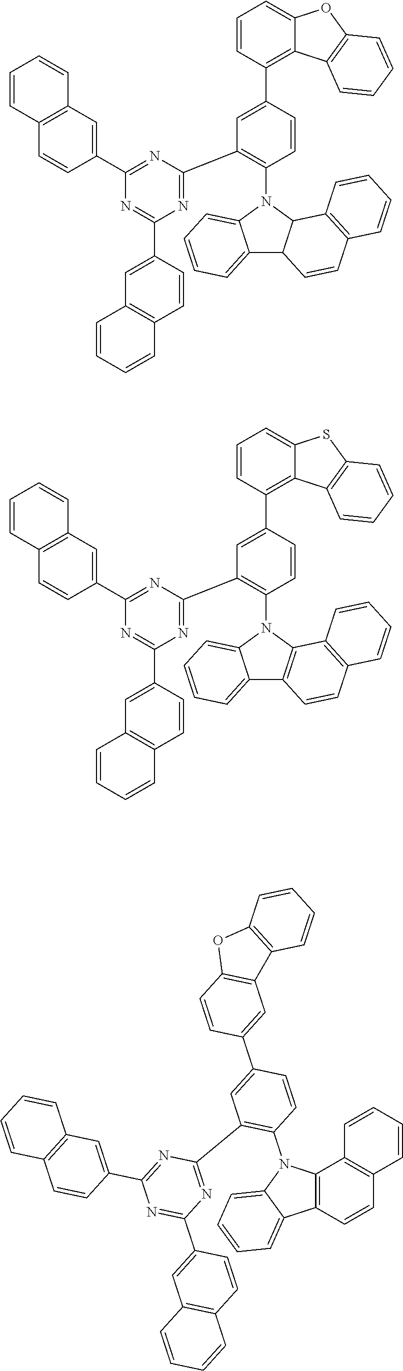

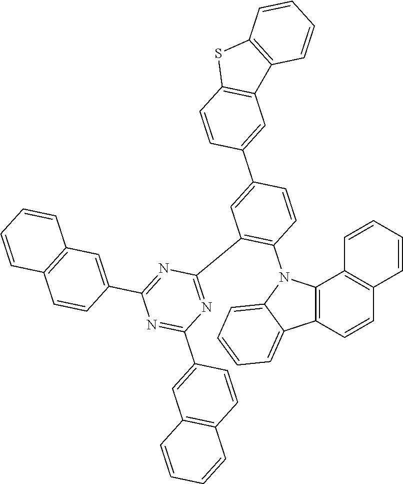

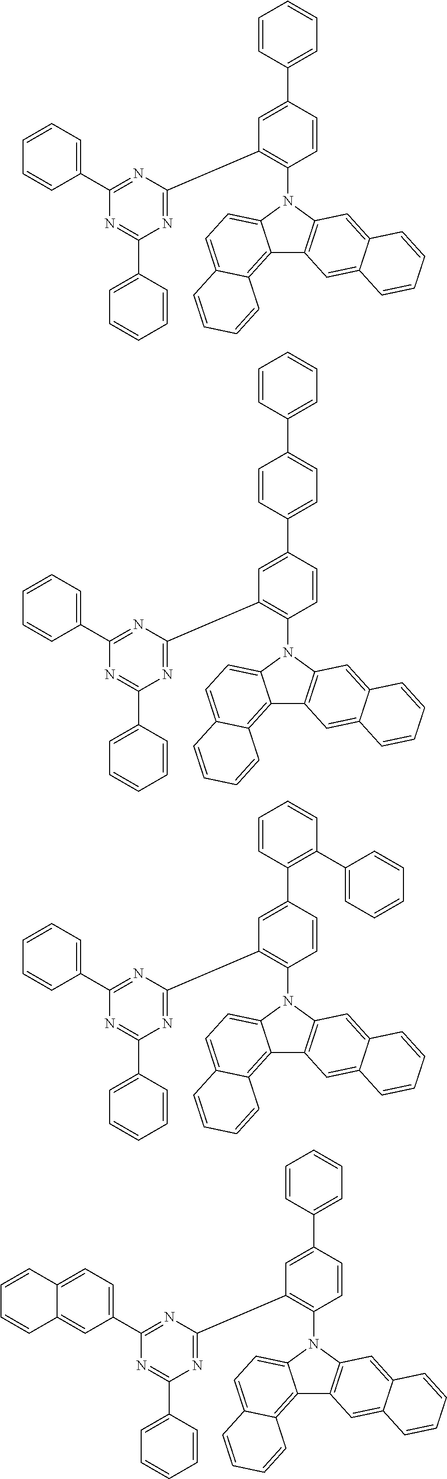

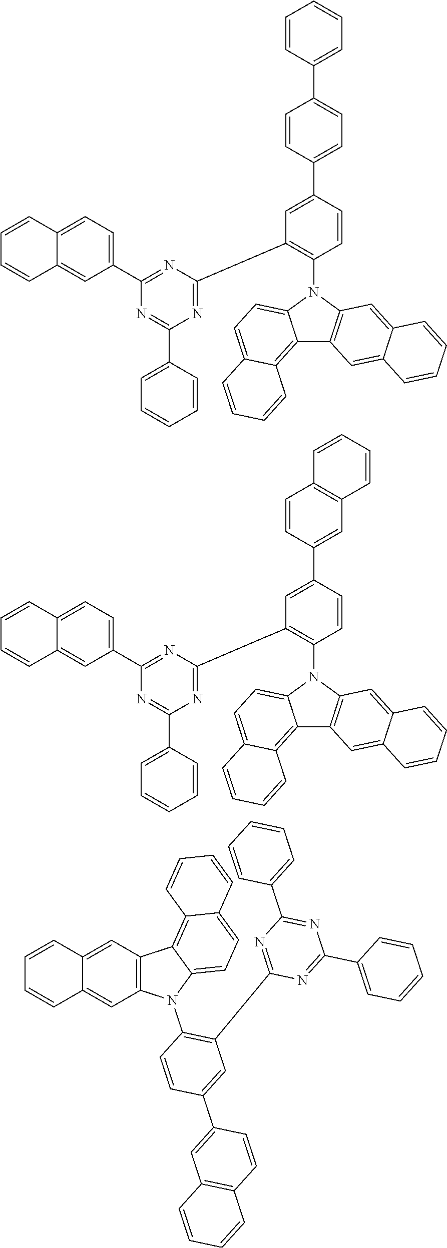

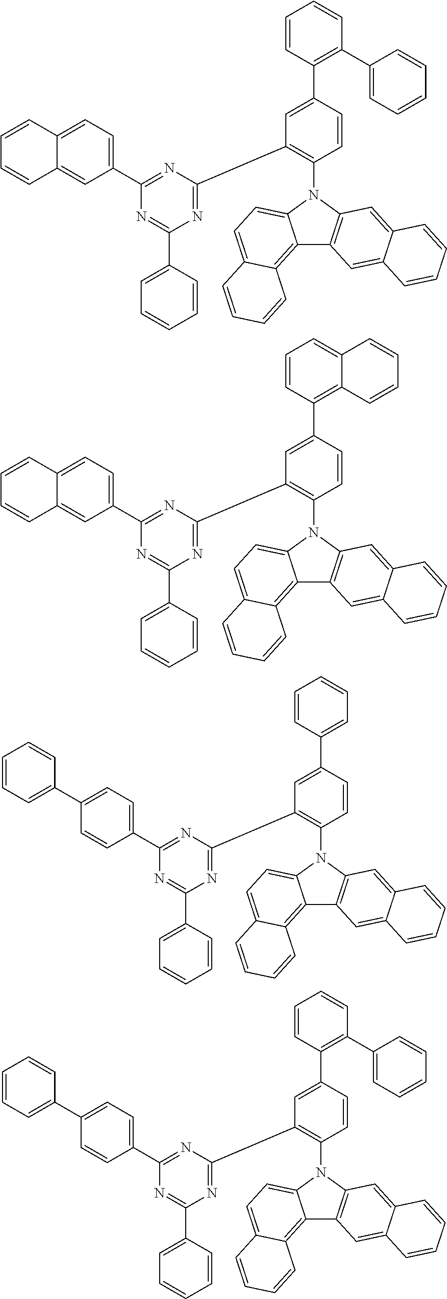

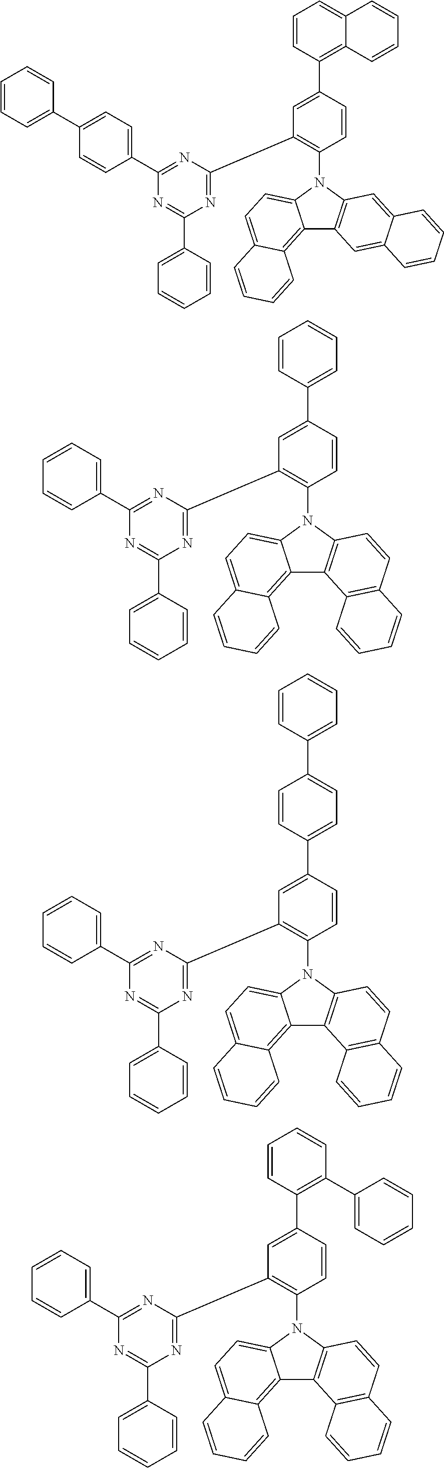

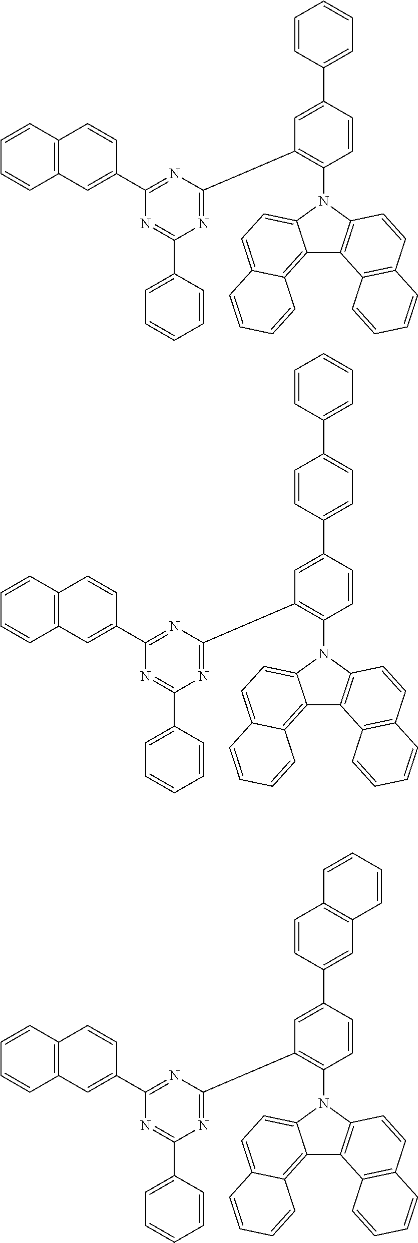

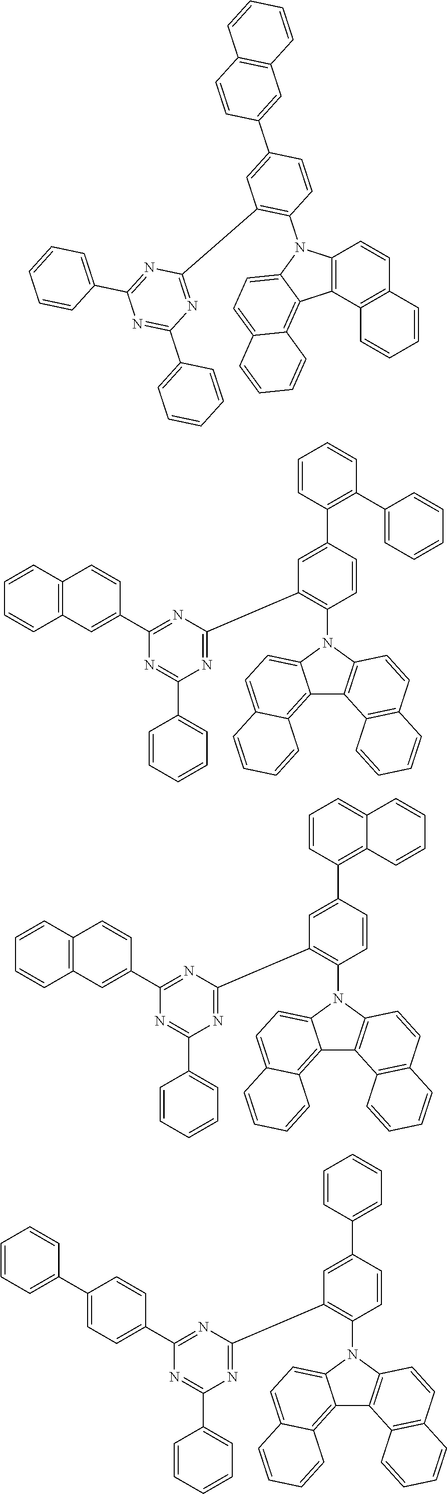

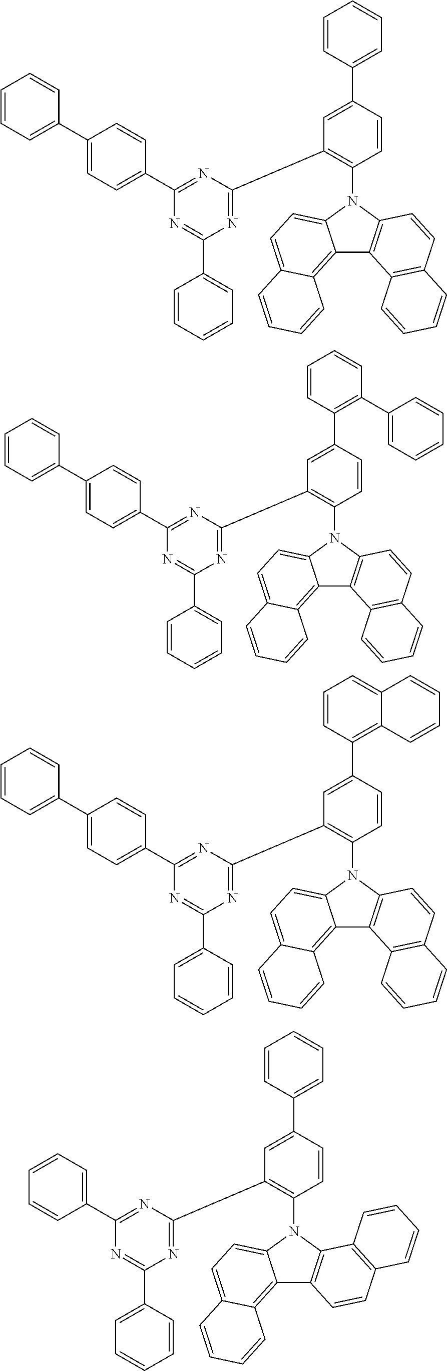

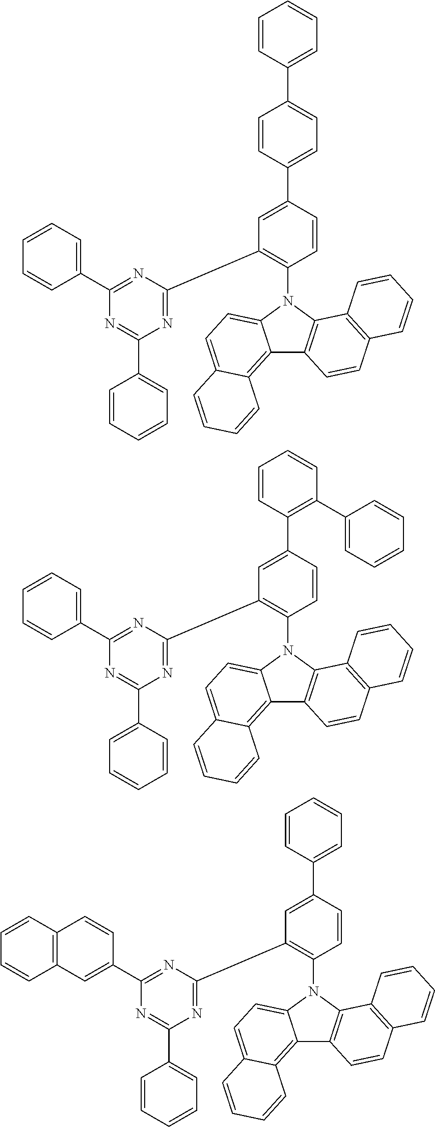

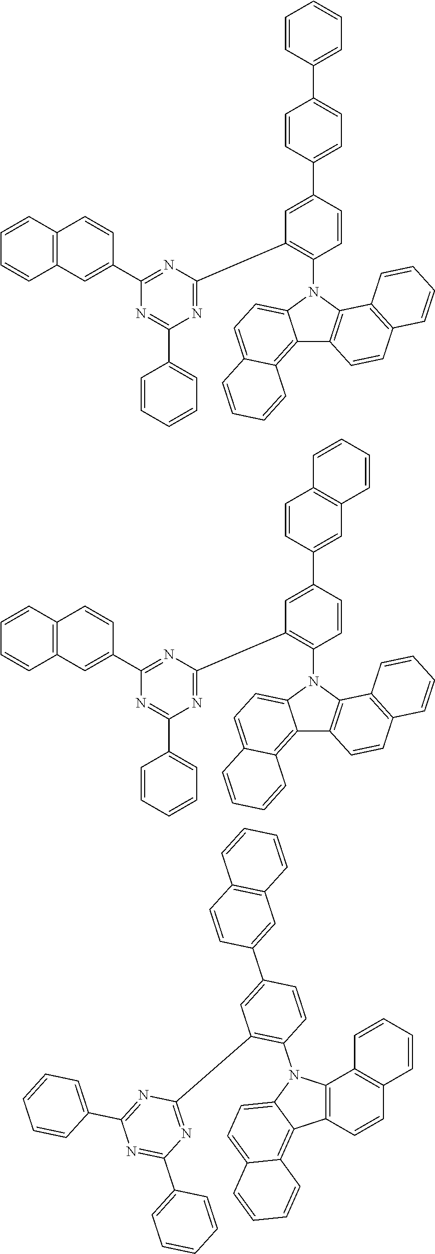

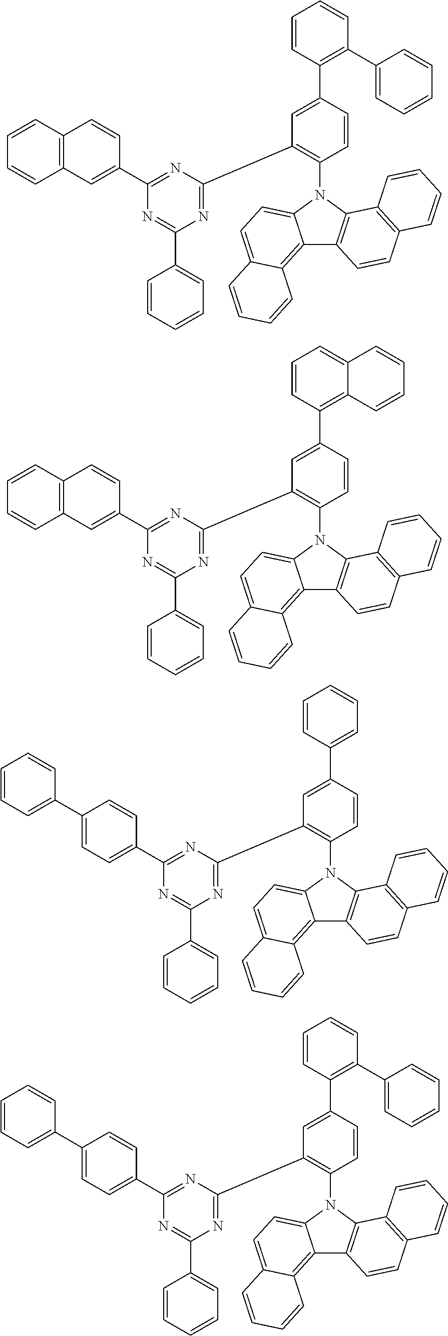

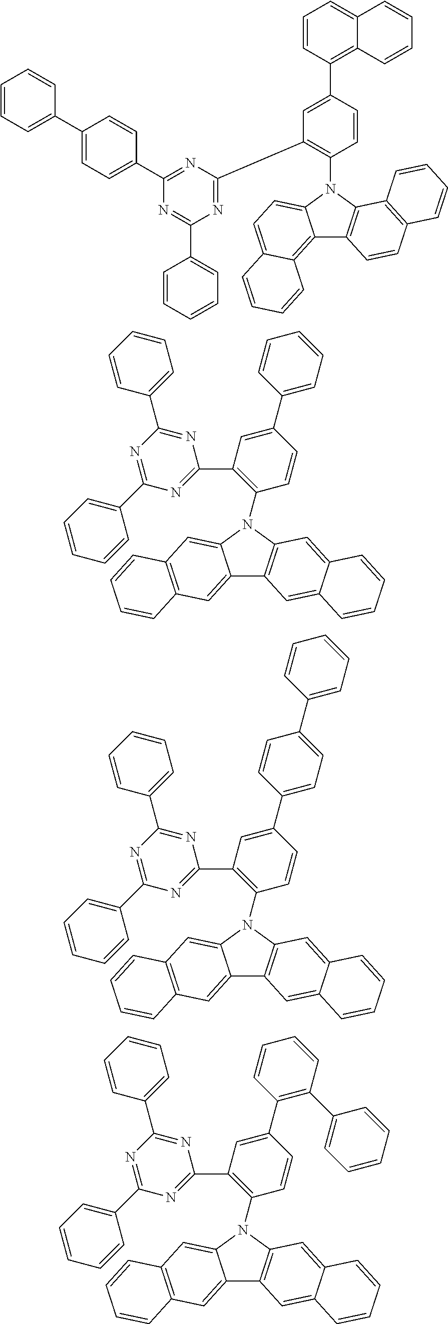

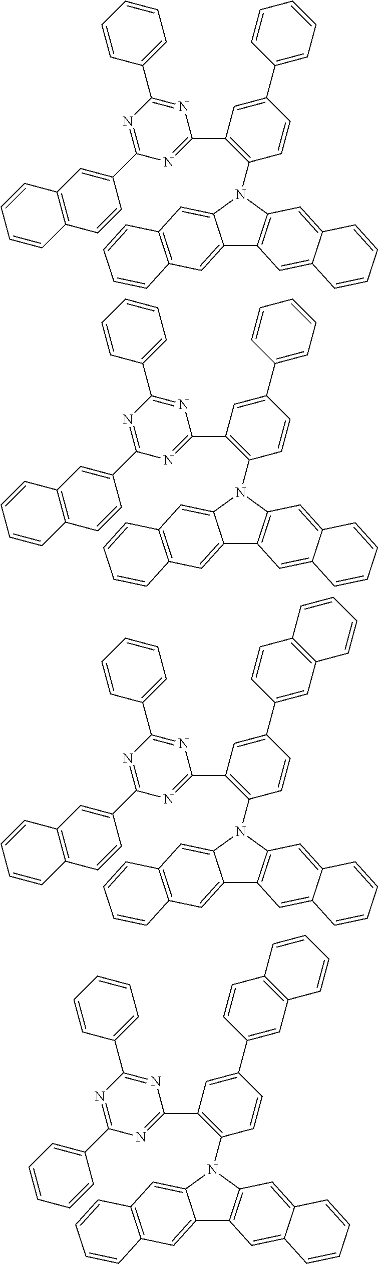

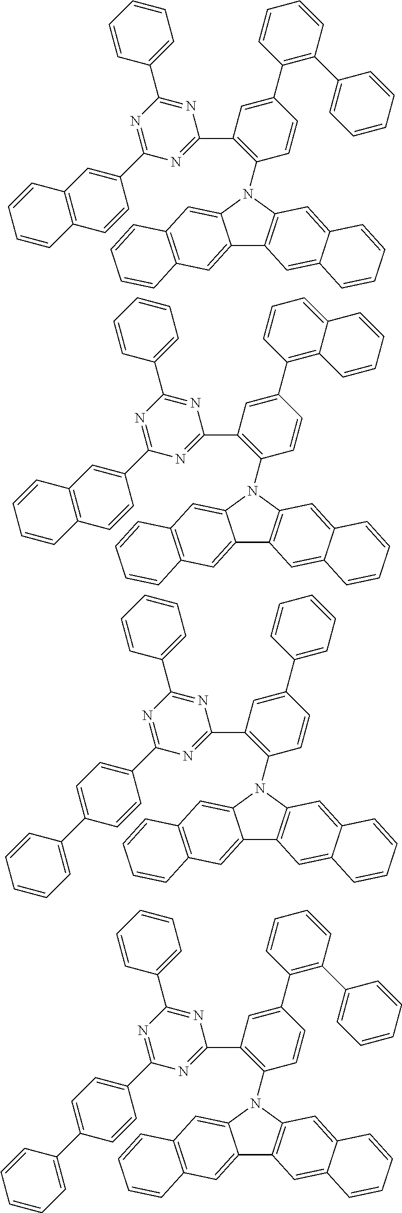

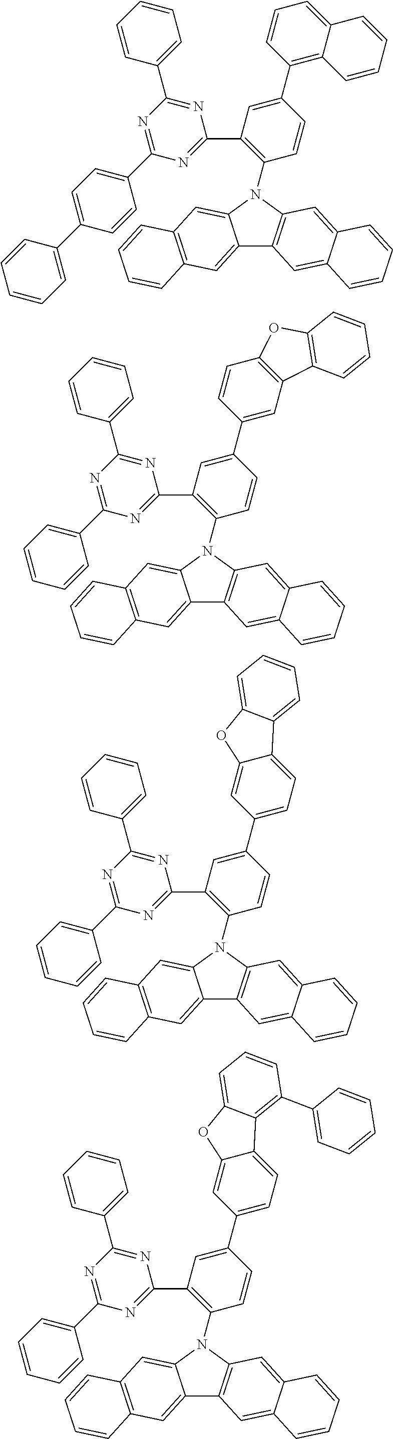

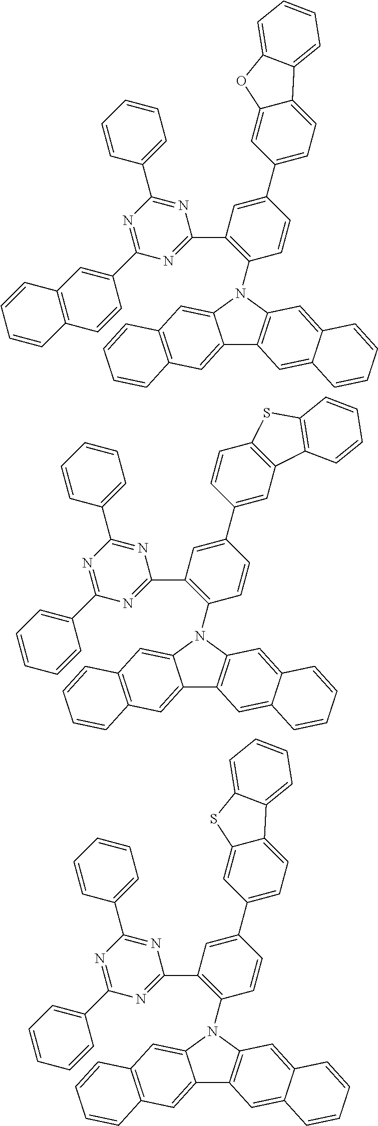

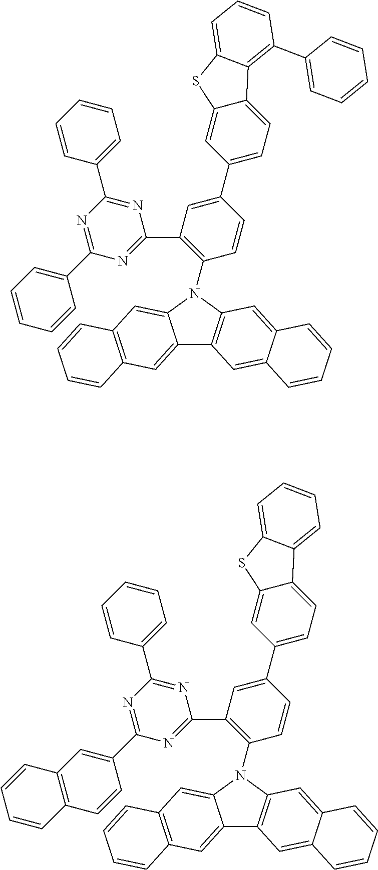

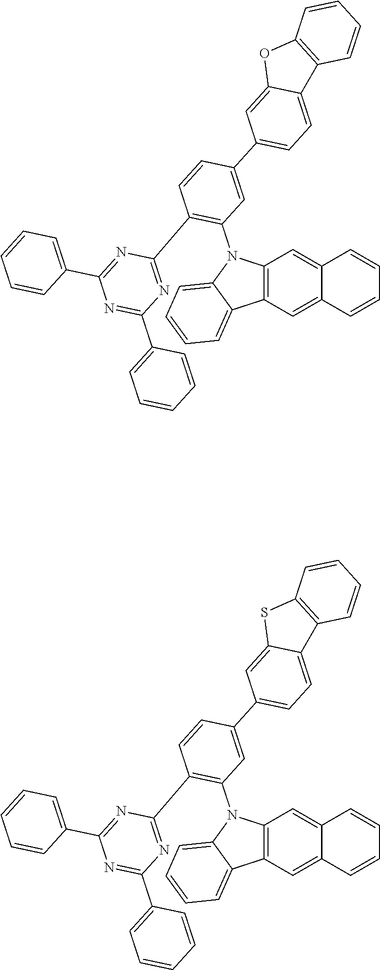

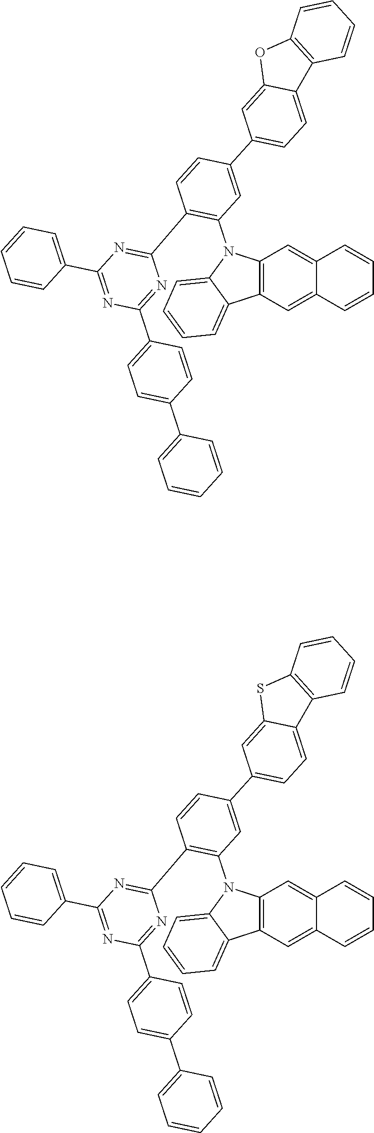

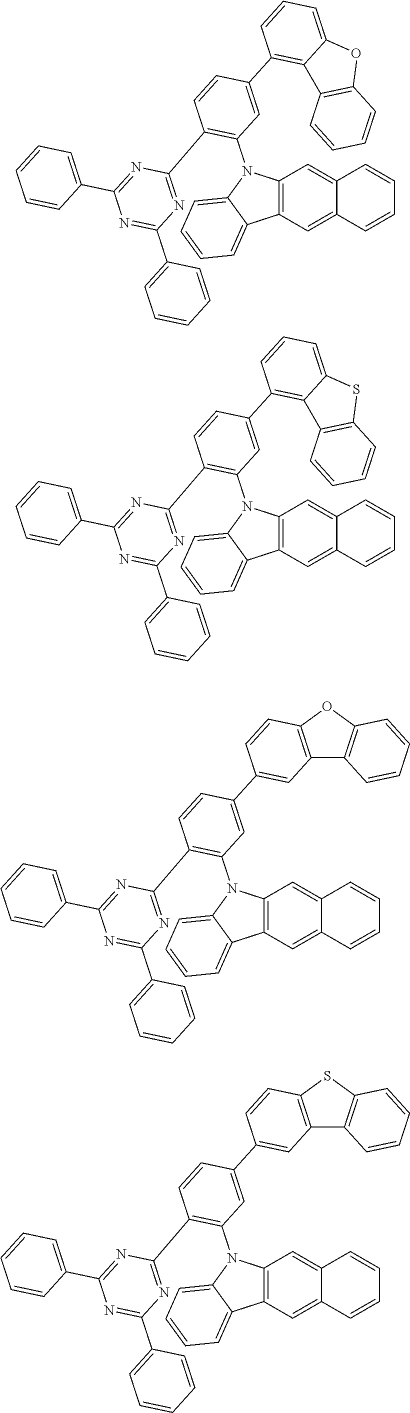

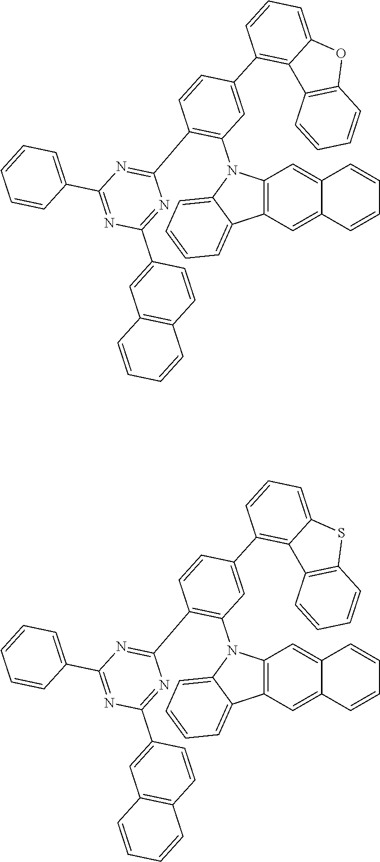

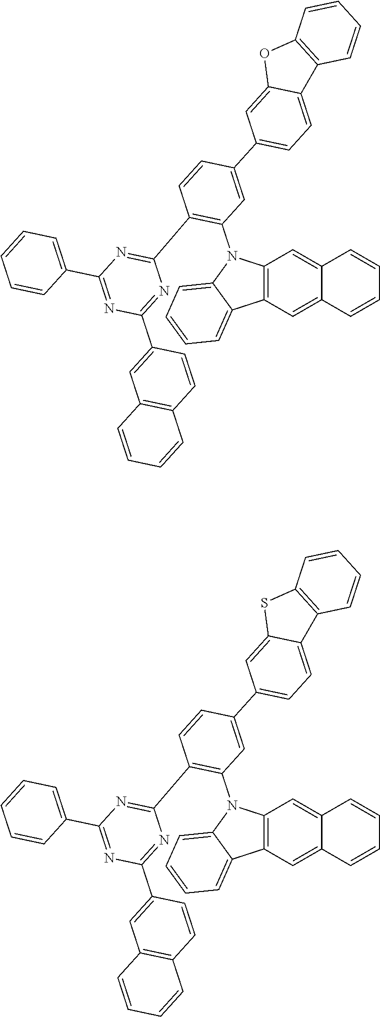

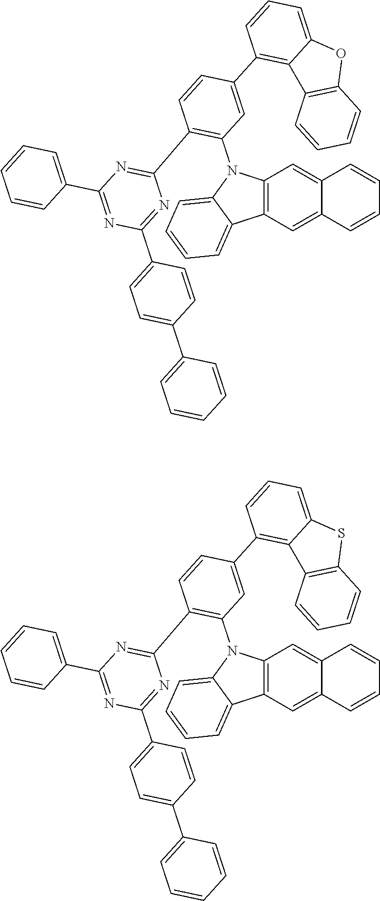

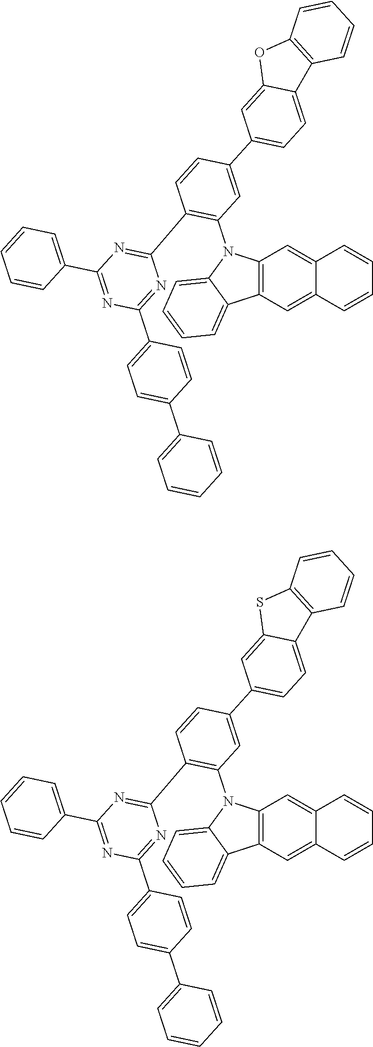

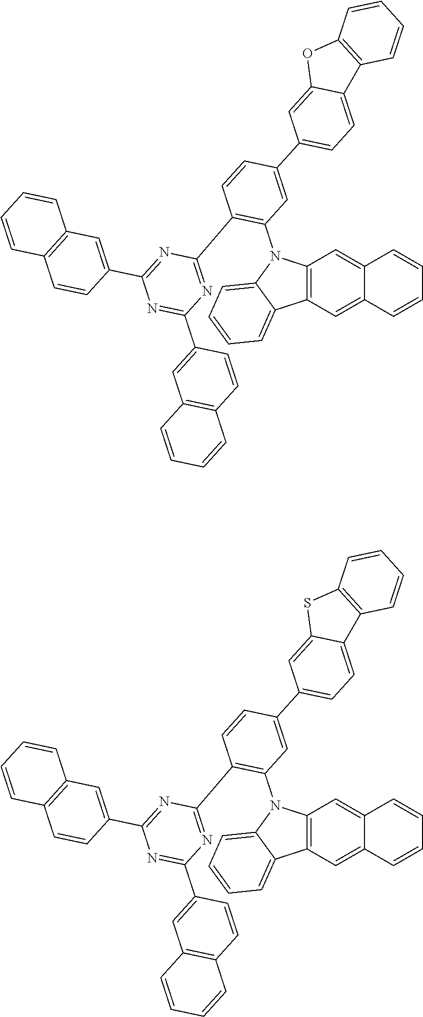

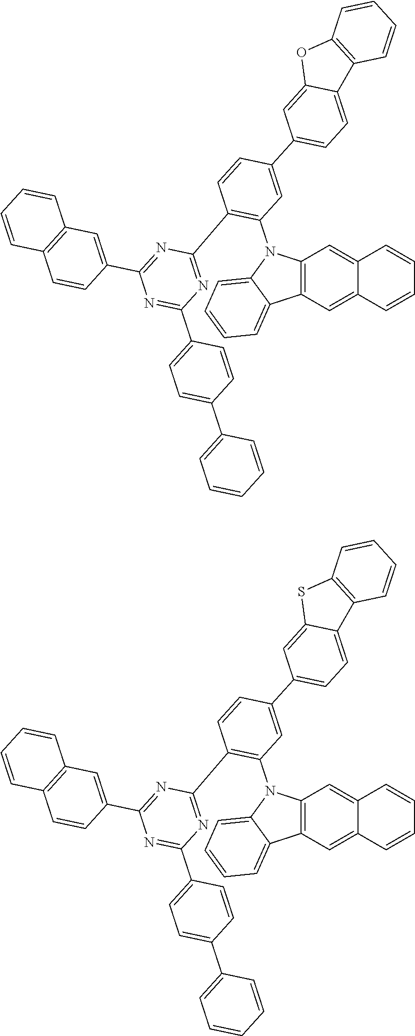

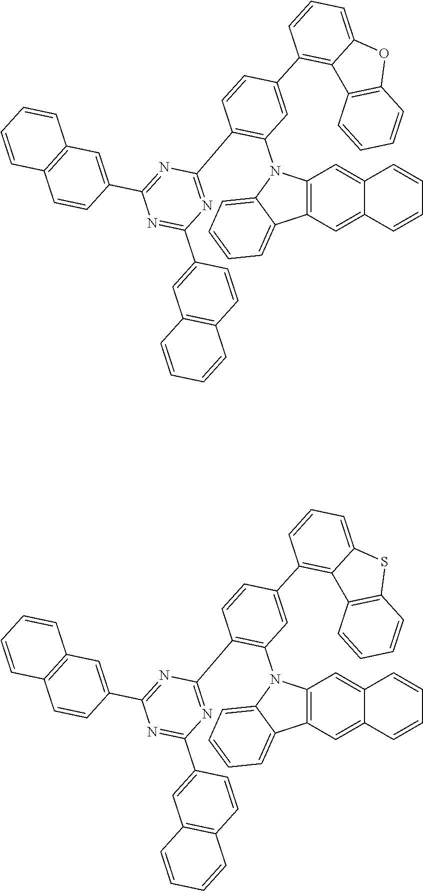

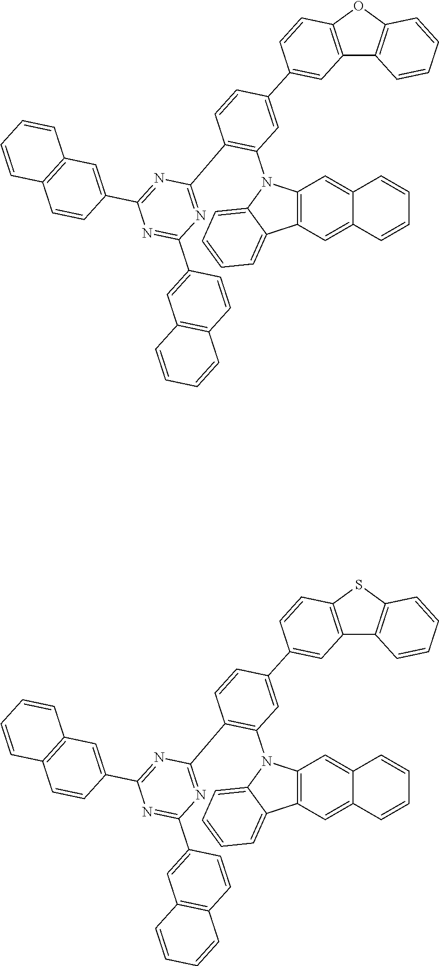

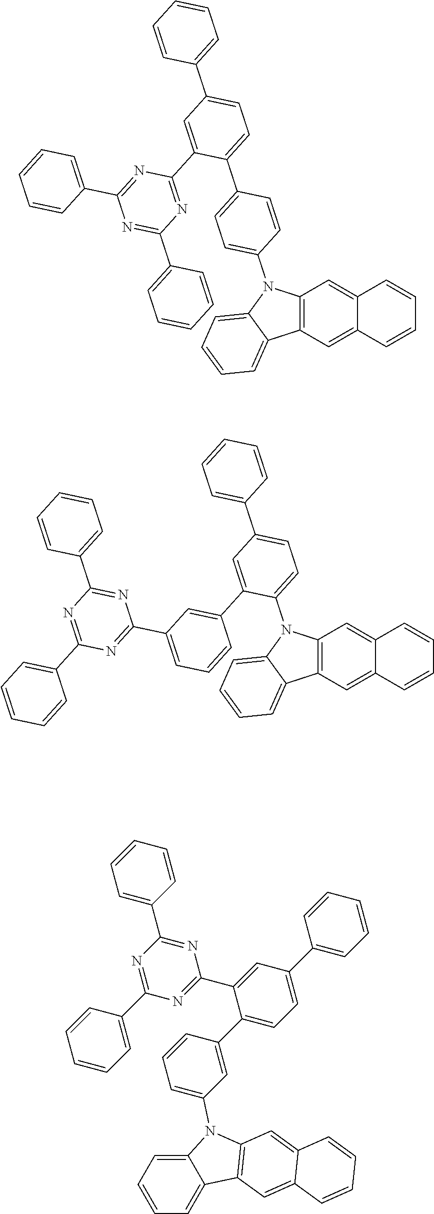

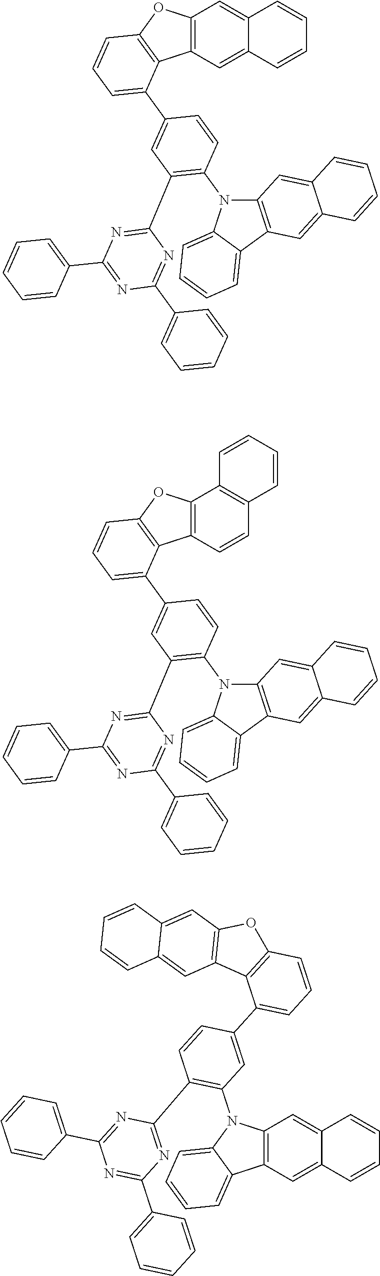

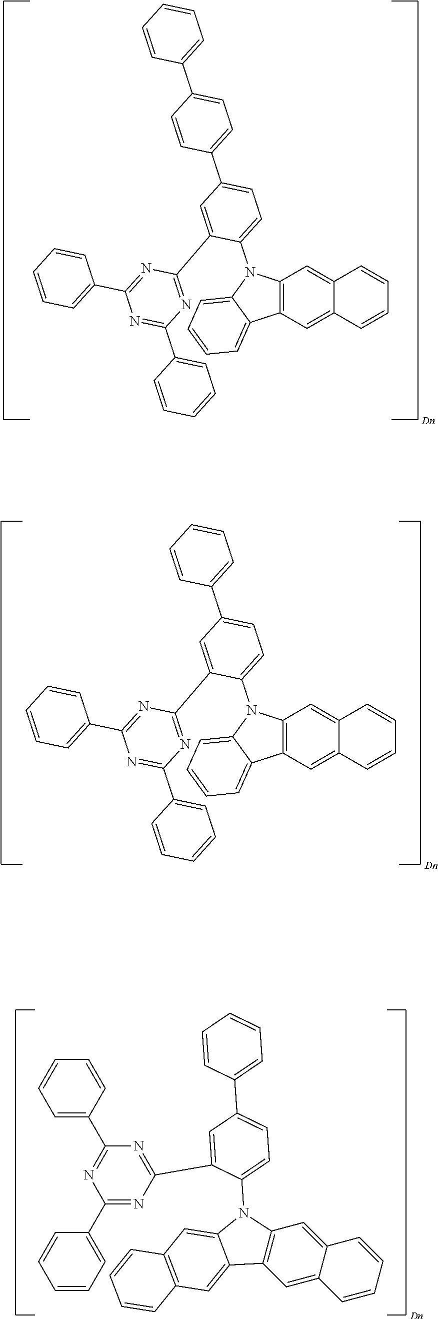

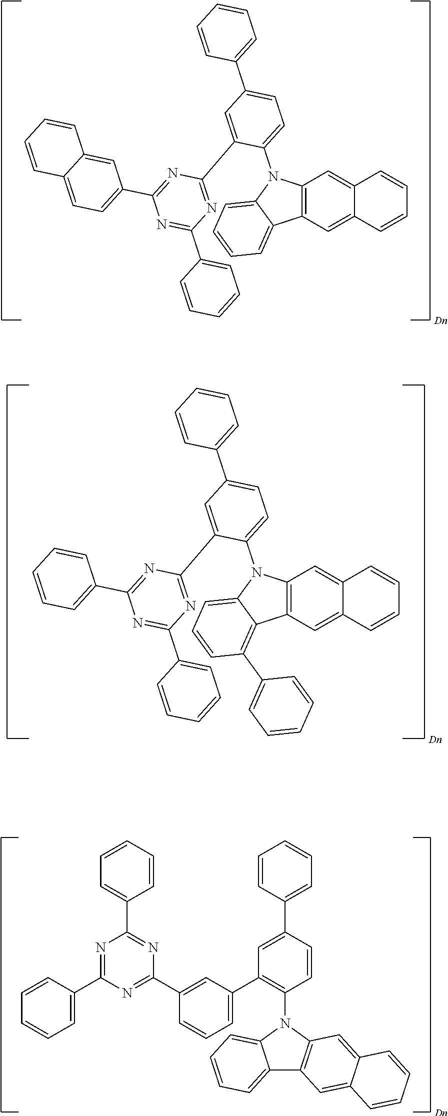

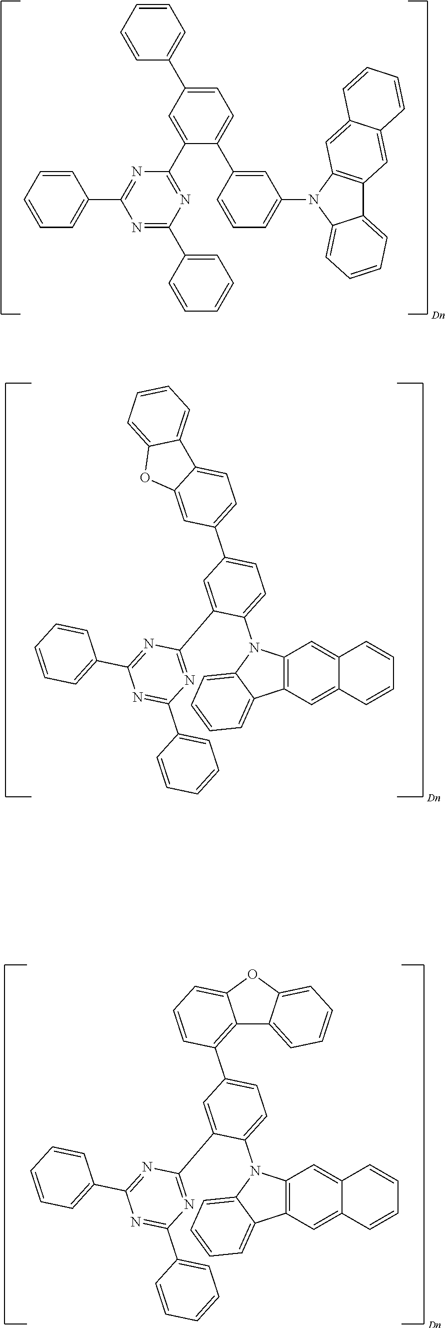

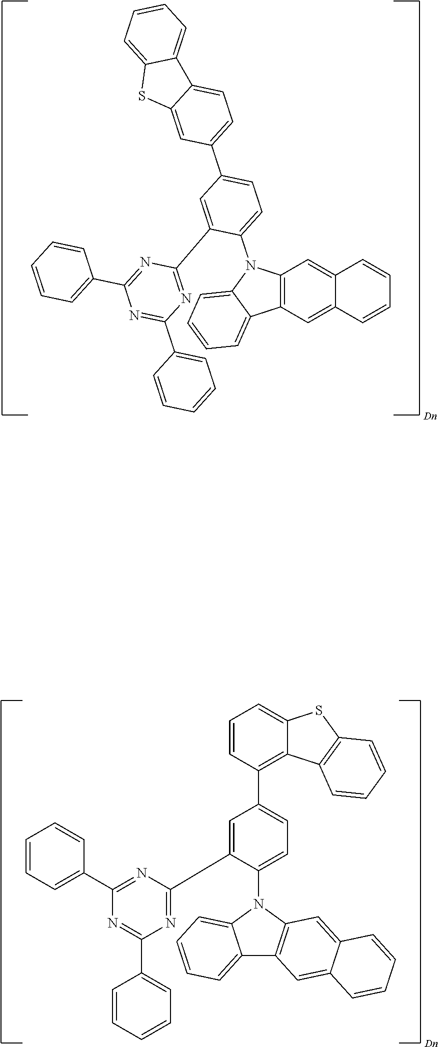

wherein,
Dn means that n hydrogens have been replaced by deuterium,
wherein n is an integer of 1 or more.

Meanwhile, the compound represented by Chemical Formula 1 can be prepared by a preparation method as shown in the following Reaction Scheme 1 as an example:

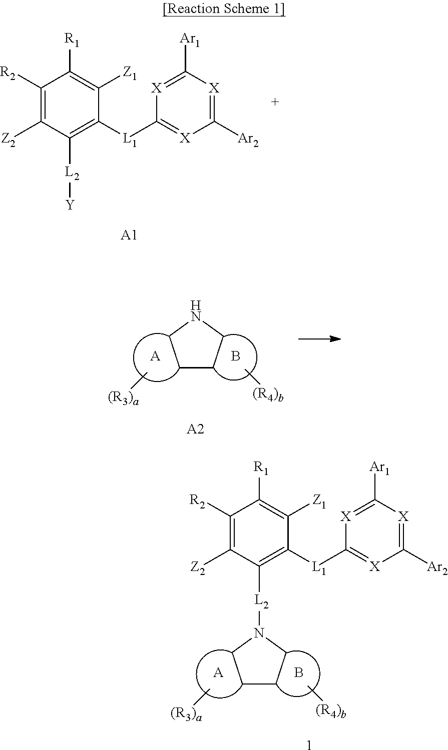

In Reaction Scheme 1, Y is halogen, preferably bromo, or chloro, and the definitions of other substituents are the same as described above.

Specifically, the compound may be prepared by an amine substitution reaction of the starting materials A1 and A2. Such an amine substitution reaction is preferably carried out in the presence of a palladium catalyst and a base, and a reactive group for the amine substitution reaction can be appropriately changed.

Further, the compound represented by Chemical formula 1 having at least one deuterium (i.e., a compound in which n is an integer greater than or equal to 1 in Chemical Formula 1D) can be prepared by a method as shown in the following Reaction Scheme 2.

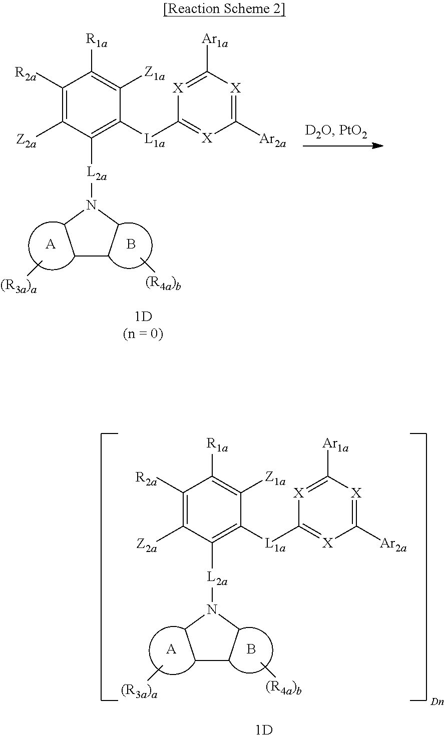

In Reaction Scheme 2, Y is halogen, preferably bromo, or chloro, and the definitions of other substituents are the same as described above.

Specifically, the compound represented by Chemical Formula 1 containing at least one deuterium can be prepared by deuterating a compound represented by Chemical Formula 1 that is not substituted with deuterium. This deuterium substitution reaction can be performed by reacting the non-deuterium-substituted compound with a deuterium-modified compound, such as deuterium oxide or a benzene-D₆ (C₆D₆) solution, in the presence of PtO₂. The deuterium substitution rate of the compound can be controlled by varying the reaction time and temperature conditions.

Meanwhile, the compound represented by Chemical Formula 1 can be prepared by appropriately modifying the reactants in Reaction Schemes 1 and 2, and the preparation methods thereof will be further detailed in the Preparation Examples described below.

### (Composition for an Organic Light Emitting Device)

In one embodiment of the present disclosure, a composition for an organic light emitting device is provided, including a compound represented by the Chemical formula 1 and a compound represented by the following Chemical formula 2. The compound represented by Chemical formula 1 and the compound represented by Chemical formula 2 can serve as a host in a light emitting layer of the organic light emitting device. Specifically, the compound represented by Chemical formula 2 functions as a P-type host material having a hole transport ability superior to an electron transport ability, and the compound represented by Chemical formula 1 functions as an N-type host material having an electron transport ability superior to a hole transport ability, thereby forming an exciplex. Accordingly, excitons can emit light evenly throughout the light emitting layer, thereby simultaneously improving the light emitting efficiency and lifespan characteristics of the organic light emitting device.

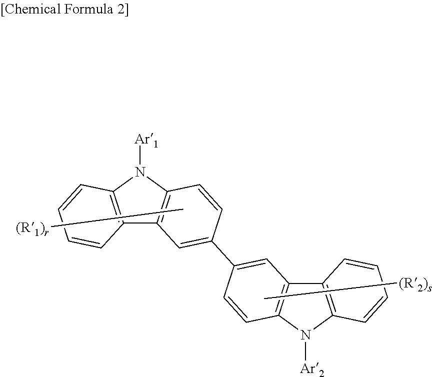

wherein in Chemical Formula 2,
Ar′₁ and Ar′₂ are each independently substituted or unsubstituted C₆₋₆₀ aryl, or substituted or unsubstituted C₂₋₆₀ heteroaryl comprising one or more heteroatoms among N, O and S,
R′₁ and R′₂ are each independently hydrogen, deuterium, C₁₋₆₀ alkyl, C₆₋₆₀ aryl, or C₂₋₆₀ heteroaryl comprising one or more heteroatoms among N, O and S, and
r and s are each independently an integer from 0 to 7.

When the organic light emitting device further comprises a compound represented by Chemical Formula 2, which can efficiently transfer holes from the host material of the light emitting layer to the dopant material, the probability of recombination of holes and electrons within the light emitting layer increases, along with the compound represented by Chemical Formula 1, which has excellent electron transport capabilities, thereby improving the efficiency and lifespan of the organic light emitting device.

According to one embodiment, the compound represented by Chemical Formula 2 may be represented by the following Chemical Formula 2′:

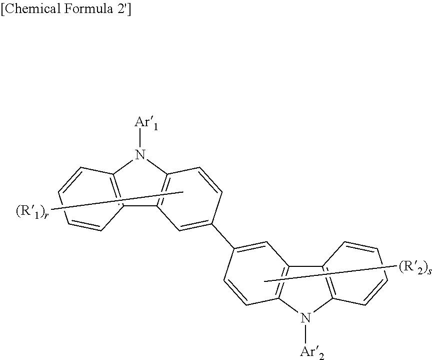

wherein in Chemical Formula 2′,
Ar′₁, Ar′₂, R′₁, R′₂, r and s are as defined in Chemical Formula 22.

Further, in Chemical Formula 2, Ar′₁ and Ar′₂ may be each independently, C₆₋₂₀ aryl, or C₂₋₂₀ heteroaryl comprising one heteroatom among N, O and S,
Wherein, Ar′₁ and Ar′₂ are unsubstituted, or substituted with one or more substituents selected from the group consisting of deuterium and C₆₋₂₀ aryl unsubstituted or substituted with deuterium.

For example, Ar′₁ and Ar′₂ may be each independently, phenyl, biphenylyl, terphenylyl, naphthyl, dibenzofuranyl, or dibenzothiophenyl,
Wherein, Ar′₁ and Ar′₂ are unsubstituted, or substituted with one or more substituents selected from the group consisting of deuterium and C₆₋₂₀ aryl unsubstituted or substituted with deuterium.

In this case, at least one of Ar′₁ and Ar′₂ may be phenyl unsubstituted or substituted with deuterium; or biphenylyl unsubstituted or substituted with deuterium

Further, in Chemical Formula 2, R′₁ and R′₂ may be each independently, hydrogen; deuterium; or C₆₋₂₀ aryl unsubstituted or substituted with deuterium.

For example, R′₁ and R′₂ may be each independently, hydrogen; deuterium; or phenyl unsubstituted or substituted with deuterium, but are not limited thereto.

Further, r and s, which represent the number of R′₁ and R′₂, respectively, may each independently be 0, 1, 2, 3, 4, 5, 6, or 7.

More specifically, r and s may be each independently, 0, 1, or 7.

For example, r+s is 0 or 1.

The compound represented by the Chemical formula 2 is any one selected from the group consisting of the following compounds and compounds substituted with deuterium thereof:

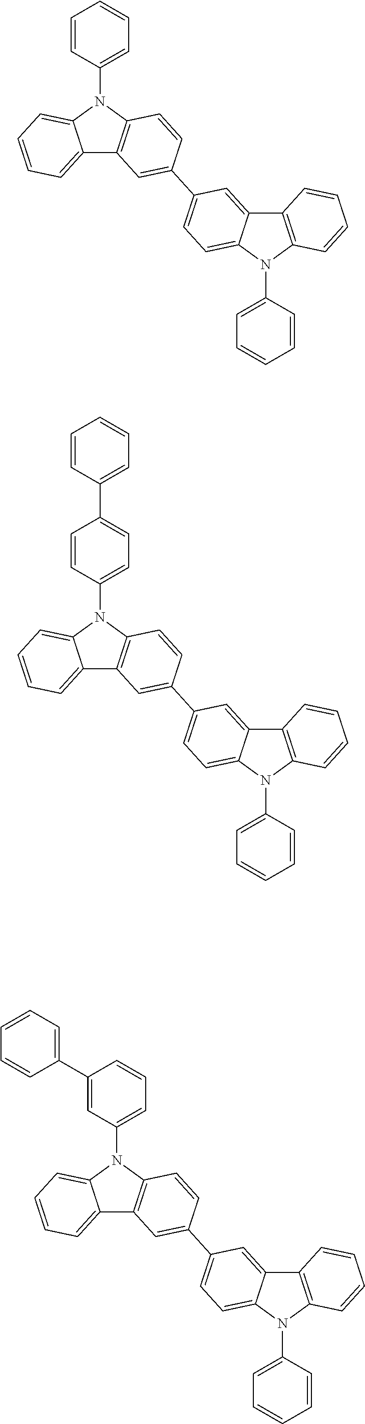 [SMILES: c1ccc(-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cccc(-n3c4ccccc4c4cc(-c5ccc6c(c5)c5ccccc5n6-c5ccccc5)ccc43)c2)cc1]

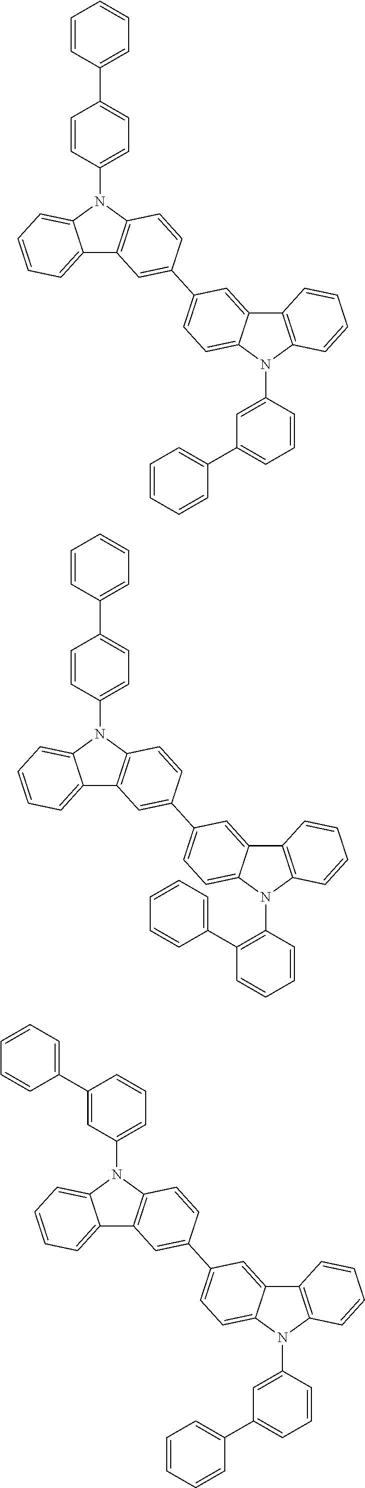 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5-c5ccccc5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1]

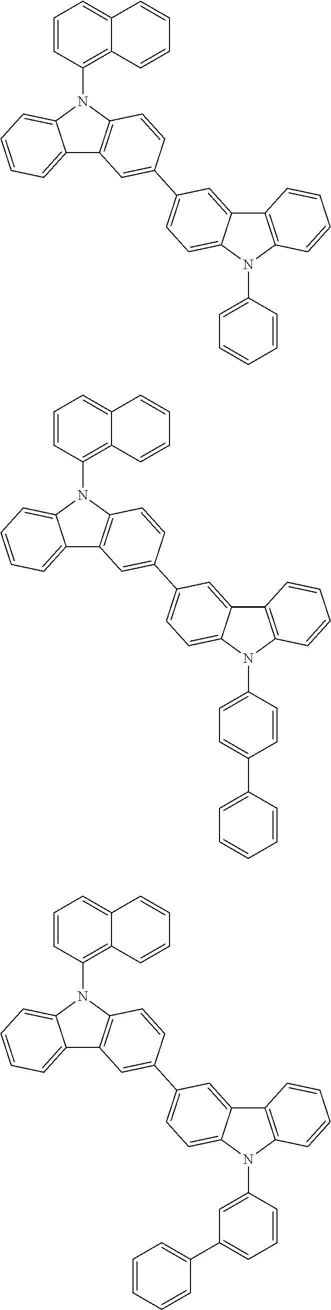 [SMILES: c1ccc(-n2c3ccccc3c3cc(-c4ccc5c(c4)c4ccccc4n5-c4cccc5ccccc45)ccc32)cc1 ; c1ccc(-c2ccc(-n3c4ccccc4c4cc(-c5ccc6c(c5)c5ccccc5n6-c5cccc6ccccc56)ccc43)cc2)cc1 ; c1ccc(-c2cccc(-n3c4ccccc4c4cc(-c5ccc6c(c5)c5ccccc5n6-c5cccc6ccccc56)ccc43)c2)cc1]

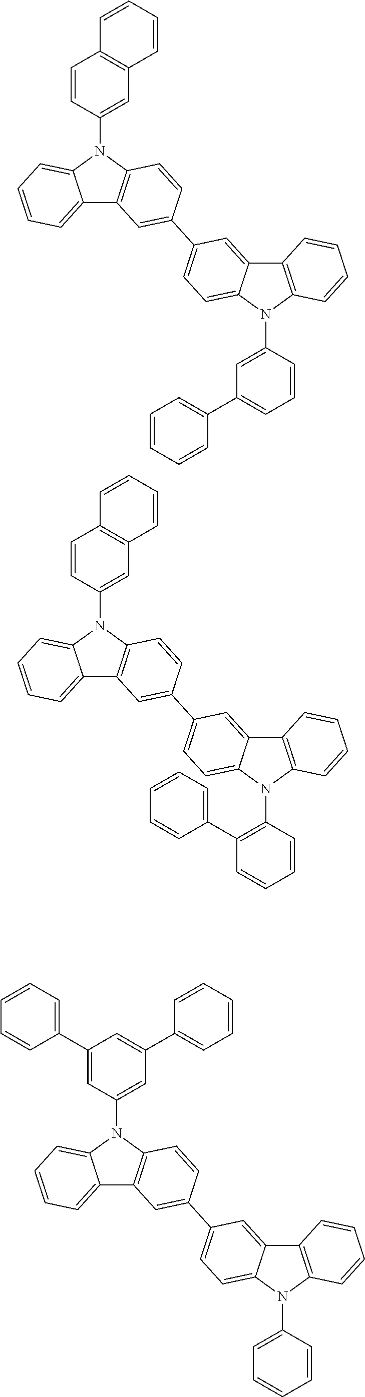

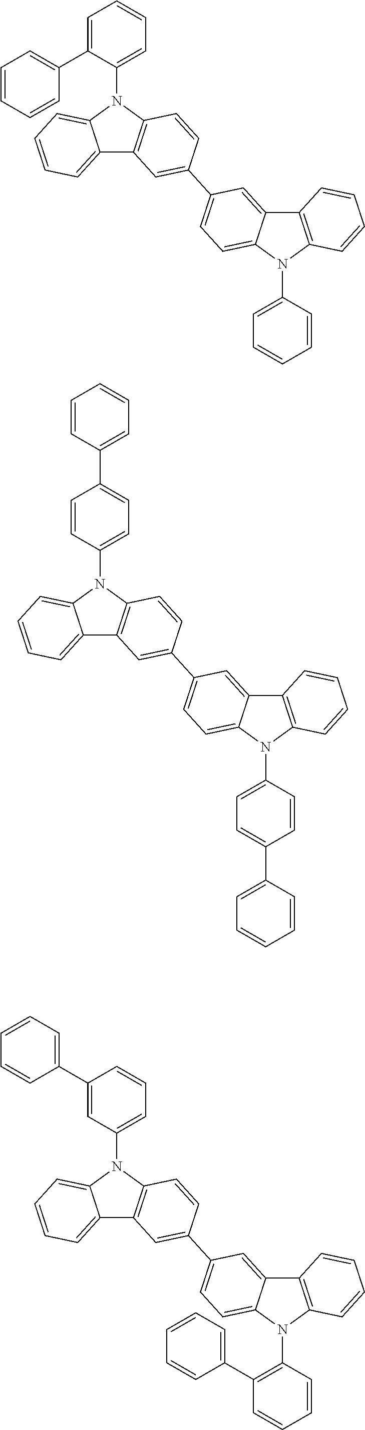 [SMILES: c1ccc(-c2ccccc2-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2cccc(-n3c4ccccc4c4cc(-c5ccc6c(c5)c5ccccc5n6-c5ccccc5-c5ccccc5)ccc43)c2)cc1]

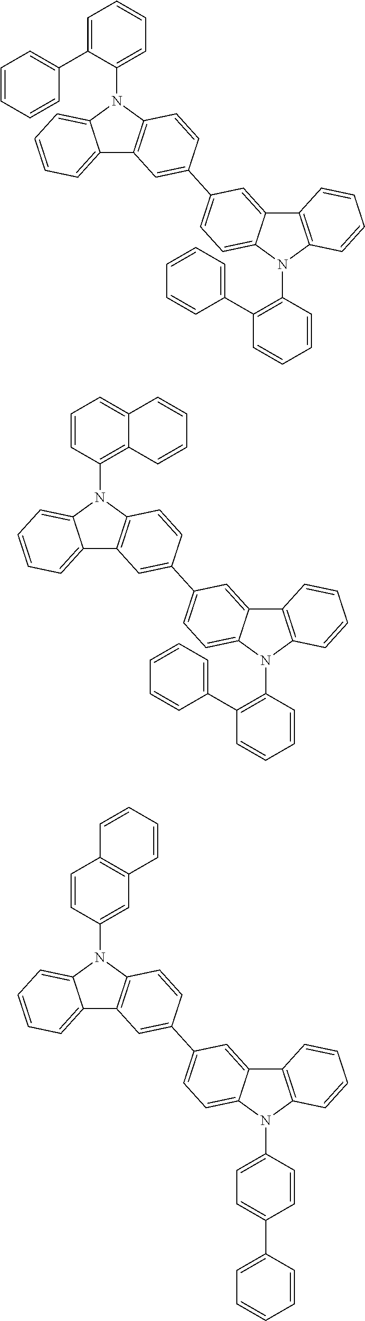 [SMILES: c1ccc(-c2ccccc2-n2c3ccccc3c3cc(-c4ccc5c(c4)c4ccccc4n5-c4ccccc4-c4ccccc4)ccc32)cc1 ; c1ccc(-c2ccccc2-n2c3ccccc3c3cc(-c4ccc5c(c4)c4ccccc4n5-c4cccc5ccccc45)ccc32)cc1 ; c1ccc(-c2ccc(-n3c4ccccc4c4cc(-c5ccc6c(c5)c5ccccc5n6-c5ccc6ccccc6c5)ccc43)cc2)cc1]

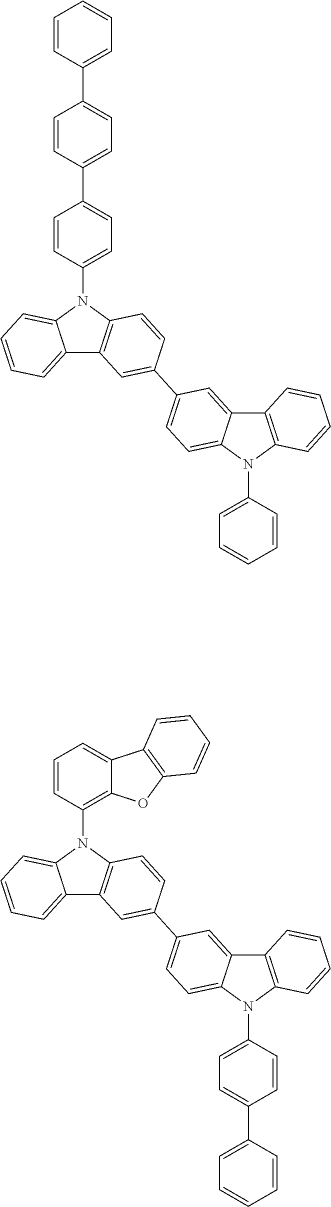 [SMILES: c1ccc(-c2ccc(-c3ccc(-n4c5ccccc5c5cc(-c6ccc7c(c6)c6ccccc6n7-c6ccccc6)ccc54)cc3)cc2)cc1 ; c1ccc(-c2ccc(-n3c4ccccc4c4cc(-c5ccc6c(c5)c5ccccc5n6-c5cccc6c5oc5ccccc56)ccc43)cc2)cc1]

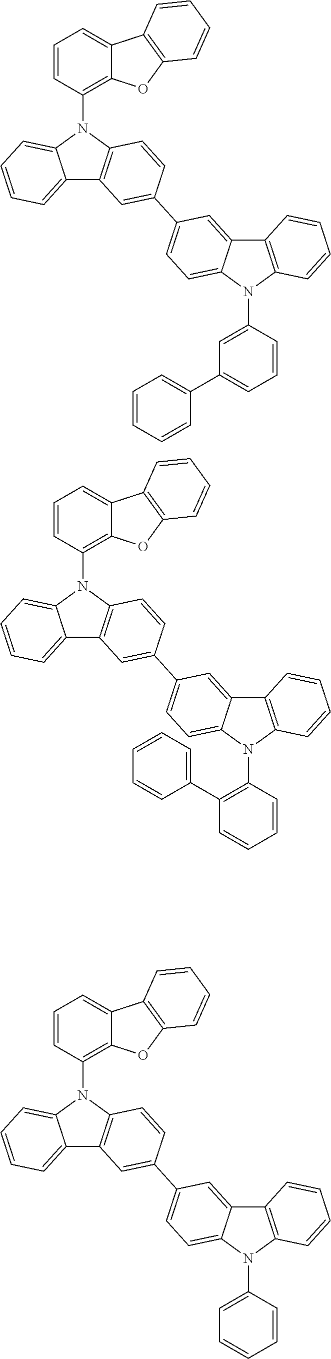

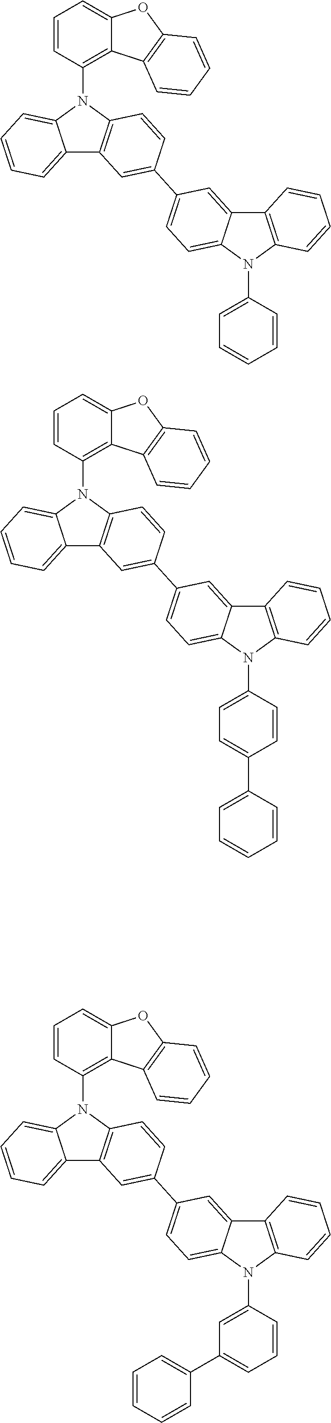 [SMILES: c1ccc(-n2c3ccccc3c3cc(-c4ccc5c(c4)c4ccccc4n5-c4cccc5oc6ccccc6c45)ccc32)cc1 ; c1ccc(-c2ccc(-n3c4ccccc4c4cc(-c5ccc6c(c5)c5ccccc5n6-c5cccc6oc7ccccc7c56)ccc43)cc2)cc1 ; c1ccc(-c2cccc(-n3c4ccccc4c4cc(-c5ccc6c(c5)c5ccccc5n6-c5cccc6oc7ccccc7c56)ccc43)c2)cc1]

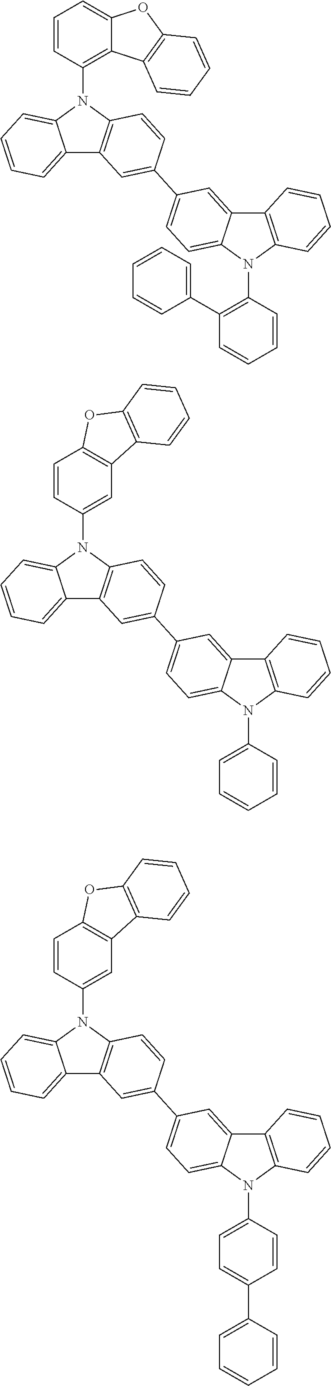 [SMILES: c1ccc(-c2ccccc2-n2c3ccccc3c3cc(-c4ccc5c(c4)c4ccccc4n5-c4cccc5oc6ccccc6c45)ccc32)cc1 ; c1ccc(-n2c3ccccc3c3cc(-c4ccc5c(c4)c4ccccc4n5-c4ccc5oc6ccccc6c5c4)ccc32)cc1 ; c1ccc(-c2ccc(-n3c4ccccc4c4cc(-c5ccc6c(c5)c5ccccc5n6-c5ccc6oc7ccccc7c6c5)ccc43)cc2)cc1]

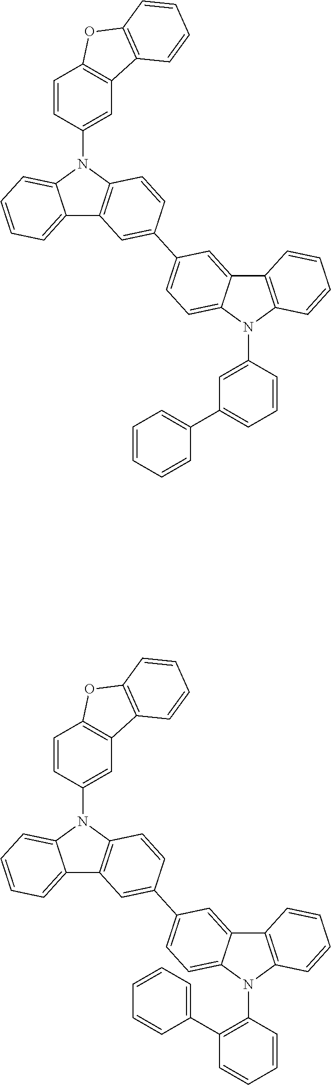 [SMILES: c1ccc(-c2cccc(-n3c4ccccc4c4cc(-c5ccc6c(c5)c5ccccc5n6-c5ccc6oc7ccccc7c6c5)ccc43)c2)cc1 ; c1ccc(-c2ccccc2-n2c3ccccc3c3cc(-c4ccc5c(c4)c4ccccc4n5-c4ccc5oc6ccccc6c5c4)ccc32)cc1]

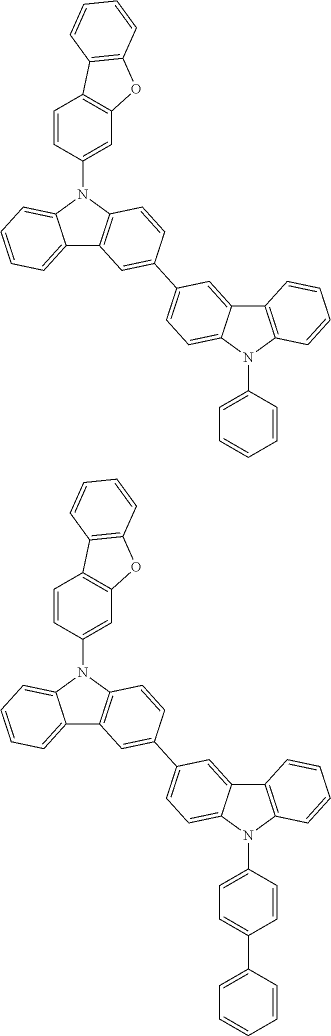 [SMILES: c1ccc(-n2c3ccccc3c3cc(-c4ccc5c(c4)c4ccccc4n5-c4ccc5c(c4)oc4ccccc45)ccc32)cc1 ; c1ccc(-c2ccc(-n3c4ccccc4c4cc(-c5ccc6c(c5)c5ccccc5n6-c5ccc6c(c5)oc5ccccc56)ccc43)cc2)cc1]

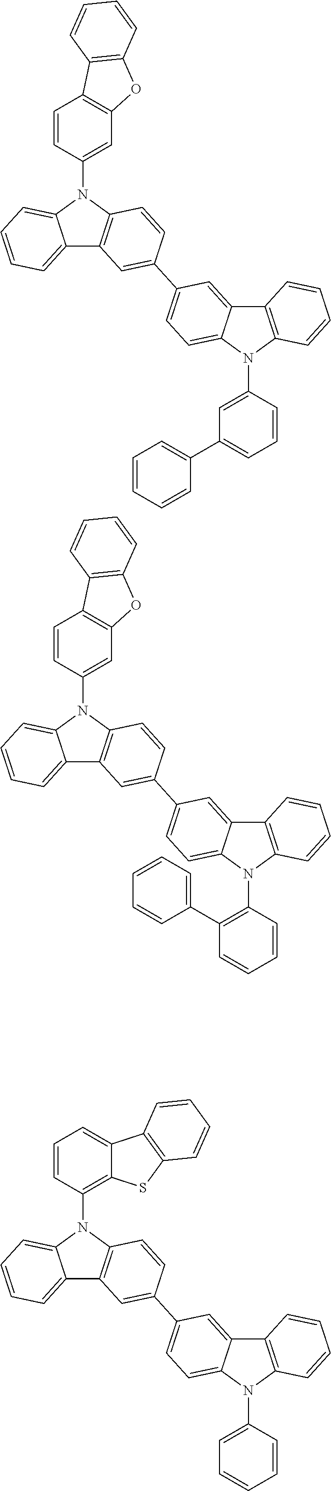

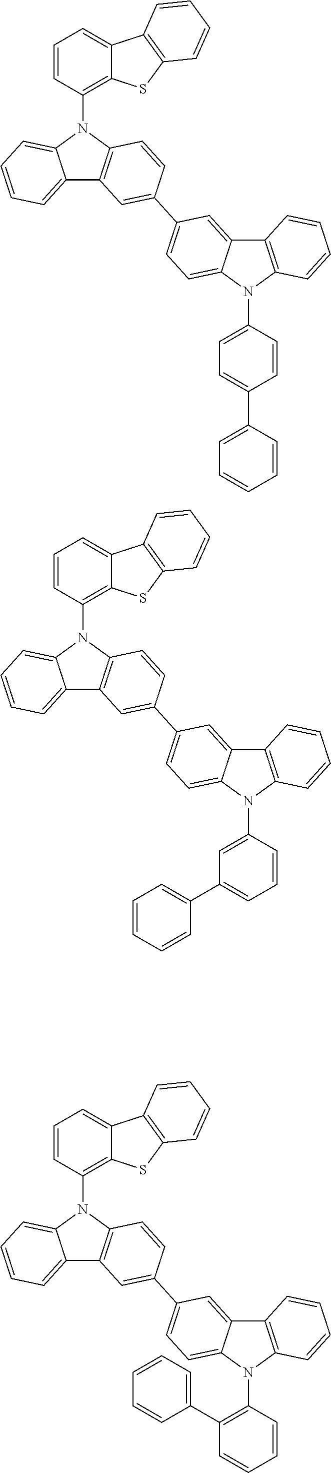

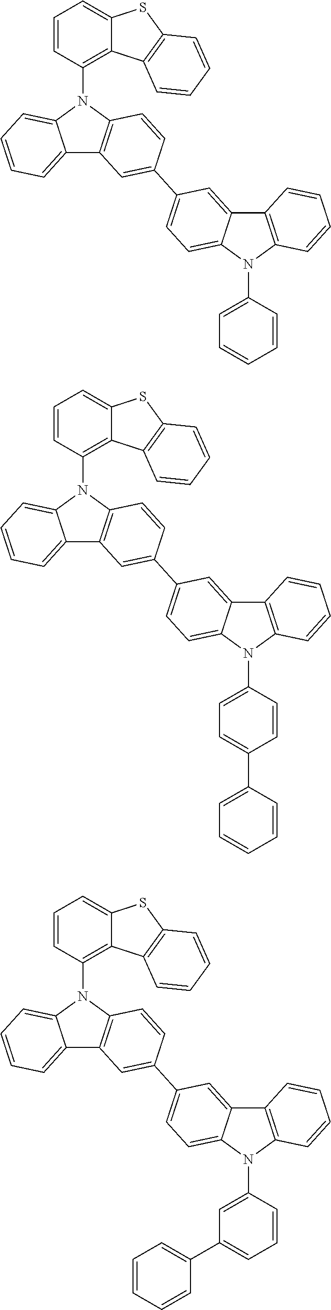 [SMILES: c1ccc(-n2c3ccccc3c3cc(-c4ccc5c(c4)c4ccccc4n5-c4cccc5sc6ccccc6c45)ccc32)cc1 ; c1ccc(-c2ccc(-n3c4ccccc4c4cc(-c5ccc6c(c5)c5ccccc5n6-c5cccc6sc7ccccc7c56)ccc43)cc2)cc1 ; c1ccc(-c2cccc(-n3c4ccccc4c4cc(-c5ccc6c(c5)c5ccccc5n6-c5cccc6sc7ccccc7c56)ccc43)c2)cc1]

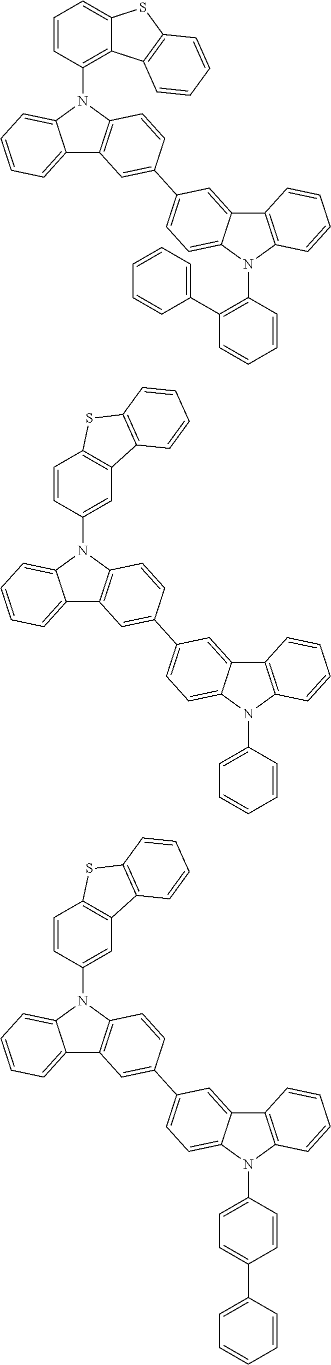 [SMILES: c1ccc(-c2ccccc2-n2c3ccccc3c3cc(-c4ccc5c(c4)c4ccccc4n5-c4cccc5sc6ccccc6c45)ccc32)cc1 ; c1ccc(-n2c3ccccc3c3cc(-c4ccc5c(c4)c4ccccc4n5-c4ccc5sc6ccccc6c5c4)ccc32)cc1 ; c1ccc(-c2ccc(-n3c4ccccc4c4cc(-c5ccc6c(c5)c5ccccc5n6-c5ccc6sc7ccccc7c6c5)ccc43)cc2)cc1]

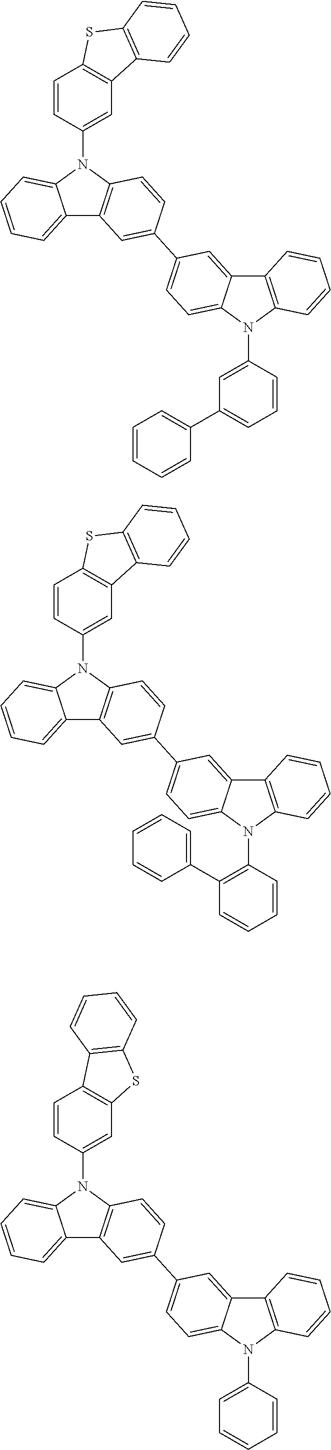

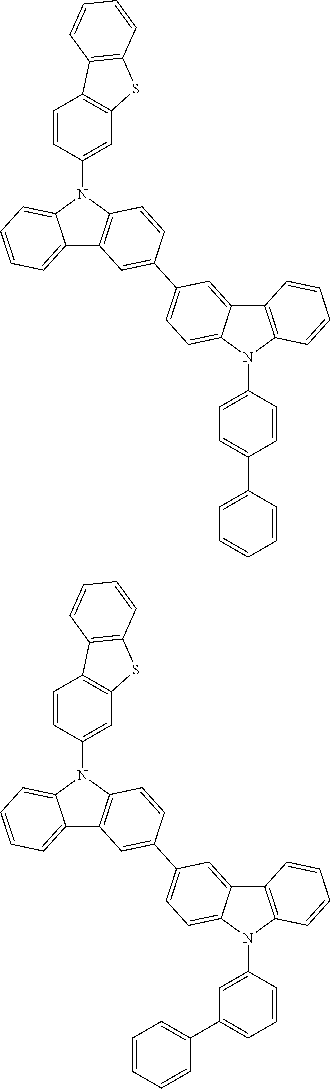 [SMILES: c1ccc(-c2ccc(-n3c4ccccc4c4cc(-c5ccc6c(c5)c5ccccc5n6-c5ccc6c(c5)sc5ccccc56)ccc43)cc2)cc1 ; c1ccc(-c2cccc(-n3c4ccccc4c4cc(-c5ccc6c(c5)c5ccccc5n6-c5ccc6c(c5)sc5ccccc56)ccc43)c2)cc1]

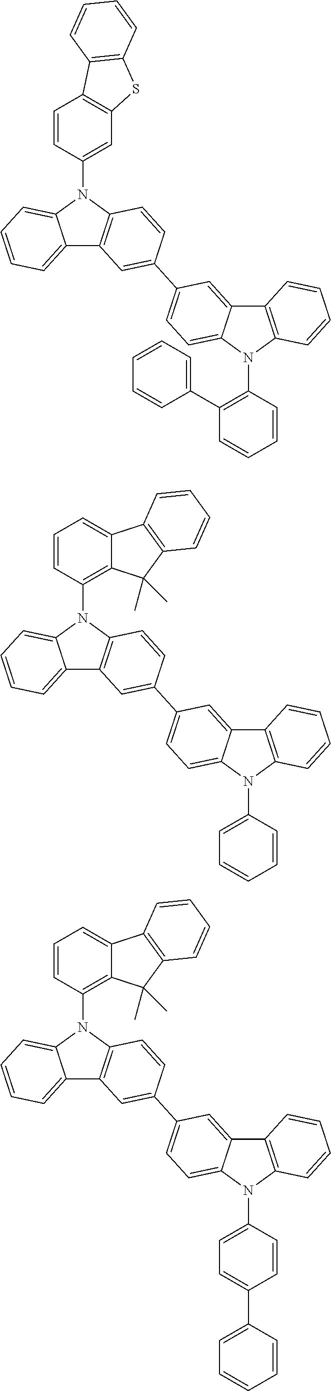

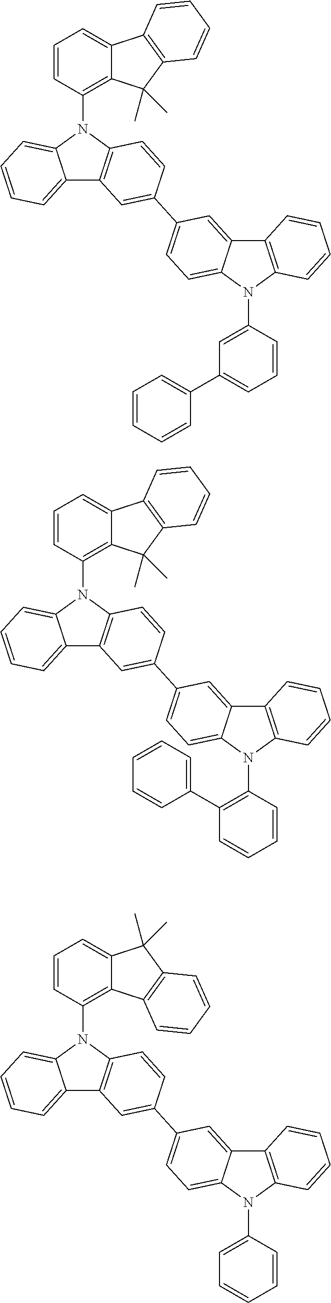

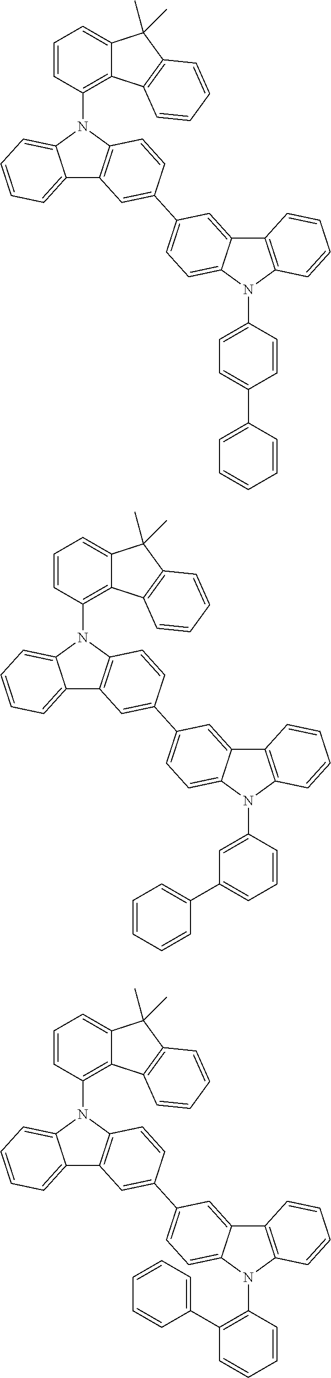

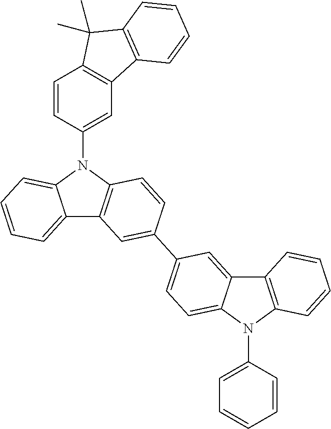 [SMILES: CC1(C)c2ccccc2-c2cc(-n3c4ccccc4c4cc(-c5ccc6c(c5)c5ccccc5n6-c5ccccc5)ccc43)ccc21]

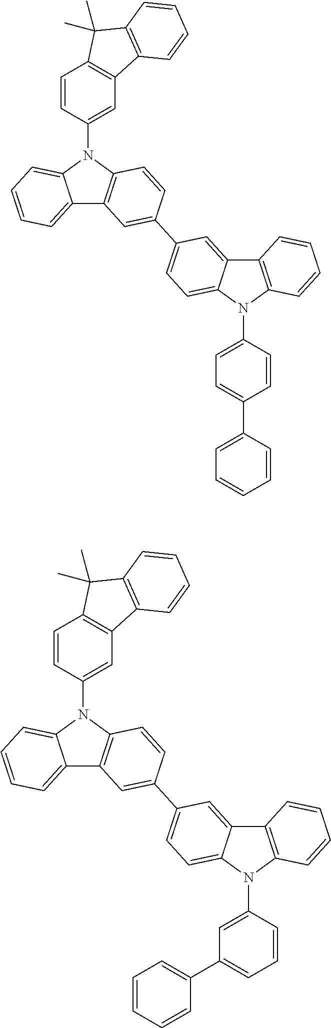 [SMILES: CC1(C)c2ccccc2-c2cc(-n3c4ccccc4c4cc(-c5ccc6c(c5)c5ccccc5n6-c5ccc(-c6ccccc6)cc5)ccc43)ccc21 ; CC1(C)c2ccccc2-c2cc(-n3c4ccccc4c4cc(-c5ccc6c(c5)c5ccccc5n6-c5cccc(-c6ccccc6)c5)ccc43)ccc21]

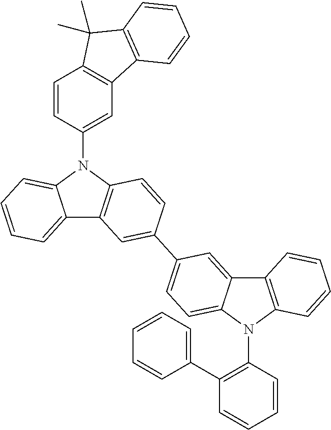 [SMILES: CC1(C)c2ccccc2-c2cc(-n3c4ccccc4c4cc(-c5ccc6c(c5)c5ccccc5n6-c5ccccc5-c5ccccc5)ccc43)ccc21]

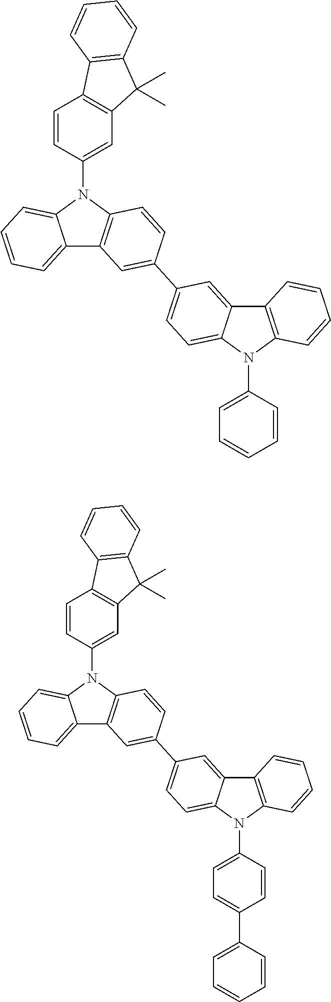 [SMILES: CC1(C)c2ccccc2-c2ccc(-n3c4ccccc4c4cc(-c5ccc6c(c5)c5ccccc5n6-c5ccccc5)ccc43)cc21 ; CC1(C)c2ccccc2-c2ccc(-n3c4ccccc4c4cc(-c5ccc6c(c5)c5ccccc5n6-c5ccc(-c6ccccc6)cc5)ccc43)cc21]

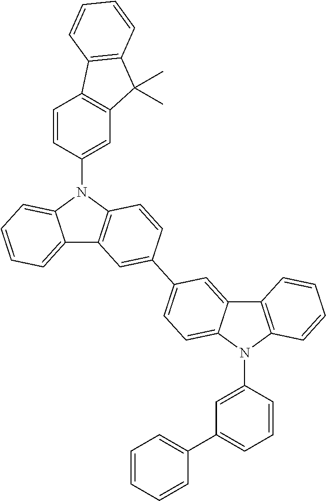 [SMILES: CC1(C)c2ccccc2-c2ccc(-n3c4ccccc4c4cc(-c5ccc6c(c5)c5ccccc5n6-c5cccc(-c6ccccc6)c5)ccc43)cc21]

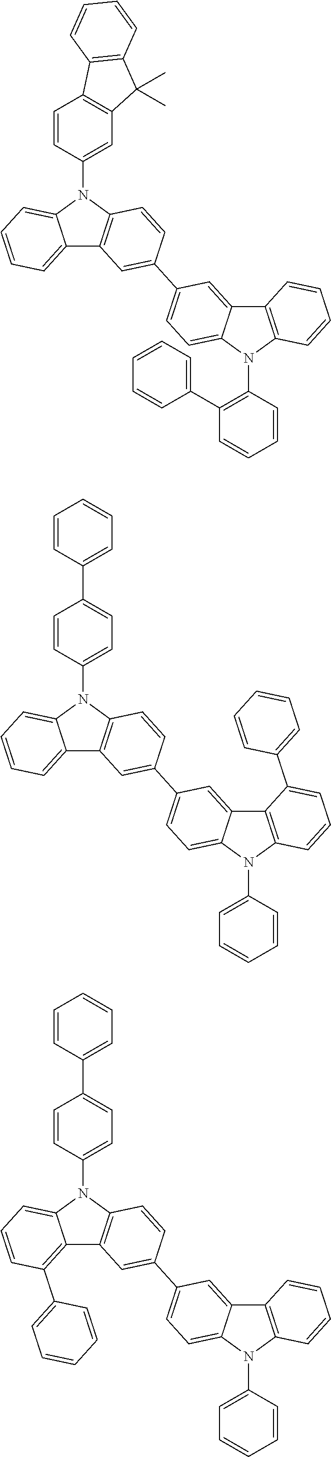 [SMILES: CC1(C)c2ccccc2-c2ccc(-n3c4ccccc4c4cc(-c5ccc6c(c5)c5ccccc5n6-c5ccccc5-c5ccccc5)ccc43)cc21 ; c1ccc(-c2ccc(-n3c4ccccc4c4cc(-c5ccc6c(c5)c5c(-c7ccccc7)cccc5n6-c5ccccc5)ccc43)cc2)cc1 ; c1ccc(-c2ccc(-n3c4ccc(-c5ccc6c(c5)c5ccccc5n6-c5ccccc5)cc4c4c(-c5ccccc5)cccc43)cc2)cc1]

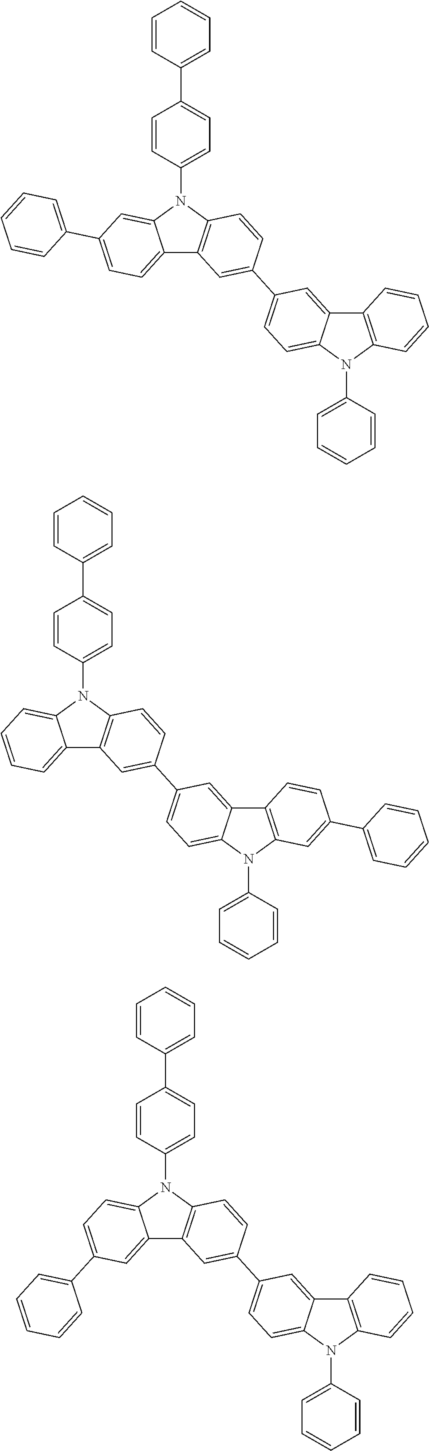 [SMILES: c1ccc(-c2ccc(-n3c4ccc(-c5ccc6c(c5)c5ccccc5n6-c5ccccc5)cc4c4ccc(-c5ccccc5)cc43)cc2)cc1 ; c1ccc(-c2ccc(-n3c4ccccc4c4cc(-c5ccc6c(c5)c5ccc(-c7ccccc7)cc5n6-c5ccccc5)ccc43)cc2)cc1 ; c1ccc(-c2ccc(-n3c4ccc(-c5ccccc5)cc4c4cc(-c5ccc6c(c5)c5ccccc5n6-c5ccccc5)ccc43)cc2)cc1]

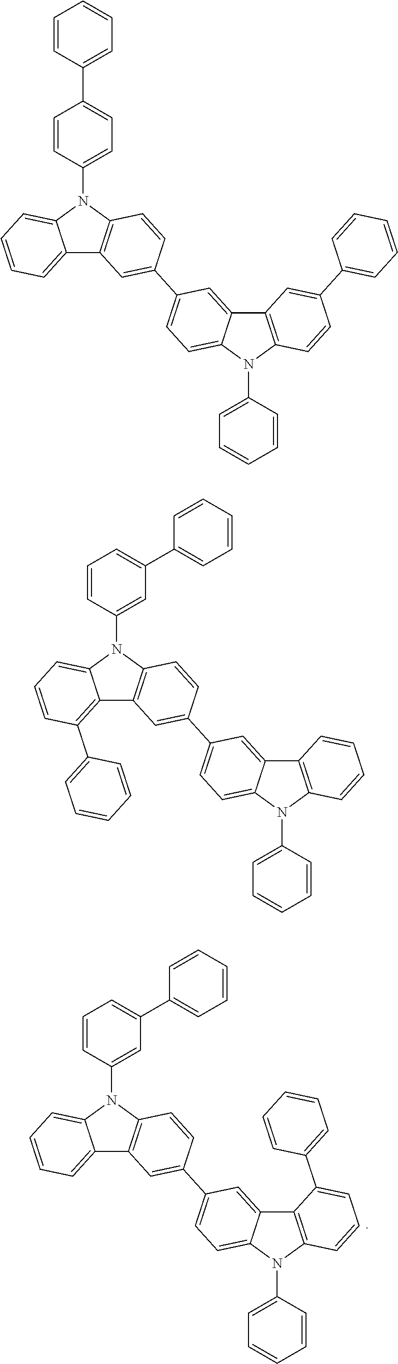 [SMILES: c1ccc(-c2ccc(-n3c4ccccc4c4cc(-c5ccc6c(c5)c5cc(-c7ccccc7)ccc5n6-c5ccccc5)ccc43)cc2)cc1 ; c1ccc(-c2cccc(-n3c4ccc(-c5ccc6c(c5)c5ccccc5n6-c5ccccc5)cc4c4c(-c5ccccc5)cccc43)c2)cc1 ; c1ccc(-c2cccc(-n3c4ccccc4c4cc(-c5ccc6c(c5)c5c(-c7ccccc7)cccc5n6-c5ccccc5)ccc43)c2)cc1]

The compound represented by Chemical Formula 1 and the compound represented by Chemical Formula 2 may be included in the composition in a weight ratio of 10:90 to 90:10, for example, 50:50.

Furthermore, the composition may be a mixture or an organic alloy.

In one embodiment, the composition may be a mixture in which the compound represented by Chemical Formula 1 and the compound represented by Chemical Formula 2 are simply mixed. This mixture, in which each compound is physically homogeneously mixed without any separate pretreatment, can be prepared using a mixer commonly known in the art.

In this way, when the compound represented by Chemical Formula 1 and the compound represented by Chemical Formula 2 are applied to an organic light emitting device as composition in the form of a mixture, the organic layer is formed by supplying each compound from a single source rather than separate sources. This simplifies the process, eliminating the need for process control steps for multiple sources.

In another embodiment, the composition may be an organic alloy in which the compound represented by Chemical Formula 1 and the compound represented by Chemical Formula 2 have chemical interactions through pretreatment. The pretreatment may, for example, involve heating and/or sublimation of a mixture of the compounds and then cooling them, but is not limited thereto.

Such an organic alloy may exhibit emission wavelengths, colors, glass transition temperatures (Tg), crystallization temperatures (Tc), and melting temperatures (Tm) that differ from those of a mixture in which no chemical interaction exists between the compounds.

Further, when the compound represented by Chemical Formula 1 and the compound represented by Chemical Formula 2 is applied to an organic light emitting device as composition in the form of an organic alloy, all compounds can be supplied from a single source during the formation of the organic layer, simplifying the process and ensuring uniformity and consistency of the deposited material. Accordingly, when forming multiple organic layers in a continuous process, organic layers having components in substantially the same ratio can be continuously produced, and thus the reproducibility and reliability of the organic layers can be improved.

### (Organic Light Emitting Device)

Meanwhile, according to the present disclosure, there is provided an organic light emitting device comprising a compound represented by Chemical Formula 1. In one example, the present disclosure provides an organic light emitting device comprising: a first electrode; a second electrode that is provided opposite to the first electrode; and one or more organic material layers that are provided between the first electrode and the second electrode, wherein one or more layers of the organic material layers includes the compound represented by Chemical Formula 1.

The organic material layer of the organic light emitting device of the present disclosure may have a single-layer structure, or it may have a multilayered structure in which two or more organic material layers are stacked. For example, the organic light emitting device of the present disclosure may have a structure comprising a hole injection layer, a hole transport layer, a light emitting layer, an electron transport layer, an electron injection layer and the like as the organic material layer. However, the structure of the organic light emitting device is not limited thereto, and it may include a smaller number of organic layers.

In this case, the organic material layer may include a light emitting layer, wherein the organic material layers comprising the compound may be a light emitting layer.

In another embodiment, the organic material layer may include a hole injection layer, a hole transport layer, a light emitting layer, and an electron injection and transport layer, wherein the organic material layer comprising the compound may be a light emitting layer, or an electron injection and transport layer.

In yet another embodiment, the organic material layer may include a hole injection layer, a hole transport layer, an electron blocking layer, a light emitting layer, and an electron injection and transport layer, wherein the organic material layer comprising the compound may be a light emitting layer, or an electron injection and transport layer.

In yet another embodiment, the organic material layer may include a hole injection layer, a hole transport layer, an electron blocking layer, a light emitting layer, a hole blocking layer, and an electron injection and transport layer, wherein the organic material layer comprising the compound may be a light emitting layer, a hole blocking layer, or an electron injection and transport layer.

Further, in one example, the present disclosure provides an organic light emitting device comprising: a first electrode; a second electrode that is provided opposite to the first electrode; and one or more organic material layers that are provided between the first electrode and the second electrode, wherein one or more layers of the organic material layers comprises the composition comprising the compound represented by Chemical Formula 1 and the compound represented by Chemical Formula 2

In this case, the organic material layer may include a light emitting layer, wherein the organic material layers comprising the composition are a light emitting layer.

The organic layer of the organic light emitting device of the present disclosure may be formed as a single layer structure, but may be formed as a multilayer structure in which two or more organic layers are laminated. For example, the organic light emitting device of the present disclosure may have a structure that further includes, in addition to the light emitting layer as the organic layer, a hole injection layer and a hole transport layer between the first electrode and the light emitting layer, and an electron transport layer and an electron injection layer between the light emitting layer and the second electrode. However, the structure of the organic light emitting device is not limited thereto and may include a smaller or larger number of organic layers.

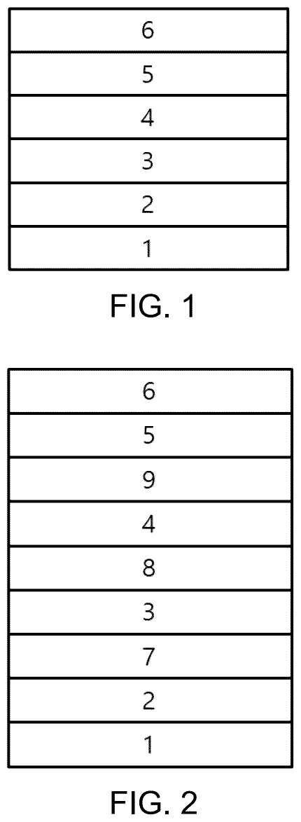

Further, the organic light emitting device according to the present disclosure may be a normal type organic light emitting device in which an anode, one or more organic material layers and a cathode are sequentially stacked on a substrate, wherein the first electrode is an anode, and the second electrode is a cathode. Further, the organic light emitting device according to the present disclosure may be an inverted type organic light emitting device in which a cathode, one or more organic material layers and an anode are sequentially stacked on a substrate, wherein the first electrode is a cathode and the second electrode is an anode. For example, the structure of the organic light emitting device according to one embodiment of the present disclosure is illustrated in FIGS. 1 and 2.

FIG. 1 shows an example of an organic light emitting device comprising a substrate 1, an anode 2, a hole transport layer 3, a light emitting layer 4, an electron injection and transport layer 5, and a cathode 6. In such a structure, the compound represented by Chemical Formula 1 may be included in the light emitting layer.

FIG. 2 shows an example of an organic light emitting device comprising a substrate 1, an anode 2, a hole injection layer 7, a hole transport layer 3, an electron blocking layer 8, a light emitting layer 4, a hole blocking layer 9, an electron injection and transport layer 5, and a cathode 6. In such a structure, the compound represented by Chemical Formula 1 may be included in the light emitting layer.

The organic light emitting device according to the present disclosure may be manufactured by materials and methods known in the art, except that at least one of the organic material layers includes the compound represented by Chemical Formula 1. Further, when the organic light emitting device includes a plurality of organic material layers, the organic material layers can be formed of the same material or different materials.

For example, the organic light emitting device according to the present disclosure can be manufactured by sequentially stacking a first electrode, an organic material layer and a second electrode on a substrate. In this case, the organic light emitting device may be manufactured by depositing a metal, metal oxides having conductivity, or an alloy thereof on the substrate using a PVD (physical vapor deposition) method such as a sputtering method or an e-beam evaporation method to form an anode, forming organic material layers including the hole injection layer, the hole transport layer, the light emitting layer and the electron transport layer thereon, and then depositing a material that can be used as the cathode thereon. In addition to such a method, the organic light emitting device can be manufactured by sequentially depositing a cathode material, an organic material layer and an anode material on a substrate.

Further, the compound represented by Chemical Formula 1 can be formed into an organic layer by a solution coating method as well as a vacuum deposition method at the time of manufacturing an organic light emitting device. Herein, the solution coating method means a spin coating, a dip coating, a doctor blading, an inkjet printing, a screen printing, a spray method, a roll coating, or the like, but is not limited thereto.

In addition to such a method, the organic light emitting device may be manufactured by sequentially depositing a cathode material, an organic material layer and an anode material on a substrate (International Publication WO2003/012890). However, the manufacturing method is not limited thereto.

As an example, the first electrode is an anode, and the second electrode is a cathode, or alternatively, the first electrode is a cathode and the second electrode is an anode.

As the anode material, generally, a material having a large work function is preferably used so that holes can be smoothly injected into the organic material layer. Specific examples of the anode material include metals such as vanadium, chrome, copper, zinc, and gold, or an alloy thereof; metal oxides such as zinc oxides, indium oxides, indium tin oxides (ITO), and indium zinc oxides (IZO); a combination of metals and oxides, such as ZnO:Al or SnO₂:Sb; conductive compounds such as poly(3-methylthiophene), poly[3,4-(ethylene-1,2-dioxy)thiophene](PEDOT), polypyrrole, and polyaniline, and the like, but are not limited thereto.

As the cathode material, generally, a material having a small work function is preferably used so that electrons can be easily injected into the organic material layer. Specific examples of the cathode material include metals such as magnesium, calcium, sodium, potassium, titanium, indium, yttrium, lithium, gadolinium, aluminum, silver, tin, and lead, or an alloy thereof; a multilayered structure material such as LiF/Al or LiO₂/Al, and the like, but are not limited thereto.

Further, the hole injection layer is a layer for injecting holes from the electrode, and the hole injection material is preferably a compound which has a capability of transporting the holes, thus has a hole injecting effect in the anode and an excellent hole injecting effect to the light emitting layer or the light emitting material, prevents excitons produced in the light emitting layer from moving to a hole injection layer or the electron injection material, and further is excellent in the ability to form a thin film. It is preferable that a HOMO (highest occupied molecular orbital) of the hole injection material is between the work function of the anode material and a HOMO of a peripheral organic material layer. Specific examples of the hole injection material include the compound represented by Chemical Formula 1, metal porphyrin, oligothiophene, an arylamine-based organic material, a hexanitrilehexaazatriphenylene-based organic material, a quinacridone-based organic material, a perylene-based organic material, anthraquinone, polyaniline and polythiophene-based conductive polymer, and the like, but are not limited thereto.

The hole transport layer is a layer that receives holes from a hole injection layer and transports the holes to the light emitting layer. The hole transport material is suitably a material having large mobility to the holes, which may receive holes from the anode or the hole injection layer and transfer the holes to the light emitting layer. The hole transport material includes an arylamine-based organic material, a conductive polymer, a block copolymer in which a conjugate portion and a non-conjugate portion are present together, and the like, but are not limited thereto.

Further, the electron blocking layer (electron suppression layer) refers to a layer which is formed on the hole transport layer, preferably provided in contact with the light emitting layer, and serves to adjust the hole mobility, prevent excessive movement of electrons, and increase the probabilities of hole-electron coupling, thereby improving the efficiency of the organic light emitting device. The electron blocking layer includes an electron blocking material, and examples of such electron blocking material may include an arylamine-based organic material, and the like, but is not limited thereto.

The light emitting material is preferably a material which may receive holes and electrons transported from a hole transport layer and an electron transport layer, respectively, and combine the holes and the electrons to emit light in a visible ray region, and has good quantum efficiency to fluorescence or phosphorescence. Specific examples of the light emitting material include an 8-hydroxy-quinoline aluminum complex (Alq₃); a carbazole-based compound; a dimerized styryl compound; BAlq; a 10-hydroxybenzoquinoline-metal compound; a benzoxazole, benzothiazole and benzimidazole-based compound; a poly(p-phenylenevinylene)(PPV)-based polymer; a spiro compound; polyfluorene, rubrene, and the like, but are not limited thereto.

Further, the light emitting layer may include a host material and a dopant material. The compound represented by Chemical Formula 1 can be used as such a host material. Further, the host material may further include a fused aromatic ring derivative, a heterocycle-containing compound or the like in addition to the compound represented by Chemical Formula 1. Specific examples of the fused aromatic ring derivatives include anthracene derivatives, pyrene derivatives, naphthalene derivatives, pentacene derivatives, phenanthrene compounds, fluoranthene compounds, and the like. Examples of the heterocyclic-containing compounds include carbazole derivatives, dibenzofuran derivatives, ladder-type furan compounds, pyrimidine derivatives, and the like, but are not limited thereto.

In one embodiment, the light emitting layer may further include a compound represented by the Chemical formula 2 in addition to the compound represented by the Chemical formula 1.

Examples of the dopant material include an aromatic amine derivative, a styrylamine compound, a boron complex, a fluoranthene compound, a metal complex, and the like. Specifically, the aromatic amine derivative is a substituted or unsubstituted fused aromatic ring derivative having an arylamino group, and examples thereof include pyrene, anthracene, chrysene, periflanthene and the like, which have an arylamino group. The styrylamine compound is a compound where at least one arylvinyl group is substituted in substituted or unsubstituted arylamine, in which one or two or more substituent groups selected from the group consisting of an aryl group, a silyl group, an alkyl group, a cycloalkyl group, and an arylamino group are substituted or unsubstituted. Specific examples thereof include styrylamine, styryldiamine, styryltriamine, styryltetramine, and the like, but are not limited thereto. Further, the metal complex includes an iridium complex, a platinum complex, and the like, but is not limited thereto.

More specifically, the following compounds may be used as the dopant material, but are not limited thereto:

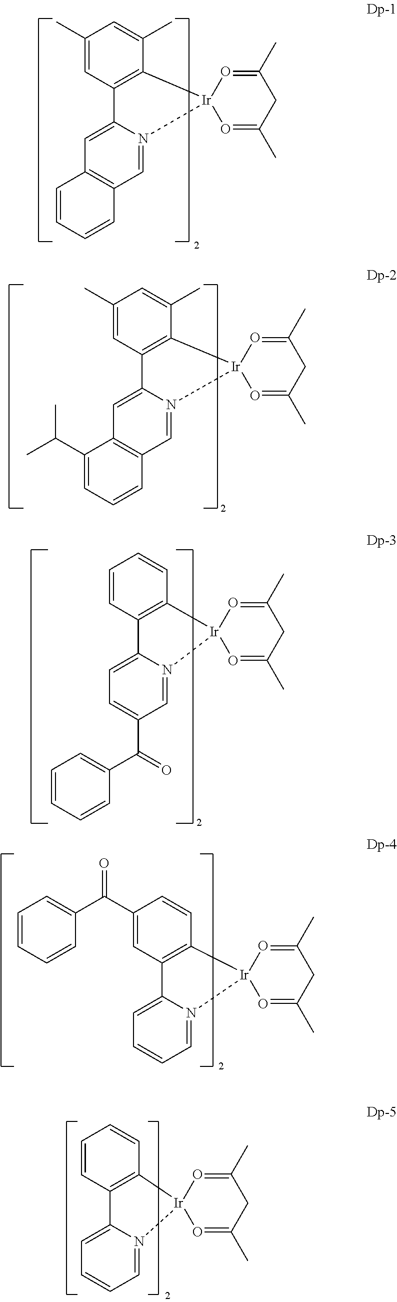

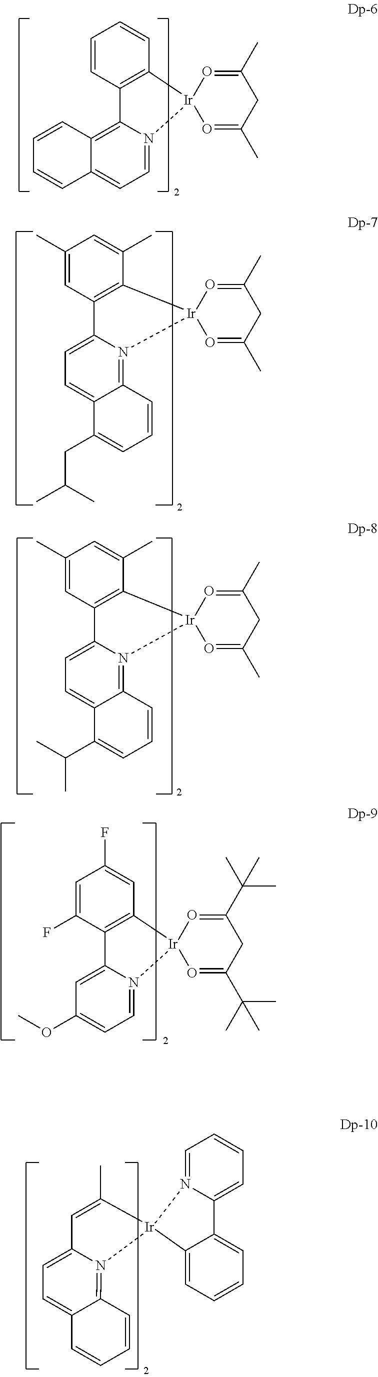

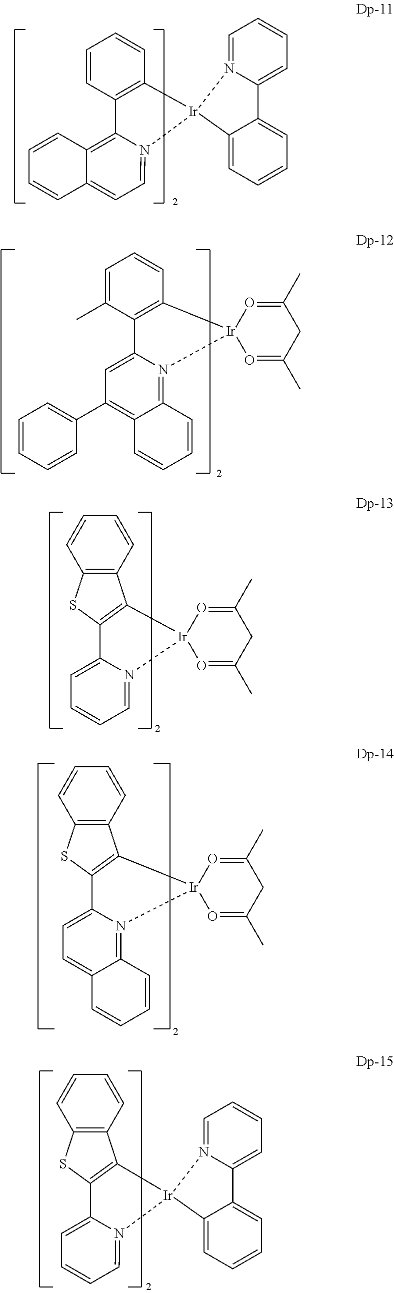

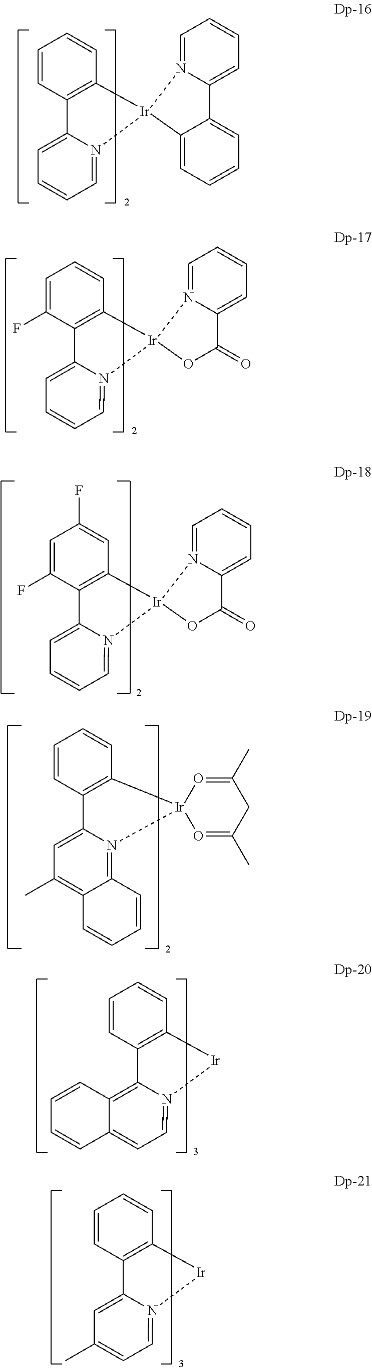

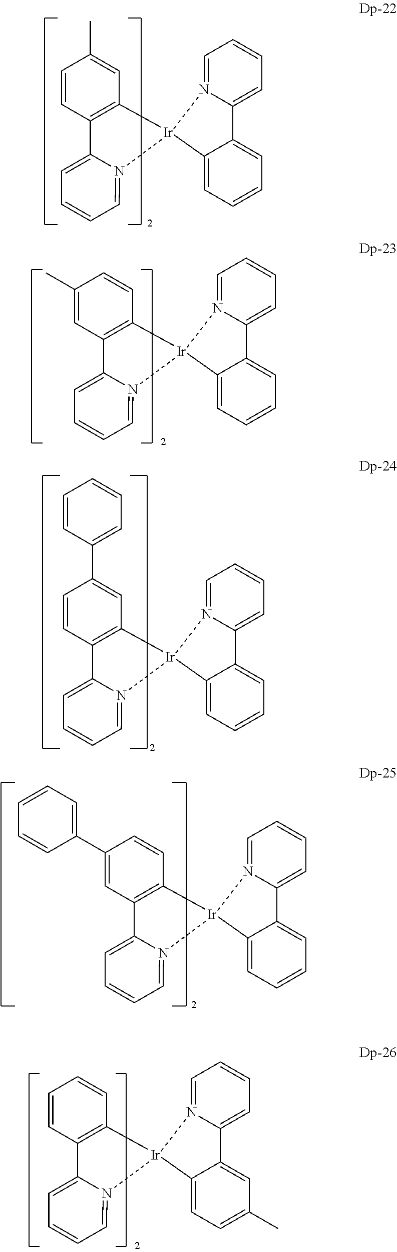

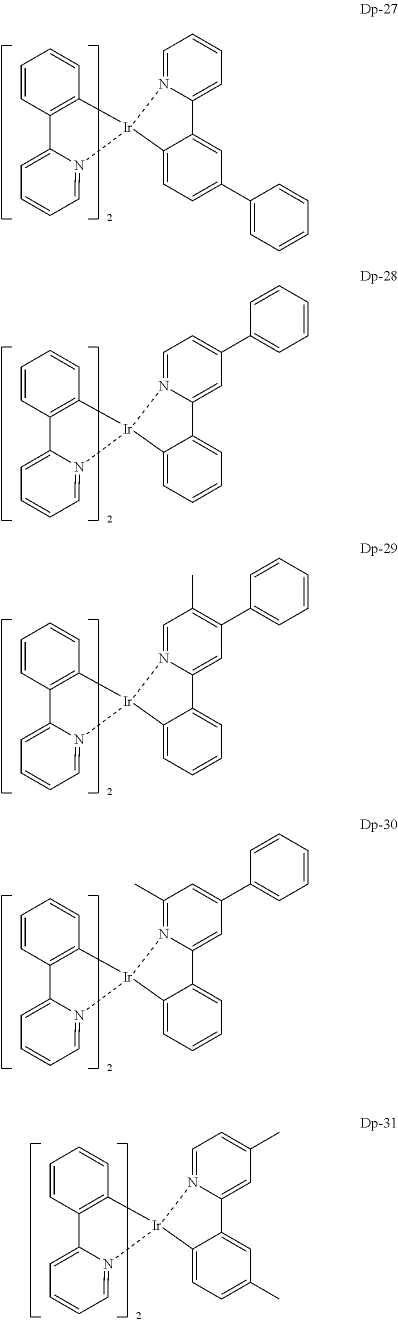

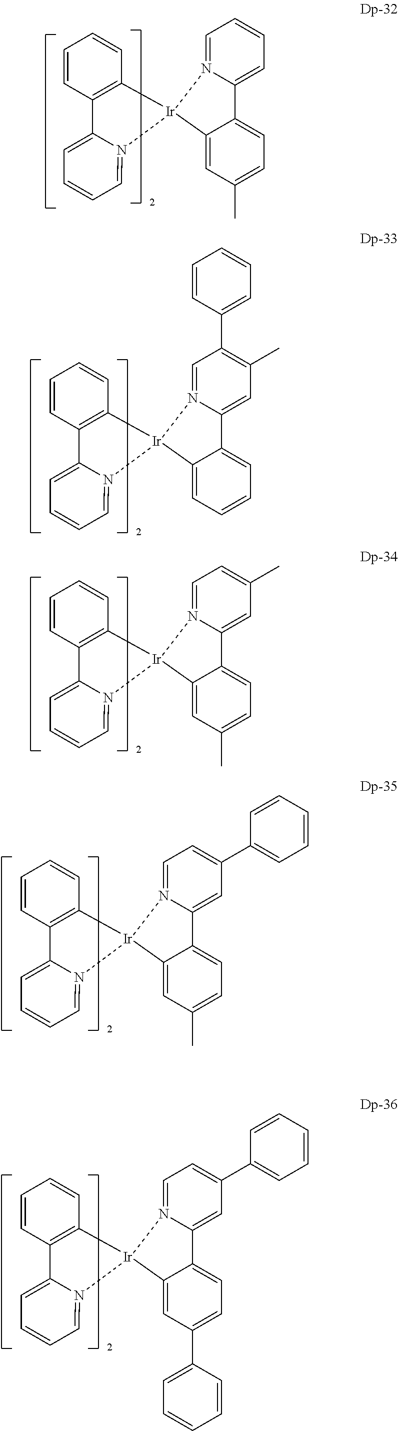

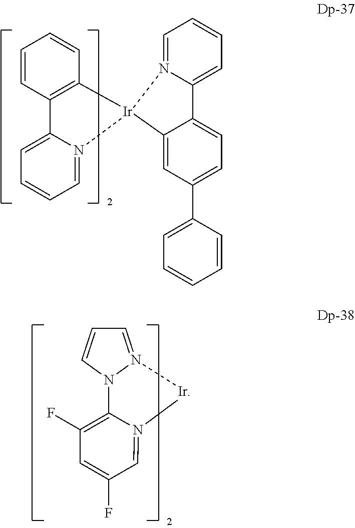

The hole blocking layer refers to a layer which is formed on the light emitting layer, and preferably, is provided in contact with the light emitting layer, and thus severs to control electron mobility, to prevent excessive movement of holes, and to increase the probabilities of hole-electron bonding, thereby improving the efficiency of the organic light emitting device. The hole blocking layer includes a hole blocking material, and as an example of such hole blocking material, a compound into which an electron-withdrawing group is introduced, such as azine derivatives including triazine; triazole derivatives; oxadiazole derivatives; phenanthroline derivatives; phosphine oxide derivatives can be used, but is not limited thereto.

The electron injection and transport layer is a layer for simultaneously performing the roles of an electron transport layer and an electron injection layer that inject electrons from an electrode and transport the received electrons up to the light emitting layer, and is formed on the light emitting layer or the hole blocking layer. The electron injection and transport material is suitably a material which can receive electrons well from a cathode and transfer the electrons to a light emitting layer, and has a large mobility for electrons. Specific examples of the electron injection and transport material include: an Al complex of 8-hydroxyquinoline; a complex including Alq₃; an organic radical compound; a hydroxyflavone-metal complex, a triazine derivative, and the like, but are not limited thereto. Alternatively, it may be used together with fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like, but are not limited thereto.

The electron injection and transport layer may also be formed as a separate layer such as an electron injection layer and an electron transport layer. In such a case, the electron transport layer is formed on the light emitting layer or the hole blocking layer, and the above-mentioned electron injection and transport material may be used as the electron transport material included in the electron transport layer. In addition, the electron injection layer is formed on the electron transport layer, and examples of the electron injection material included in the electron injection layer include LiF, NaCl, CsF, Li₂O, BaO, fluorenone, anthraquinodimethane, diphenoquinone, thiopyran dioxide, oxazole, oxadiazole, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like.

The metal complex compound includes 8-hydroxyquinolinato lithium, bis(8-hydroxyquinolinato)zinc, bis(8-hydroxyquinolinato)copper, bis(8-hydroxyquinolinato)manganese, tris(8-hydroxyquinolinato)aluminum, tris(2-methyl-8-hydroxyquinolinato)aluminum, tris(8-hydroxyquinolinato)gallium, bis(10-hydroxybenzo[h]quinolinato)beryllium, bis(10-hydroxybenzo[h]quinolinato)zinc, bis(2-methyl-8-quinolinato)chlorogallium, bis(2-methyl-8-quinolinato)(o-cresolato)gallium, bis(2-methyl-8-quinolinato)(1-naphtholato)aluminum, bis(2-methyl-8-quinolinato)(2-naphtholato)gallium, and the like, but is not limited thereto.

The organic light emitting device according to the present disclosure may be a bottom emission type device, a top emission type device, or a double side emission type device, and in particular, it may be a bottom emission type light emitting device that requires relatively high luminous efficiency.

In addition, the compound represented by Chemical Formula 1 may be included in an organic solar cell or an organic transistor in addition to an organic light emitting device.

The manufacture of the compound represented by the Chemical formula 1 and the organic light emitting device comprising the same is specifically described in the following examples. However, the following examples are intended to illustrate the present disclosure, and the scope of the present invention is not limited thereto.

### SYNTHESIS EXAMPLE

### Synthesis Example A: Preparation of Compound P-1_1

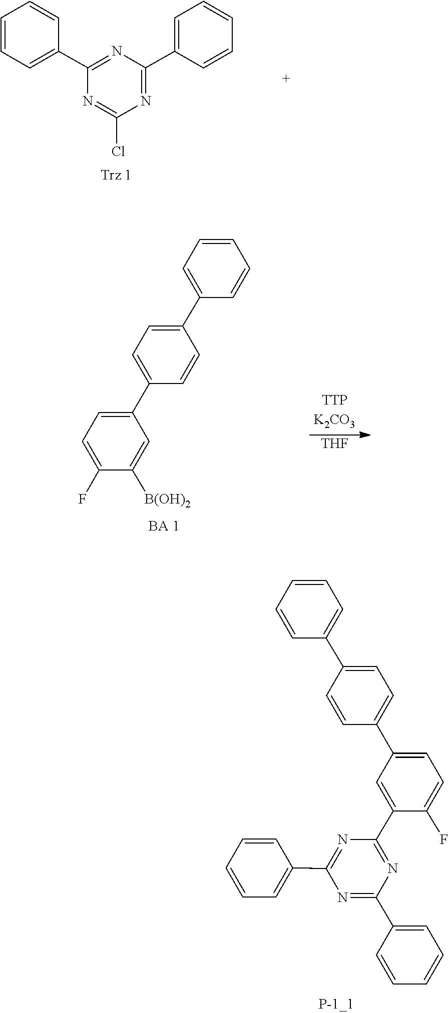 [SMILES: Clc1nc(-c2ccccc2)nc(-c2ccccc2)n1 ; OB(O)c1cc(-c2ccc(-c3ccccc3)cc2)ccc1F ; Fc1ccc(-c2ccc(-c3ccccc3)cc2)cc1-c1nc(-c2ccccc2)nc(-c2ccccc2)n1 ; "THF"]

Trz 1 (30 g, 112.1 mmol), BA 1 (33.7 g, 115.4 mmol), and potassium carbonate (31 g, 224.1 mmol) were added to 300 mL of tetrahydrofuran under a nitrogen atmosphere, and the mixture was stirred and refluxed. Then, Tetrakis(triphenylphosphine)palladium(0) (0.3 g, 2.2 mmol) was added thereto. When the reaction was terminated after 5 hours, the reaction mixture was cooled to room temperature, and the solvent was removed under reduced pressure. Then, the compound was completely dissolved in toluene, washed twice with water, and the organic layer was separated, treated with anhydrous magnesium sulfate and then filtered, and the filtrate was distilled under reduced pressure. The concentrated compound was purified by silica gel column chromatography to obtain 36.5 g of Compound P-1_1. (Yield: 68%, MS: [M+H]⁺=481)

### Synthesis Example B: Preparation of Compound P-1_2

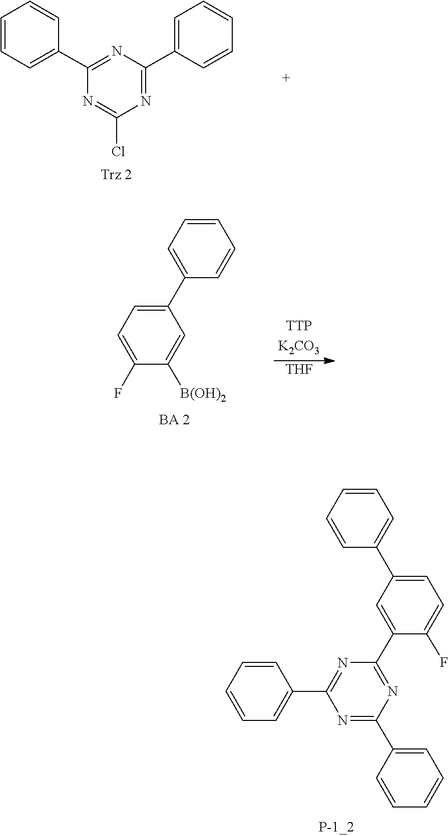 [SMILES: Clc1nc(-c2ccccc2)nc(-c2ccccc2)n1 ; OB(O)c1cc(-c2ccccc2)ccc1F ; Fc1ccc(-c2ccccc2)cc1-c1nc(-c2ccccc2)nc(-c2ccccc2)n1 ; "THF"]

Trz 2 (30 g, 112.1 mmol), BA 2 (24.9 g, 115.4 mmol), and potassium carbonate (31 g, 224.1 mmol) were added to 300 mL of tetrahydrofuran under a nitrogen atmosphere, and the mixture was stirred and refluxed. Tetrakis(triphenylphosphine)palladium(0) (0.3 g, 2.2 mmol) was added thereto. When the reaction was terminated after 5 hours, the reaction mixture was cooled to room temperature, and the solvent was removed under reduced pressure. Then, the compound was completely dissolved in toluene, washed twice with water, and the organic layer was separated, treated with anhydrous magnesium sulfate and then filtered, and the filtrate was distilled under reduced pressure. The concentrated compound was purified by silica gel column chromatography to obtain 27.6 g of Compound P-1_2. (Yield: 61%, MS: [M+H]⁺=404)

### Synthesis Example C: Preparation of Compound P-1_3

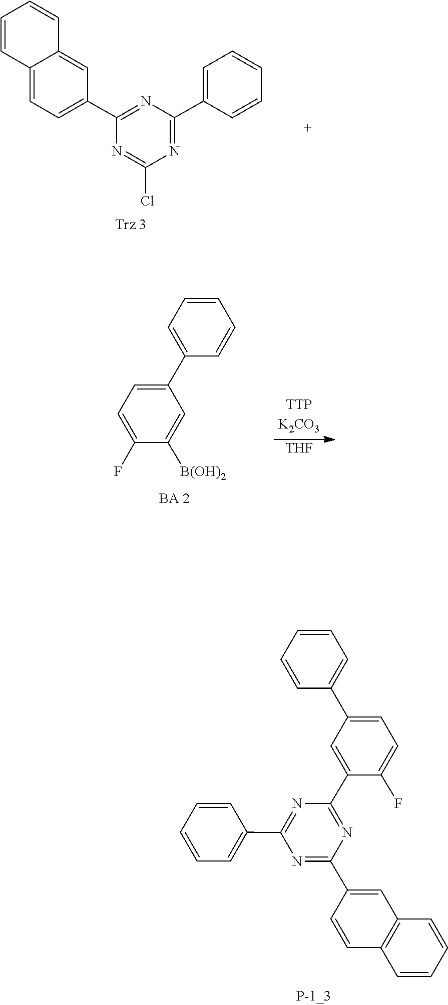 [SMILES: Clc1nc(-c2ccccc2)nc(-c2ccc3ccccc3c2)n1 ; OB(O)c1cc(-c2ccccc2)ccc1F ; Fc1ccc(-c2ccccc2)cc1-c1nc(-c2ccccc2)nc(-c2ccc3ccccc3c2)n1 ; "THF"]

Trz 3 (30 g, 94.4 mmol), BA 2 (21 g, 97.2 mmol), and potassium carbonate (26.1 g, 188.8 mmol) were added to 300 mL of tetrahydrofuran under a nitrogen atmosphere, and the mixture was stirred and refluxed. Tetrakis(triphenylphosphine)palladium(0) (0.3 g, 1.9 mmol) was added thereto. When the reaction was terminated after 5 hours, the reaction mixture was cooled to room temperature, and the solvent was removed under reduced pressure. Then, the compound was completely dissolved in toluene, washed twice with water, and the organic layer was separated, treated with anhydrous magnesium sulfate and then filtered, and the filtrate was distilled under reduced pressure. The concentrated compound was purified by silica gel column chromatography to obtain 22.7 g of Compound P-1_3. (Yield: 53%, MS: [M+H]⁺=455)

### Synthesis Example D: Preparation of Compound P-1_4

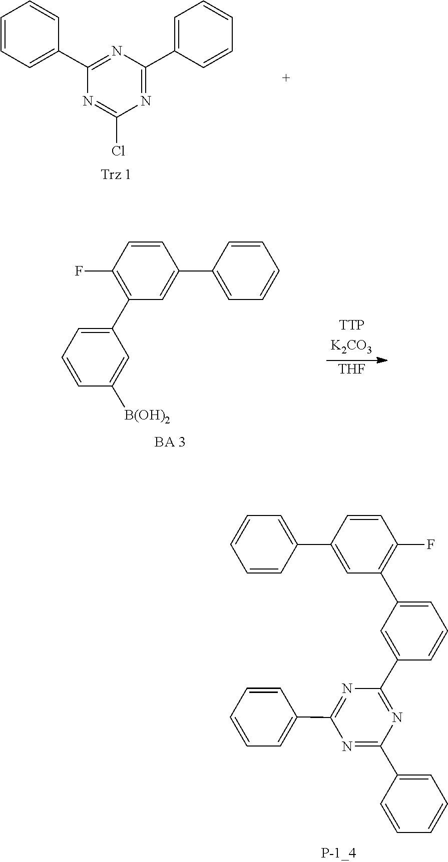 [SMILES: Clc1nc(-c2ccccc2)nc(-c2ccccc2)n1 ; OB(O)c1cccc(-c2cc(-c3ccccc3)ccc2F)c1 ; Fc1ccc(-c2ccccc2)cc1-c1cccc(-c2nc(-c3ccccc3)nc(-c3ccccc3)n2)c1 ; "THF"]

Trz 1 (30 g, 112.1 mmol), BA 3 (33.7 g, 115.4 mmol), and potassium carbonate (31 g, 224.1 mmol) were added to 300 mL of tetrahydrofuran under a nitrogen atmosphere, and the mixture was stirred and refluxed. Tetrakis(triphenylphosphine)palladium(0) (0.3 g, 2.2 mmol) was added thereto. When the reaction was terminated after 5 hours, the reaction mixture was cooled to room temperature, and the solvent was removed under reduced pressure. Then, the compound was completely dissolved in toluene, washed twice with water, and the organic layer was separated, treated with anhydrous magnesium sulfate and then filtered, and the filtrate was distilled under reduced pressure. The concentrated compound was purified by silica gel column chromatography to obtain 34.9 g of Compound P-1_4. (Yield: 65%, MS: [M+H]⁺=481)

### Synthesis Example E: Preparation of Compound P-1_5

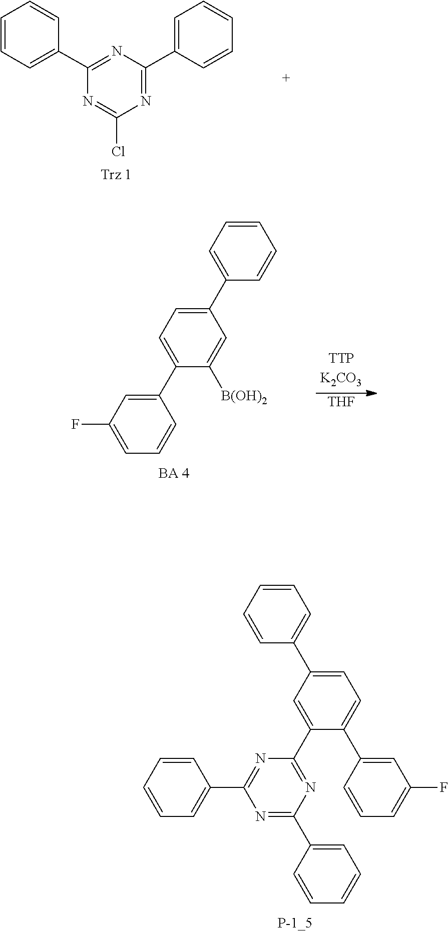 [SMILES: Clc1nc(-c2ccccc2)nc(-c2ccccc2)n1 ; OB(O)c1cc(-c2ccccc2)ccc1-c1cccc(F)c1 ; Fc1cccc(-c2ccc(-c3ccccc3)cc2-c2nc(-c3ccccc3)nc(-c3ccccc3)n2)c1 ; "THF"]

Trz 1 (30 g, 112.1 mmol), BA 4 (36.7 g, 115.4 mmol), and potassium carbonate (31 g, 224.1 mmol) were added to 300 mL of tetrahydrofuran under a nitrogen atmosphere, and the mixture was stirred and refluxed. Tetrakis(triphenylphosphine)palladium(0) (0.3 g, 2.2 mmol) was added thereto. When the reaction was terminated after 5 hours, the reaction mixture was cooled to room temperature, and the solvent was removed under reduced pressure. Then, the compound was completely dissolved in toluene, washed twice with water, and the organic layer was separated, treated with anhydrous magnesium sulfate and then filtered, and the filtrate was distilled under reduced pressure. The concentrated compound was purified by silica gel column chromatography to obtain 37.6 g of Compound P-1_5. (Yield: 70%, MS: [M+H]⁺=481)

### Synthesis Example F: Preparation of Compound P-1_6

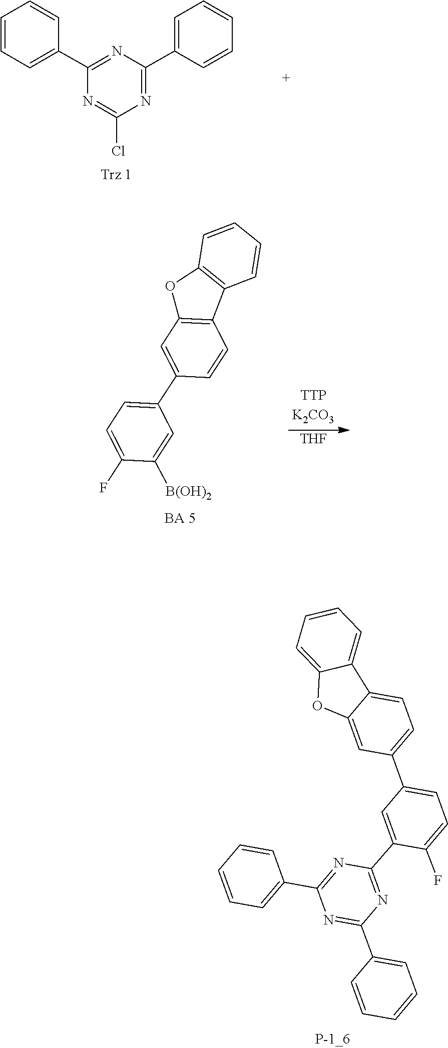 [SMILES: Clc1nc(-c2ccccc2)nc(-c2ccccc2)n1 ; OB(O)c1cc(-c2ccc3c(c2)oc2ccccc23)ccc1F ; Fc1ccc(-c2ccc3c(c2)oc2ccccc23)cc1-c1nc(-c2ccccc2)nc(-c2ccccc2)n1 ; "THF"]

Trz 1 (30 g, 112.1 mmol), BA 5 (35.3 g, 115.4 mmol), and potassium carbonate (31 g, 224.1 mmol) were added to 300 mL of THE under a nitrogen atmosphere, and the mixture was stirred and refluxed. Tetrakis(triphenylphosphine)palladium(0) (2.6 g, 2.2 mmol) was added thereto. When the reaction was terminated after 1 hour, the reaction mixture was cooled to room temperature, and the solvent was removed under reduced pressure. Then, the compound was completely dissolved in toluene, washed twice with water, and the organic layer was separated, treated with anhydrous magnesium sulfate and then filtered, and the filtrate was distilled under reduced pressure. The concentrated compound was purified by silica gel column chromatography to obtain 32.6 g of Compound P-1_6. (Yield: 59%, MS: [M+H]⁺=495)

### Synthesis Example G: Preparation of Compound P-1_7

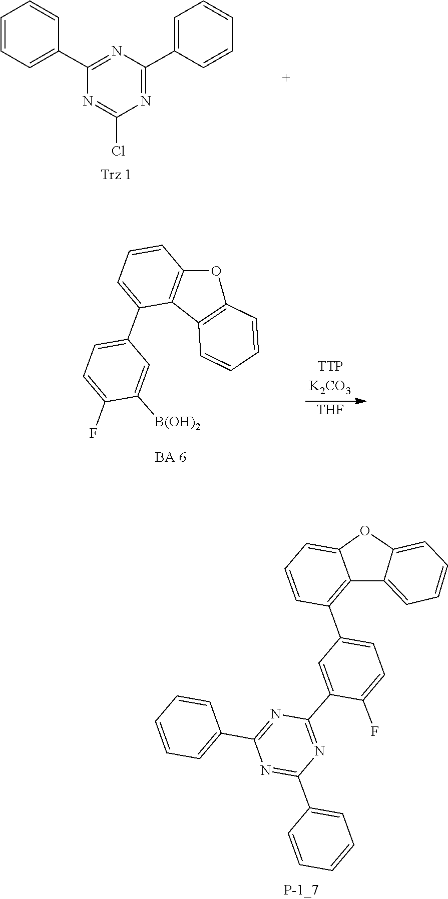 [SMILES: Clc1nc(-c2ccccc2)nc(-c2ccccc2)n1 ; OB(O)c1cc(-c2cccc3oc4ccccc4c23)ccc1F ; Fc1ccc(-c2cccc3oc4ccccc4c23)cc1-c1nc(-c2ccccc2)nc(-c2ccccc2)n1 ; "THF"]

Trz 1 (30 g, 112.1 mmol), BA 6 (35.3 g, 115.4 mmol), and potassium carbonate (31 g, 224.1 mmol) were added to 300 mL of THE under a nitrogen atmosphere, and the mixture was stirred and refluxed. Tetrakis(triphenylphosphine)palladium(0) (2.6 g, 2.2 mmol) was added thereto. When the reaction was terminated after 1 hour, the reaction mixture was cooled to room temperature, and the solvent was removed under reduced pressure. Then, the compound was completely dissolved in toluene, washed twice with water, and the organic layer was separated, treated with anhydrous magnesium sulfate and then filtered, and the filtrate was distilled under reduced pressure. The concentrated compound was purified by silica gel column chromatography to obtain 38.7 g of Compound P-1_7. (Yield: 70%, MS: [M+H]⁺=495)

### Synthesis Example H: Preparation of Compound P-1_8

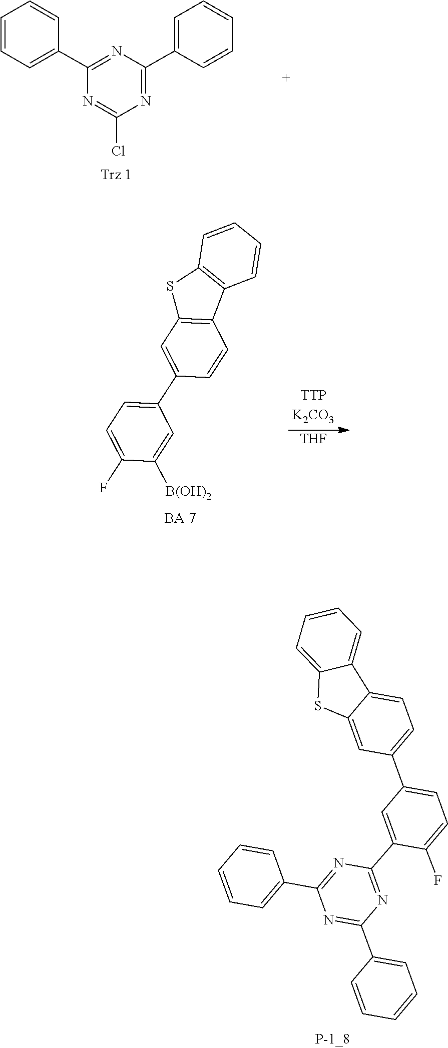 [SMILES: Clc1nc(-c2ccccc2)nc(-c2ccccc2)n1 ; OB(O)c1cc(-c2ccc3c(c2)sc2ccccc23)ccc1F ; Fc1ccc(-c2ccc3c(c2)sc2ccccc23)cc1-c1nc(-c2ccccc2)nc(-c2ccccc2)n1 ; "THF"]

Trz 1 (30 g, 112.1 mmol), BA 7 (37.2 g, 115.4 mmol), and potassium carbonate (31 g, 224.1 mmol) were added to 300 mL of THE under a nitrogen atmosphere, and the mixture was stirred and refluxed. Tetrakis(triphenylphosphine)palladium(0) (2.6 g, 2.2 mmol) was added thereto. When the reaction was terminated after 1 hour, the reaction mixture was cooled to room temperature, and the solvent was removed under reduced pressure. Then, the compound was completely dissolved in toluene, washed twice with water, and the organic layer was separated, treated with anhydrous magnesium sulfate and then filtered, and the filtrate was distilled under reduced pressure. The concentrated compound was purified by silica gel column chromatography to obtain 38.8 g of Compound P-1_8. (Yield: 68%, MS: [M+H]⁺=511)

### Synthesis Example I: Preparation of Compound P-1_9

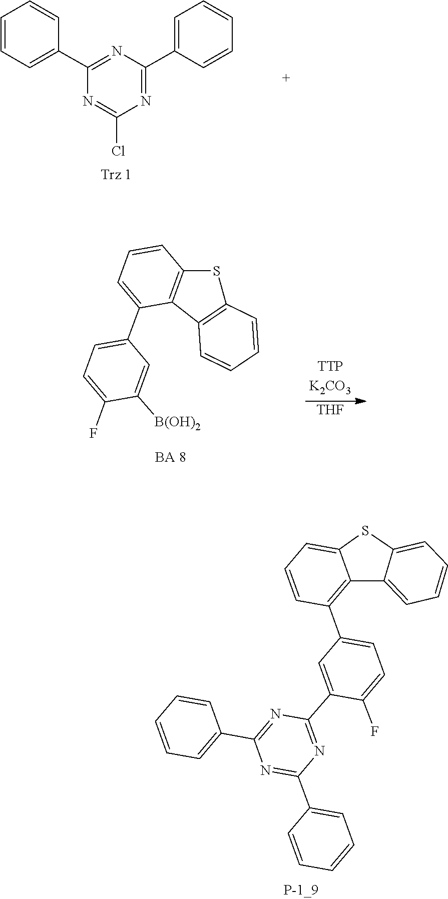 [SMILES: Clc1nc(-c2ccccc2)nc(-c2ccccc2)n1 ; OB(O)c1cc(-c2cccc3sc4ccccc4c23)ccc1F ; Fc1ccc(-c2cccc3sc4ccccc4c23)cc1-c1nc(-c2ccccc2)nc(-c2ccccc2)n1 ; "THF"]

Trz 1 (30 g, 112.1 mmol), BA 8 (37.2 g, 115.4 mmol), and potassium carbonate (31 g, 224.1 mmol) were added to 300 mL of THE under a nitrogen atmosphere, and the mixture was stirred and refluxed. Tetrakis(triphenylphosphine)palladium(0) (2.6 g, 2.2 mmol) was added thereto. When the reaction was terminated after 1 hour, the reaction mixture was cooled to room temperature, and the solvent was removed under reduced pressure. Then, the compound was completely dissolved in toluene, washed twice with water, and the organic layer was separated, treated with anhydrous magnesium sulfate and then filtered, and the filtrate was distilled under reduced pressure. The concentrated compound was purified by silica gel column chromatography to obtain 33.1 g of Compound P-1_9. (Yield: 58%, MS: [M+H]⁺=511)

### Synthesis Example 1: Preparation of Compound 1

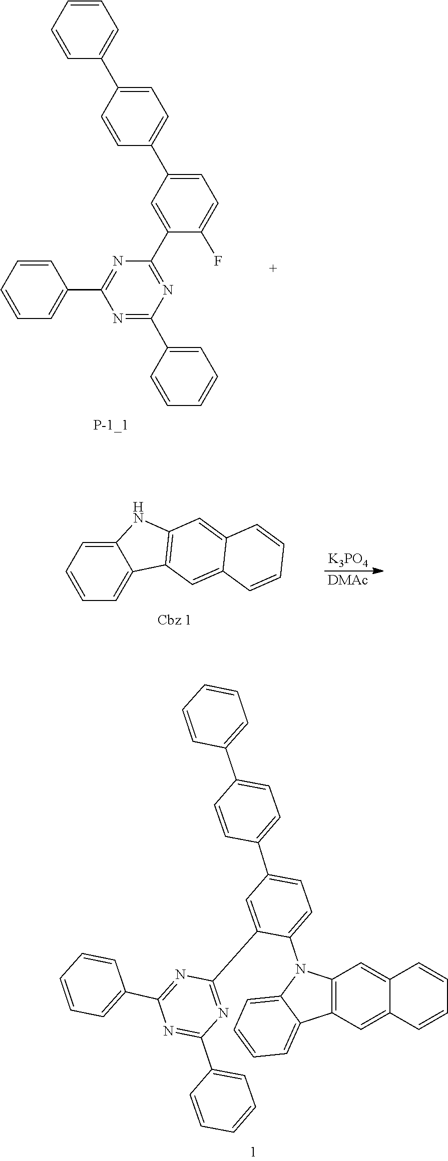

Compound P-1_1 (30 g, 62.6 mmol), Cbz 1 (14 g, 64.4 mmol), and potassium phosphate (39.8 g, 187.7 mmol) were added to 210 mL of dimethylacetamide under a nitrogen atmosphere, and the mixture was stirred and refluxed. When the reaction was terminated after 1 hour, the reaction mixture was cooled to room temperature, and the solvent was removed under reduced pressure. Then, the compound was completely dissolved in toluene, washed twice with water, and the organic layer was separated, treated with anhydrous magnesium sulfate and then filtered, and the filtrate was distilled under reduced pressure. The concentrated compound was purified by silica gel column chromatography to obtain 22.4 g of Compound 1. (Yield: 53%, MS: [M+H]⁺=678)

### Synthesis Example 2: Preparation of Compound 2

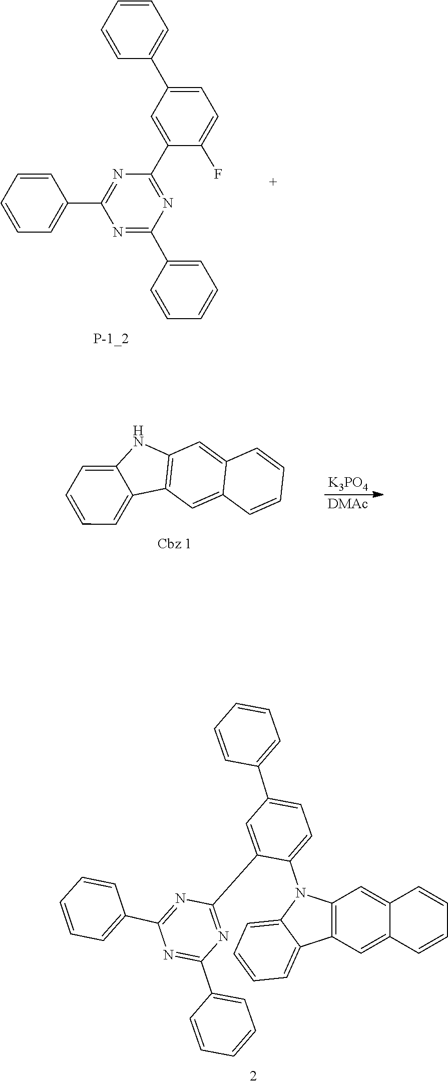

Compound P-1_2 (30 g, 74.4 mmol), Cbz 1 (16.6 g, 76.6 mmol), and potassium phosphate (47.3 g, 223.1 mmol) were added to 210 mL of dimethylacetamide under a nitrogen atmosphere, and the mixture was stirred and refluxed. When the reaction was terminated after 1 hour, the reaction mixture was cooled to room temperature, and the solvent was removed under reduced pressure. Then, the compound was completely dissolved in toluene, washed twice with water, and the organic layer was separated, treated with anhydrous magnesium sulfate and then filtered, and the filtrate was distilled under reduced pressure. The concentrated compound was purified by silica gel column chromatography to obtain 23.7 g of Compound 2. (Yield: 53%, MS: [M+H]⁺=602)

### Synthesis Example 3: Preparation of Compound 3

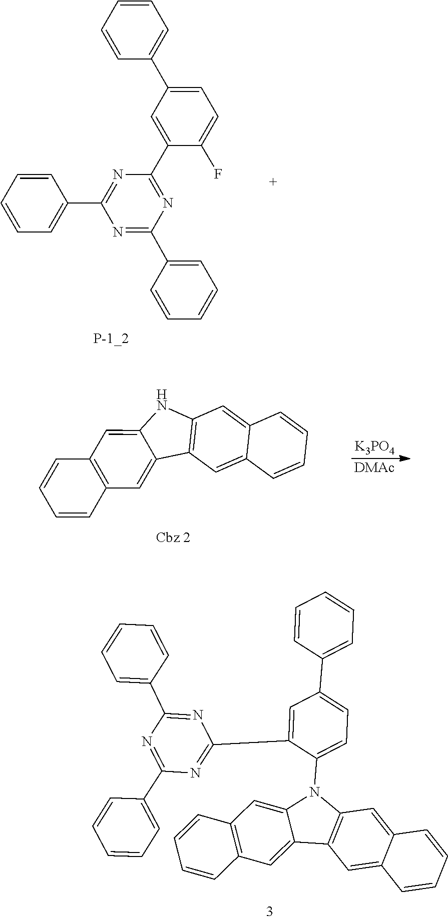

Compound P-1_2 (30 g, 74.4 mmol), Cbz 2 (20.5 g, 76.6 mmol), and potassium phosphate (47.3 g, 223.1 mmol) were added to 210 mL of dimethylacetamide under a nitrogen atmosphere, and the mixture was stirred and refluxed. When the reaction was terminated after 1 hour, the reaction mixture was cooled to room temperature, and the solvent was removed under reduced pressure. Then, the compound was completely dissolved in toluene, washed twice with water, and the organic layer was separated, treated with anhydrous magnesium sulfate and then filtered, and the filtrate was distilled under reduced pressure. The concentrated compound was purified by silica gel column chromatography to obtain 30 g of Compound 3. (Yield: 62%, MS: [M+H]⁺=652)

### Synthesis Example 4: Preparation of Compound 4

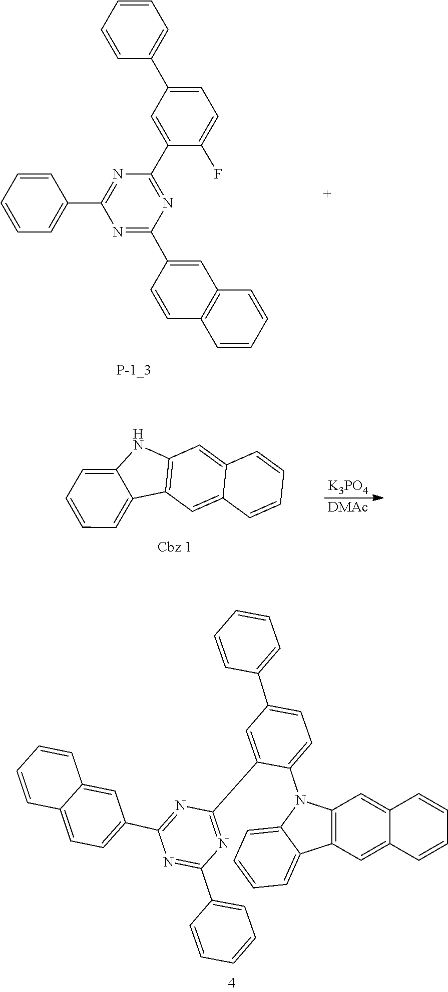

compound P-1_3 (30 g, 66.1 mmol), Cbz 1 (14.5 g, 68.1 mmol), and potassium phosphate (42.1 g, 198.4 mmol) were added to 210 mL of dimethylacetamide under a nitrogen atmosphere, and the mixture was stirred and refluxed. When the reaction was terminated after 1 hour, the reaction mixture was cooled to room temperature, and the solvent was removed under reduced pressure. Then, the compound was completely dissolved in toluene, washed twice with water, and the organic layer was separated, treated with anhydrous magnesium sulfate and then filtered, and the filtrate was distilled under reduced pressure. The concentrated compound was purified by silica gel column chromatography to obtain 28 g of Compound 4. (Yield: 65%, MS: [M+H]⁺=652)

### Synthesis Example 5: Preparation of Compound 5

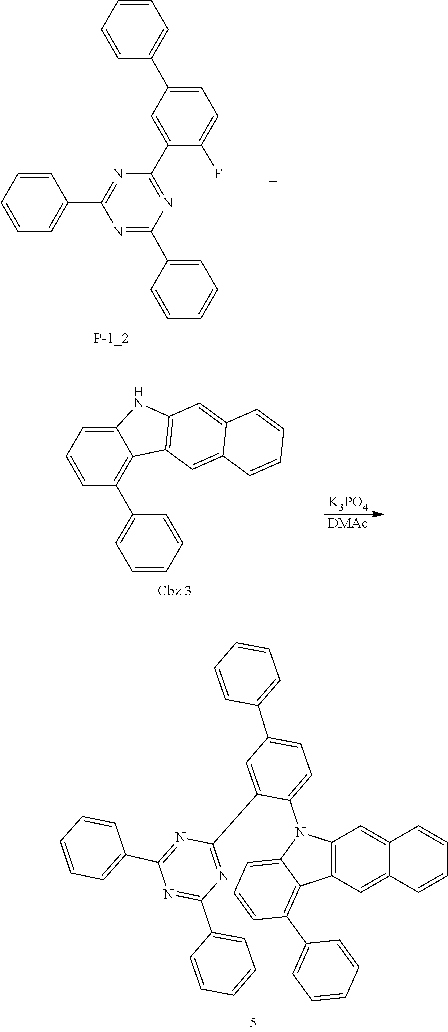

compound P-1_2 (30 g, 74.4 mmol), Cbz 3 (22.5 g, 76.6 mmol), and potassium phosphate (47.3 g, 223.1 mmol) were added to 210 mL of dimethylacetamide under a nitrogen atmosphere, and the mixture was stirred and refluxed. When the reaction was terminated after 1 hour, the reaction mixture was cooled to room temperature, and the solvent was removed under reduced pressure. Then, the compound was completely dissolved in toluene, washed twice with water, and the organic layer was separated, treated with anhydrous magnesium sulfate and then filtered, and the filtrate was distilled under reduced pressure. The concentrated compound was purified by silica gel column chromatography to obtain 28.2 g of Compound 5. (Yield: 56%, MS: [M+H]⁺=678)

### Synthesis Example 6: Preparation of Compound 6

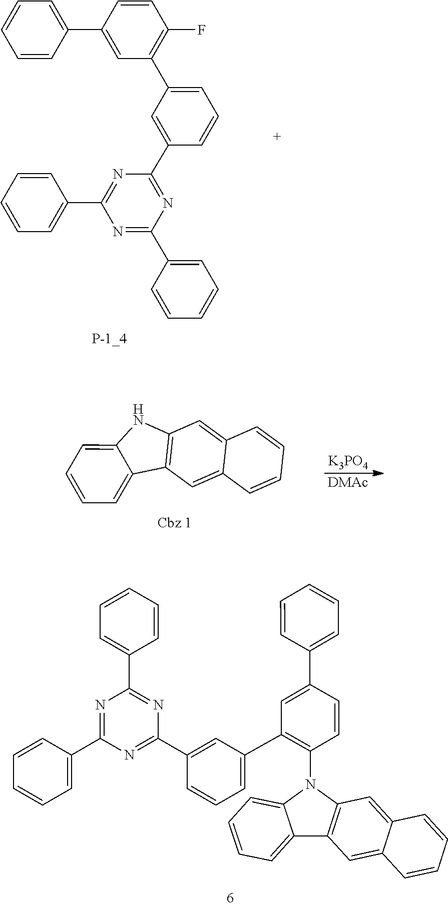

Compound 6 was obtained using the same method as in Synthesis Example 1, except that P-1_4 was used instead of P-1_1 in Synthesis Example 1. (Yield: 78%, MS: [M+H]⁺=678)

### Synthesis Example 7: Preparation of Compound 7

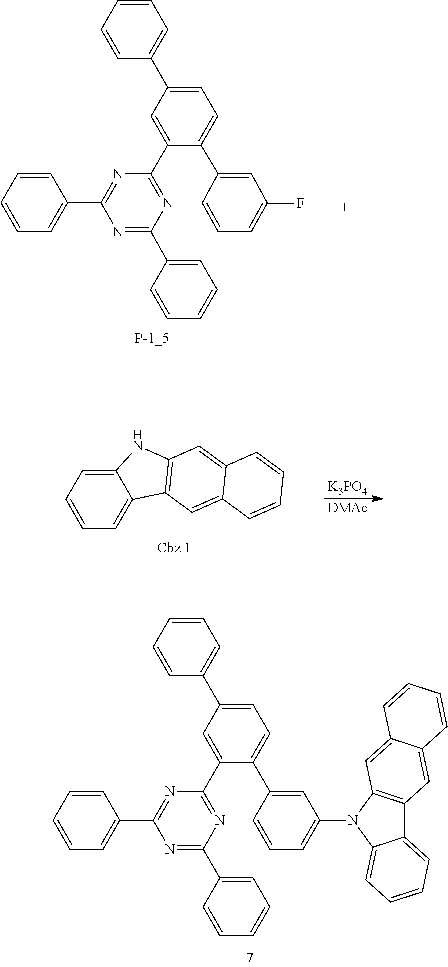

Compound 7 was obtained using the same method as in Synthesis Example 1, except that P-1_5 was used instead of P-1_1 in Synthesis Example 1. (Yield: 84%, MS: [M+H]⁺=678)

### Synthesis Example 8: Preparation of Compound 8

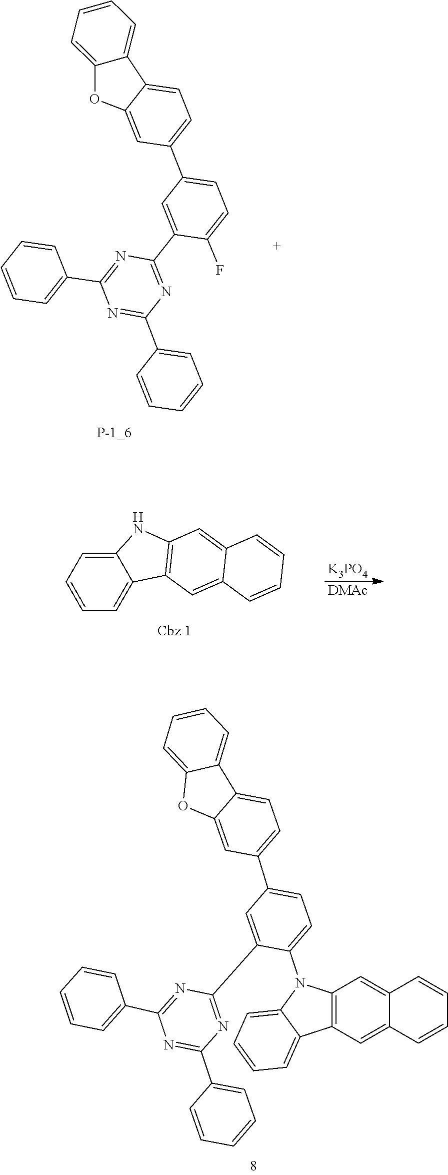

Compound P-1_6 (30 g, 60.8 mmol), Cbz 1 (13.6 g, 62.6 mmol), and potassium phosphate (38.7 g, 182.4 mmol) were added to 210 mL of dimethylacetamide under a nitrogen atmosphere, and the mixture was stirred and refluxed. When the reaction was terminated after 1 hour, the reaction mixture was cooled to room temperature, and the solvent was removed under reduced pressure. Then, the compound was completely dissolved in toluene, washed twice with water, and the organic layer was separated, treated with anhydrous magnesium sulfate and then filtered, and the filtrate was distilled under reduced pressure. The concentrated compound was purified by silica gel column chromatography to obtain 26.9 g of Compound 8. (Yield: 64%, MS: [M+H]⁺=692)

### Synthesis Example 9: Preparation of Compound 9

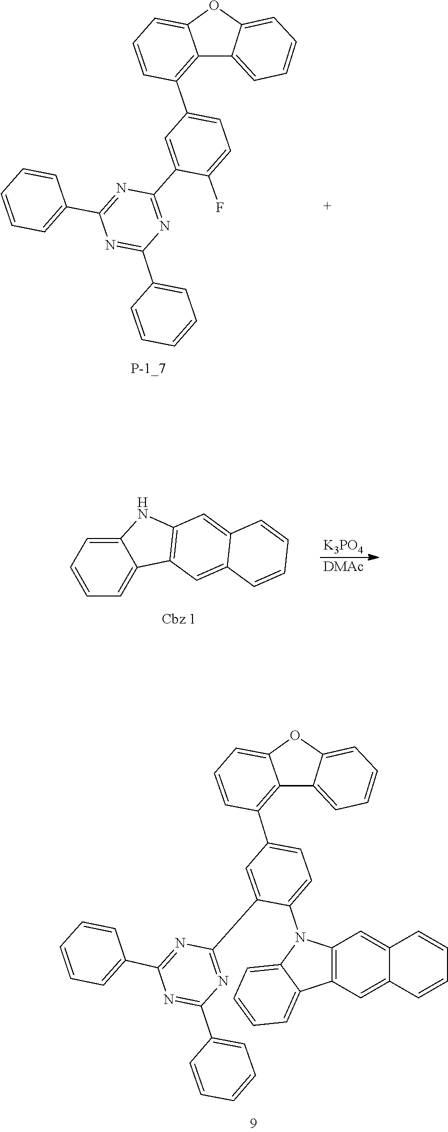

compound P-1_7 (30 g, 60.8 mmol), Cbz 1 (13.6 g, 62.6 mmol), and potassium phosphate (38.7 g, 182.4 mmol) were added to 210 mL of dimethylacetamide under a nitrogen atmosphere, and the mixture was stirred and refluxed. When the reaction was terminated after 1 hour, the reaction mixture was cooled to room temperature, and the solvent was removed under reduced pressure. Then, the compound was completely dissolved in toluene, washed twice with water, and the organic layer was separated, treated with anhydrous magnesium sulfate and then filtered, and the filtrate was distilled under reduced pressure. The concentrated compound was purified by silica gel column chromatography to obtain 26.9 g of Compound 9. (Yield: 64%, MS: [M+H]⁺=692)

### Synthesis Example 10: Preparation of Compound 10

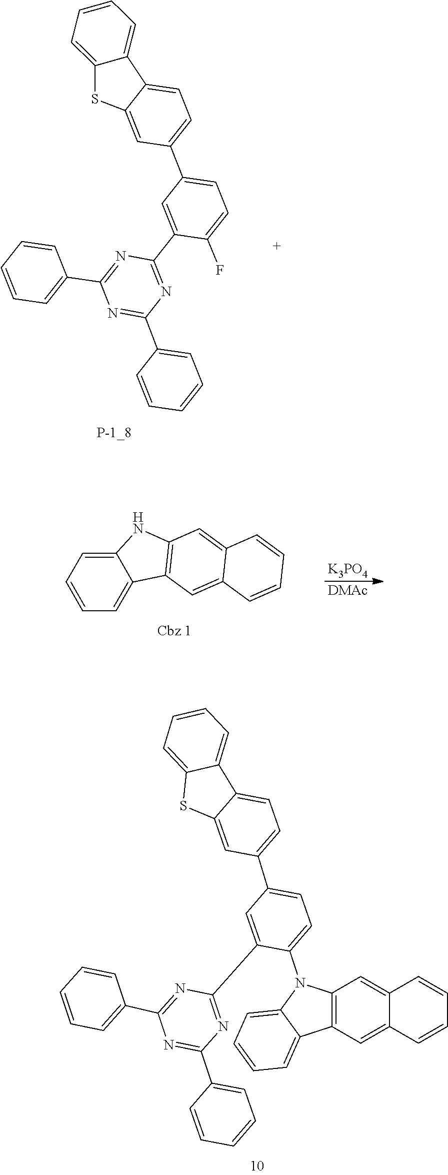

Compound 10 was obtained using the same method as in Synthesis Example 1, except that P-1_8 was used instead of P-1_1 in Synthesis Example 1. (Yield: 89%, MS: [M+H]⁺=708)

### Synthesis Example 11: Preparation of Compound 11

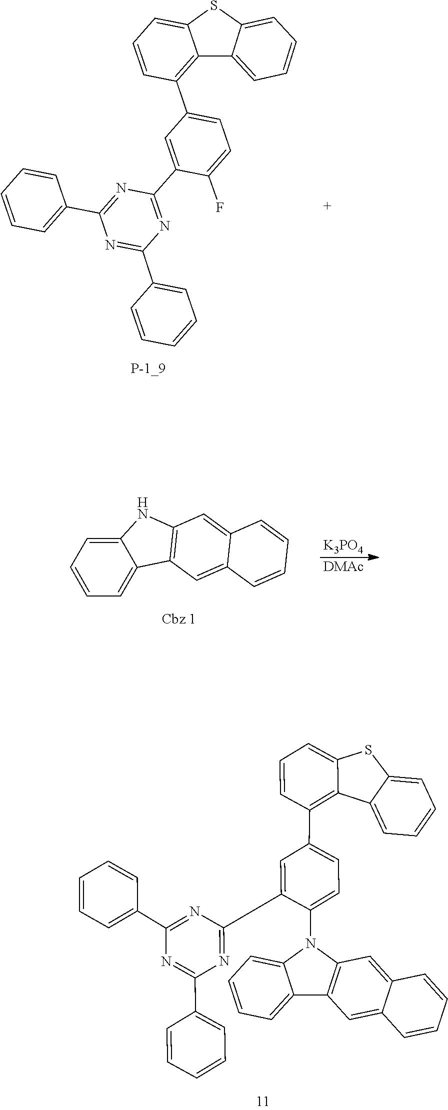

Compound P-1_9 (30 g, 58.9 mmol), Cbz 1 (13.2 g, 60.6 mmol), and potassium phosphate (37.5 g, 176.6 mmol) were added to 210 mL of dimethylacetamide under a nitrogen atmosphere, and the mixture was stirred and refluxed. Tetrakis(triphenylphosphine)palladium(0) (1.4 g, 1.2 mmol) was added thereto. When the reaction was terminated after 1 hour, the reaction mixture was cooled to room temperature, and the solvent was removed under reduced pressure. Then, the compound was completely dissolved in toluene, washed twice with water, and the organic layer was separated, treated with anhydrous magnesium sulfate and then filtered, and the filtrate was distilled under reduced pressure. The concentrated compound was purified by silica gel column chromatography to obtain 24.1 g of Compound 11. (Yield: 58%, MS: [M+H]⁺=708)

### Synthesis Example 12: Preparation of Compound 12

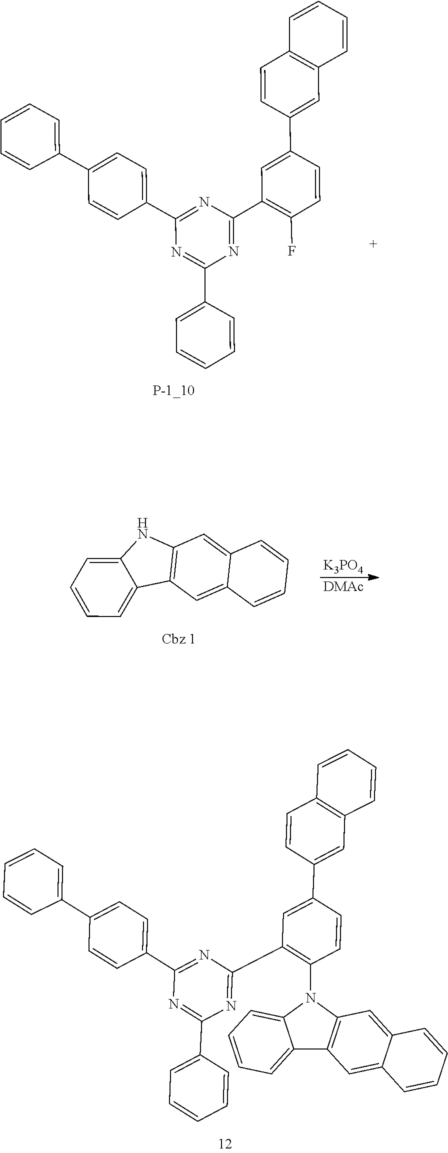

P-1_10 (30 g, 56.6 mmol), Cbz 1 (12.7 g, 58.3 mmol), and potassium phosphate (36.1 g, 169.9 mmol) were added to 210 mL of dimethylacetamide under a nitrogen atmosphere, and the mixture was stirred and refluxed. When the reaction was terminated after 1 hour, the reaction mixture was cooled to room temperature, and the solvent was removed under reduced pressure. Then, the compound was completely dissolved in toluene, washed twice with water, and the organic layer was separated, treated with anhydrous magnesium sulfate and then filtered, and the filtrate was distilled under reduced pressure. The concentrated compound was purified by silica gel column chromatography to obtain 23.9 g of Compound 12. (Yield: 58%, MS: [M+H]⁺=728)

### Synthesis Example 13: Preparation of Compound 13

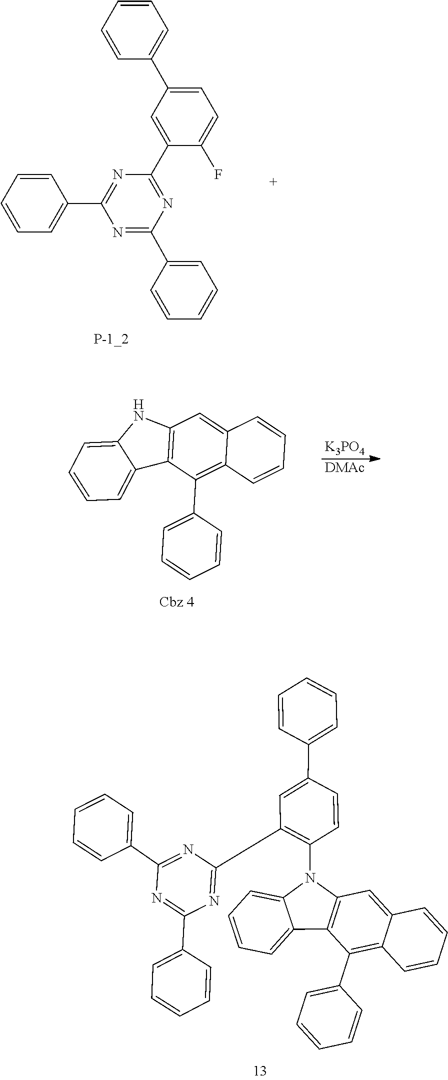

P-1_2 (30 g, 74.4 mmol), Cbz 4 (22.5 g, 76.6 mmol), and potassium phosphate (47.3 g, 223.1 mmol) were added to 210 mL of dimethylacetamide under a nitrogen atmosphere, and the mixture was stirred and refluxed. When the reaction was terminated after 1 hour, the reaction mixture was cooled to room temperature, and the solvent was removed under reduced pressure. Then, the compound was completely dissolved in toluene, washed twice with water, and the organic layer was separated, treated with anhydrous magnesium sulfate and then filtered, and the filtrate was distilled under reduced pressure. The concentrated compound was purified by silica gel column chromatography to obtain 31.7 g of Compound 13. (Yield: 63%, MS: [M+H]⁺=677)

### Synthesis Example 14: Preparation of Compound 14

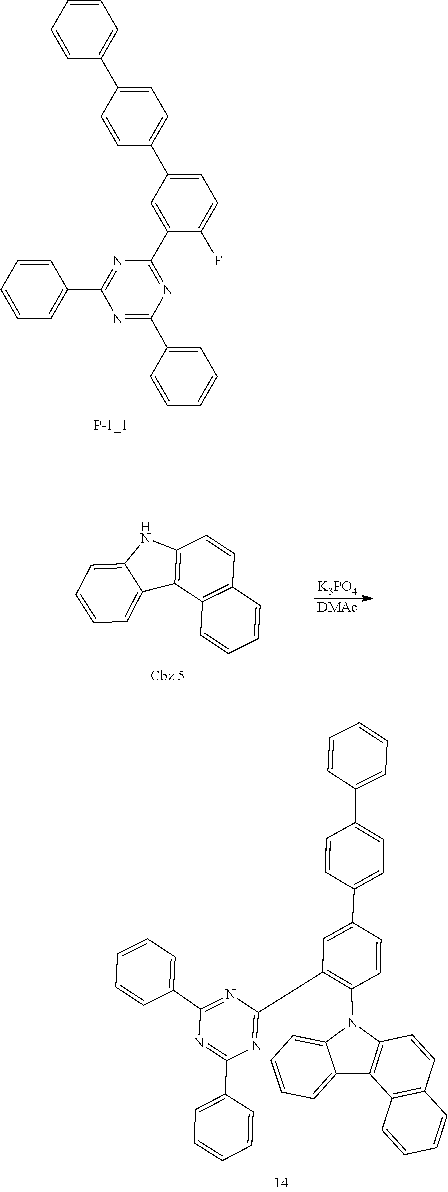

P-1_1 (30 g, 62.6 mmol), Cbz 5 (14 g, 64.4 mmol), and potassium phosphate (39.8 g, 187.7 mmol) were added to 210 mL of dimethylacetamide under a nitrogen atmosphere, and the mixture was stirred and refluxed. When the reaction was terminated after 1 hour, the reaction mixture was cooled to room temperature, and the solvent was removed under reduced pressure. Then, the compound was completely dissolved in toluene, washed twice with water, and the organic layer was separated, treated with anhydrous magnesium sulfate and then filtered, and the filtrate was distilled under reduced pressure. The concentrated compound was purified by silica gel column chromatography to obtain 22.9 g of Compound 14. (Yield: 54%, MS: [M+H]⁺=677)

### Synthesis Example 15: Preparation of Compound 15

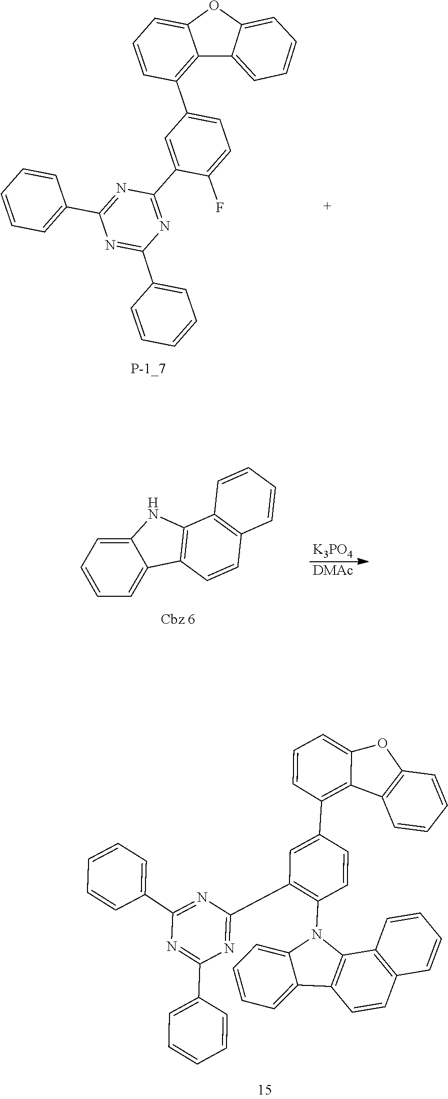

P-1_7 (30 g, 60.8 mmol), Cbz 6 (13.6 g, 62.6 mmol), and potassium phosphate (38.7 g, 182.4 mmol) were added to 210 mL of dimethylacetamide under a nitrogen atmosphere, and the mixture was stirred and refluxed. When the reaction was terminated after 1 hour, the reaction mixture was cooled to room temperature, and the solvent was removed under reduced pressure. Then, the compound was completely dissolved in toluene, washed twice with water, and the organic layer was separated, treated with anhydrous magnesium sulfate and then filtered, and the filtrate was distilled under reduced pressure. The concentrated compound was purified by silica gel column chromatography to obtain 26.9 g of Compound 15. (Yield: 64%, MS: [M+H]⁺=691)

### Synthesis Example 16: Preparation of Compound 16

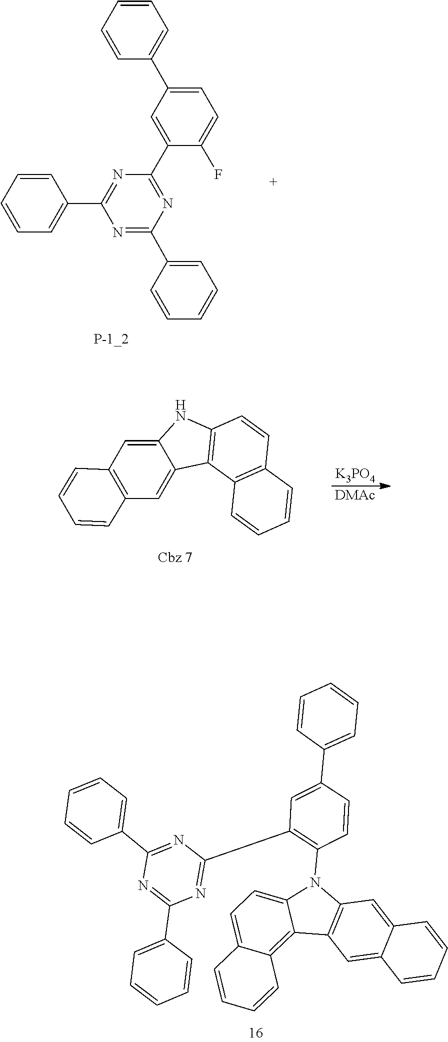

P-1_2 (30 g, 74.4 mmol), Cbz 7 (20.5 g, 76.6 mmol), and potassium phosphate (47.3 g, 223.1 mmol) were added to 210 mL of dimethylacetamide under a nitrogen atmosphere, and the mixture was stirred and refluxed. When the reaction was terminated after 1 hour, the reaction mixture was cooled to room temperature, and the solvent was removed under reduced pressure. Then, the compound was completely dissolved in toluene, washed twice with water, and the organic layer was separated, treated with anhydrous magnesium sulfate and then filtered, and the filtrate was distilled under reduced pressure. The concentrated compound was purified by silica gel column chromatography to obtain 32.9 g of Compound 16. (Yield: 68%, MS: [M+H]⁺=651)

### Synthesis Example 17: Preparation of Compound 17

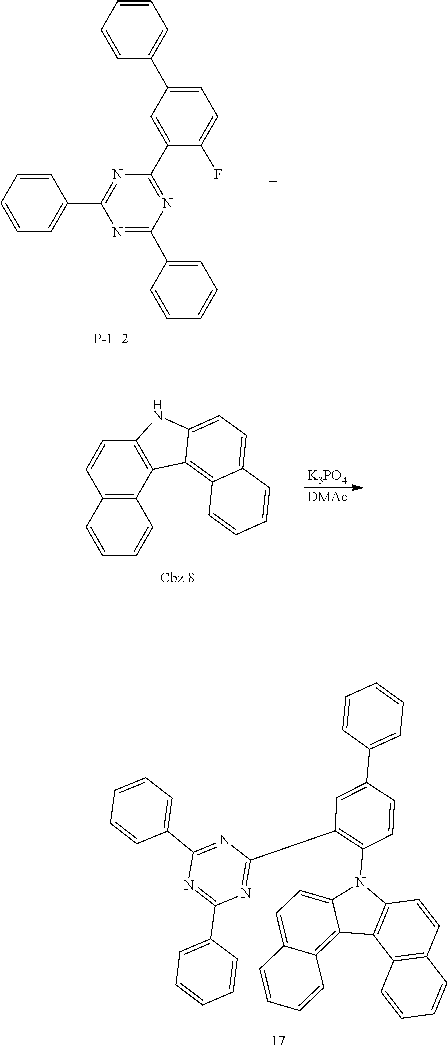

P-1_2 (30 g, 74.4 mmol), Cbz 8 (20.5 g, 76.6 mmol), and potassium phosphate (47.3 g, 223.1 mmol) were added to 210 mL of dimethylacetamide under a nitrogen atmosphere, and the mixture was stirred and refluxed. When the reaction was terminated after 1 hour, the reaction mixture was cooled to room temperature, and the solvent was removed under reduced pressure. Then, the compound was completely dissolved in toluene, washed twice with water, and the organic layer was separated, treated with anhydrous magnesium sulfate and then filtered, and the filtrate was distilled under reduced pressure. The concentrated compound was purified by silica gel column chromatography to obtain 29 g of Compound 17. (Yield: 60%, MS: [M+H]⁺=651)

### Synthesis Example 18: Preparation of Compound 18

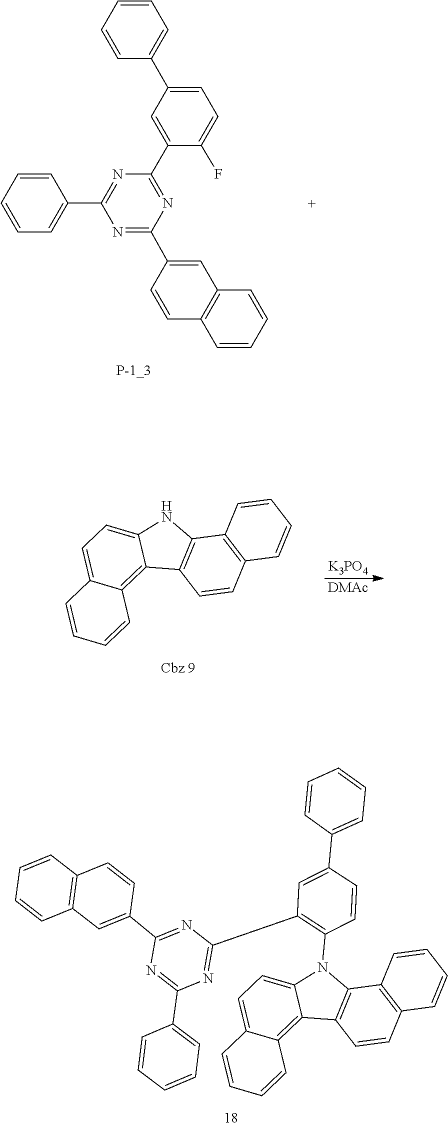

P-1_3 (30 g, 66.1 mmol), Cbz 9 (18.2 g, 68.1 mmol), and potassium phosphate (42.1 g, 198.4 mmol) were added to 210 mL of dimethylacetamide under a nitrogen atmosphere, and the mixture was stirred and refluxed. When the reaction was terminated after 1 hour, the reaction mixture was cooled to room temperature, and the solvent was removed under reduced pressure. Then, the compound was completely dissolved in toluene, washed twice with water, and the organic layer was separated, treated with anhydrous magnesium sulfate and then filtered, and the filtrate was distilled under reduced pressure. The concentrated compound was purified by silica gel column chromatography to obtain 24.6 g of Compound 18. (Yield: 53%, MS: [M+H]⁺=701)

### Synthesis Example 19: Preparation of Compound 19

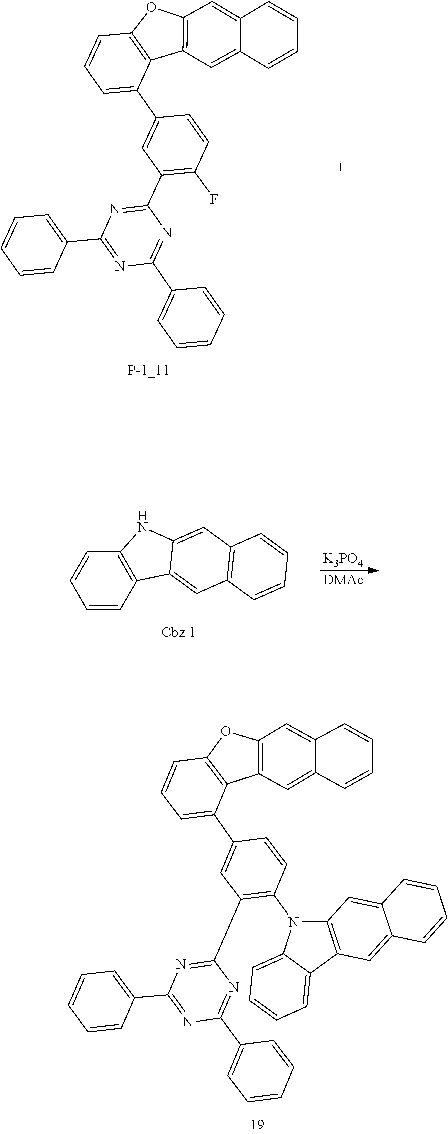

P-1_11 (30 g, 55.2 mmol), Cbz 1 (12.4 g, 56.8 mmol), and potassium phosphate (35.1 g, 165.6 mmol) were added to 210 mL of dimethylacetamide under a nitrogen atmosphere, and the mixture was stirred and refluxed. When the reaction was terminated after 1 hour, the reaction mixture was cooled to room temperature, and the solvent was removed under reduced pressure. Then, the compound was completely dissolved in toluene, washed twice with water, and the organic layer was separated, treated with anhydrous magnesium sulfate and then filtered, and the filtrate was distilled under reduced pressure. The concentrated compound was purified by silica gel column chromatography to obtain 20.4 g of Compound 19. (Yield: 50%, MS: [M+H]⁺=741)

### Synthesis Example 20: Preparation of Compound 20

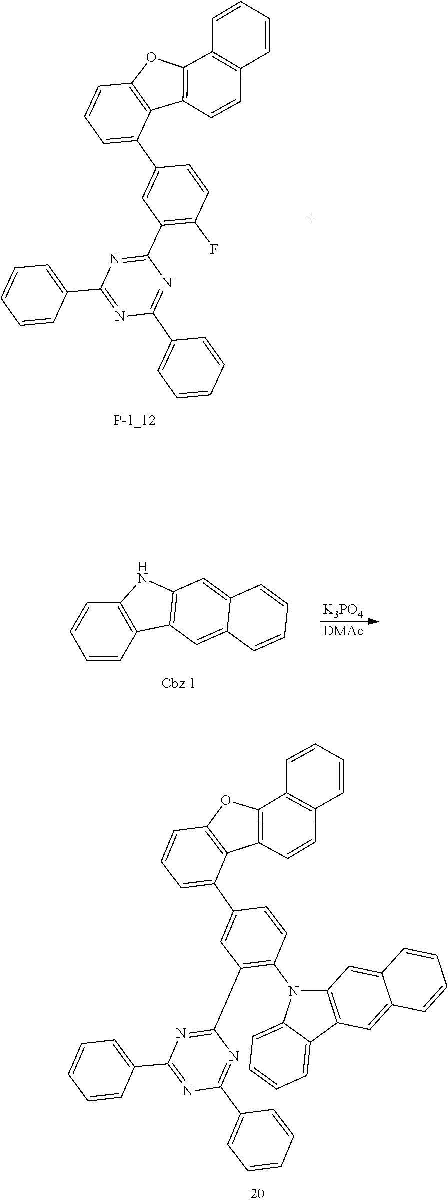

P-1_12 (30 g, 55.2 mmol), Cbz 1 (12.4 g, 56.8 mmol), and potassium phosphate (35.1 g, 165.6 mmol) were added to 210 mL of dimethylacetamide under a nitrogen atmosphere, and the mixture was stirred and refluxed. When the reaction was terminated after 1 hour, the reaction mixture was cooled to room temperature, and the solvent was removed under reduced pressure. Then, the compound was completely dissolved in toluene, washed twice with water, and the organic layer was separated, treated with anhydrous magnesium sulfate and then filtered, and the filtrate was distilled under reduced pressure. The concentrated compound was purified by silica gel column chromatography to obtain 26.2 g of Compound 20. (Yield: 64%, MS: [M+H]⁺=741)

### Synthesis Example 21: Preparation of Compound 21

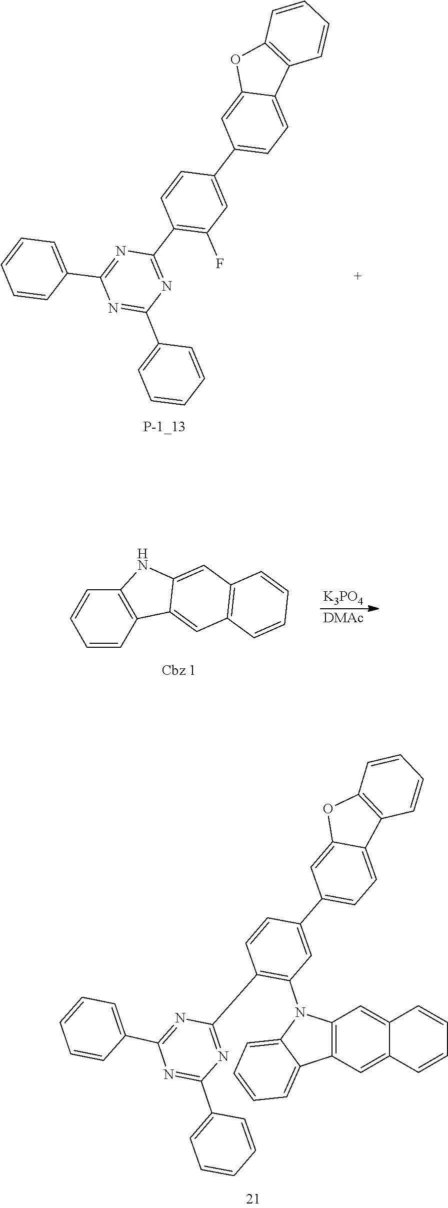

P-1_13 (30 g, 60.8 mmol), Cbz 1 (13.6 g, 62.6 mmol), and potassium phosphate (38.7 g, 182.4 mmol) were added to 210 mL of dimethylacetamide under a nitrogen atmosphere, and the mixture was stirred and refluxed. When the reaction was terminated after 1 hour, the reaction mixture was cooled to room temperature, and the solvent was removed under reduced pressure. Then, the compound was completely dissolved in toluene, washed twice with water, and the organic layer was separated, treated with anhydrous magnesium sulfate and then filtered, and the filtrate was distilled under reduced pressure. The concentrated compound was purified by silica gel column chromatography to obtain 25.6 g of Compound 21. (Yield: 61%, MS: [M+H]⁺=691)

### Synthesis Example 22: Preparation of Compound 22

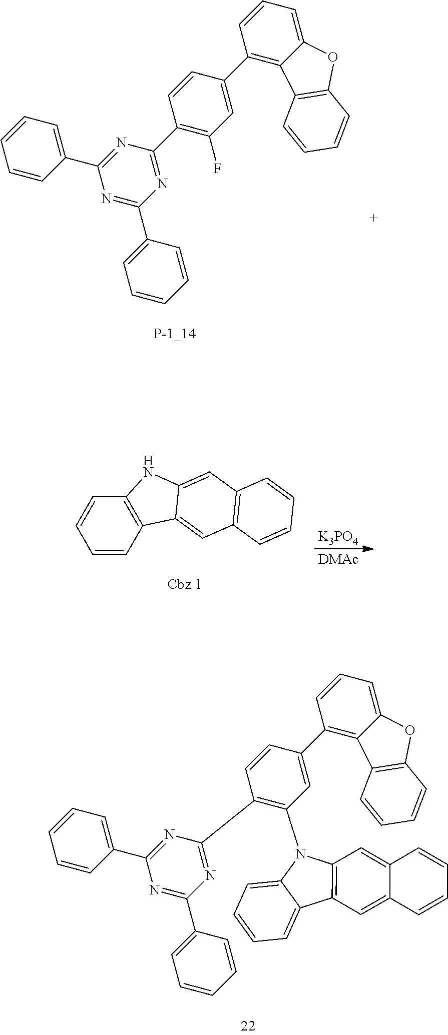

P-1_14 (30 g, 60.8 mmol), Cbz 1 (13.6 g, 62.6 mmol), and potassium phosphate (38.7 g, 182.4 mmol) were added to 210 mL of dimethylacetamide under a nitrogen atmosphere, and the mixture was stirred and refluxed. When the reaction was terminated after 1 hour, the reaction mixture was cooled to room temperature, and the solvent was removed under reduced pressure. Then, the compound was completely dissolved in toluene, washed twice with water, and the organic layer was separated, treated with anhydrous magnesium sulfate and then filtered, and the filtrate was distilled under reduced pressure. The concentrated compound was purified by silica gel column chromatography to obtain 24.4 g of Compound 22. (Yield: 58%, MS: [M+H]⁺=691)

### Synthesis Example 23: Preparation of Compound 23

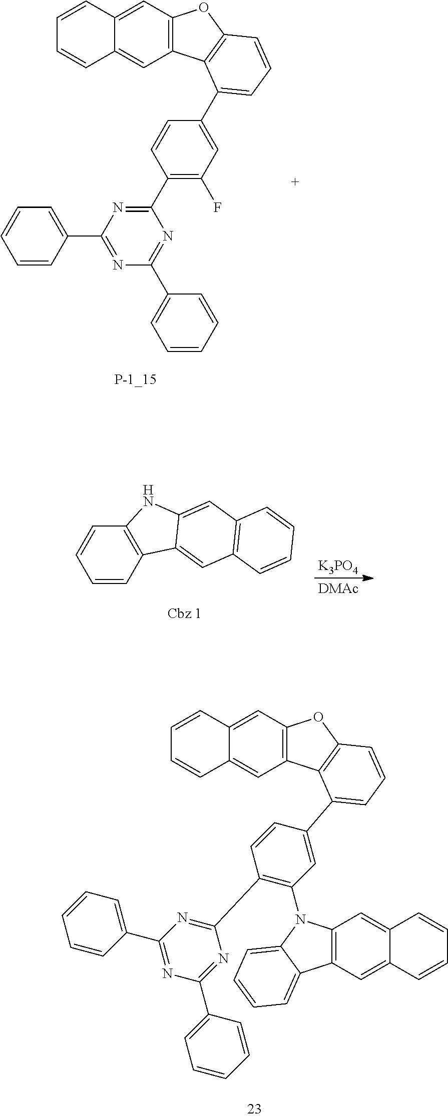

P-1_15 (30 g, 54.9 mmol), Cbz 1 (12.3 g, 56.5 mmol), and potassium phosphate (34.9 g, 164.7 mmol) were added to 210 mL of dimethylacetamide under a nitrogen atmosphere, and the mixture was stirred and refluxed. When the reaction was terminated after 1 hour, the reaction mixture was cooled to room temperature, and the solvent was removed under reduced pressure. Then, the compound was completely dissolved in toluene, washed twice with water, and the organic layer was separated, treated with anhydrous magnesium sulfate and then filtered, and the filtrate was distilled under reduced pressure. The concentrated compound was purified by silica gel column chromatography to obtain 22.4 g of Compound 23. (Yield: 55%, MS: [M+H]⁺=741)

### Synthesis Example 24: Preparation of Compound 1D

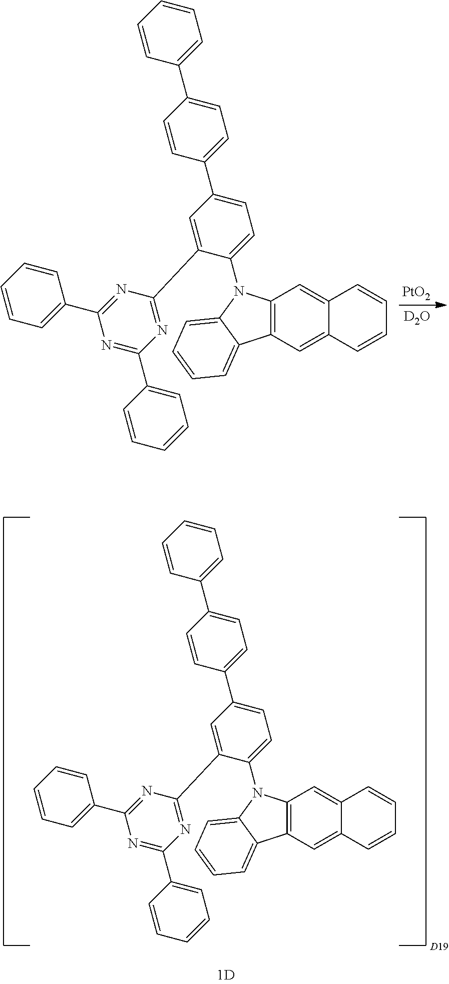

Compound 1 (10 g, 14.7 mmol), PtO₂ (0.97 g, 4.42 mmol), and 74 mL of D₂O were added to a shaker tube, and the shaker tube was sealed and heated at 250° C. and 600 psi for 12 hours. When the reaction was complete, chloroform was added thereto, and the reaction solution was transferred to a separatory funnel for extraction. The extract was dried over MgSO₄ and concentrated, and the sample was purified by silica gel column chromatography to prepare 5.3 g of Compound 1D. (Yield: 52%, [M+H]⁺=696)

### Synthesis Example 25: Preparation of Compound 2D

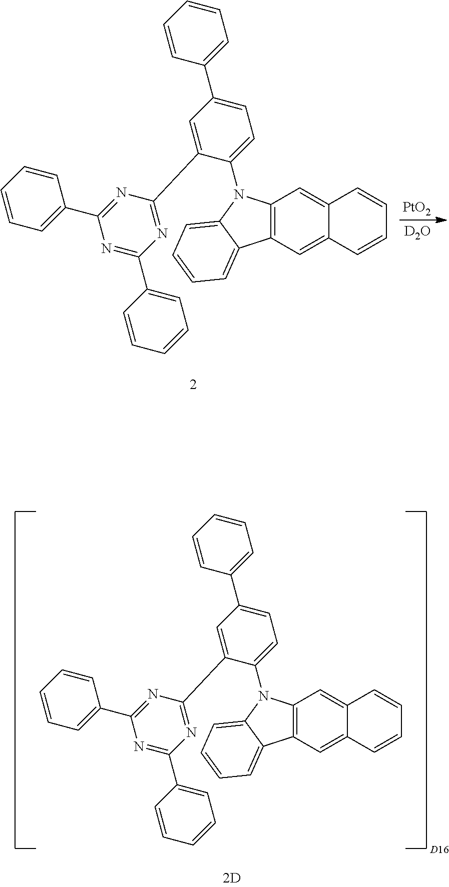

Compound 2 (10 g, 16.6 mmol), PtO₂ (1.1 g, 5 mmol), and 83 mL of D₂O were added to a shaker tube, and the shaker tube was sealed and heated at 250° C. and 600 psi for 12 hours. When the reaction was complete, chloroform was added thereto, and the reaction solution was transferred to a separatory funnel for extraction. The extract was dried over MgSO₄ and concentrated, and the sample was purified by silica gel column chromatography to produce 4.3 g of Compound 2D. (Yield: 42%, [M+H]⁺=617)

### Synthesis Example 26: Preparation of Compound 3D

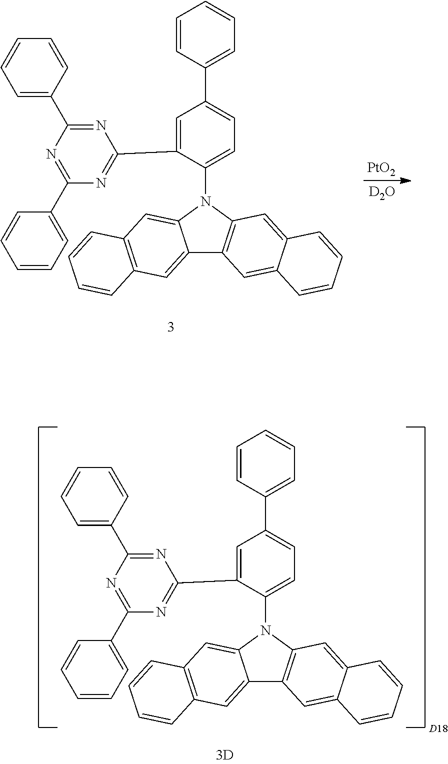

Compound 3 (10 g, 15.3 mmol), PtO₂ (1.0 g, 4.6 mmol), and 76 mL of D₂O were added to a shaker tube, and the shaker tube was sealed and heated at 250° C. and 600 psi for 12 hours. When the reaction was complete, chloroform was added thereto, and the reaction solution was transferred to a separatory funnel for extraction. The extract was dried over MgSO₄ and concentrated, and the sample was purified by silica gel column chromatography to produce 5.6 g of Compound 3D. (Yield: 55%, [M+H]⁺=669)

### Synthesis Example 27: Preparation of Compound 4D

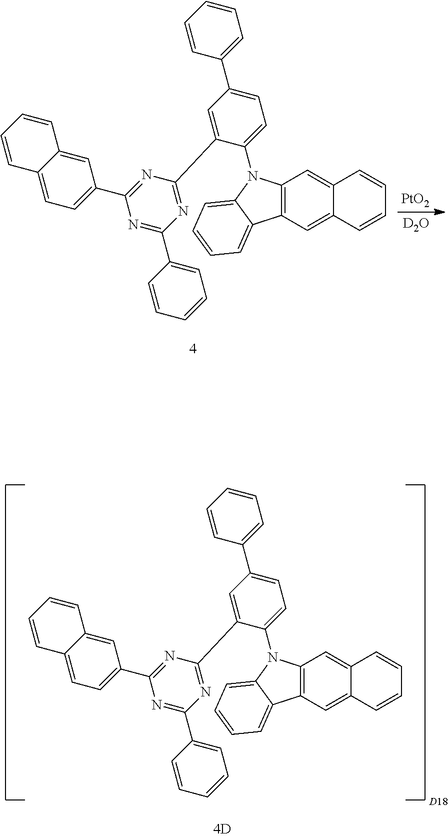

Compound 4 (10 g, 15.3 mmol), PtO₂ (1.0 g, 4.6 mmol), and 76 mL of D₂O were added to a shaker tube, and the shaker tube was sealed and heated at 250° C. and 600 psi for 12 hours. When the reaction was complete, chloroform was added thereto, and the reaction solution was transferred to a separatory funnel for extraction. The extract was dried over MgSO₄ and concentrated, and the sample was purified by silica gel column chromatography to produce 7.7 g of Compound 4D. (Yield: 75%, [M+H]⁺=669)

### Synthesis Example 28: Preparation of Compound 5D

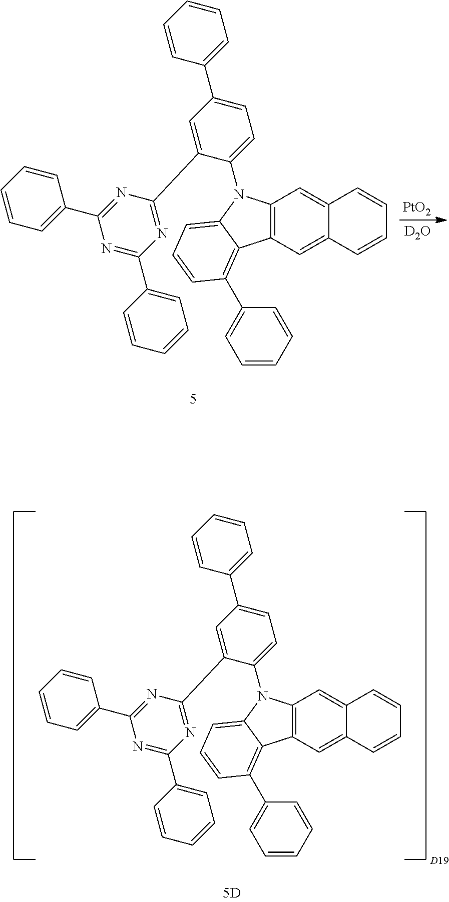

Compound 5 (10 g, 14.8 mmol), PtO₂ (0.98 g, 4.5 mmol), and 74 mL of D₂O were added to a shaker tube, and the shaker tube was sealed and heated at 250° C. and 600 psi for 12 hours. When the reaction was complete, chloroform was added thereto, and the reaction solution was transferred to a separatory funnel for extraction. The extract was dried over MgSO₄ and concentrated, and the sample was purified by silica gel column chromatography to prepare 6.4 g of Compound 5D. (Yield: 63%, [M+H]⁺=696)

### Synthesis Example 29: Preparation of Compound 6D

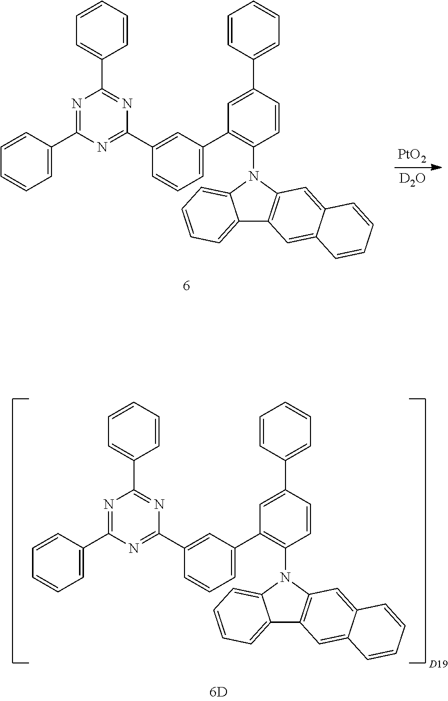

Compound 6 (10 g, 14.8 mmol), PtO₂ (0.98 g, 4.5 mmol), and 74 mL of D₂O were added to a shaker tube, and the shaker tube was sealed and heated at 250° C. and 600 psi for 12 hours. When the reaction was complete, chloroform was added thereto, and the reaction solution was transferred to a separatory funnel for extraction. The extract was dried over MgSO₄ and concentrated, and the sample was purified by silica gel column chromatography to produce 7.6 g of Compound 6D. (Yield: 75%, [M+H]⁺=696)

### Synthesis Example 30: Preparation of Compound 7D

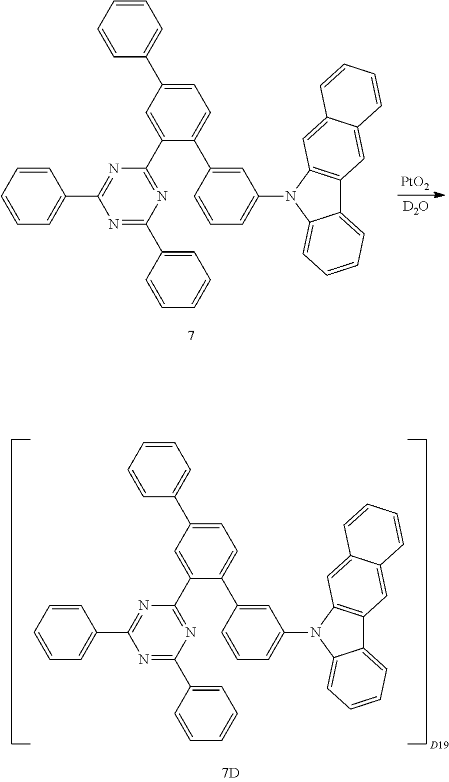

Compound 7 (10 g, 14.8 mmol), PtO₂ (0.98 g, 4.5 mmol), and 74 mL of D₂O were added to a shaker tube, and the shaker tube was sealed and heated at 250° C. and 600 psi for 12 hours. When the reaction was complete, chloroform was added thereto, and the reaction solution was transferred to a separatory funnel for extraction. The extract was dried over MgSO₄ and concentrated, and the sample was purified by silica gel column chromatography to produce 5.6 g of Compound 7D. (Yield: 55%, [M+H]⁺=696)

### Synthesis Example 31: Preparation of Compound 8D

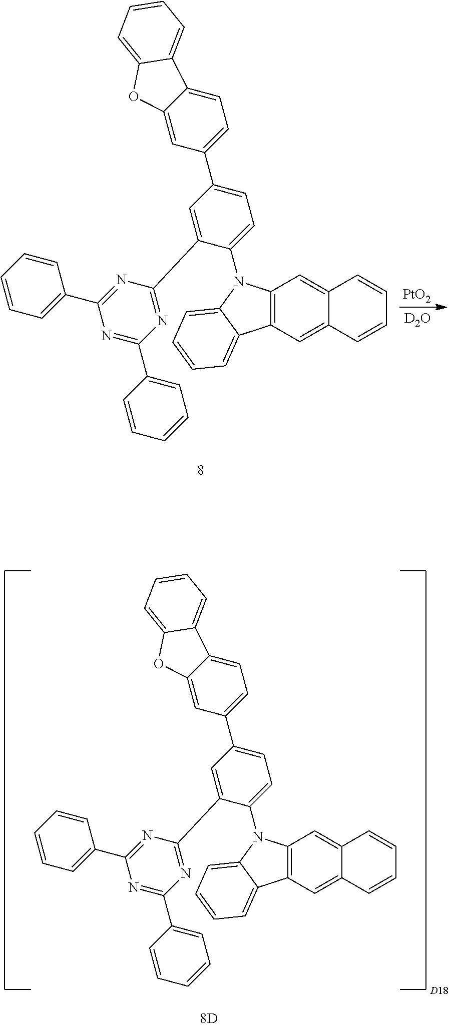

Compound 8 (10 g, 14.4 mmol), PtO₂ (0.95 g, 4.35 mmol), and 72 mL of D₂O were added to a shaker tube, and the shaker tube was sealed and heated at 250° C. and 600 psi for 12 hours. When the reaction was complete, chloroform was added thereto, and the reaction solution was transferred to a separatory funnel for extraction. The extract was dried over MgSO₄ and concentrated, and the sample was purified by silica gel column chromatography to produce 6.0 g of Compound 8D. (Yield: 59%, [M+H]⁺=709)

### Synthesis Example 32: Preparation of Compound 9D

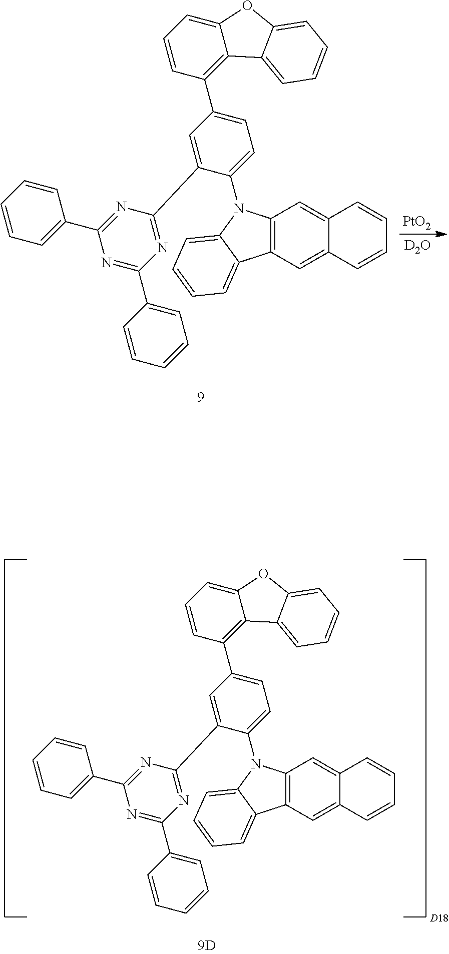

Compound 9 (10 g, 14.4 mmol), PtO₂ (0.95 g, 4.35 mmol), and 72 mL of D₂O were added to a shaker tube, and the shaker tube was sealed and heated at 250° C. and 600 psi for 12 hours. When the reaction was complete, chloroform was added thereto, and the reaction solution was transferred to a separatory funnel for extraction. The extract was dried over MgSO₄ and concentrated, and the sample was purified by silica gel column chromatography to produce 8.8 g of Compound 9D. (Yield: 87%, [M+H]⁺=709)

### Synthesis Example 33: Preparation of Compound 10D

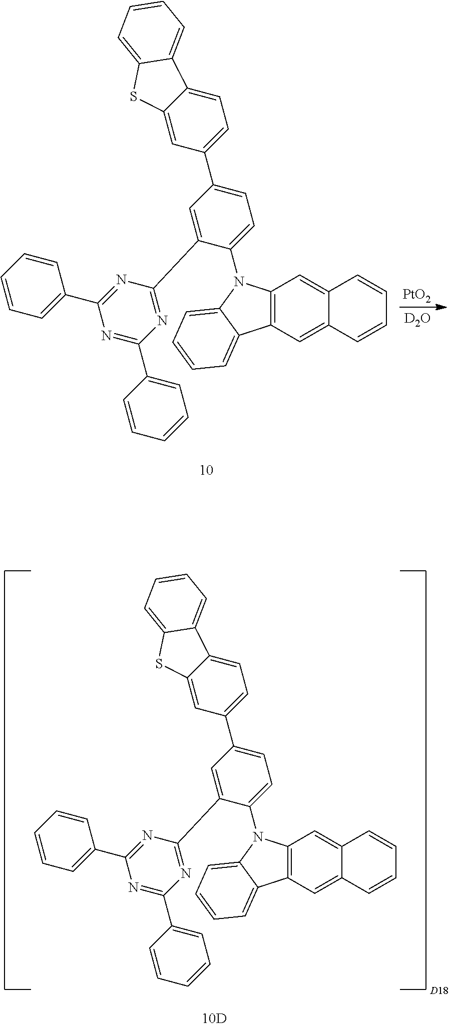

Compound 10 (10 g, 14.1 mmol), PtO₂ (0.93 g, 4.25 mmol), and 70 mL of D₂O were added to a shaker tube, and the shaker tube was sealed and heated at 250° C. and 600 psi for 12 hours. When the reaction was complete, chloroform was added thereto, and the reaction solution was transferred to a separatory funnel for extraction. The extract was dried over MgSO₄ and concentrated, and the sample was purified by silica gel column chromatography to produce 9.3 g of Compound 10D. (Yield: 92%, [M+H]⁺=725)

### Synthesis Example 34: Preparation of Compound 11D

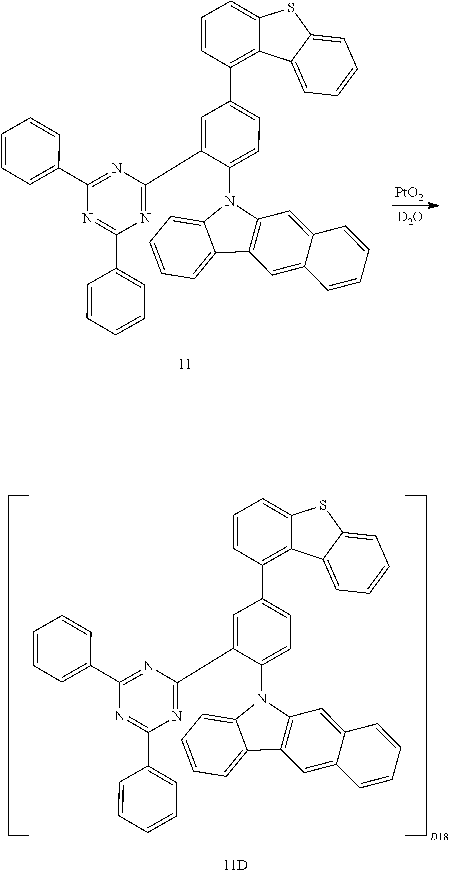

Compound 11 (10 g, 14.1 mmol), PtO₂ (0.93 g, 4.25 mmol), and 70 mL of D₂O were added to a shaker tube, and the shaker tube was sealed and heated at 250° C. and 600 psi for 12 hours. When the reaction was complete, chloroform was added thereto, and the reaction solution was transferred to a separatory funnel for extraction. The extract was dried over MgSO₄ and concentrated, and the sample was purified by silica gel column chromatography to produce 8.1 g of Compound 11 D. (Yield: 80%, [M+H]⁺=725)

### Example 1

A glass substrate on which ITO (indium tin oxide) was coated as a thin film to a thickness of 1,000 Å was put into distilled water in which a detergent was dissolved, and ultrasonically cleaned. A product manufactured by Fischer Co. was used as the detergent, and as the distilled water, distilled water filtered twice using a filter manufactured by Millipore Co. was used. After the ITO was cleaned for 30 minutes, ultrasonic cleaning was repeated twice using distilled water for 10 minutes. After the cleaning with distilled water was completed, the substrate was ultrasonically cleaned with solvents of isopropyl alcohol, acetone, and methanol, dried, and then transferred to a plasma cleaner. In addition, the substrate was cleaned for 5 minutes using oxygen plasma and then transferred to a vacuum depositor.

On the ITO transparent electrode thus prepared, the following compound HI-1 was formed to a thickness of 1150 Å as a hole injection layer, but the following compound A-1 was p-doped at a concentration of 1.5 wt %. The following compound HT-1 was vacuum deposited on the hole injection layer to form a hole transport layer with a layer thickness of 800 Å. Then, the following compound EB-1 was vacuum deposited on the hole transport layer to a layer thickness of 150 Å to form an electron blocking layer.

Then, a mixture of Compound 1 prepared in Synthesis Example 1 and Compound pRH (mixed in a weight ratio of Compound 1: pRH=50:50) and Dp-7 compound were vacuum deposited in a weight ratio of 98.5:1.5 on the EB-1 deposition film to form a red light emitting layer having a thickness of 400 Å.

The following Compound HB-1 was vacuum deposited on the light emitting layer to a layer thickness of 50 Å to form a hole blocking layer. Then, the following Compound ET-1 and the following Compound LiQ were vacuum deposited at a weight ratio of 2:1 on the hole blocking layer to form an electron injection and transport layer with a film thickness of 250 Å.

Lithium fluoride (LiF) and aluminum were sequentially deposited to have a thickness of 12 Å and 1,000 Å, respectively, on the electron injection and transport layer, thereby forming a cathode.

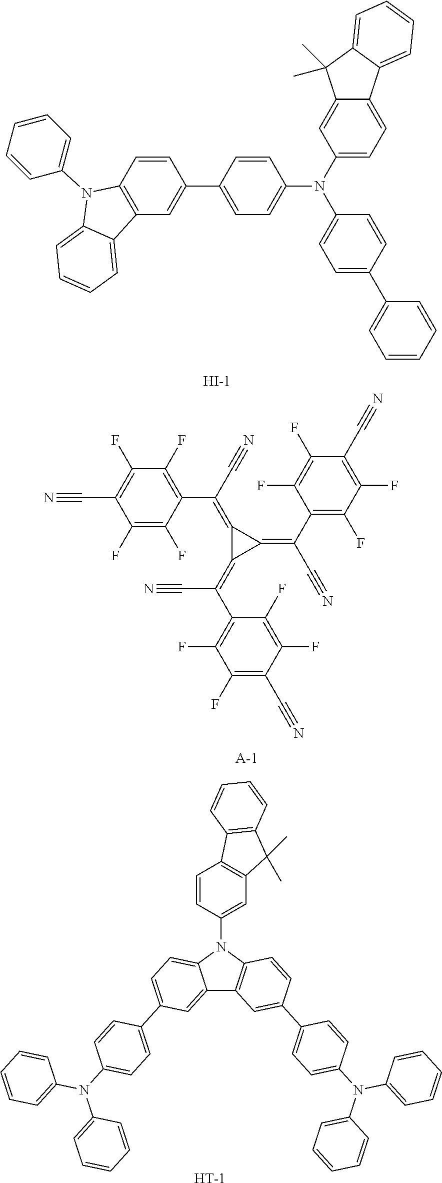

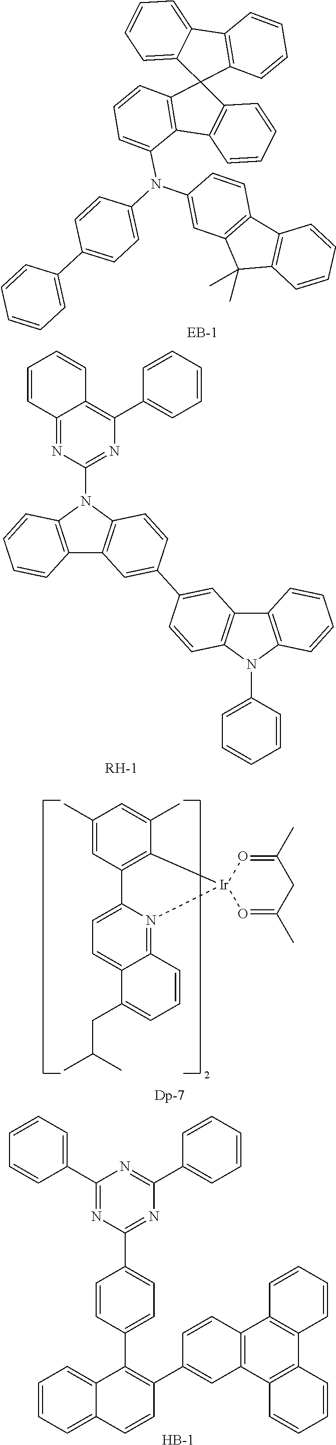

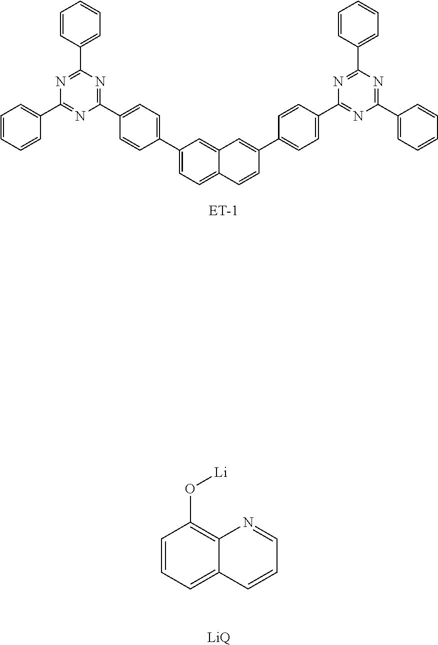

In the above-mentioned processes, the vapor deposition rate of the organic material was maintained at 0.4˜0.7 Å/sec, the deposition rates of lithium fluoride and aluminum of the cathode were maintained at 0.3 Å/sec and 2 Å/sec, respectively, and the degree of vacuum during the deposition was maintained at 2×10⁻⁷˜5×10⁻⁶ torr, thereby manufacturing an organic light emitting device.

### Examples 2 to 34

The organic light emitting device was manufactured in the same manner as in Example 1, except that the compounds listed in Table 1 below were used instead of Compound 1 as the second host compound of the light emitting layer. Herein, the structure of the compound used in the Examples is summarized as follows.

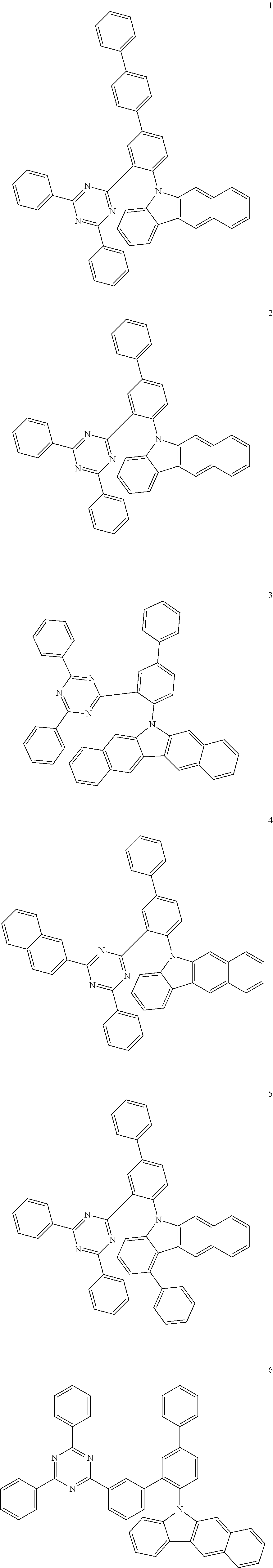

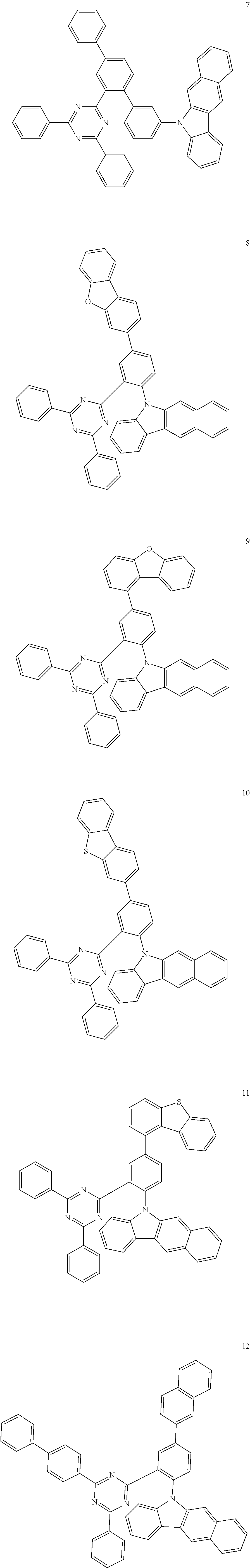

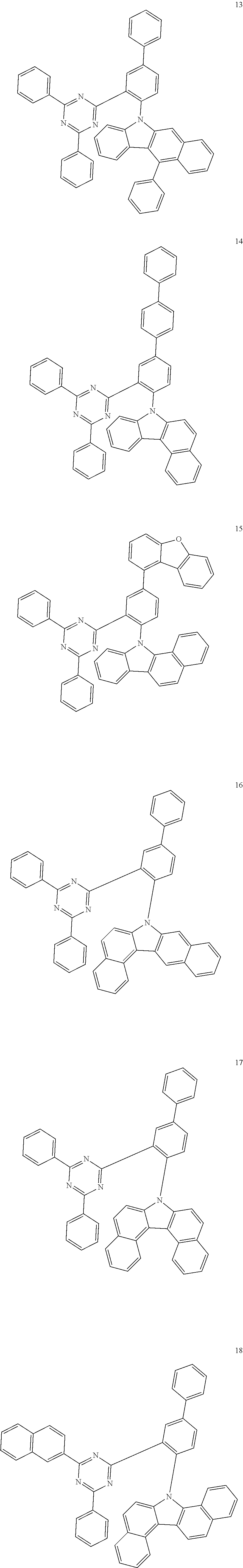

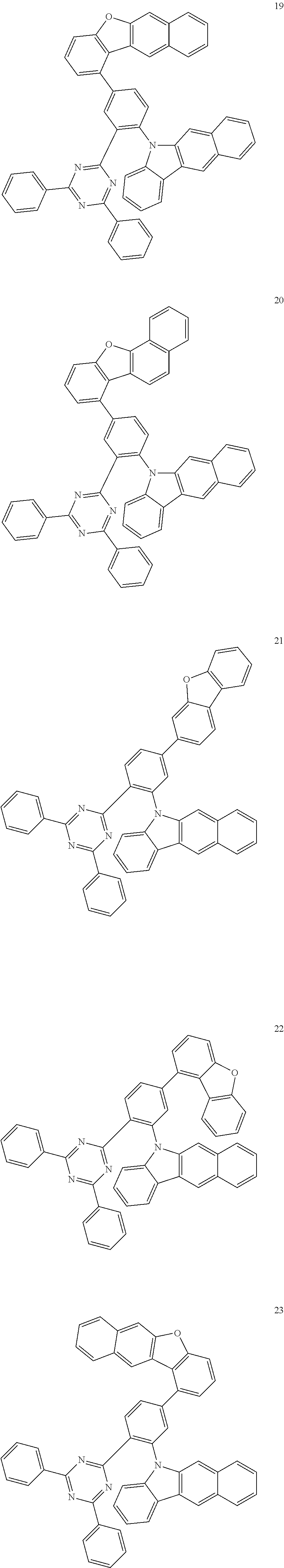 [SMILES: c1ccc(-c2nc(-c3ccccc3)nc(-c3cc(-c4cccc5oc6cc7ccccc7cc6c45)ccc3-n3c4ccccc4c4cc5ccccc5cc43)n2)cc1 ; c1ccc(-c2nc(-c3ccccc3)nc(-c3cc(-c4cccc5oc6c7ccccc7ccc6c45)ccc3-n3c4ccccc4c4cc5ccccc5cc43)n2)cc1 ; c1ccc(-c2nc(-c3ccccc3)nc(-c3ccc(-c4ccc5c(c4)oc4ccccc45)cc3-n3c4ccccc4c4cc5ccccc5cc43)n2)cc1 ; c1ccc(-c2nc(-c3ccccc3)nc(-c3ccc(-c4cccc5oc6ccccc6c45)cc3-n3c4ccccc4c4cc5ccccc5cc43)n2)cc1 ; c1ccc(-c2nc(-c3ccccc3)nc(-c3ccc(-c4cccc5oc6cc7ccccc7cc6c45)cc3-n3c4ccccc4c4cc5ccccc5cc43)n2)cc1]

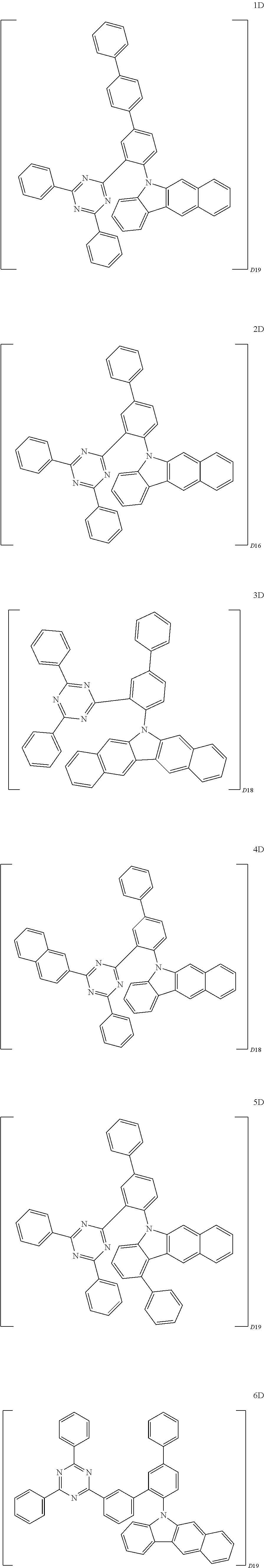

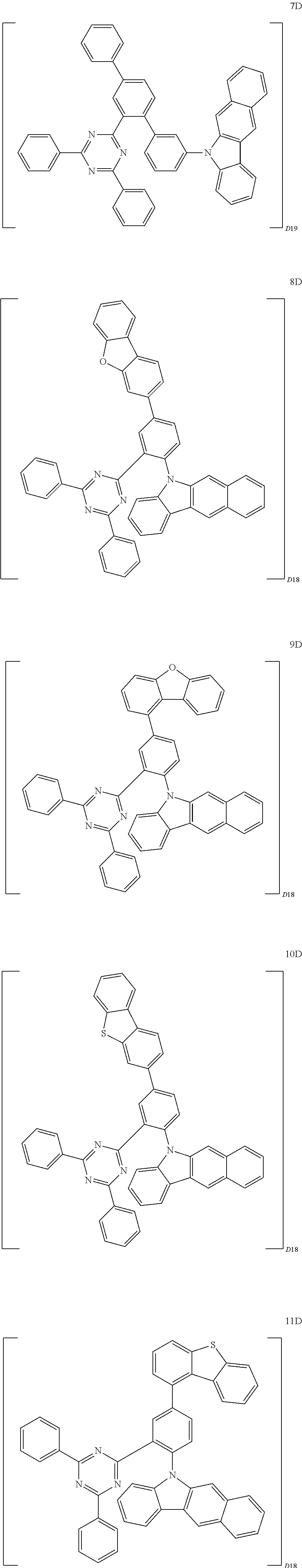

### Comparative Examples 1 to 11

The organic light emitting device was manufactured in the same manner as in Example 1, except that the compounds listed in Table 1 below were used instead of Compound 1 as the second host compound of the light emitting layer. Herein, the structure of the compound used in the Comparative Examples is summarized as follows.

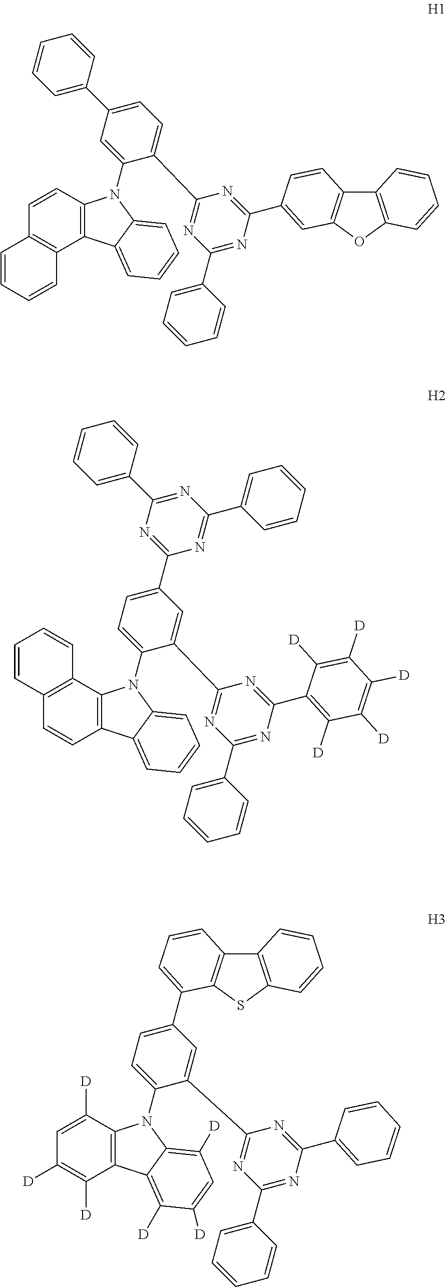 [SMILES: c1ccc(-c2ccc(-c3nc(-c4ccccc4)nc(-c4ccc5c(c4)oc4ccccc45)n3)c(-n3c4ccccc4c4c5ccccc5ccc43)c2)cc1 ; [2H]c1c([2H])c([2H])c(-c2nc(-c3ccccc3)nc(-c3cc(-c4nc(-c5ccccc5)nc(-c5ccccc5)n4)ccc3-n3c4ccccc4c4ccc5ccccc5c43)n2)c([2H])c1[2H] ; [2H]c1cc([2H])c2c(c1[2H])c1c([2H])c([2H])cc([2H])c1n2-c1ccc(-c2cccc3c2sc2ccccc23)cc1-c1nc(-c2ccccc2)nc(-c2ccccc2)n1]

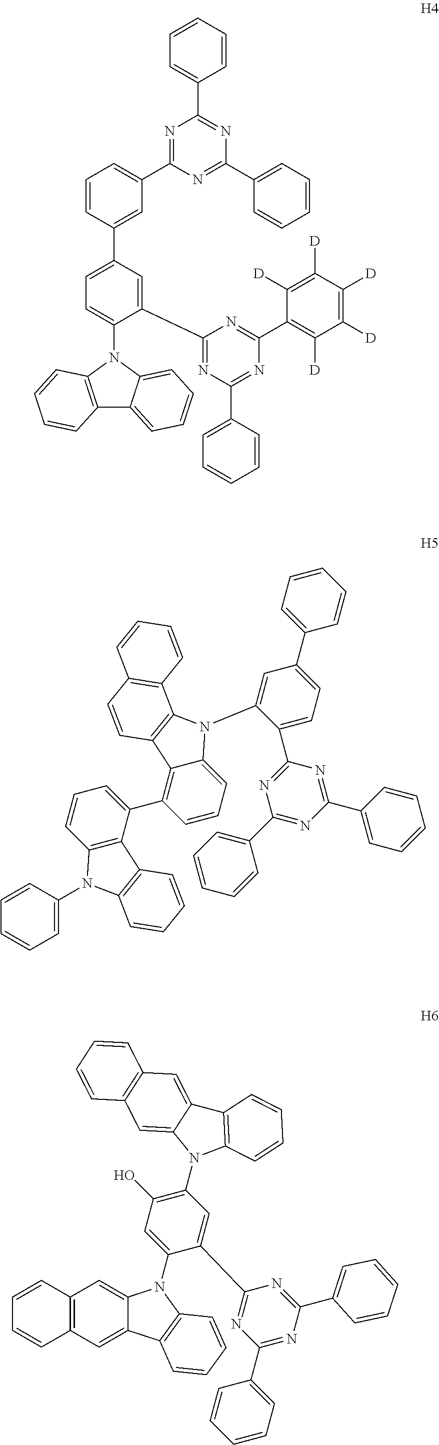 [SMILES: [2H]c1c([2H])c([2H])c(-c2nc(-c3ccccc3)nc(-c3cc(-c4cccc(-c5nc(-c6ccccc6)nc(-c6ccccc6)n5)c4)ccc3-n3c4ccccc4c4ccccc43)n2)c([2H])c1[2H] ; c1ccc(-c2ccc(-c3nc(-c4ccccc4)nc(-c4ccccc4)n3)c(-n3c4cccc(-c5cccc6c5c5ccccc5n6-c5ccccc5)c4c4ccc5ccccc5c43)c2)cc1 ; Oc1cc(-n2c3ccccc3c3cc4ccccc4cc32)c(-c2nc(-c3ccccc3)nc(-c3ccccc3)n2)cc1-n1c2ccccc2c2cc3ccccc3cc21]

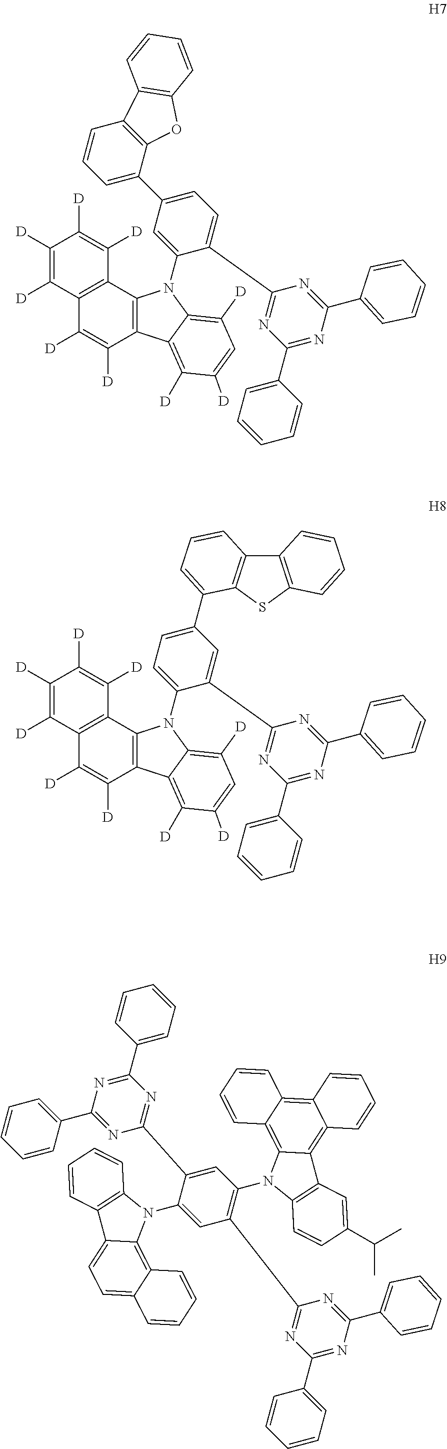 [SMILES: [2H]c1cc([2H])c2c(c1[2H])c1c([2H])c([2H])c3c([2H])c([2H])c([2H])c([2H])c3c1n2-c1cc(-c2cccc3c2oc2ccccc23)ccc1-c1nc(-c2ccccc2)nc(-c2ccccc2)n1 ; [2H]c1cc([2H])c2c(c1[2H])c1c([2H])c([2H])c3c([2H])c([2H])c([2H])c([2H])c3c1n2-c1ccc(-c2cccc3c2sc2ccccc23)cc1-c1nc(-c2ccccc2)nc(-c2ccccc2)n1 ; CC(C)c1ccc2c(c1)c1c3ccccc3c3ccccc3c1n2-c1cc(-c2nc(-c3ccccc3)nc(-c3ccccc3)n2)c(-n2c3ccccc3c3ccc4ccccc4c32)cc1-c1nc(-c2ccccc2)nc(-c2ccccc2)n1]

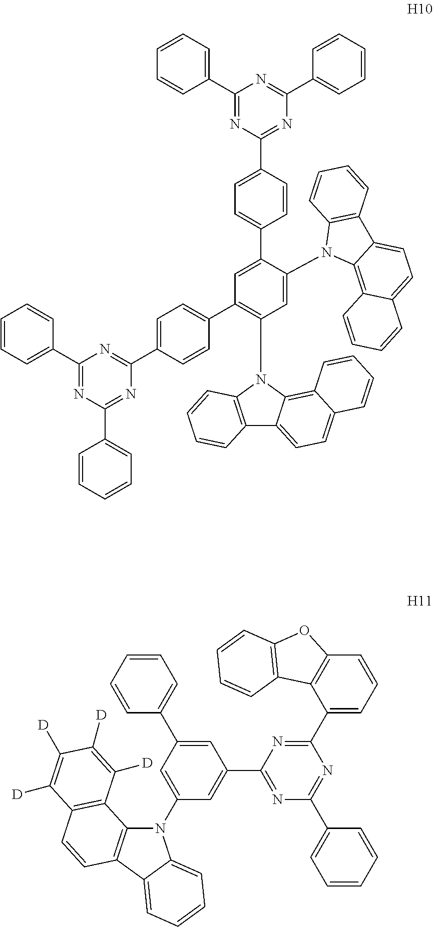 [SMILES: c1ccc(-c2nc(-c3ccccc3)nc(-c3ccc(-c4cc(-c5ccc(-c6nc(-c7ccccc7)nc(-c7ccccc7)n6)cc5)c(-n5c6ccccc6c6ccc7ccccc7c65)cc4-n4c5ccccc5c5ccc6ccccc6c54)cc3)n2)cc1 ; [2H]c1c([2H])c([2H])c2c(ccc3c4ccccc4n(-c4cc(-c5ccccc5)cc(-c5nc(-c6ccccc6)nc(-c6cccc7oc8ccccc8c67)n5)c4)c32)c1[2H]]

### EXPERIMENTAL EXAMPLE

### Experimental Example 1

The voltage, efficiency and lifetime were measured by applying a current to the organic light emitting devices manufactured in the Examples 1 to 34 and Comparative Examples 1 to 11, and the results are shown in Table 1 below. In this case, the voltage and efficiency were measured by applying a current density of 10 mA/cm², and LT95 means the time (hr) until the initial brightness (4000 nit) decreases to 95% at a current density of 50 mA/cm².

TABLE 1

| | | @ 10 mA/cm² | | @ |
|---|---|---|---|---|
| | Host compound | Driving | Efficiency | 50 mA/cm² |
| | in the light emitting layer | voltage (V) | (cd/A) | LT95(hr) |
| Comparative | H1:pRH (50:50) | 3.92 | 17.2 | 120 |
| Example 1 | | | | |
| Comparative | H2:pRH (50:50) | 4.05 | 16.5 | 142 |
| Example 2 | | | | |
| Comparative | H3:pRH (50:50) | 3.52 | 21.2 | 155 |
| Example 3 | | | | |
| Comparative | H4:pRH (50:50) | 3.35 | 18.9 | 132 |
| Example 4 | | | | |
| Comparative | H5:pRH (50:50) | 3.89 | 12.5 | 190 |
| Example 5 | | | | |
| Comparative | H6:pRH (50:50) | 3.42 | 14.2 | 35 |
| Example 6 | | | | |
| Comparative | H7:pRH (50:50) | 3.79 | 22.9 | 181 |
| Example 7 | | | | |
| Comparative | H8:pRH (50:50) | 3.88 | 25.5 | 65 |
| Example 8 | | | | |
| Comparative | H9:pRH (50:50) | 3.98 | 23.1 | 81 |
| Example 9 | | | | |
| Comparative | H10:pRH (50:50) | 3.31 | 21.3 | 129 |
| Example 10 | | | | |
| Comparative | H11:pRH (50:50) | 3.45 | 24.9 | 181 |
| Example 11 | | | | |
| Example 1 | Compound 1:pRH (50:50) | 3.20 | 38.5 | 271 |
| Example 2 | Compound 2:pRH (50:50) | 3.25 | 36.5 | 220 |
| Example 3 | Compound 3:pRH (50:50) | 3.17 | 38.1 | 265 |
| Example 4 | Compound 4:pRH (50:50) | 3.22 | 37.2 | 231 |
| Example 5 | Compound 5:pRH (50:50) | 3.32 | 35.6 | 235 |
| Example 6 | Compound 6:pRH (50:50) | 3.35 | 36.1 | 255 |
| Example 7 | Compound 7:pRH (50:50) | 3.28 | 37.9 | 262 |
| Example 8 | Compound 8:pRH (50:50) | 3.21 | 36.2 | 202 |
| Example 9 | Compound 9:pRH (50:50) | 3.15 | 37.2 | 205 |
| Example 10 | Compound 10:pRH (50:50) | 3.27 | 34.5 | 195 |
| Example 11 | Compound 11:pRH (50:50) | 3.23 | 35.7 | 186 |
| Example 12 | Compound 12:pRH (50:50) | 3.27 | 37.8 | 253 |
| Example 13 | Compound 13:pRH (50:50) | 3.35 | 38.8 | 248 |
| Example 14 | Compound 14:pRH (50:50) | 3.37 | 36.9 | 275 |
| Example 15 | Compound 15:pRH (50:50) | 3.33 | 35.9 | 206 |
| Example 16 | Compound 16:pRH (50:50) | 3.16 | 38.9 | 244 |
| Example 17 | Compound 17:pRH (50:50) | 3.38 | 36.8 | 266 |
| Example 18 | Compound 18:pRH (50:50) | 3.32 | 37.1 | 274 |
| Example 19 | Compound 19:pRH (50:50) | 3.22 | 34.2 | 185 |
| Example 20 | Compound 20:pRH (50:50) | 3.29 | 35.1 | 195 |
| Example 21 | Compound 21:pRH (50:50) | 3.38 | 34.8 | 191 |
| Example 22 | Compound 22:pRH (50:50) | 3.35 | 36.0 | 197 |
| Example 23 | Compound 23:pRH (50:50) | 3.37 | 35.1 | 178 |
| Example 24 | Compound 1D:pRH (50:50) | 3.21 | 38.2 | 332 |
| Example 25 | Compound 2D:pRH (50:50) | 3.27 | 36.3 | 272 |
| Example 26 | Compound 3D:pRH (50:50) | 3.15 | 38.0 | 302 |
| Example 27 | Compound 4D:pRH (50:50) | 3.24 | 37.2 | 289 |
| Example 28 | Compound 5D:pRH (50:50) | 3.33 | 35.5 | 291 |
| Example 29 | Compound 6D:pRH (50:50) | 3.34 | 36.4 | 302 |
| Example 30 | Compound 7D:pRH (50:50) | 3.28 | 38.5 | 312 |
| Example 31 | Compound 8D:pRH (50:50) | 3.22 | 42.3 | 223 |
| Example 32 | Compound 9D:pRH (50:50) | 3.16 | 45.1 | 262 |
| Example 33 | Compound 10D:pRH (50:50) | 3.25 | 39.5 | 252 |
| Example 34 | Compound 11D:pRH (50:50) | 3.24 | 40.0 | 267 |

As shown in Table 1, the organic light emitting device of Example, which used the compound represented by Chemical Formula 1 as the host material in the light emitting layer, exhibited superior driving voltage, luminous efficiency, and lifetime characteristics compared to the organic light emitting device of Comparative Example, which used the compound with a different structure than Chemical Formula 1.

This is believed to be because, when the compound represented by Chemical Formula 1 was used as one of the host materials, energy transfer from the red light emitting layer to the red dopant occurred more effectively, and the co-host combination of Example maintained a more stable balance within the emission layer than the co-host combination of Comparative Example.

Further, considering that the luminous efficiency and lifetime characteristics of organic light emitting devices generally have a trade-off relationship, the organic light emitting device employing the compound combination of the present disclosure exhibits significantly improved device characteristics compared to the device of the Comparative Example.

[Description of Symbols]

| | |
|---|---|
| 1: substrate | 2: anode |
| 3: hole transport layer | 4: light emitting layer |
| 5: electron injection and transport layer | 6: cathode |
| 7: hole injection layer | 8: electron blocking layer |
| 9: hole blocking layer | |

1. A compound represented by the following Chemical Formula 1: 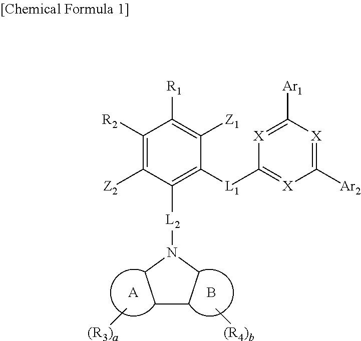
wherein in Chemical Formula 1,
A is a naphthalene ring fused with a neighboring pentagonal ring,
B is a C₆₋₂₀ aromatic ring fused with a neighboring pentagonal ring,
X are each independently N or CR, provided that at least one of X is N,
R is hydrogen, deuterium, or tritium,
R₁ is hydrogen, deuterium, tritium, substituted or unsubstituted C₆₋₆₀ aryl, or substituted or unsubstituted C₂₋₆₀ heteroaryl comprising O or S, but is not 4-dibenzofuranyl and 4-dibenzothiophenyl,
R₂ is hydrogen, deuterium, tritium, or substituted or unsubstituted C₂₋₆₀ heteroaryl comprising O or S, but is not 4-dibenzofuranyl and 4-dibenzothiophenyl,
provided that the case where R₁ and R₂ are simultaneously hydrogen, deuterium, or tritium is excluded,
Z₁ and Z₂ are each independently hydrogen, deuterium, or tritium,
L₁ and L₂ are each independently a single bond, substituted or unsubstituted C₁₋₆₀ alkylene, substituted or unsubstituted C₆₋₆₀ arylene, or substituted or unsubstituted C₂₋₆₀ heteroarylene comprising one or more heteroatoms among N, O and S,
Ar₁ and Ar₂ are each independently substituted or unsubstituted C₁₋₆₀ alkyl, substituted or unsubstituted C₆₋₆₀ aryl, or substituted or unsubstituted C₂₋₆₀ heteroaryl comprising one or more heteroatoms among N, O and S,
R₃ and R₄ are each independently hydrogen, deuterium, tritium, substituted or unsubstituted C₁₋₆₀ alkyl, or substituted or unsubstituted C₆₋₆₀ aryl,
a is an integer from 0 to 6,
b is an integer from 0 to 10, and
the “substituted or unsubstituted” means being unsubstituted or substituted with one or more substituents selected from the group consisting of deuterium, tritium, halogen, nitro, amino, alkyl, and aryl, or being unsubstituted or substituted with a substituent to which two or more substituents of the above-exemplified substituents are connected.

2. The compound of claim 1, wherein:
X is all N.

3. The compound of claim 1, wherein:
B is a benzene, naphthalene, phenanthrene, or anthracene ring, fused with the neighboring pentagonal ring.

4. The compound of claim 1, wherein:
the substituent 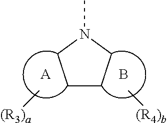
is any one of the substituents represented by the following chemical formulas 1a to 1l: 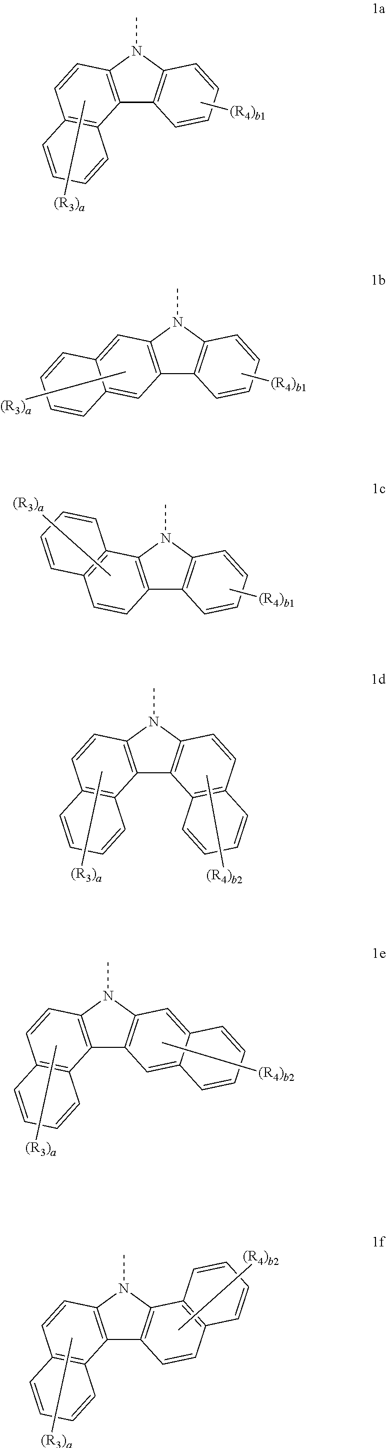 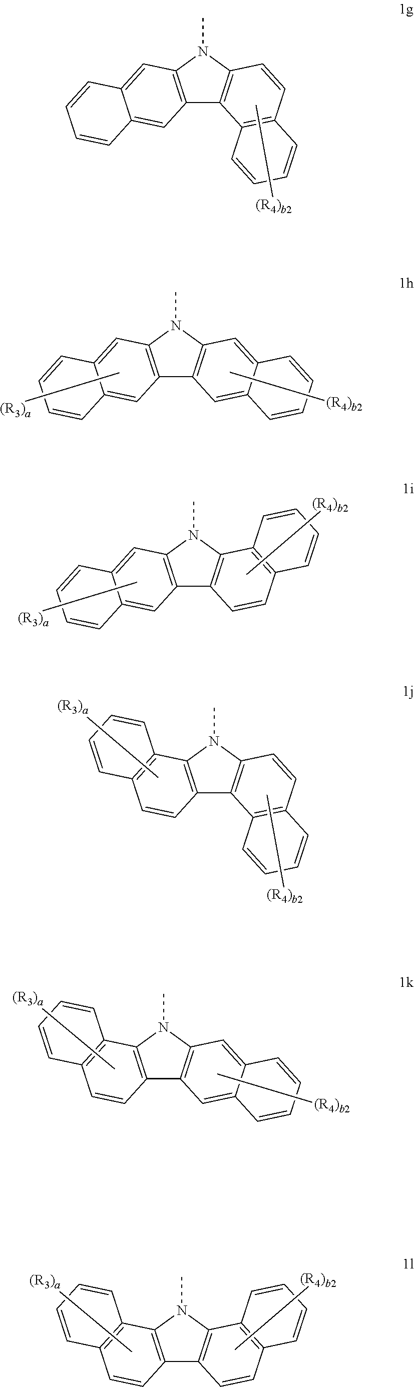
wherein in Chemical Formulas 1a to 1l,
b1 is an integer from 0 to 4,
b2 is an integer from 0 to 6, and
R₃, R₄ and a are as defined in claim 1.

5. The compound of claim 1, wherein:
R₁ is hydrogen, deuterium, tritium, substituted or unsubstituted phenyl, substituted or unsubstituted biphenylyl, substituted or unsubstituted terphenylyl, substituted or unsubstituted quaterphenylyl, substituted or unsubstituted naphthyl, substituted or unsubstituted phenanthryl, substituted or unsubstituted 1-dibenzofuranyl, substituted or unsubstituted 2-dibenzofuranyl, substituted or unsubstituted 3-dibenzofuranyl, substituted or unsubstituted 1-dibenzothiophenyl, substituted or unsubstituted 2-dibenzothiophenyl, substituted or unsubstituted 3-dibenzothiophenyl, substituted or unsubstituted benzonaphthofuranyl, or substituted or unsubstituted benzonaphthothiophenyl, and
where the “substituted or unsubstituted” means being unsubstituted or substituted with one or more substituents selected from the group consisting of deuterium, tritium, C₁₋₁₀ alkyl and C₆₋₂₀ aryl, or being unsubstituted or substituted with a substituent to which two or more substituents of the above-exemplified substituents are connected.

6. The compound of claim 1, wherein:
R₁ is C₆₋₂₄ monocyclic aryl unsubstituted or substituted with deuterium.

7. The compound of claim 1, wherein:
R₂ is hydrogen, deuterium, tritium, substituted or unsubstituted 1-dibenzofuranyl, substituted or unsubstituted 2-dibenzofuranyl, substituted or unsubstituted 3-dibenzofuranyl, substituted or unsubstituted 1-dibenzothiophenyl, substituted or unsubstituted 2-dibenzothiophenyl, substituted or unsubstituted 3-dibenzothiophenyl, substituted or unsubstituted benzonaphthofuranyl, or substituted or unsubstituted benzonaphthothiophenyl, and
where the “substituted or unsubstituted” means being unsubstituted or substituted with one or more substituents selected from the group consisting of deuterium, tritium, C₁₋₁₀ alkyl and C₆₋₂₀ aryl, or being unsubstituted or substituted with a substituent to which two or more substituents of the above-exemplified substituents are connected.

8. The compound of claim 1, wherein:
Z₁ and Z₂ are both hydrogens, or both deuteriums.

9. The compound of claim 1, wherein:
L₁ and L₂ are each independently a single bond, phenylene unsubstituted or substituted with deuterium, biphenyldiyl unsubstituted or substituted with deuterium, or naphthylene unsubstituted or substituted with deuterium.

10. The compound of claim 1, wherein:
Ar₁ and Ar₂ are each independently phenyl, biphenylyl, terphenylyl, 1-naphthyl, or 2-naphthyl,
wherein, Ar₁ and Ar₂ are unsubstituted or substituted with one or more substituents selected from the group consisting of deuterium, tritium, C₁₋₁₀ alkyl and C₆₋₂₀ aryl, or being unsubstituted or substituted with a substituent to which two or more substituents of the above-exemplified substituents are connected.

11. The compound of claim 1, wherein:
R₃ and R₄ are each independently hydrogen, deuterium, tritium, or phenyl unsubstituted or substituted with deuterium.

12. The compound of claim 1, wherein:
a+b is 0; or
a+b is 1, and one of R₃ and R₄ is phenyl unsubstituted or substituted with deuterium; or
a+b is 1 to 12, and one of R₃ and R₄ is phenyl unsubstituted or substituted with deuterium, and the rest are hydrogen or deuterium.

13. The compound of claim 1, wherein:
the compound is represented by the following Chemical Formulas 1-1 or 1-2: 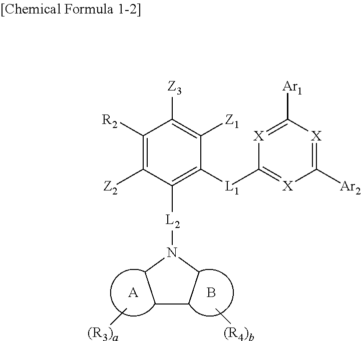
wherein in Chemical Formula 1-1,
R₁ is substituted or unsubstituted C₆₋₆₀ aryl, or substituted or unsubstituted C₂₋₆₀ heteroaryl comprising O or S, but is not 4-dibenzofuranyl and 4-dibenzothiophenyl,
Z₃ is hydrogen, deuterium, or tritium, and
A, B, X, Z₁, Z₂, L₁, L₂, Ar₁, Ar₂, R₃, R₄, a and b are as defined in claim 1, and 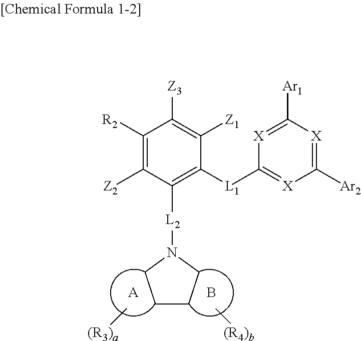
wherein in Chemical Formula 1-2,
R₂ is substituted or unsubstituted C₂₋₆₀ heteroaryl comprising O or S, but is not 4-dibenzofuranyl and 4-dibenzothiophenyl,
Z₃ is hydrogen, deuterium, or tritium, and
A, B, X, Z₁, Z₂, L₁, L₂, Ar₁, Ar₂, R₃, R₄, a and b are as defined in claim 1.

14. The compound of claim 1, wherein:
the compound is any one selected from the group consisting of the following compounds: 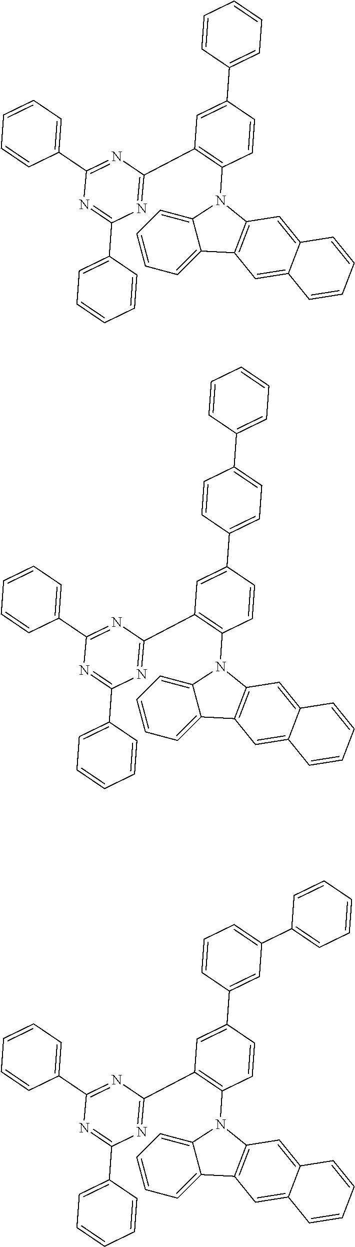 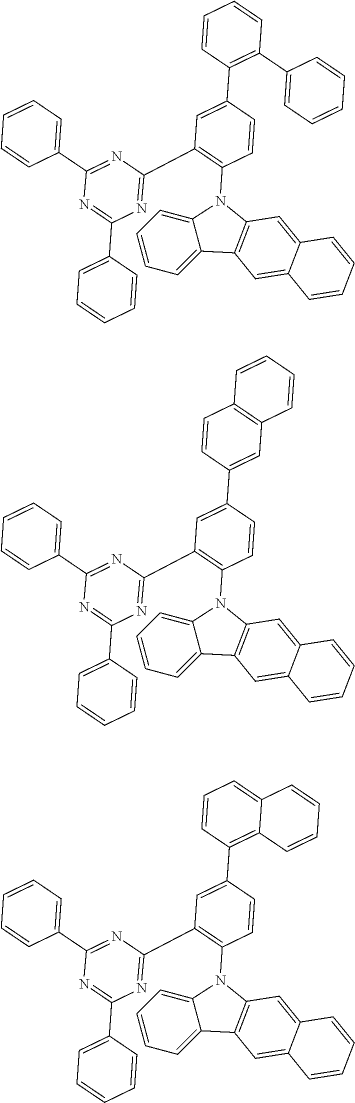 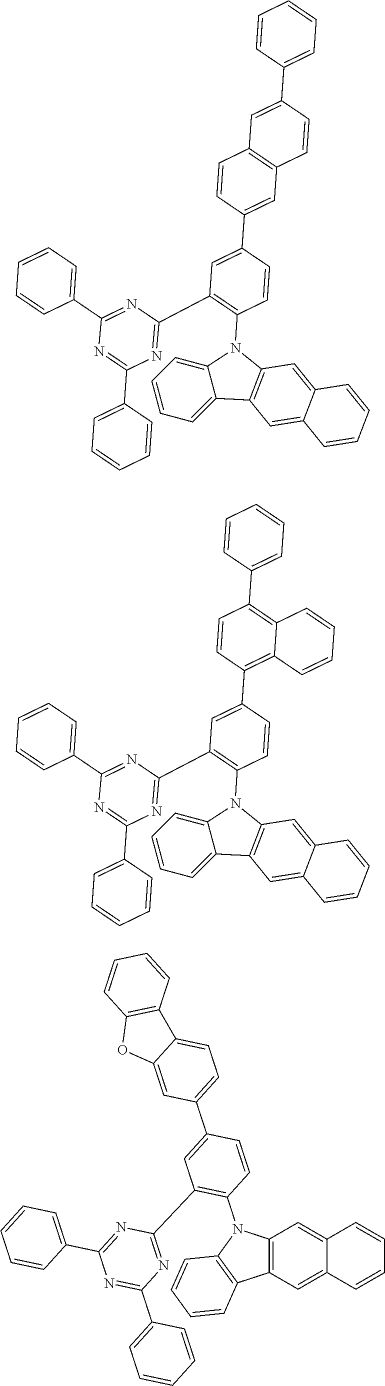 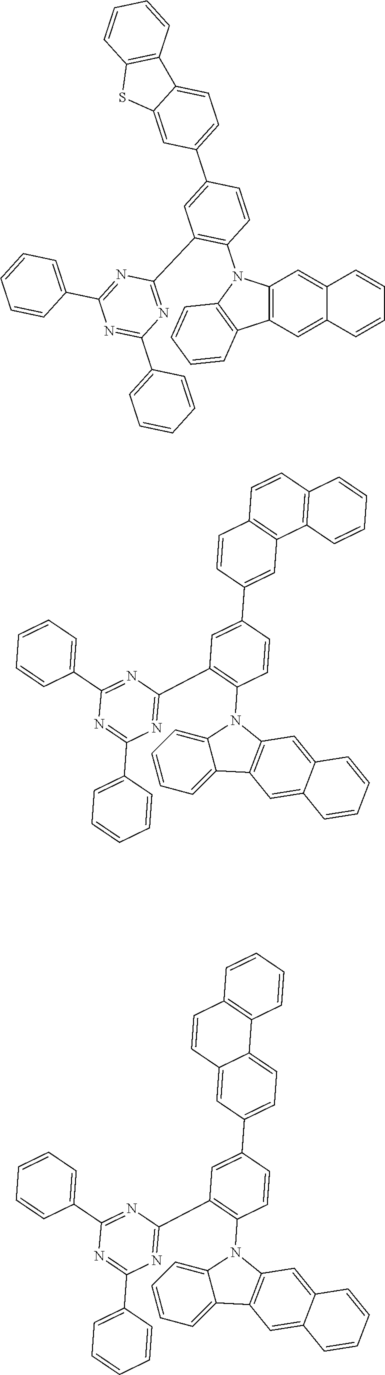 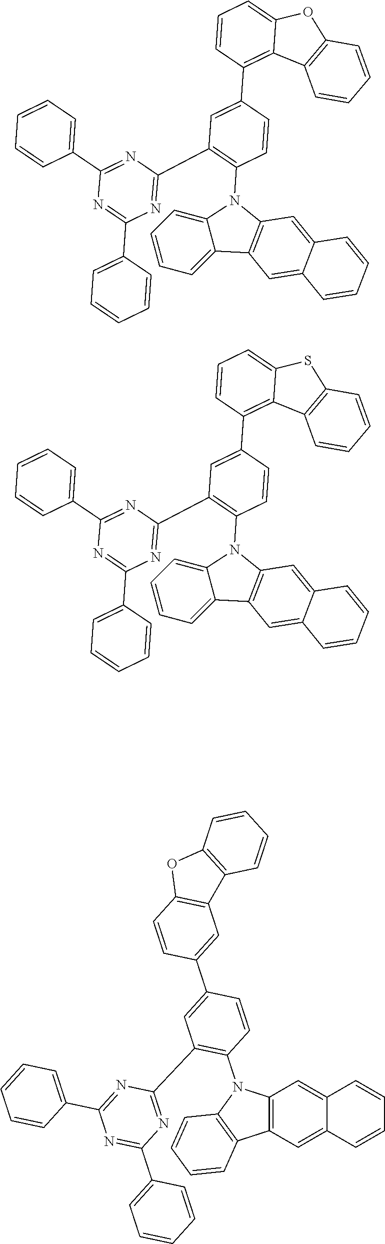 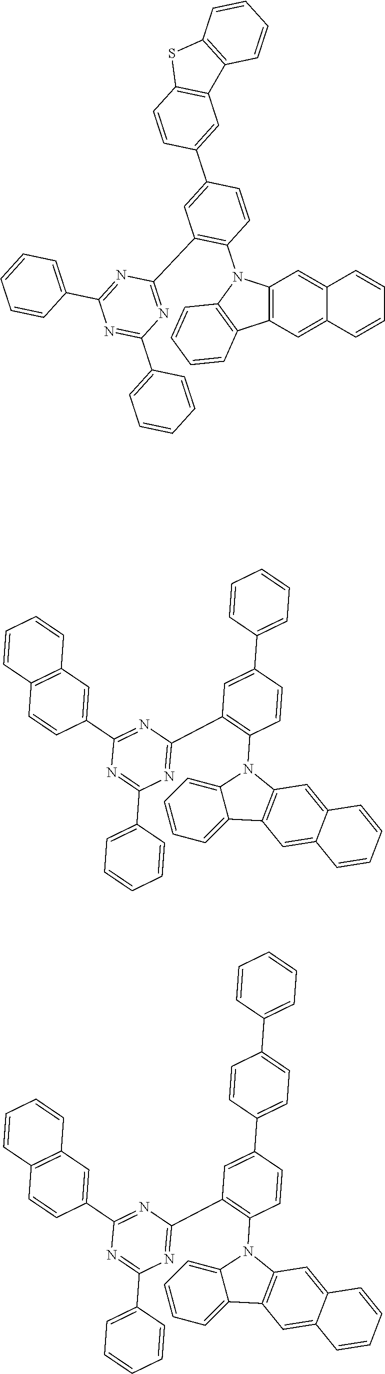 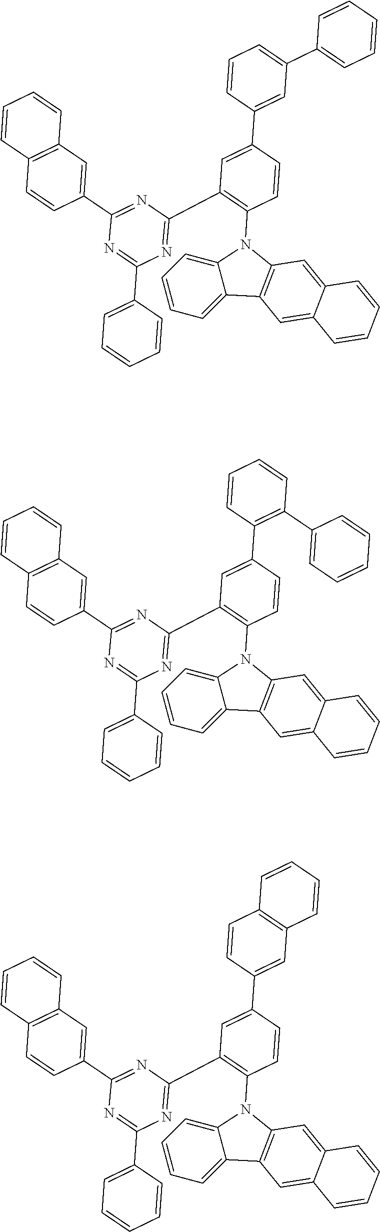 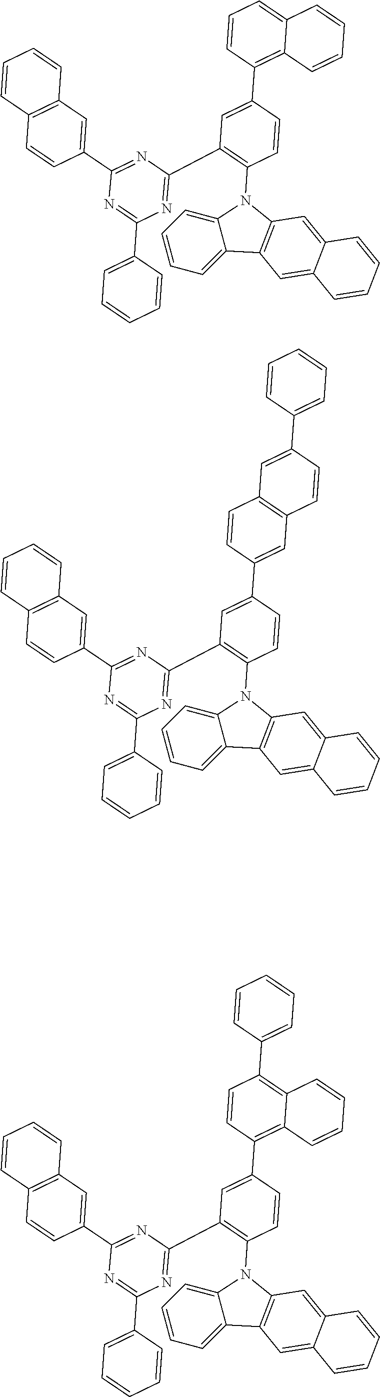 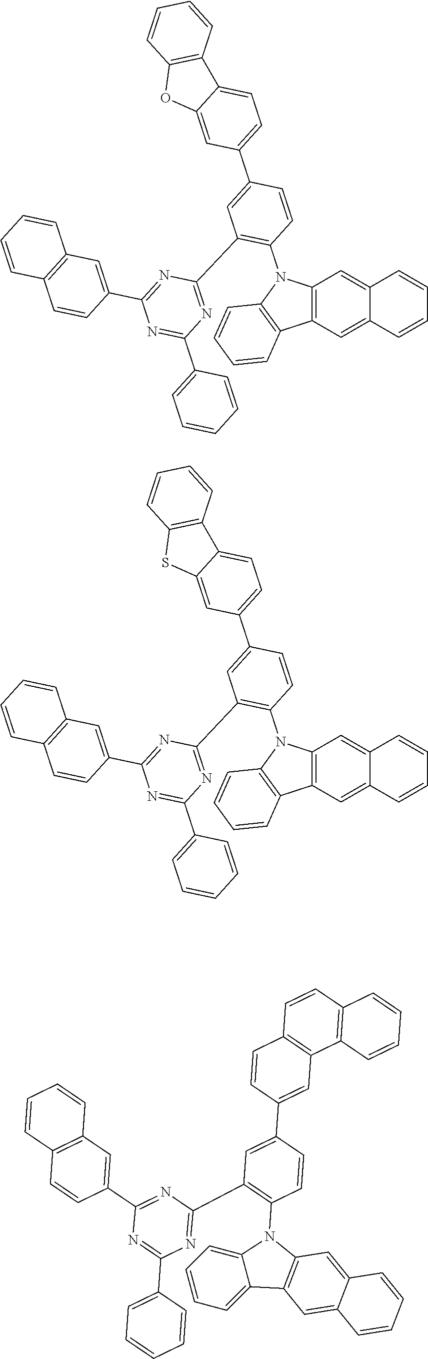 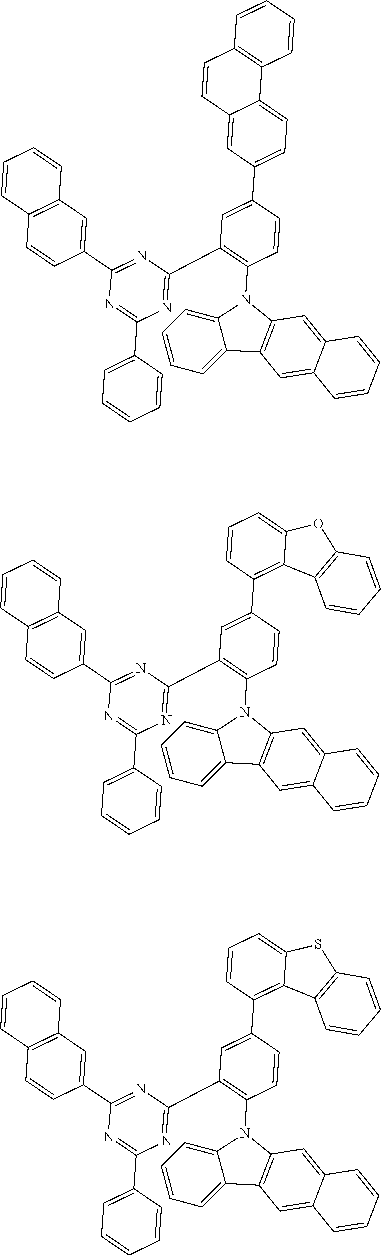 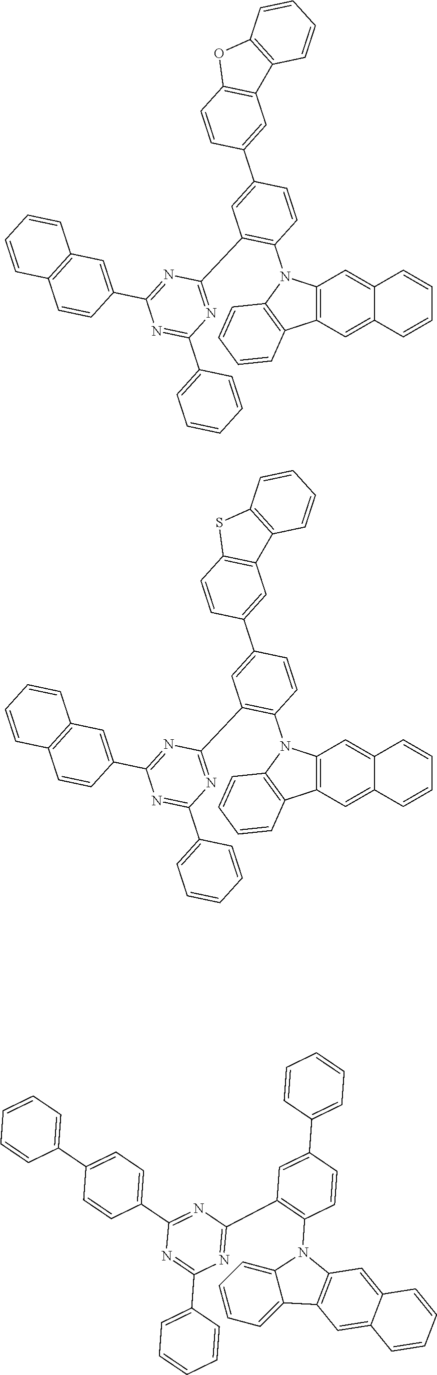 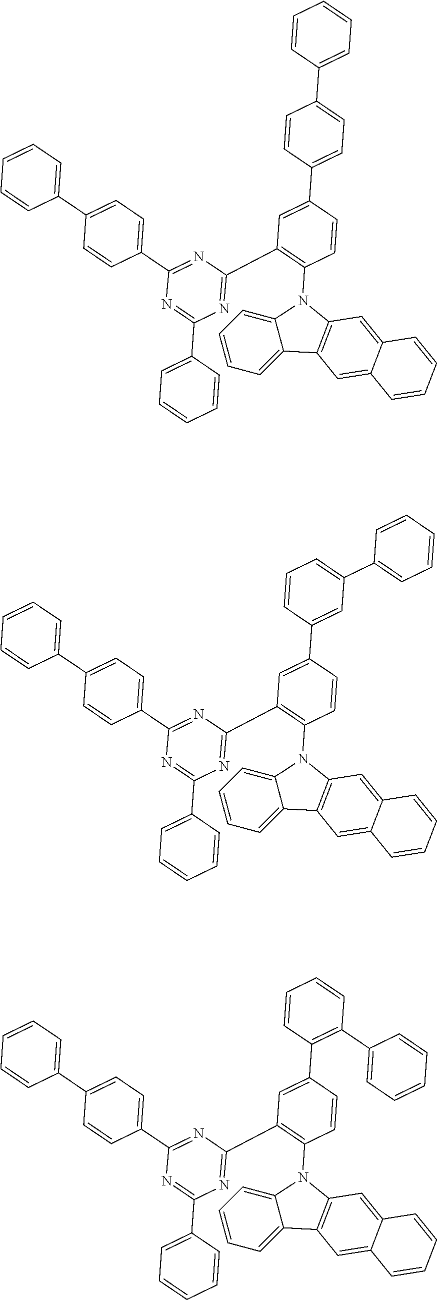 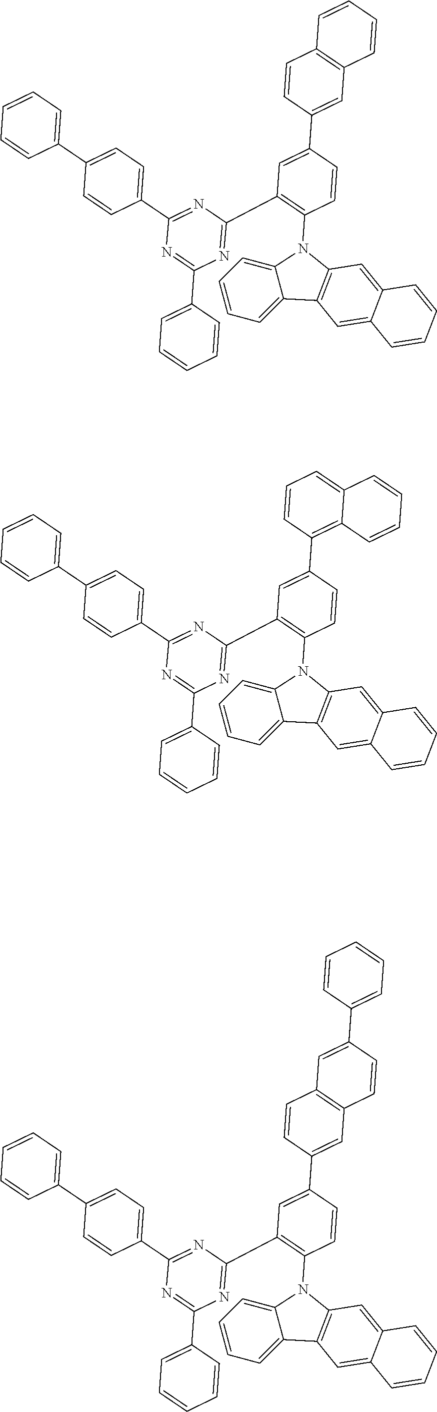 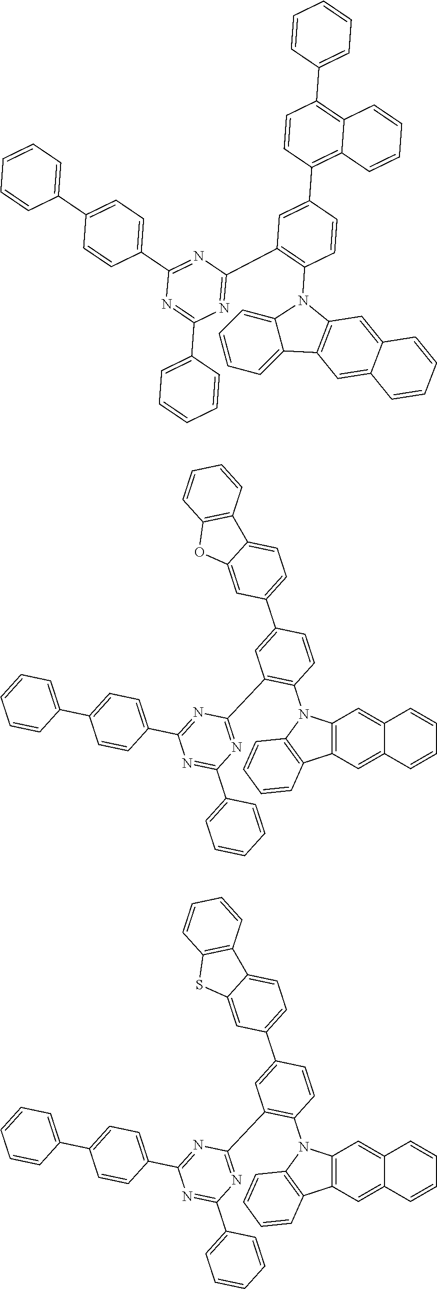 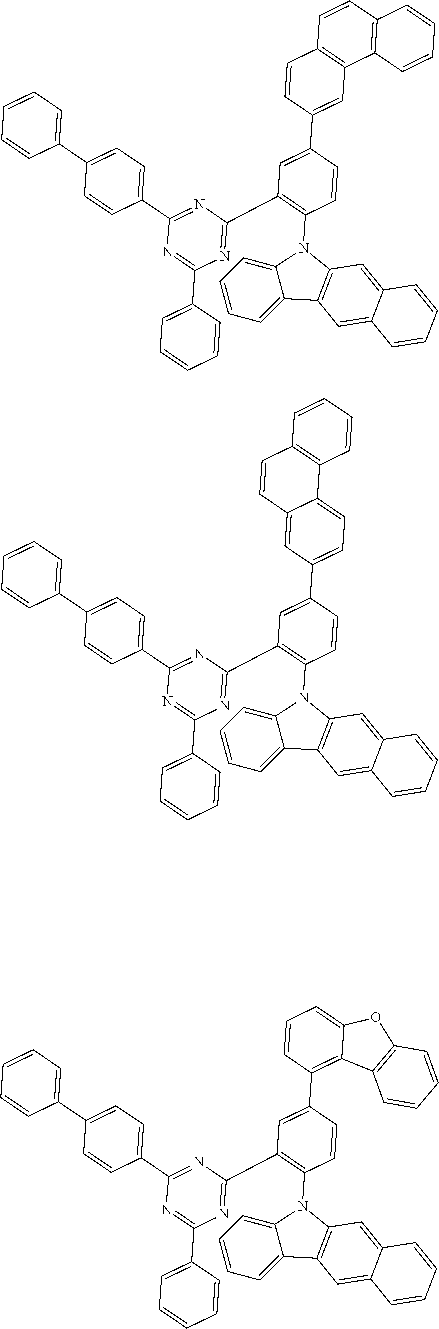 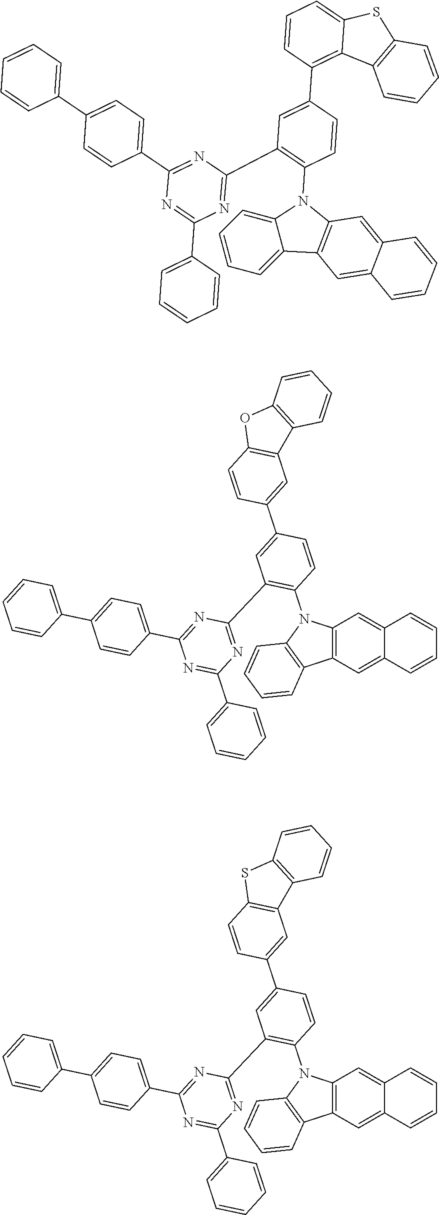 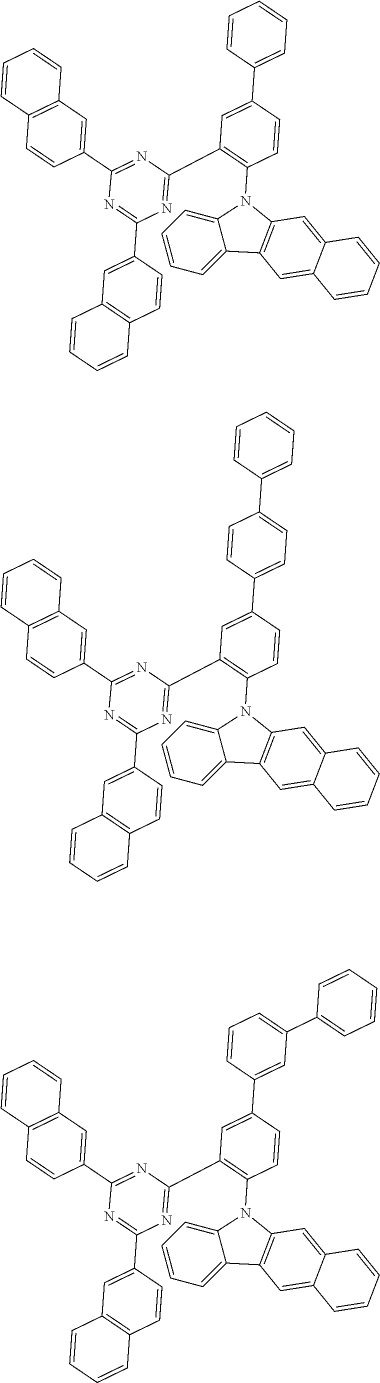 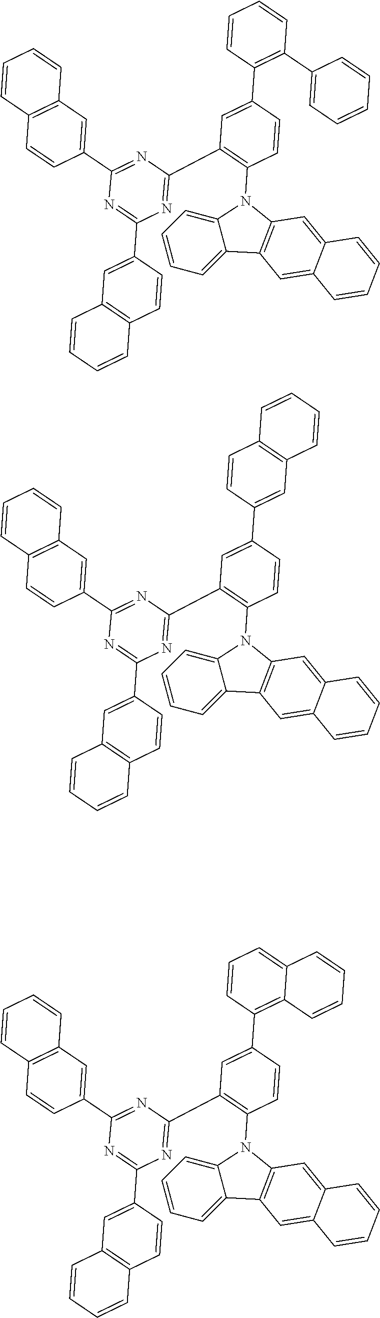 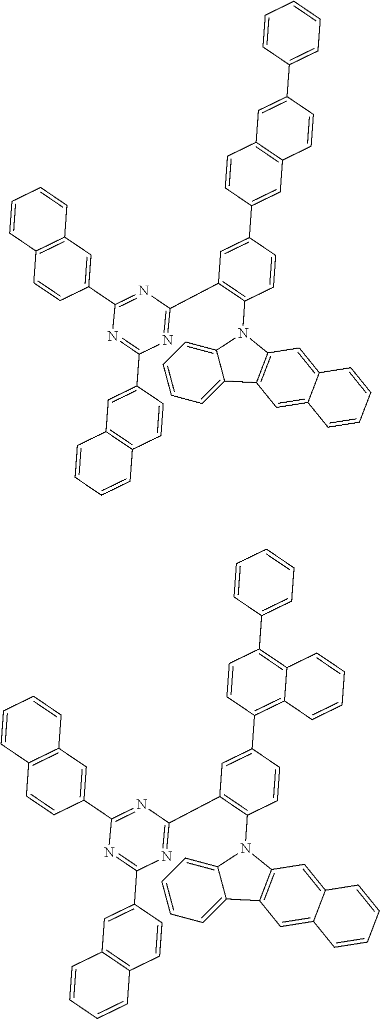 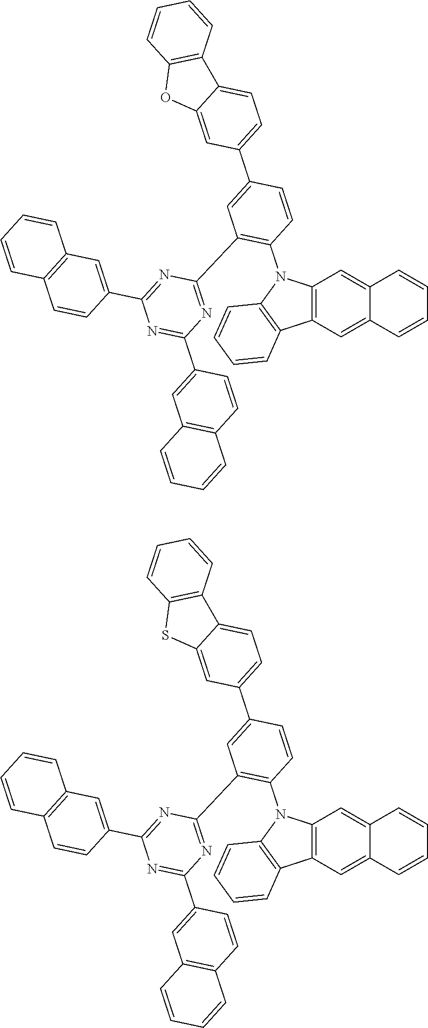 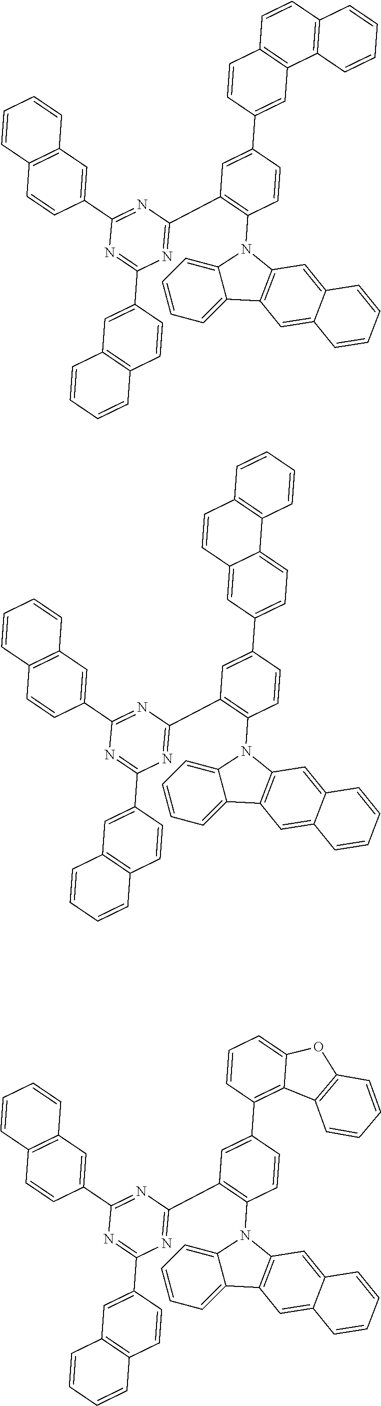 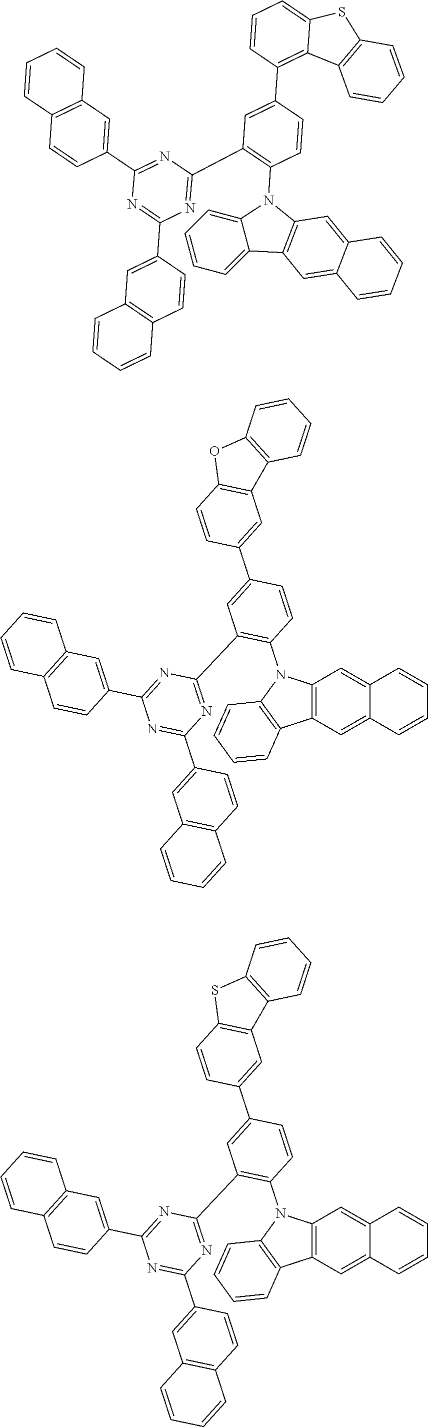 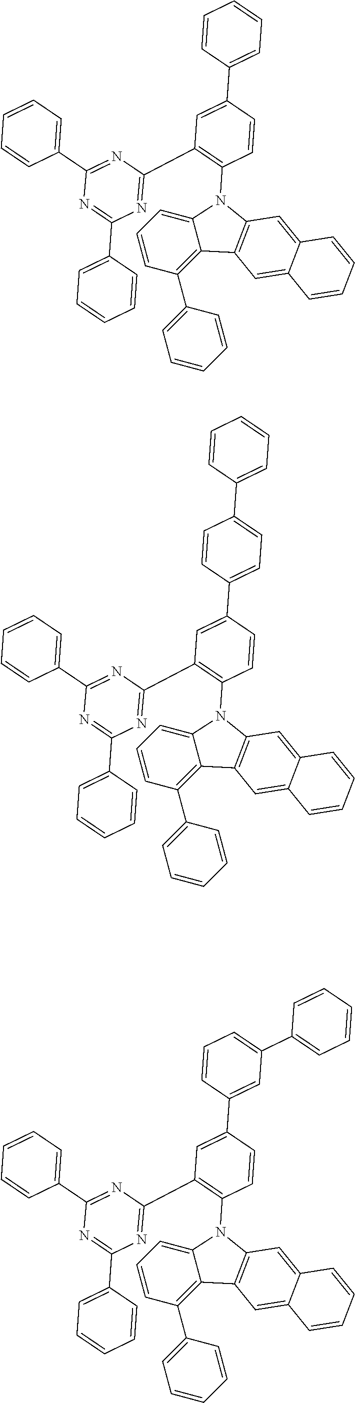 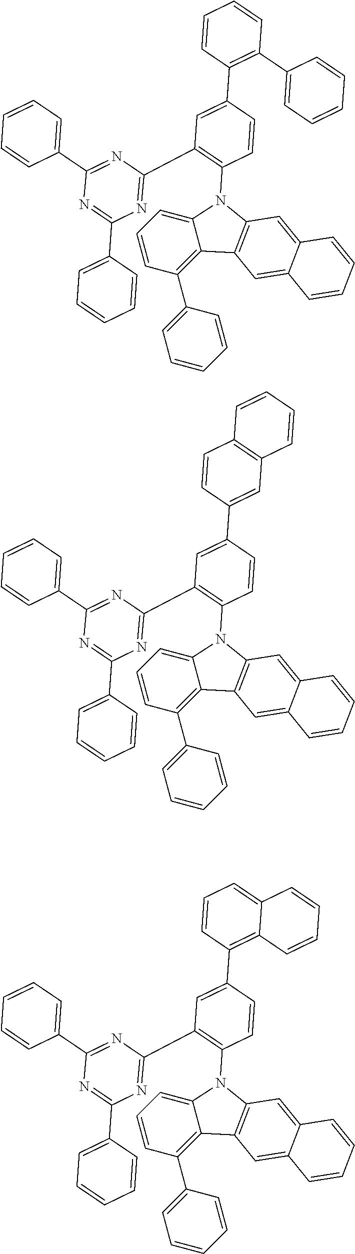 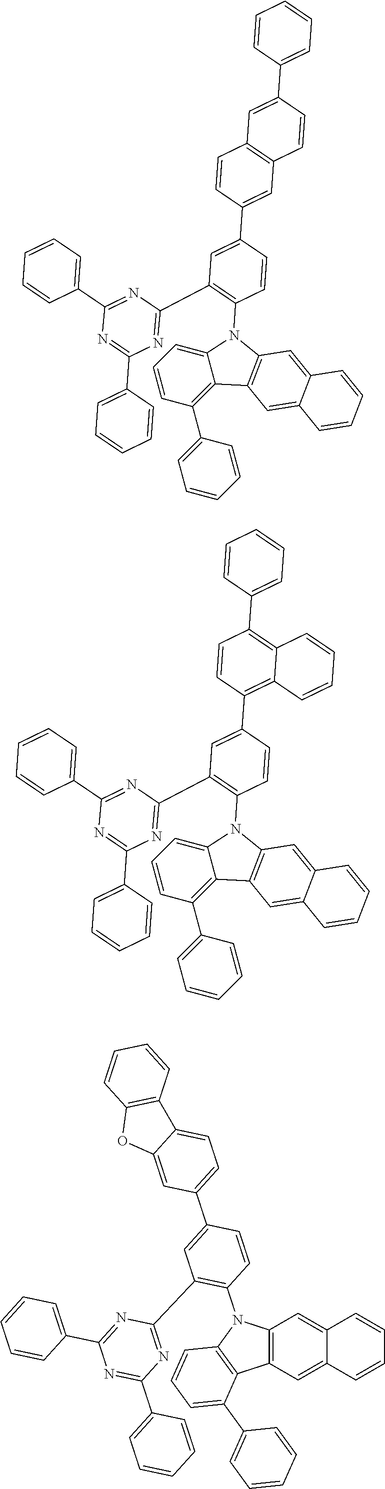 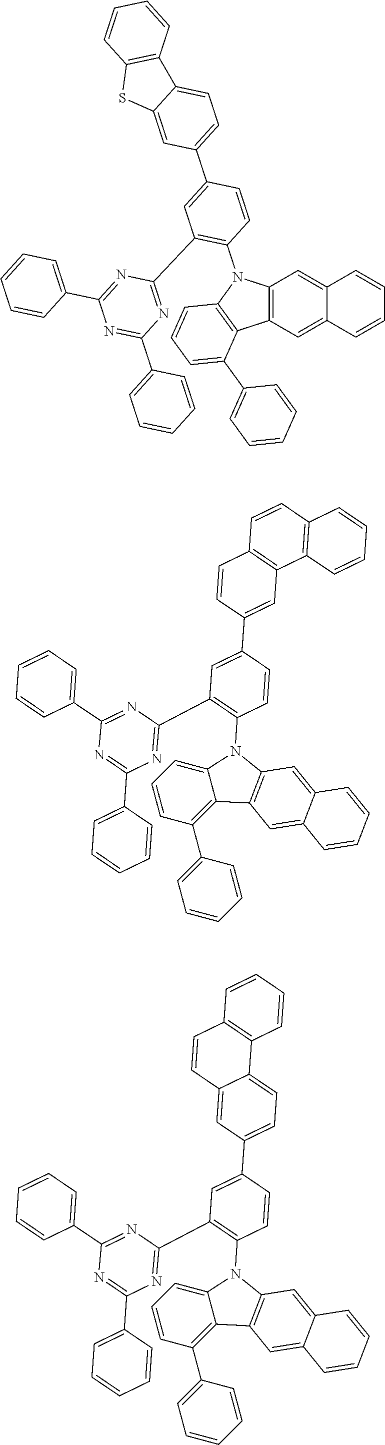 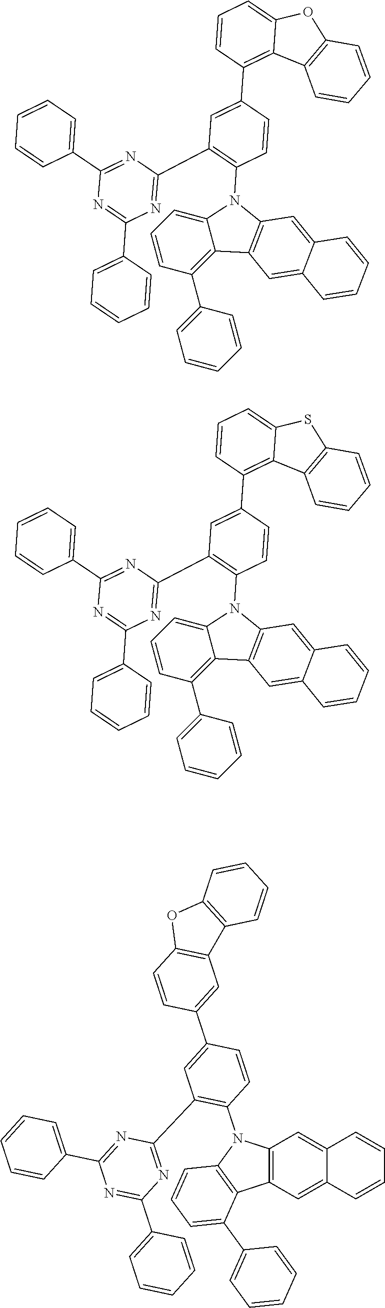 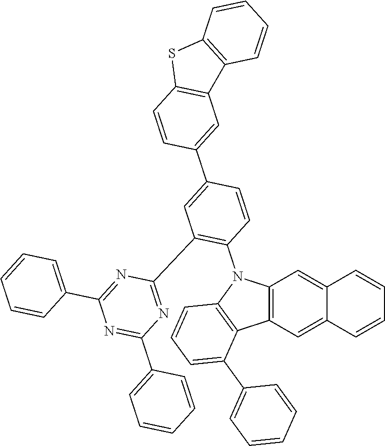 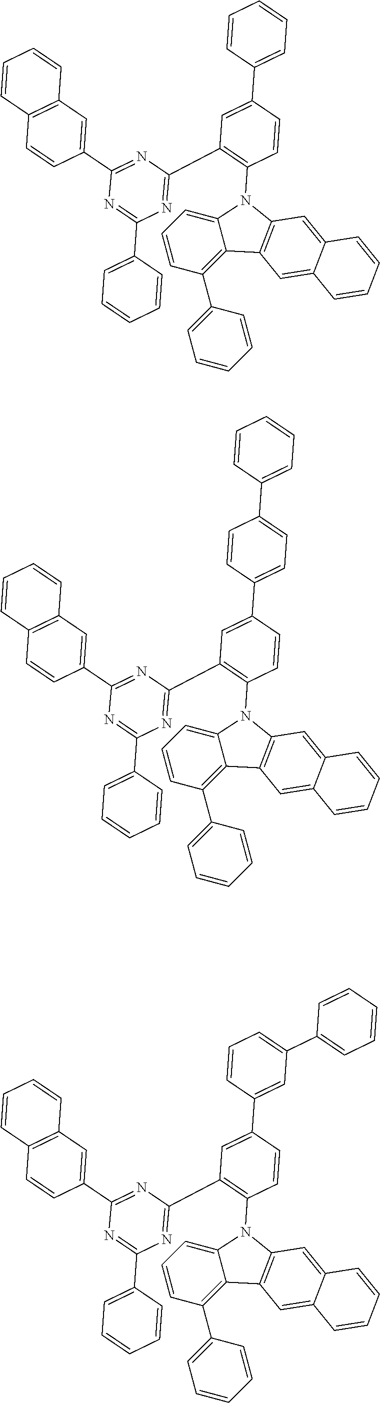 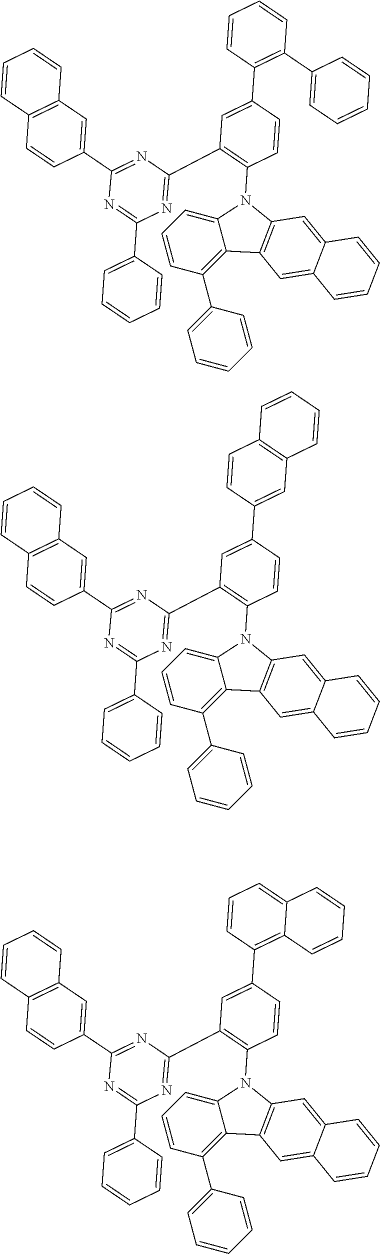 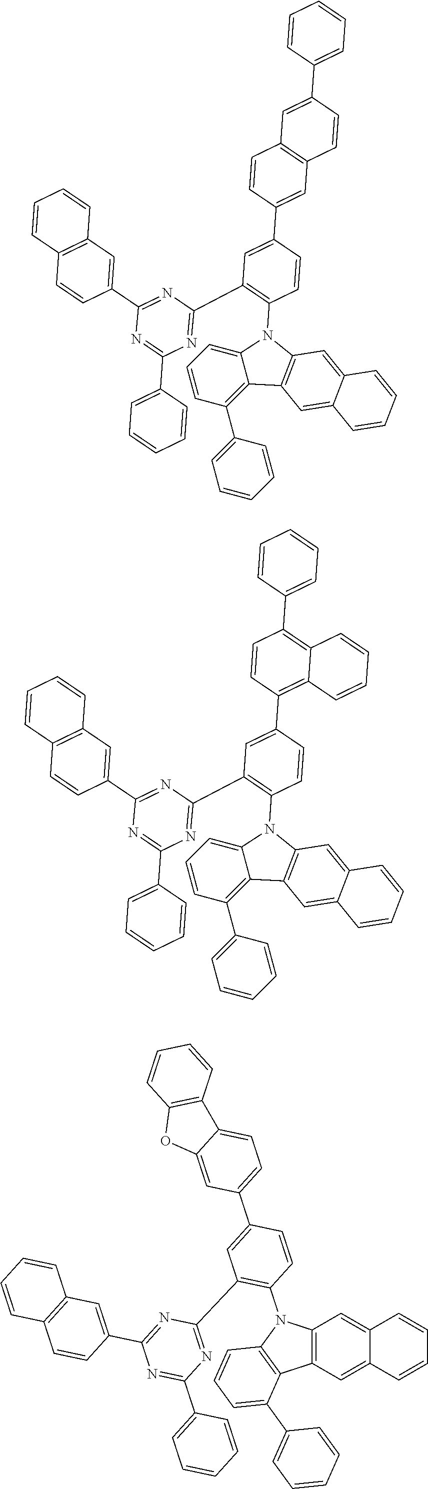 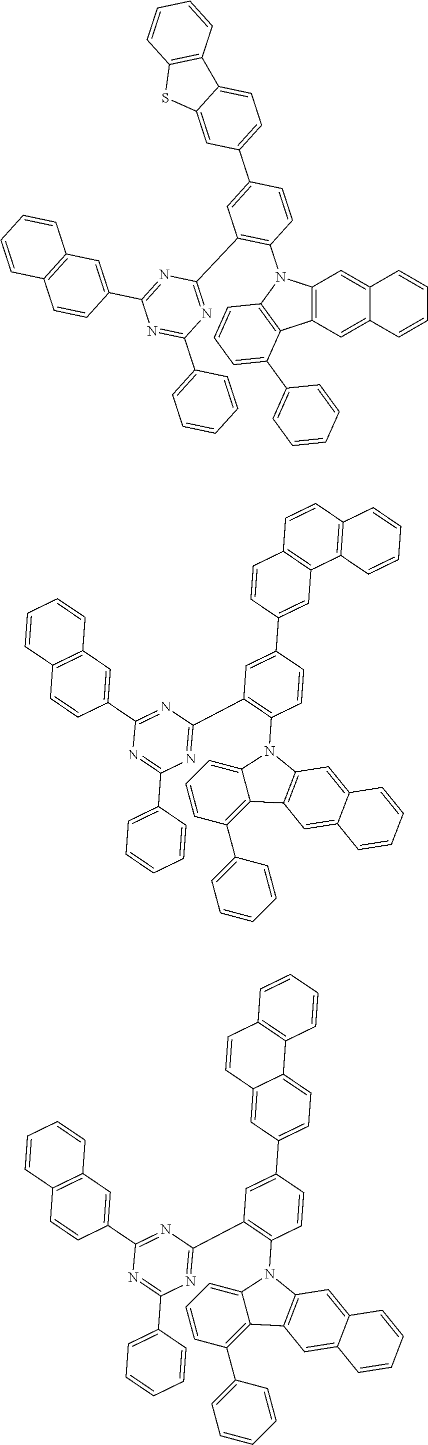 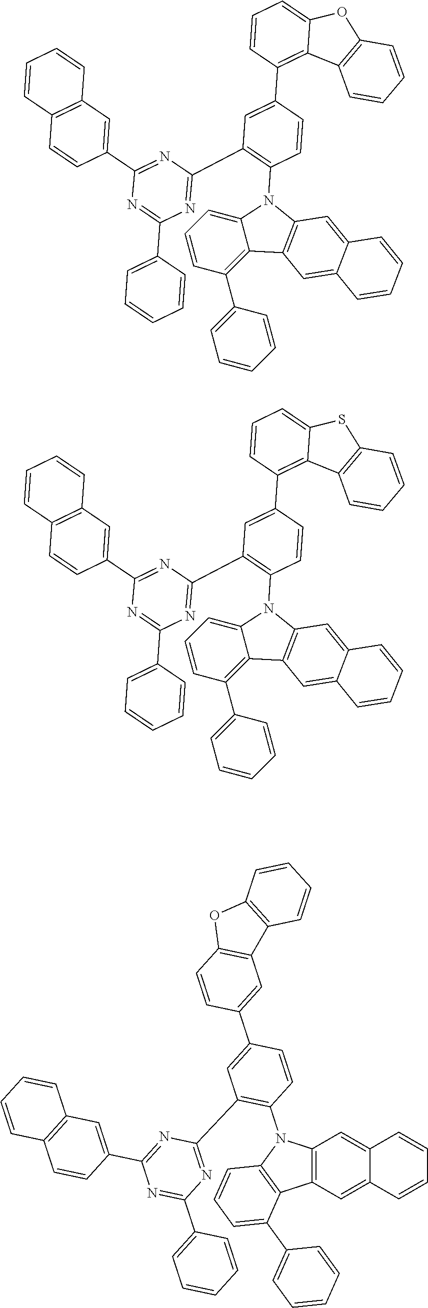 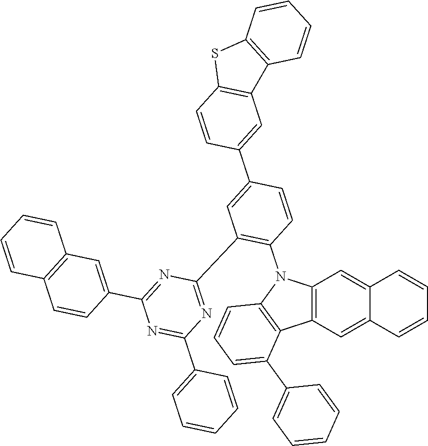 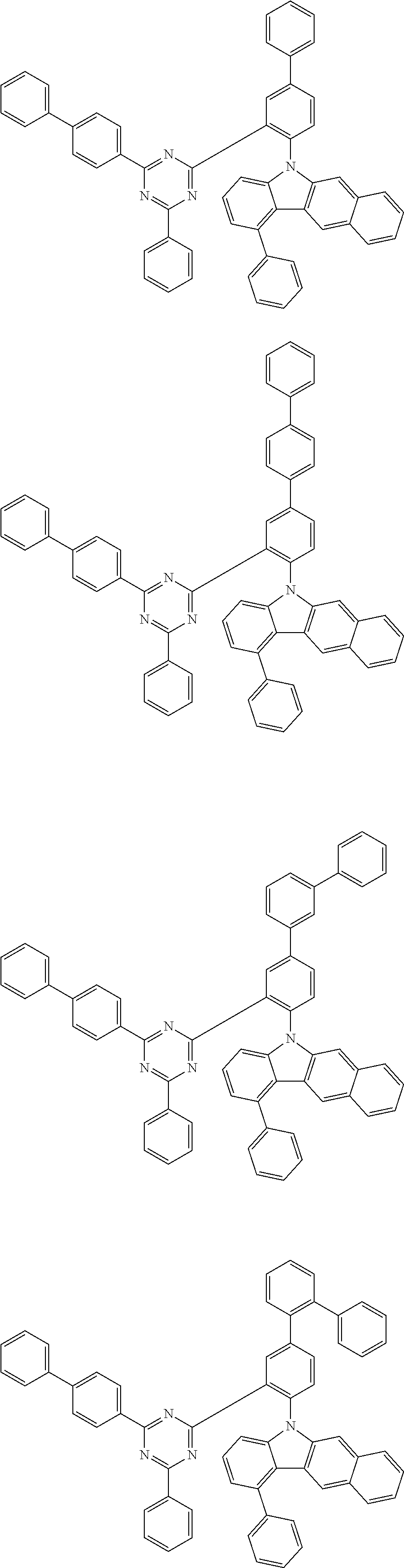 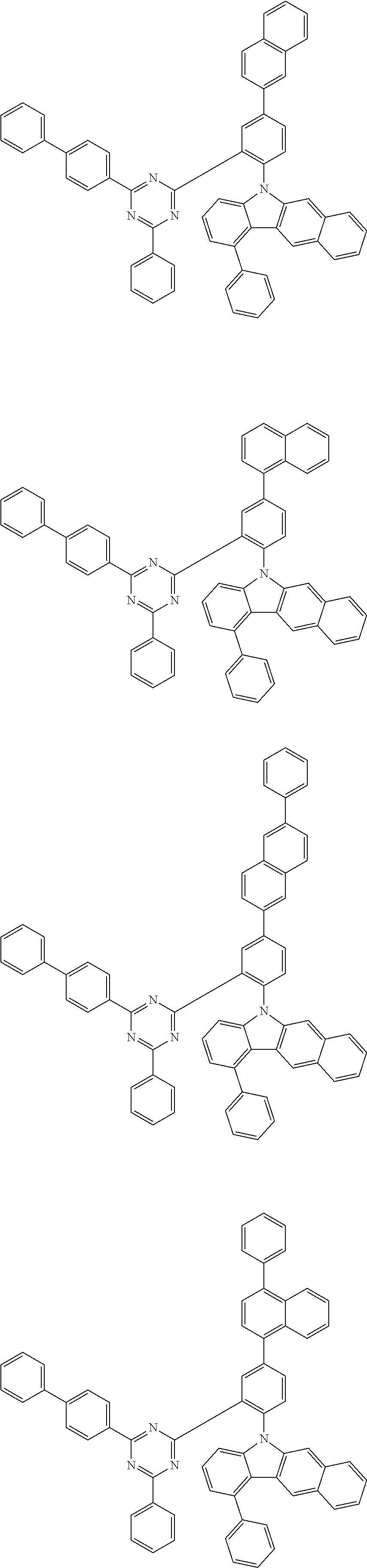 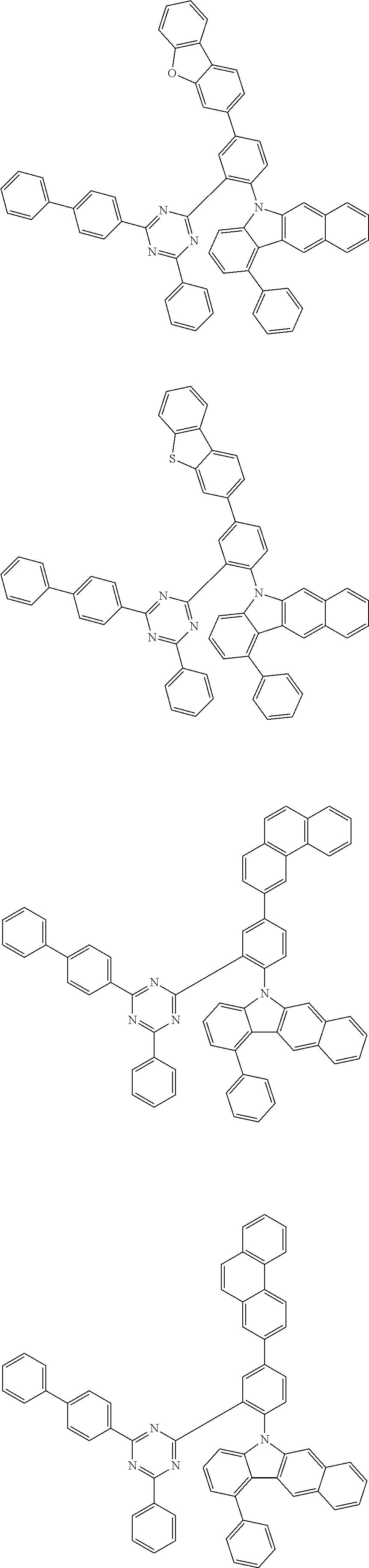 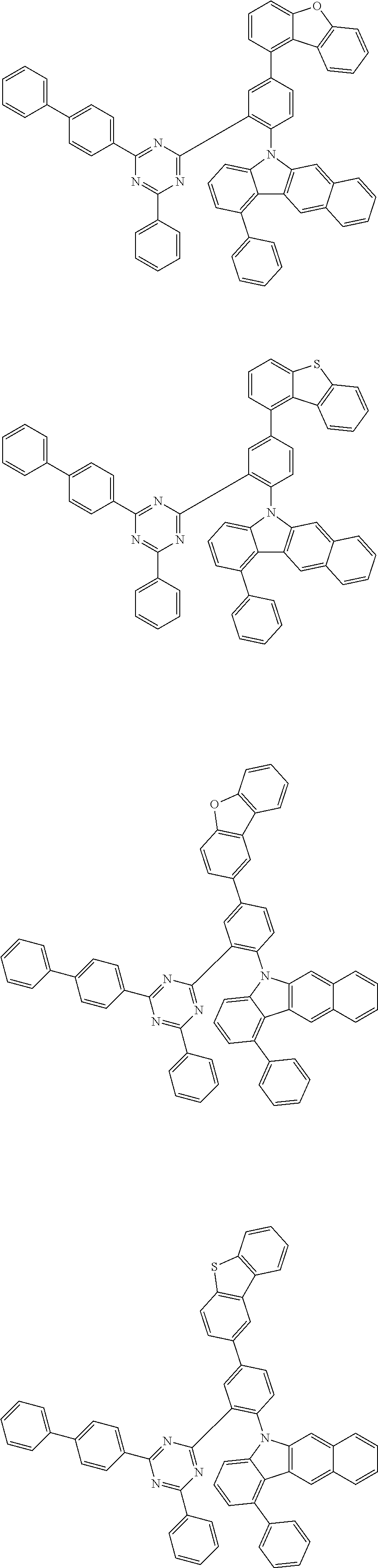 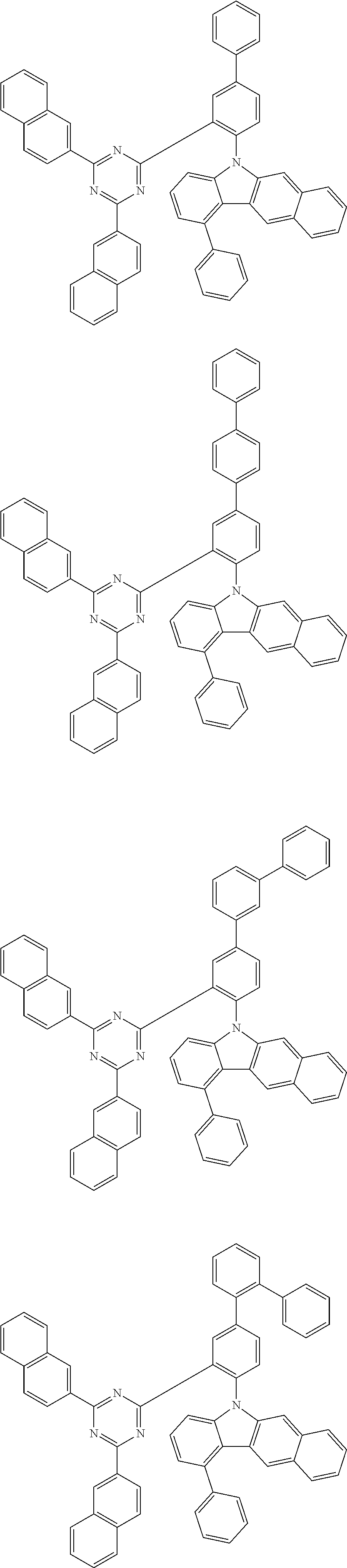 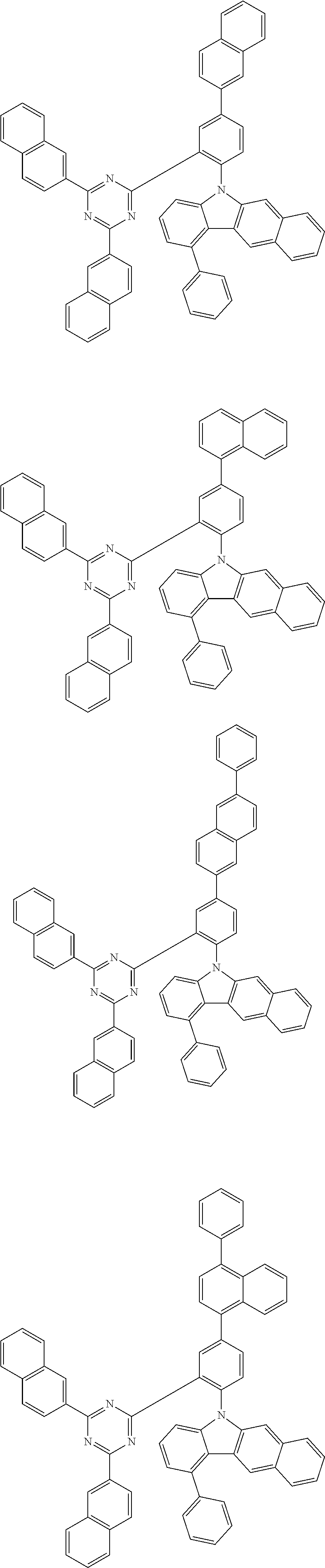 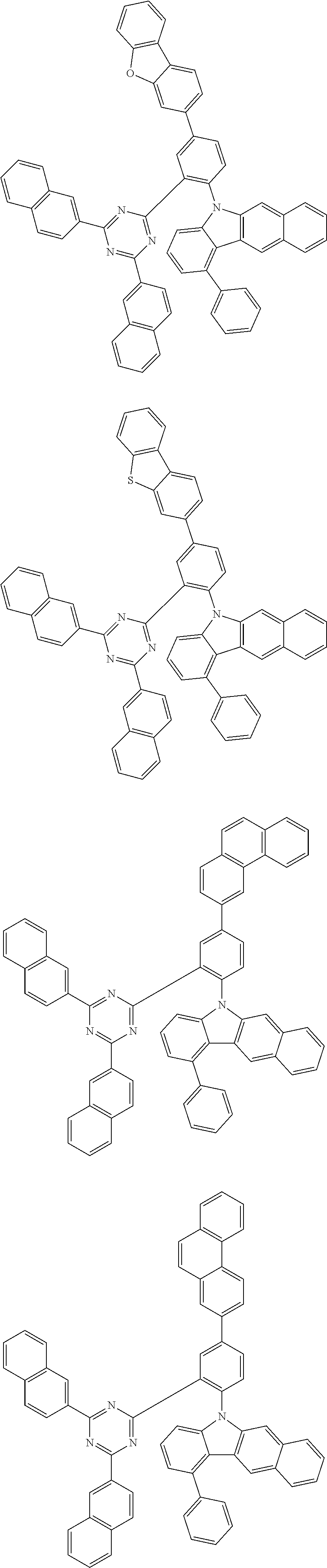 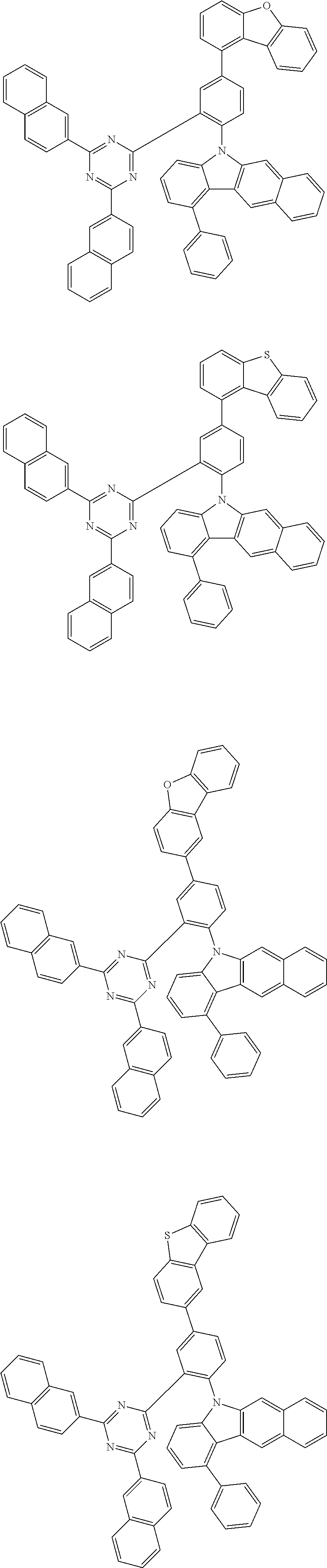 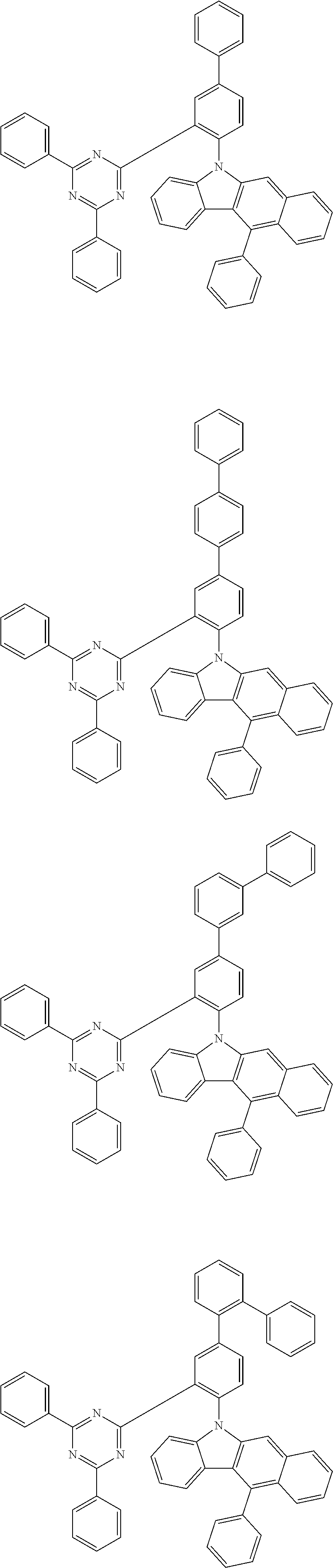 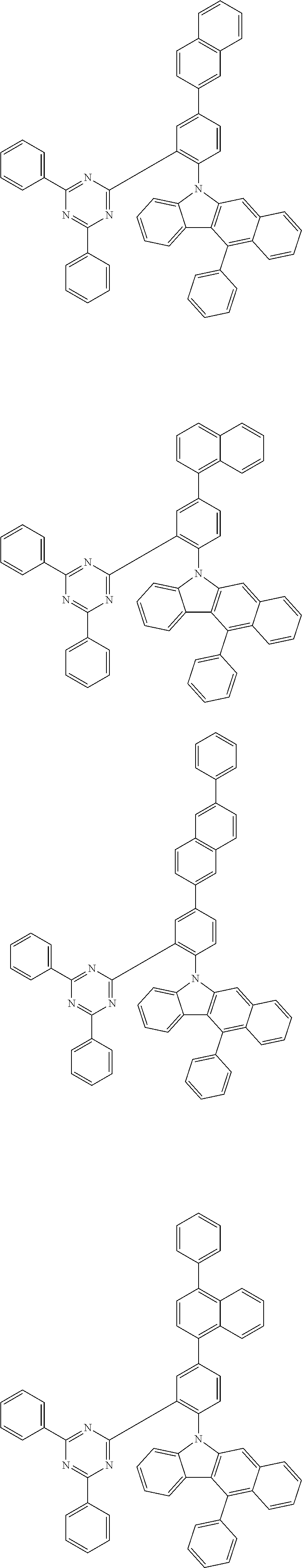 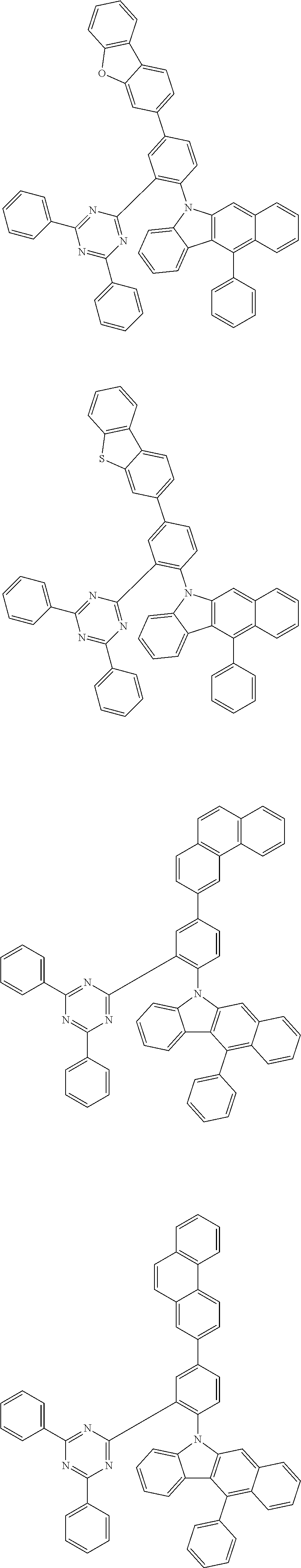 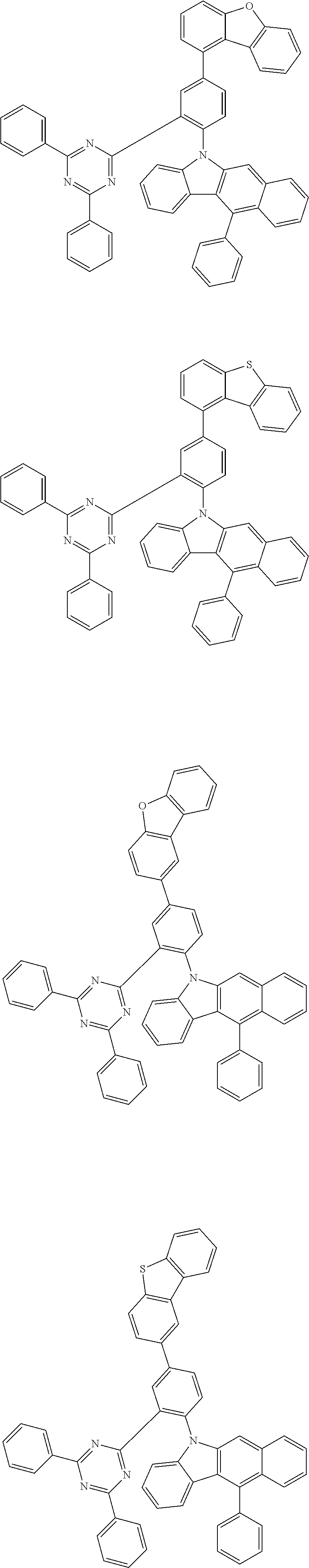 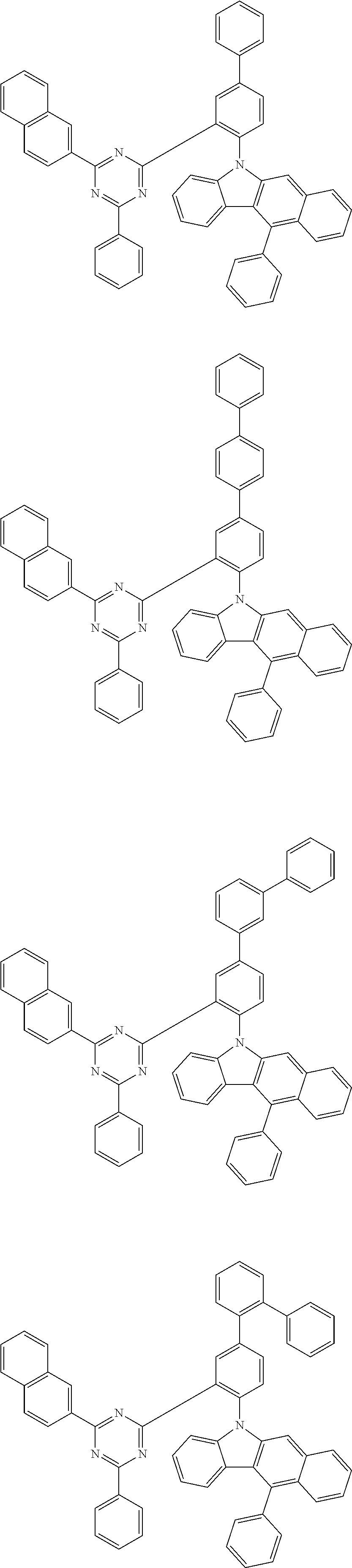 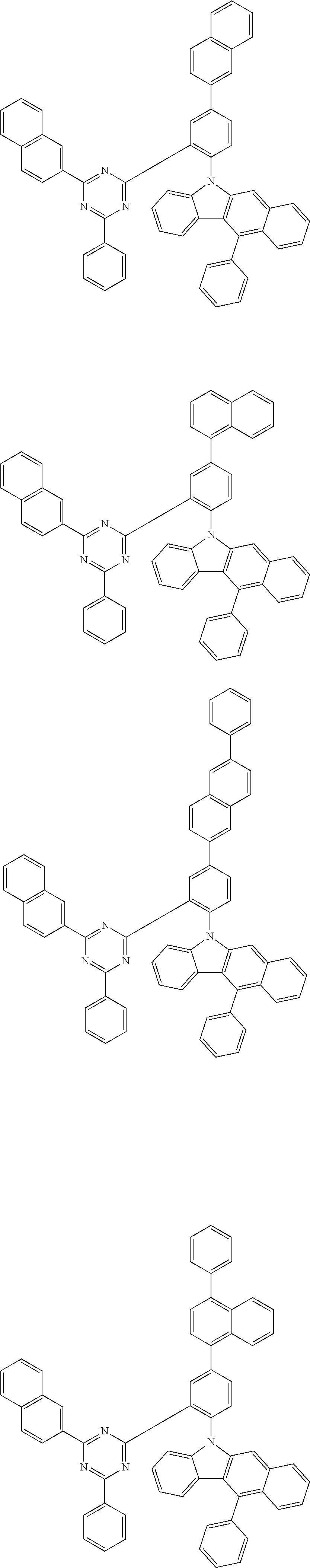 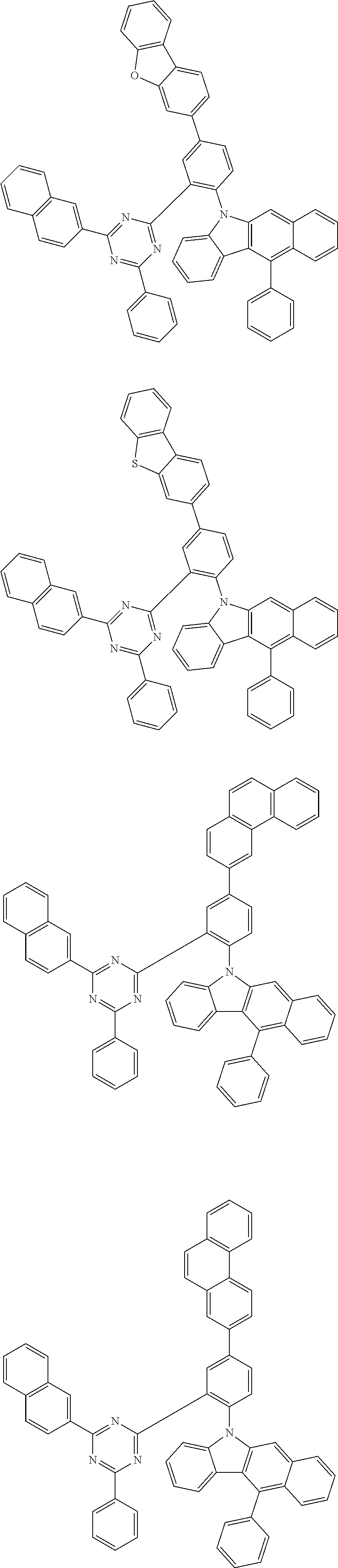 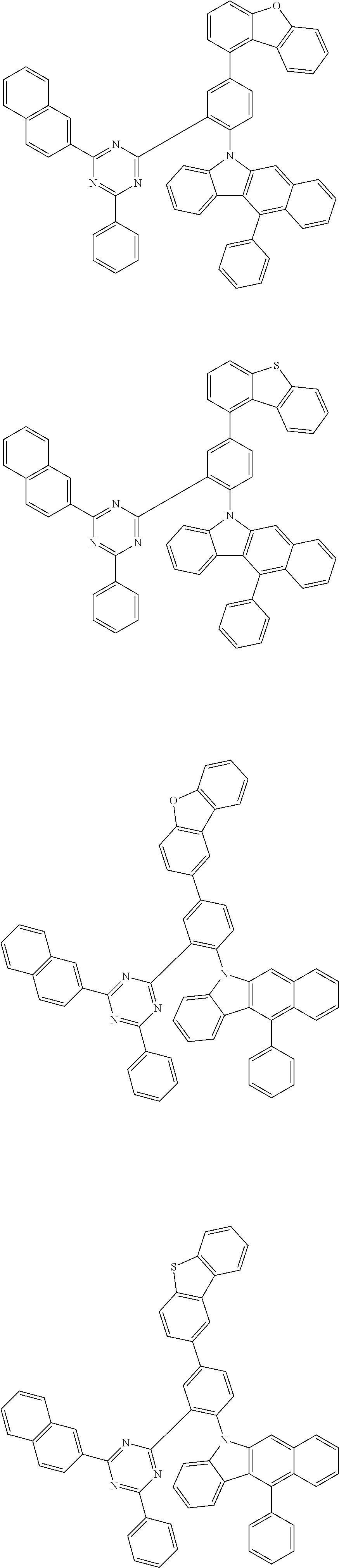 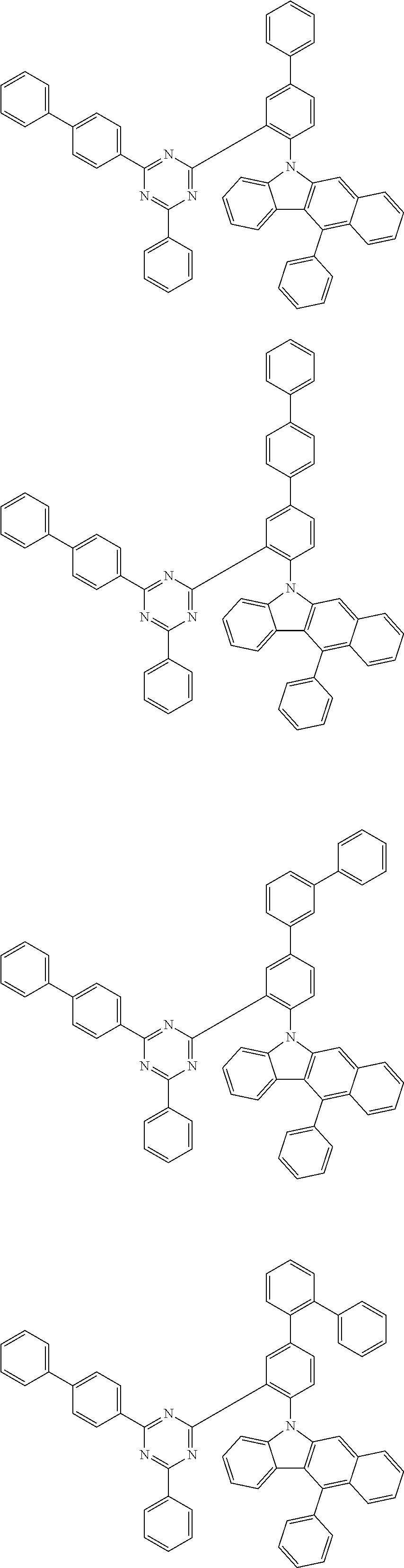 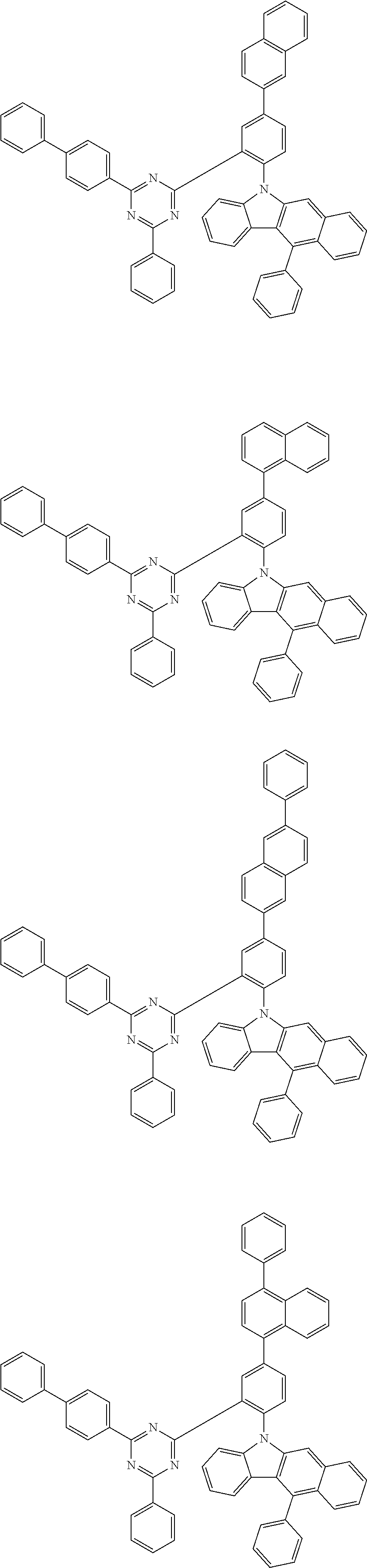 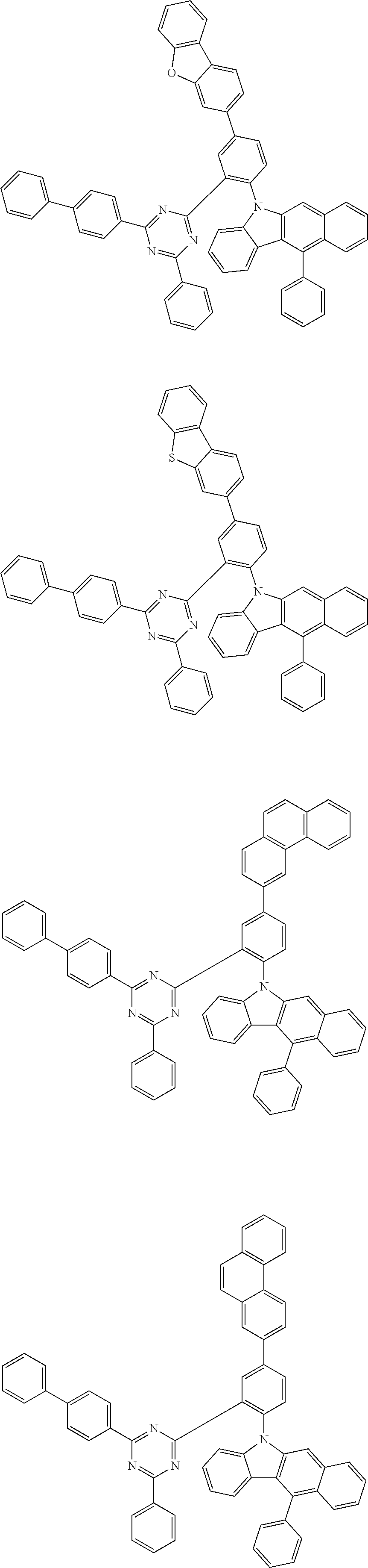 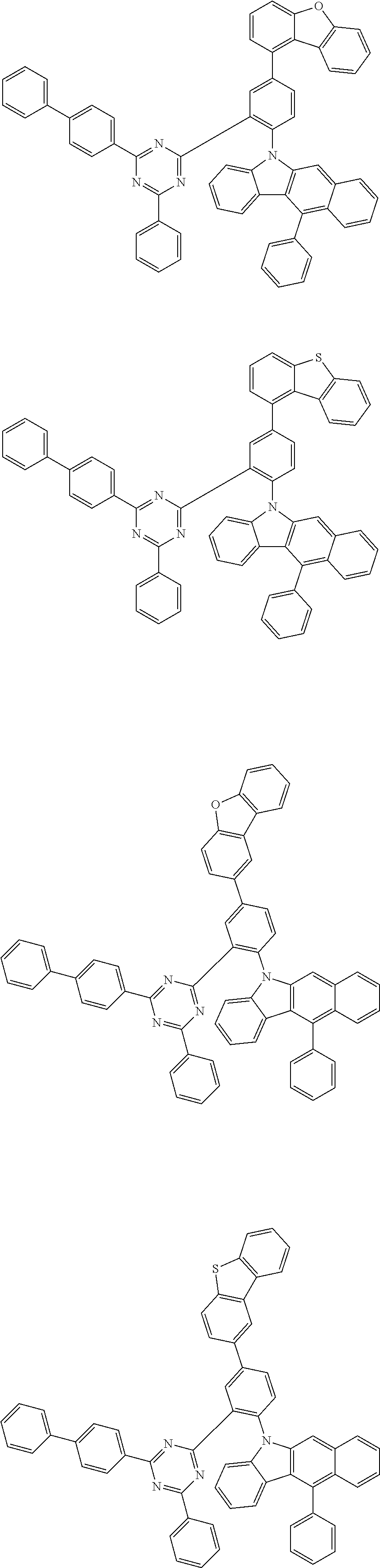 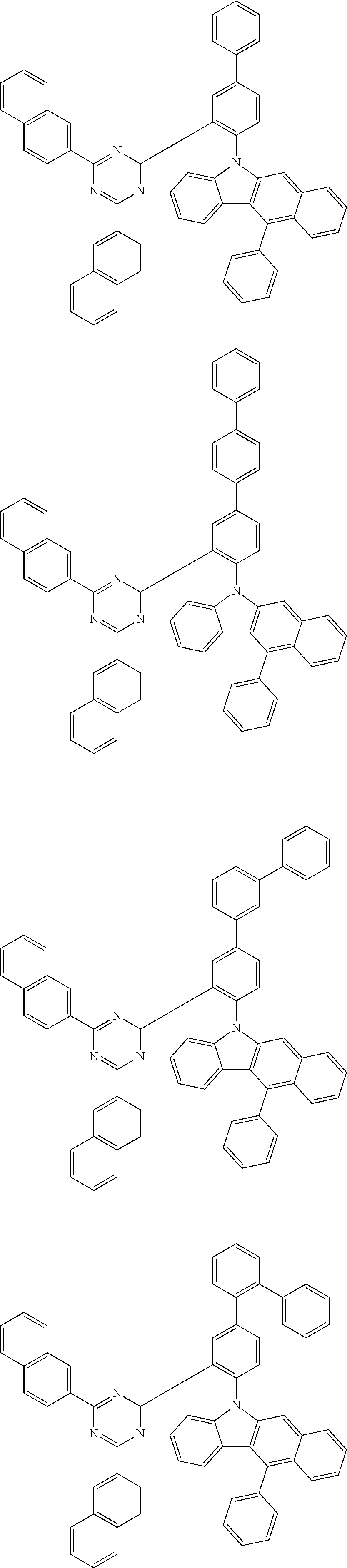 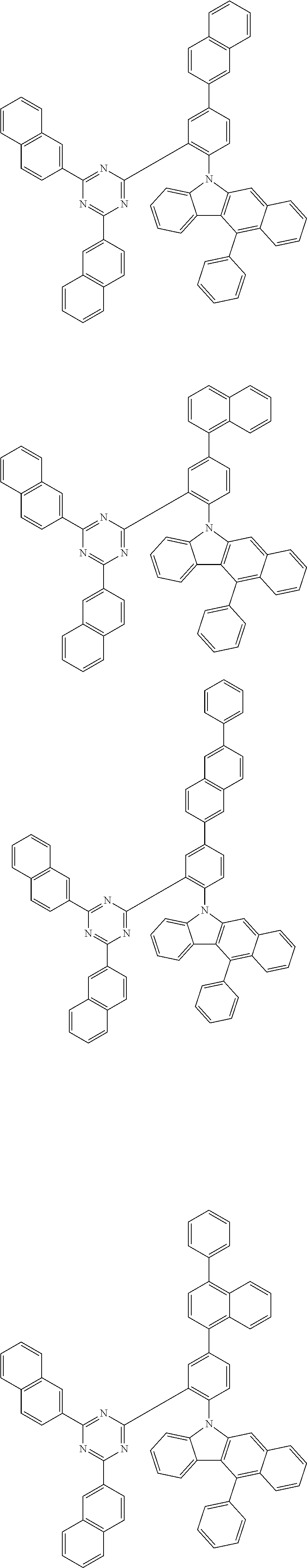 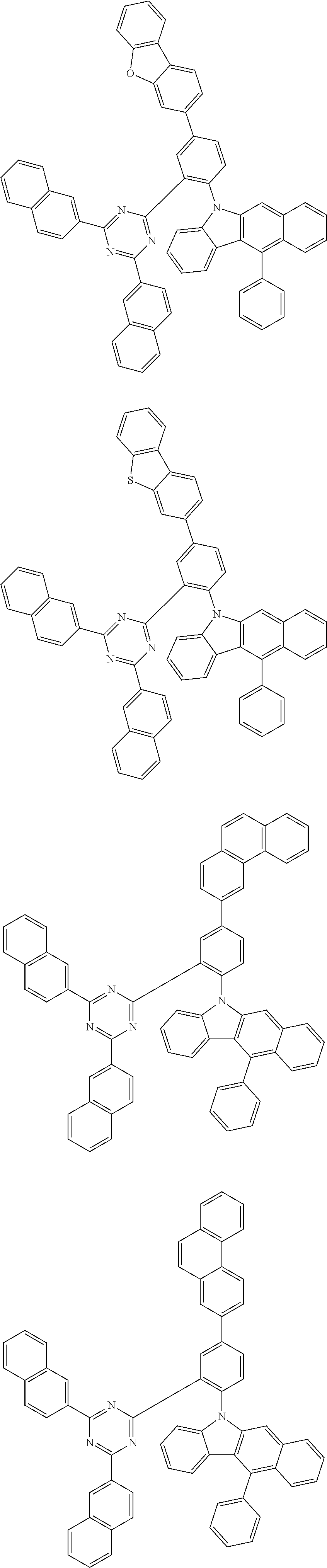 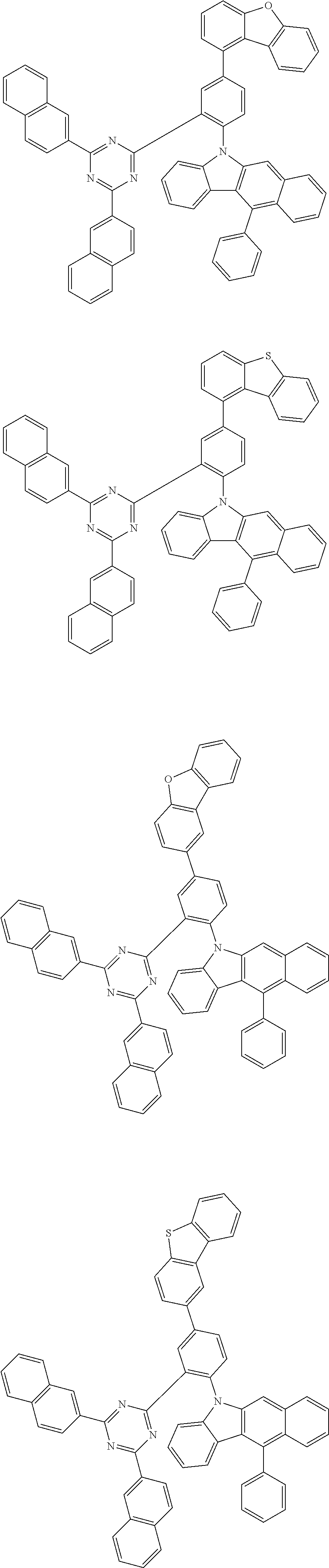 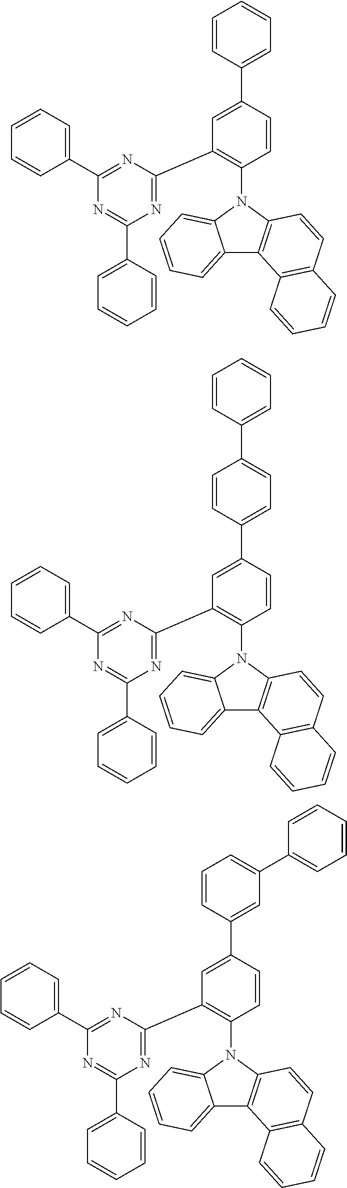 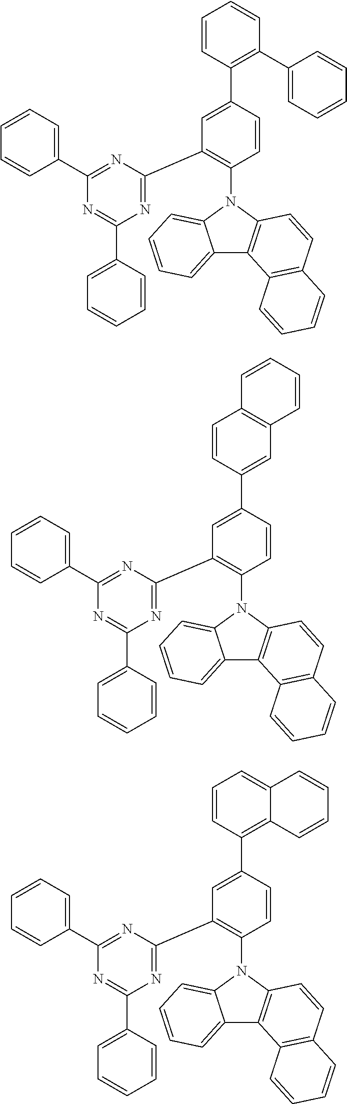 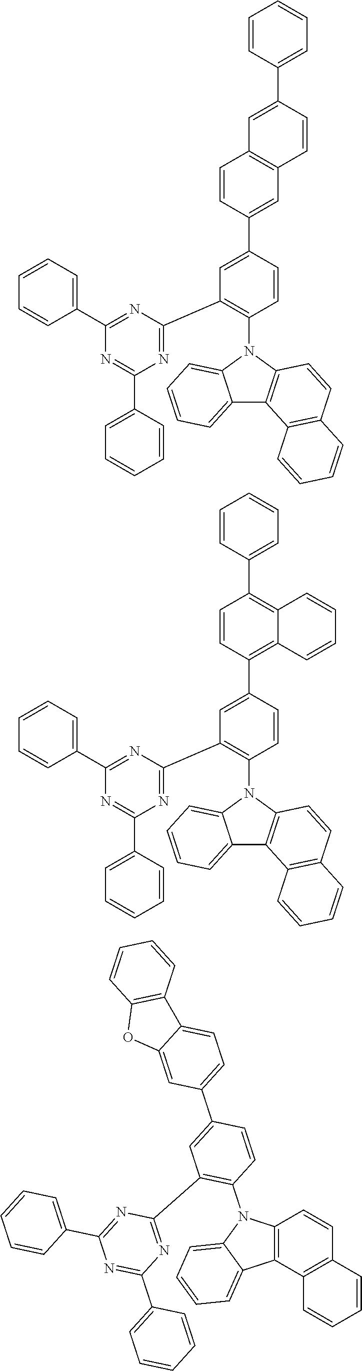 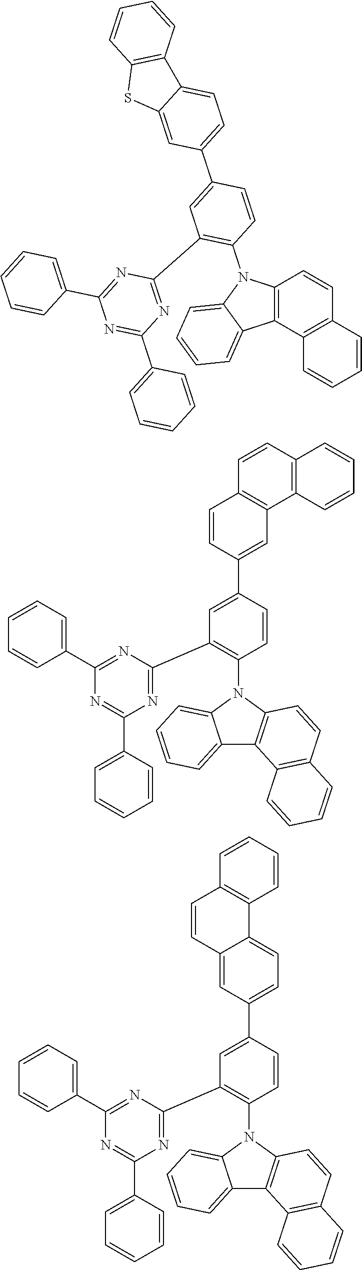 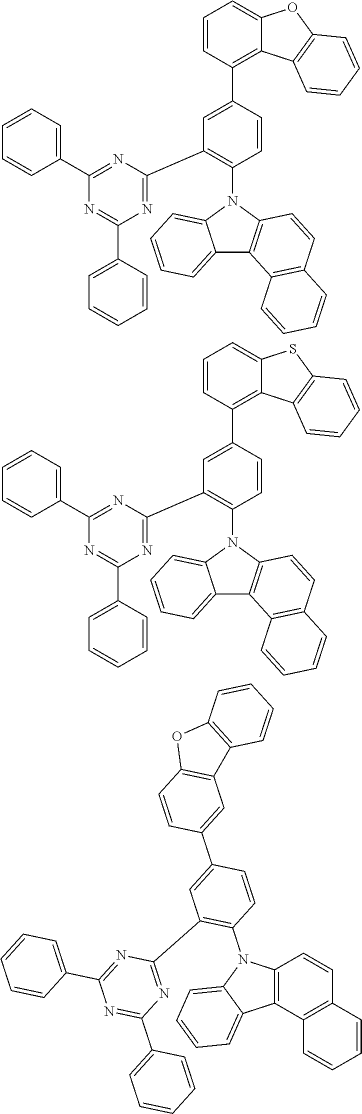 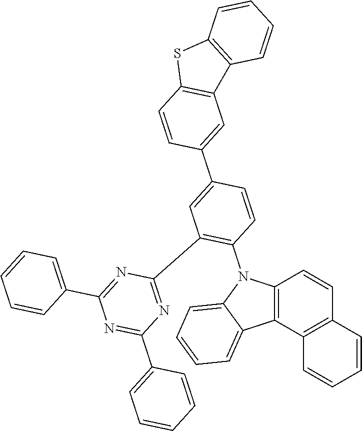 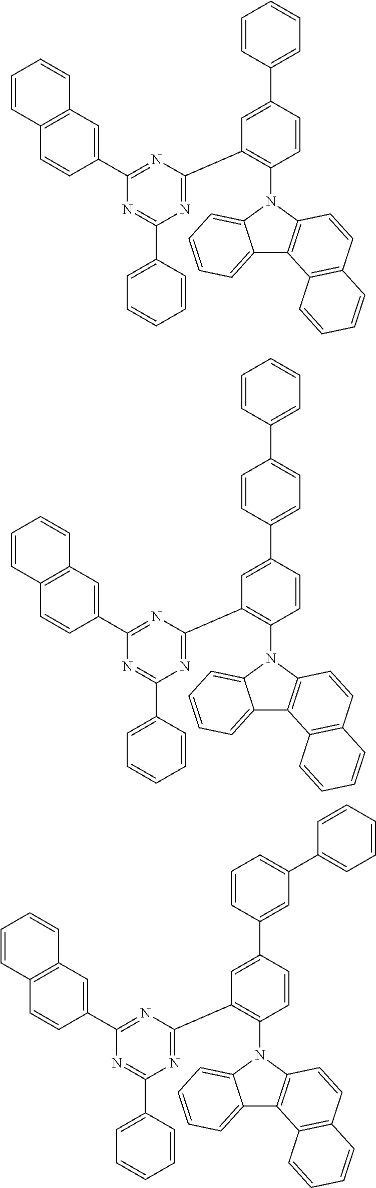 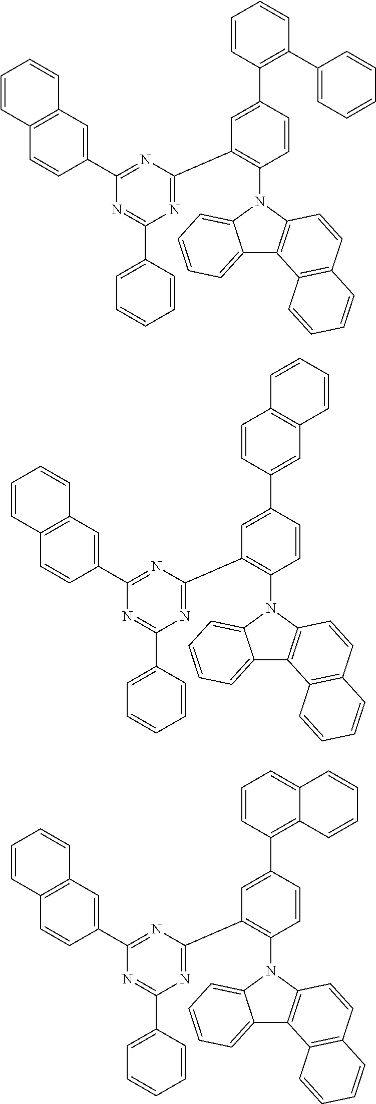 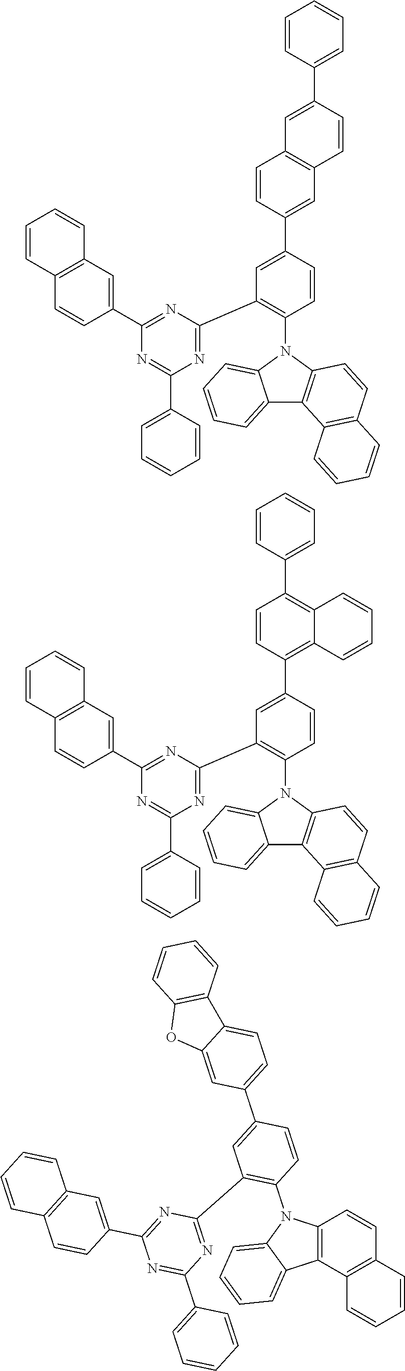 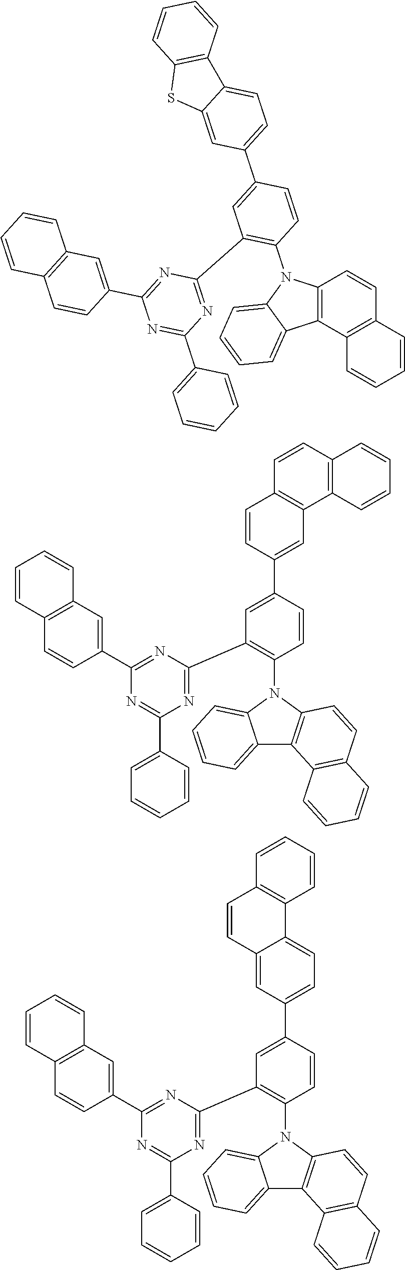 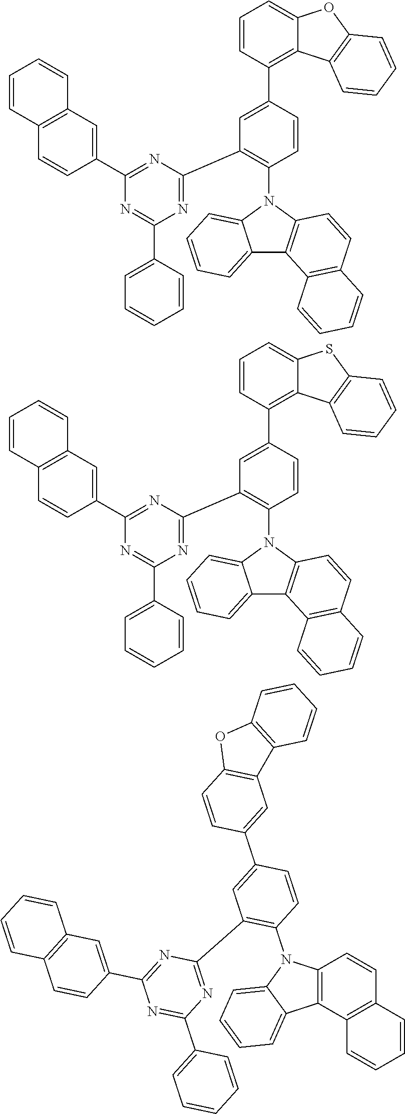 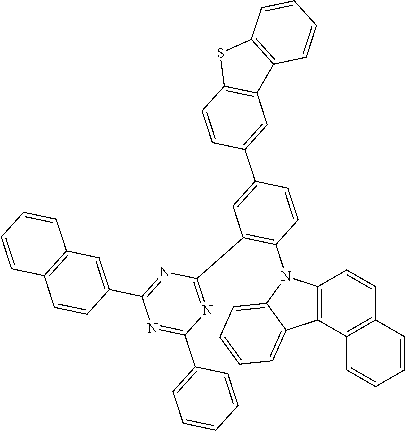 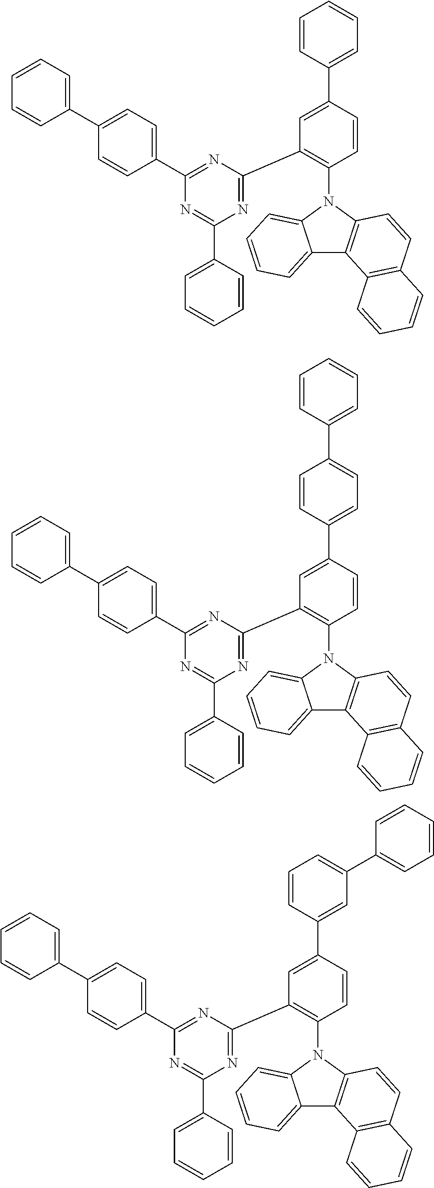 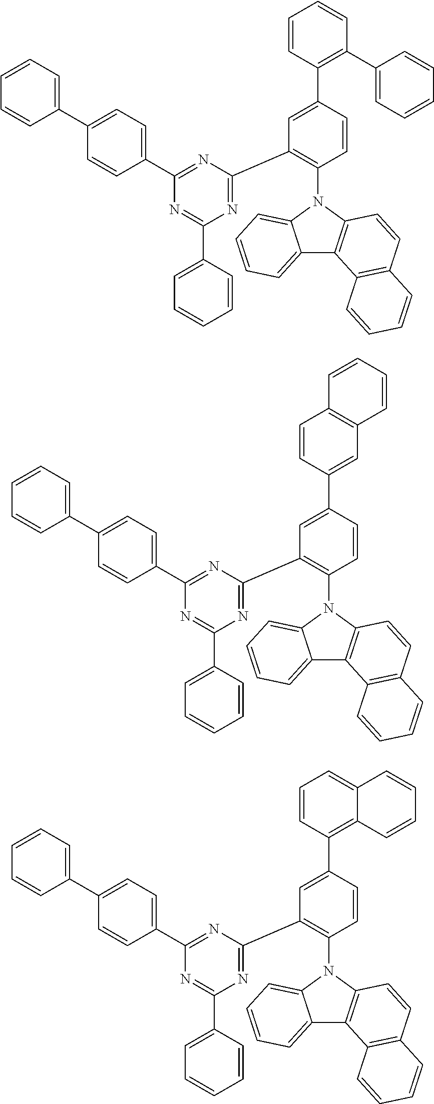 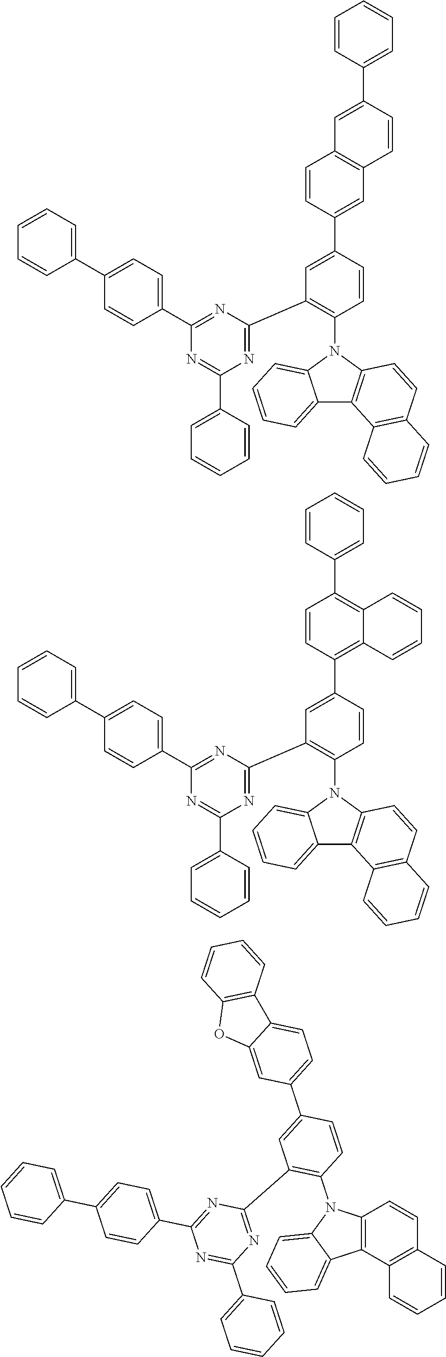 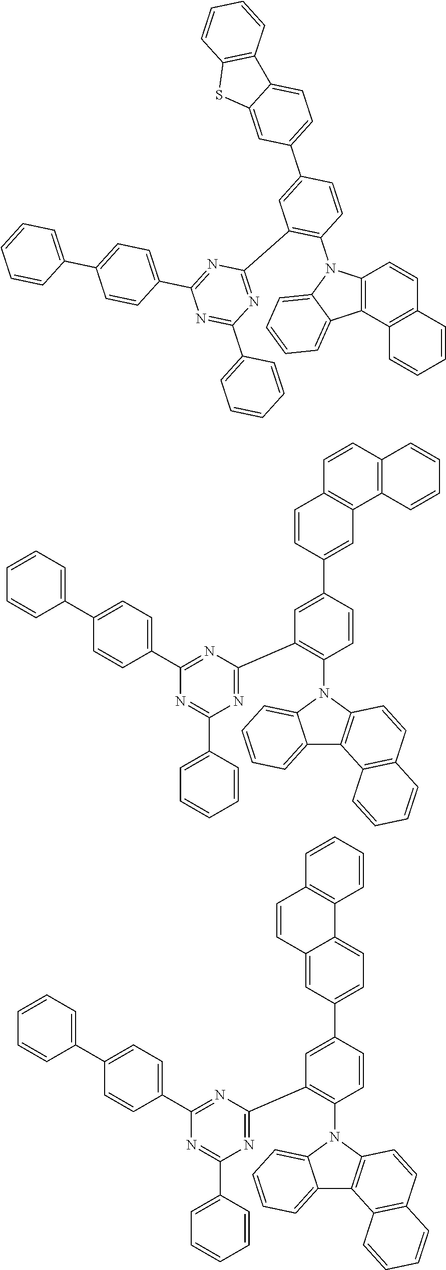 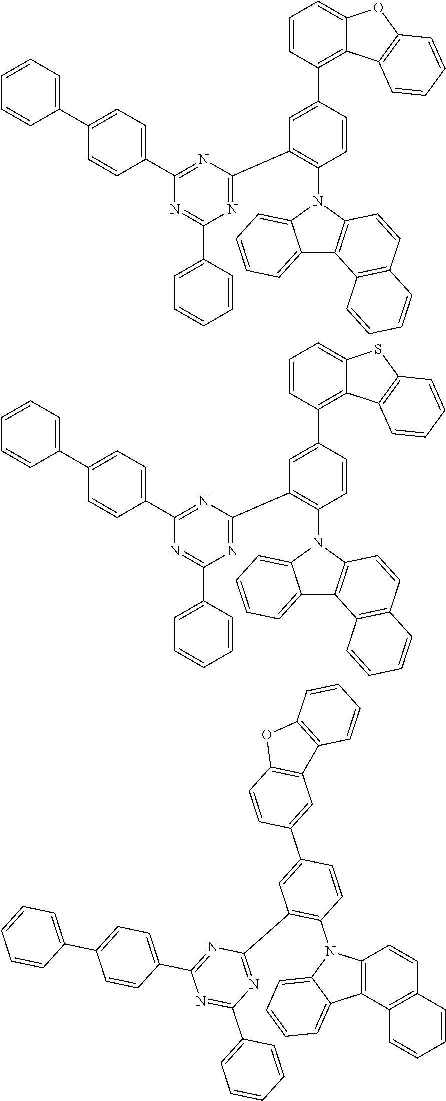 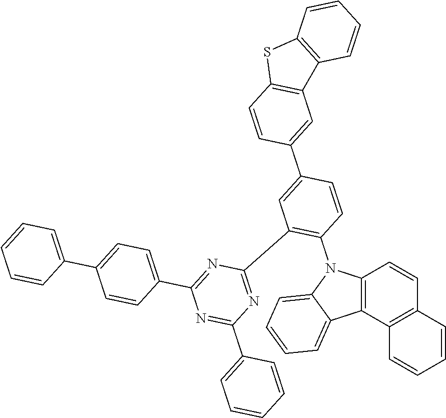 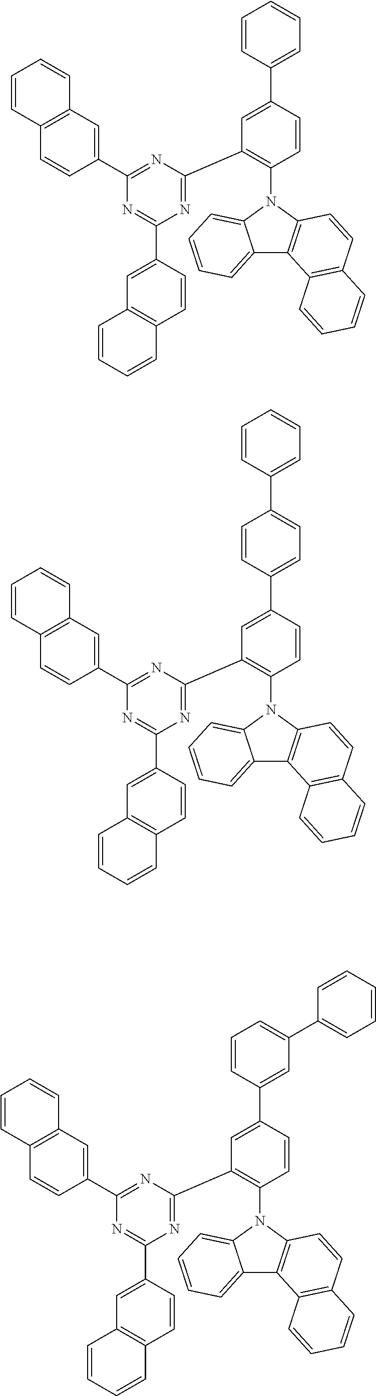 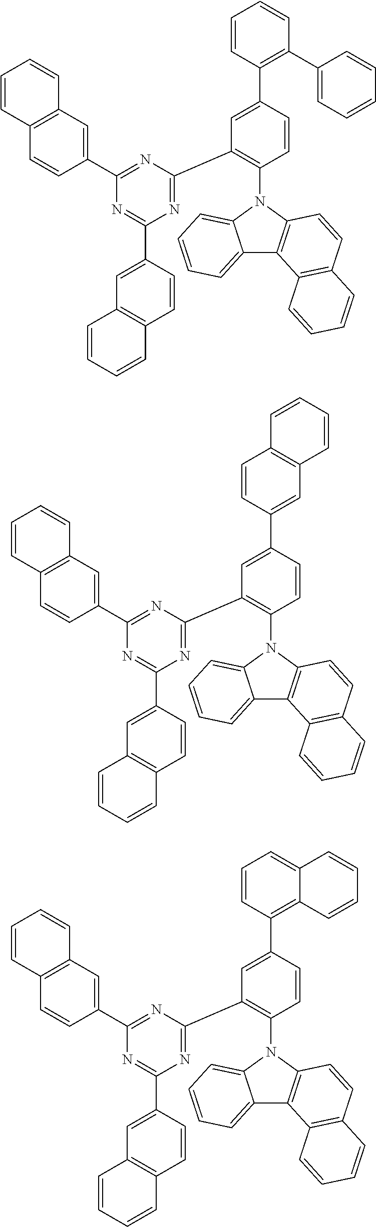 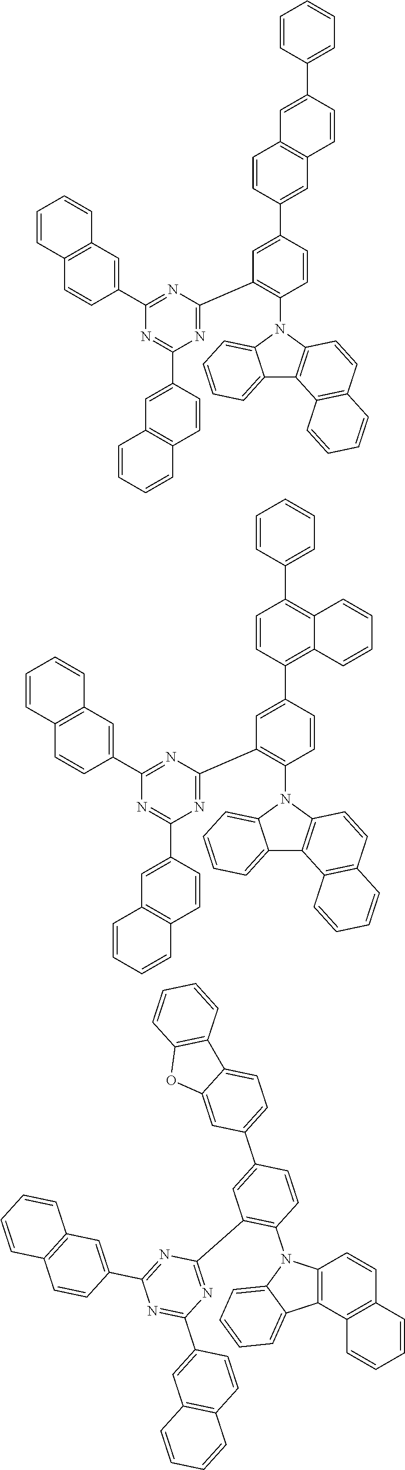 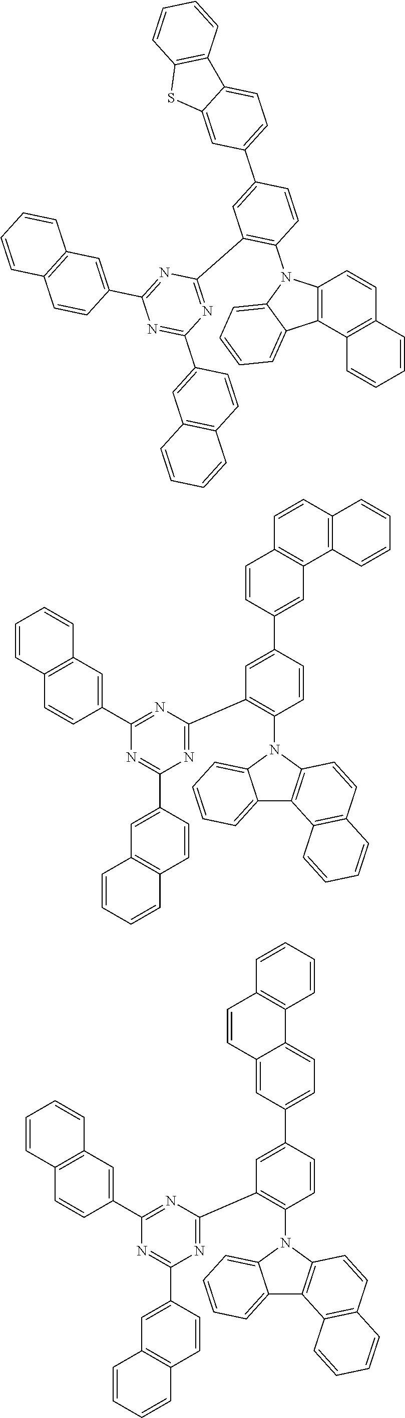 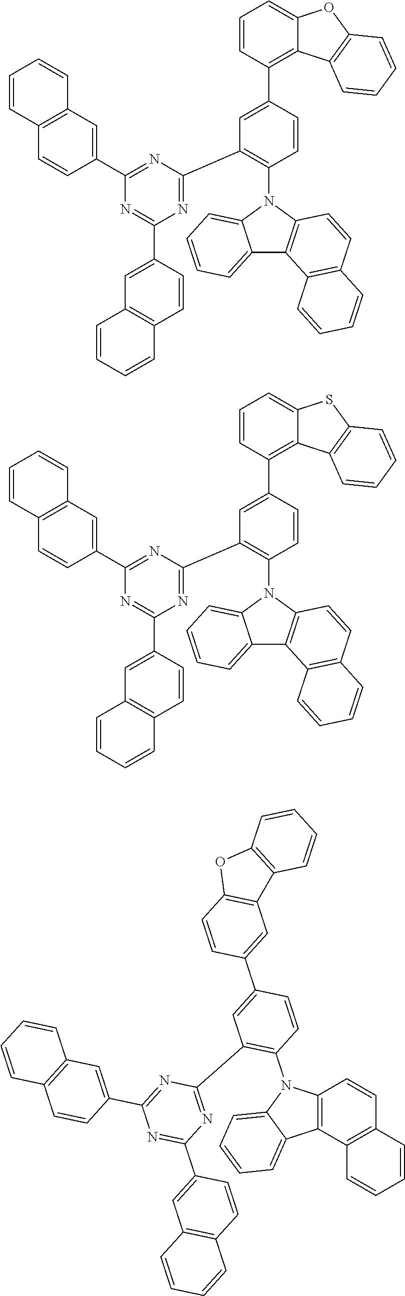 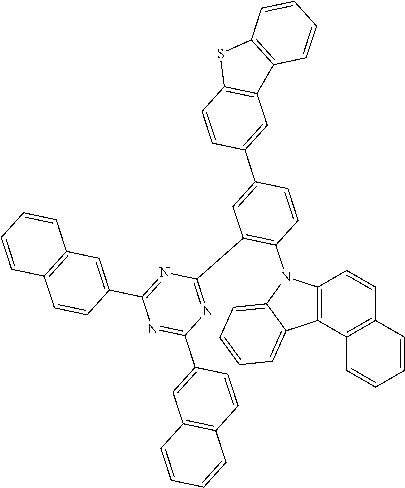 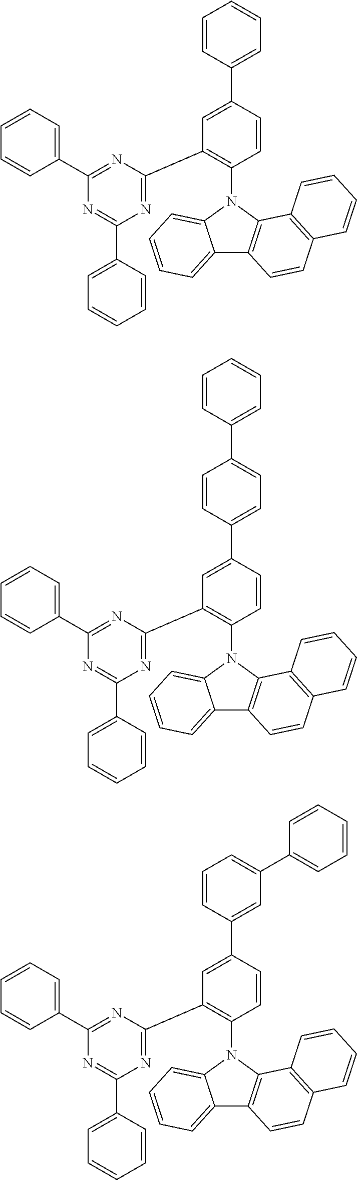 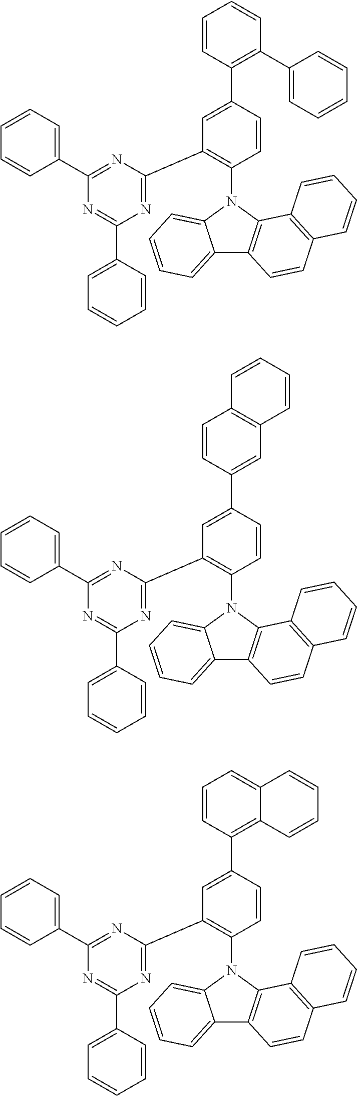 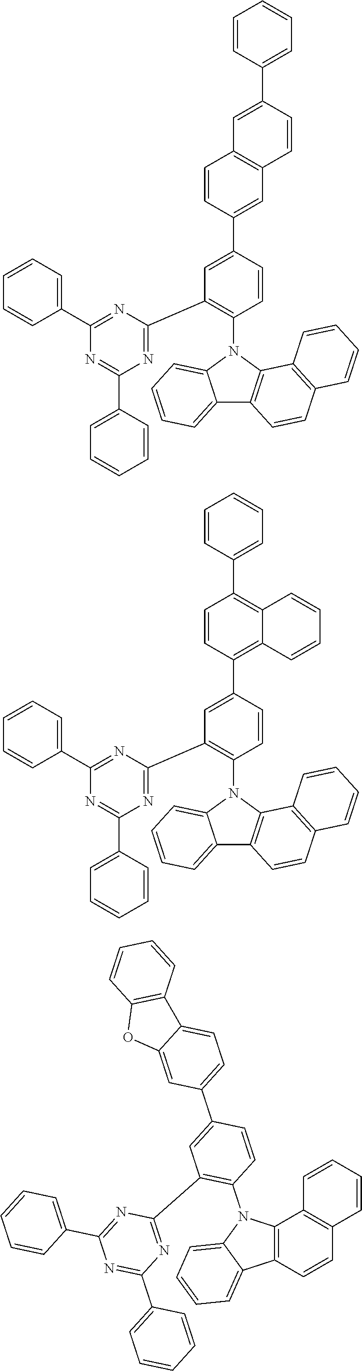 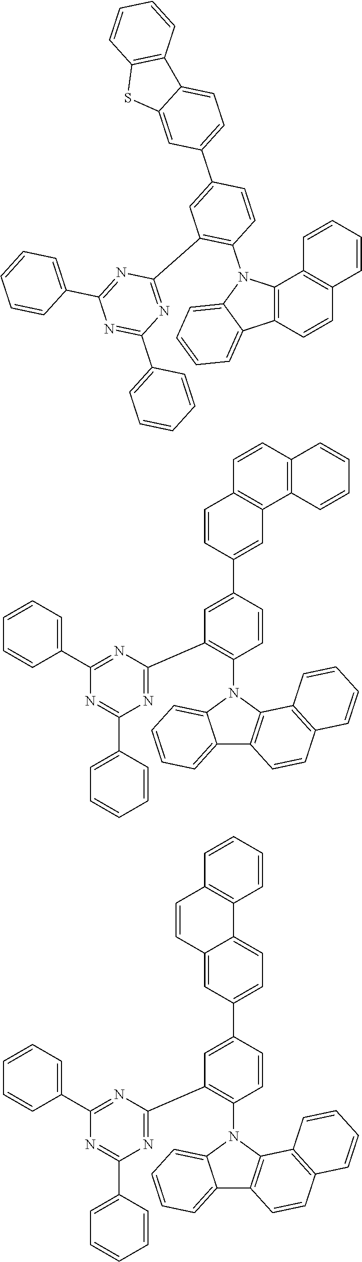 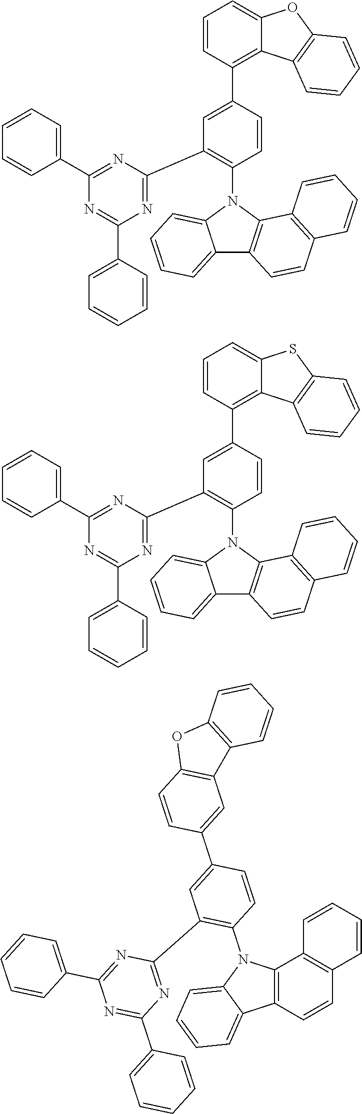 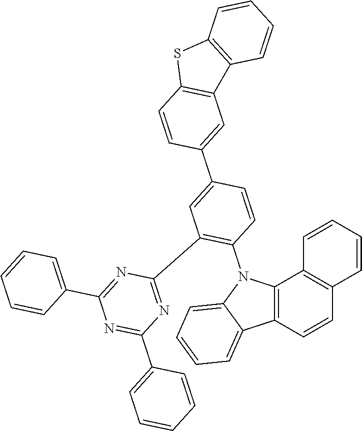 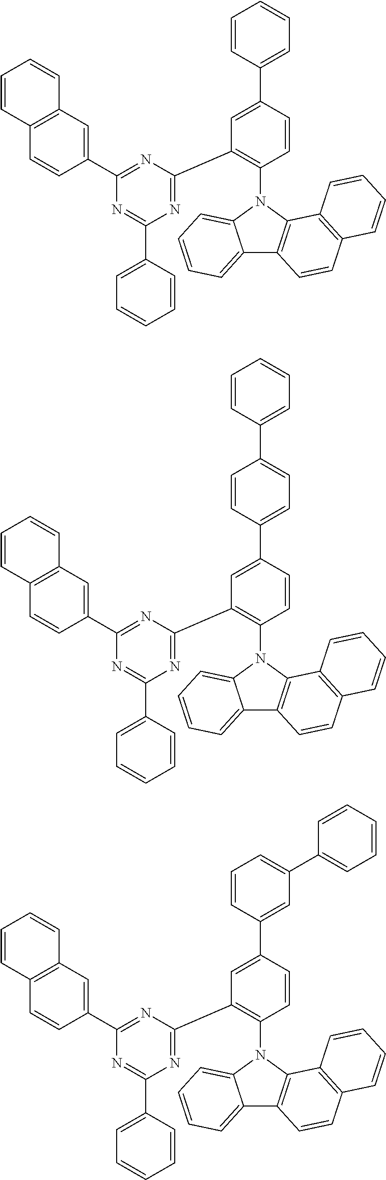 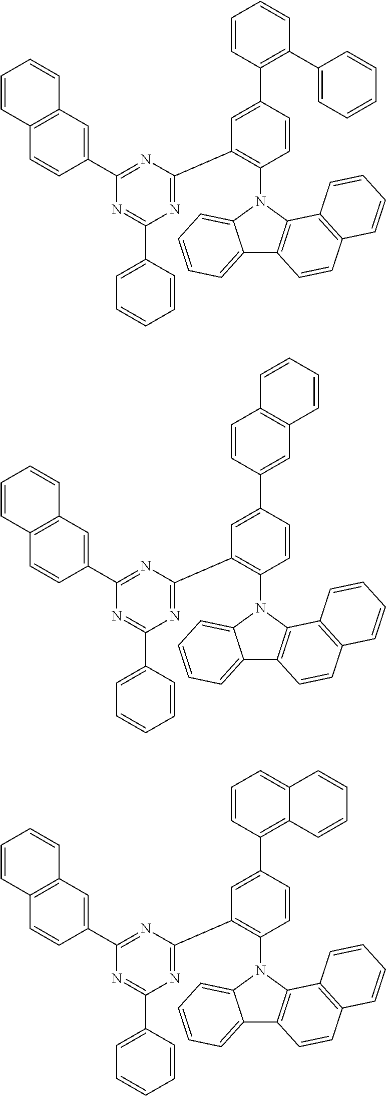 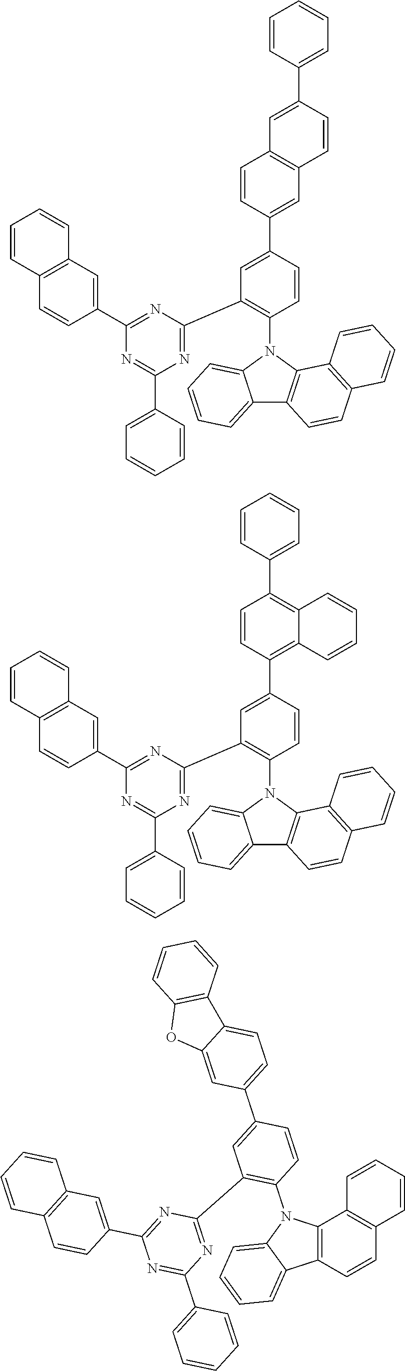 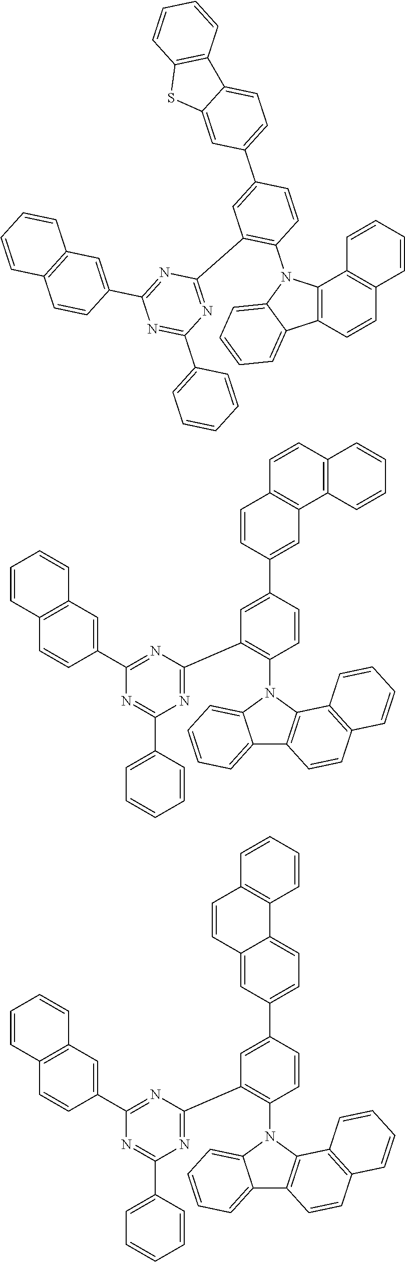 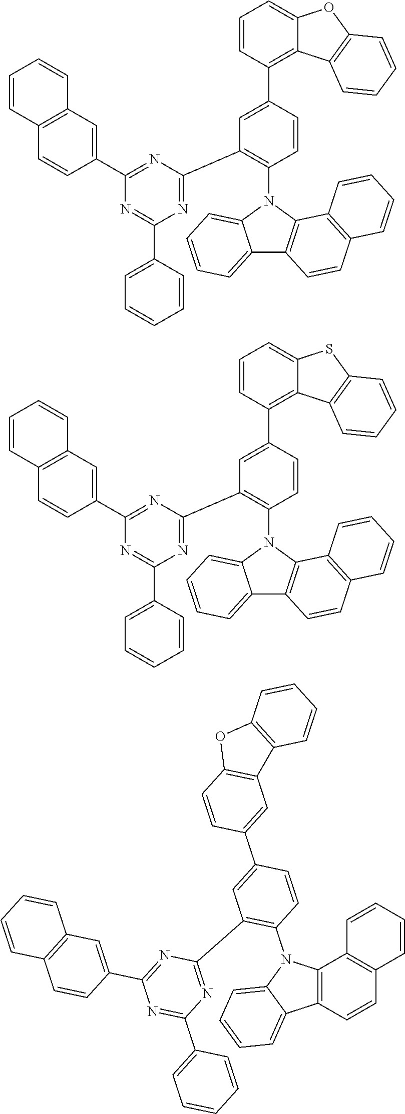 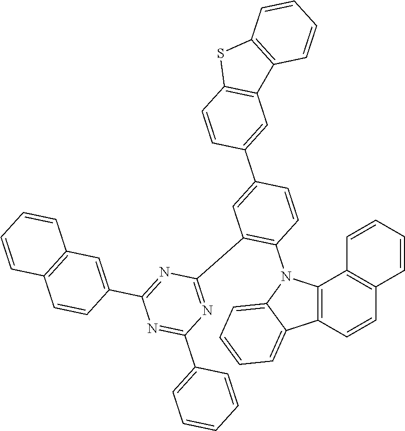 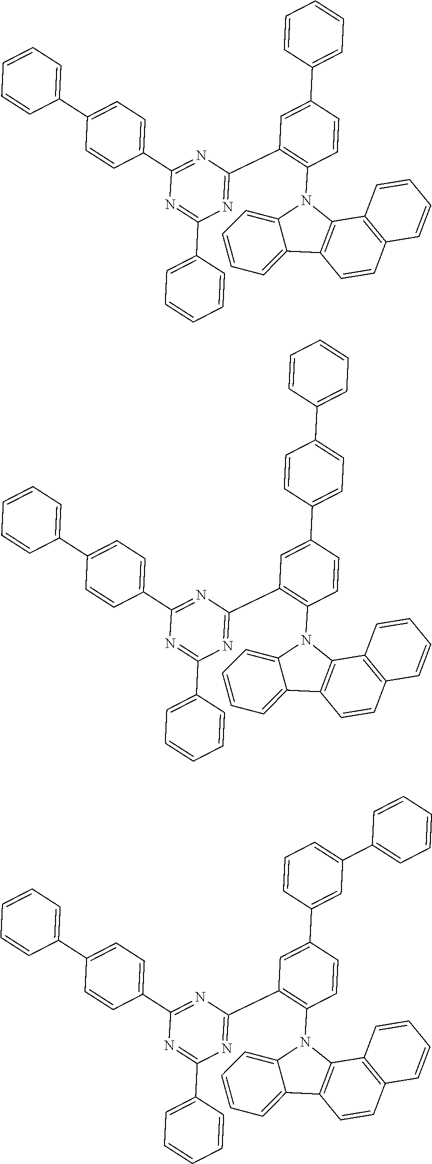 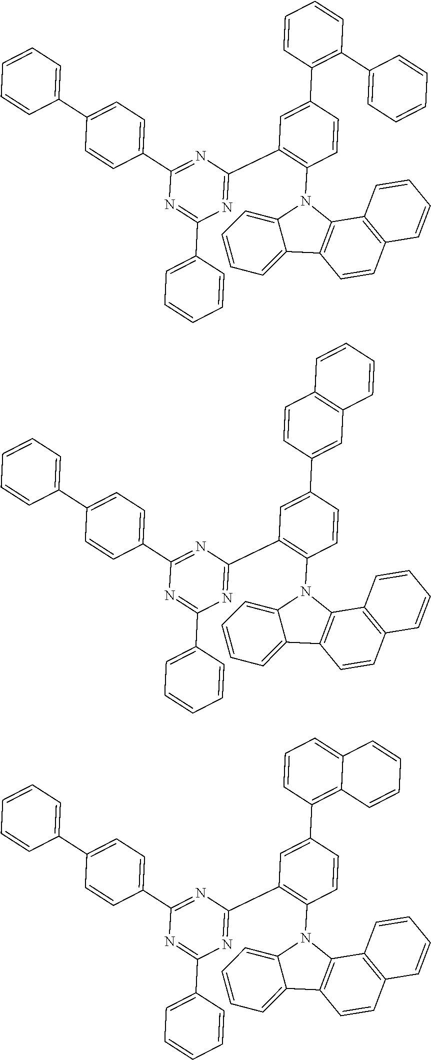 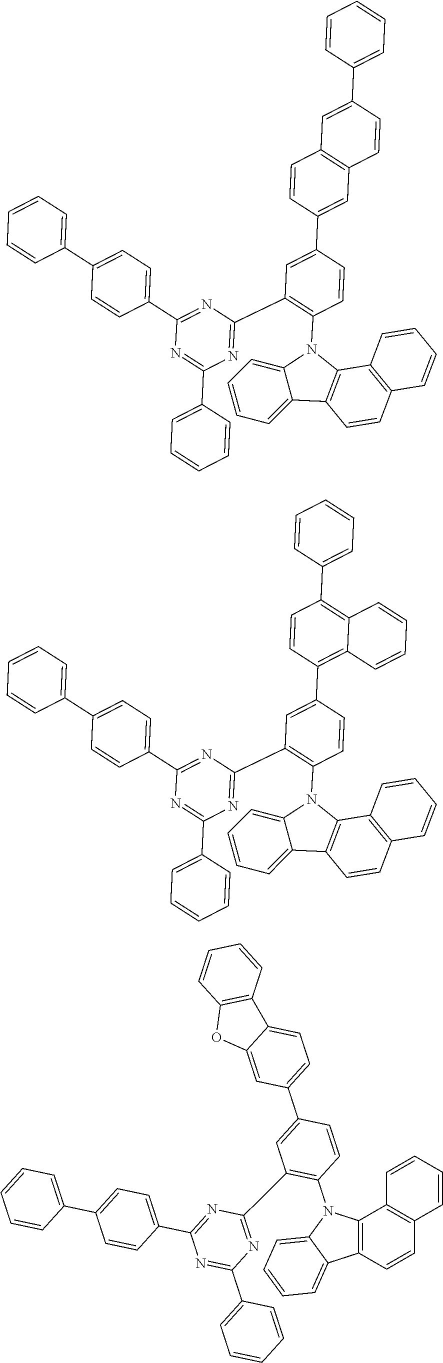 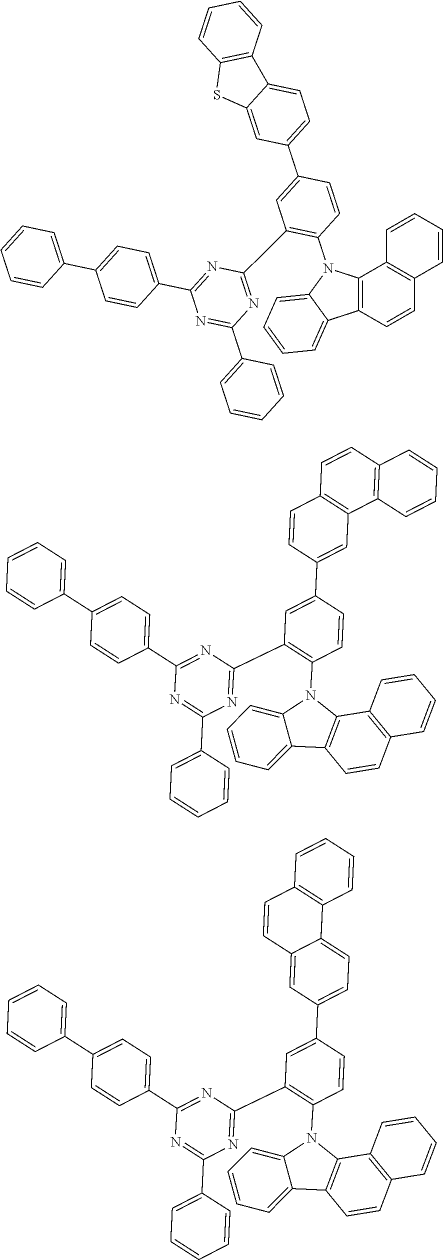 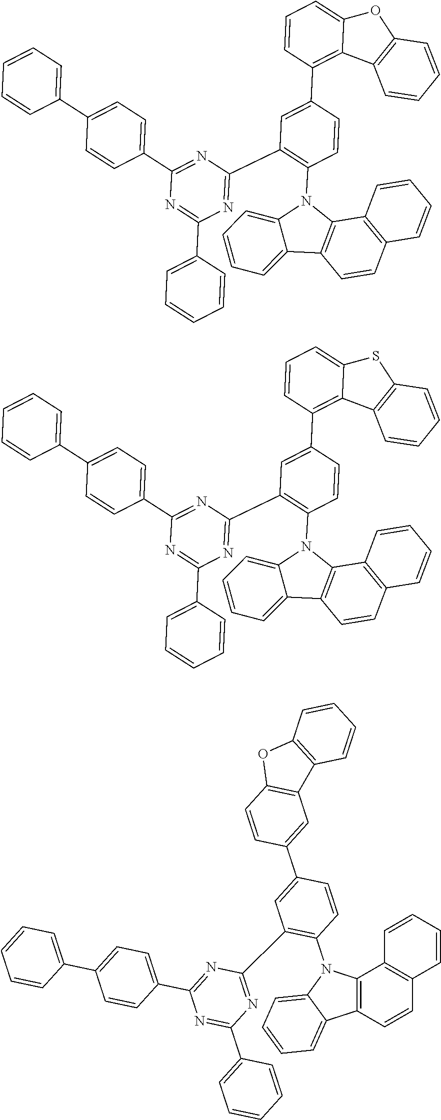 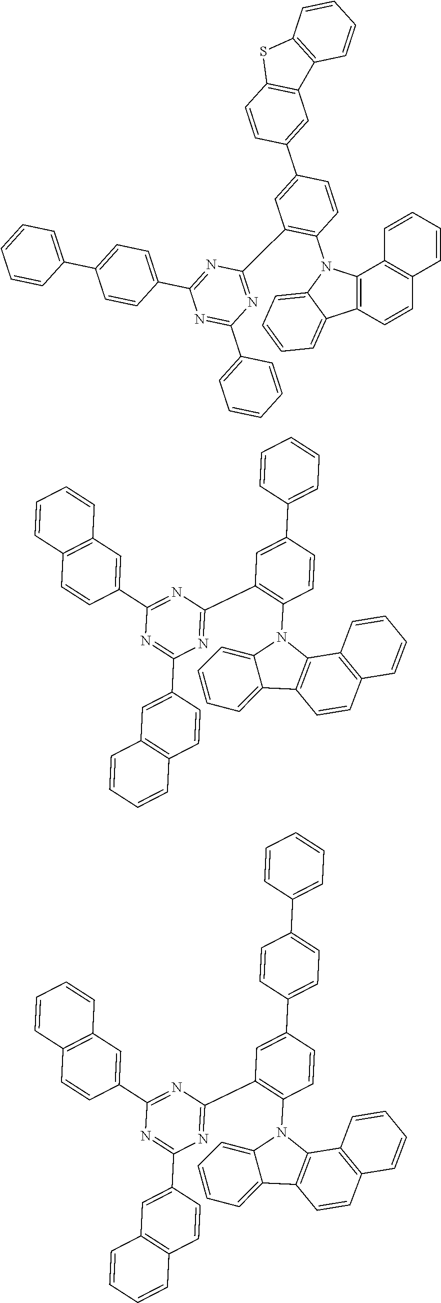 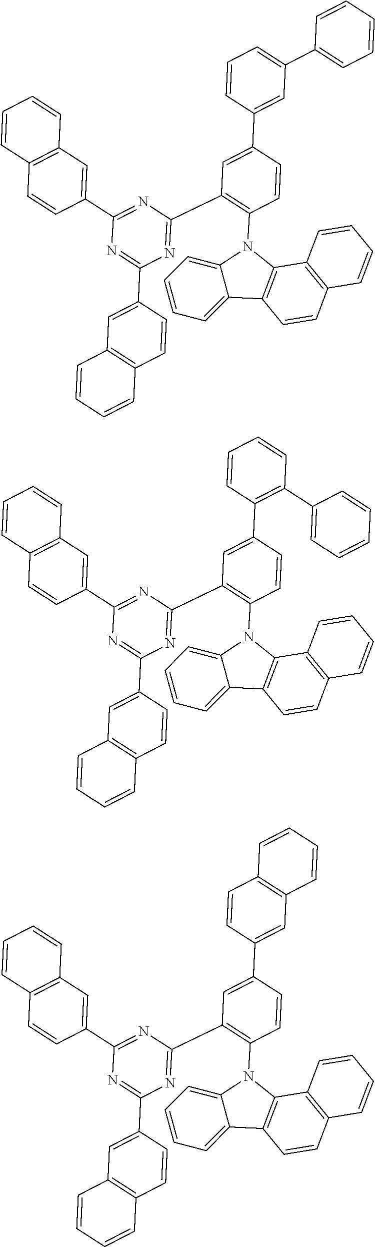 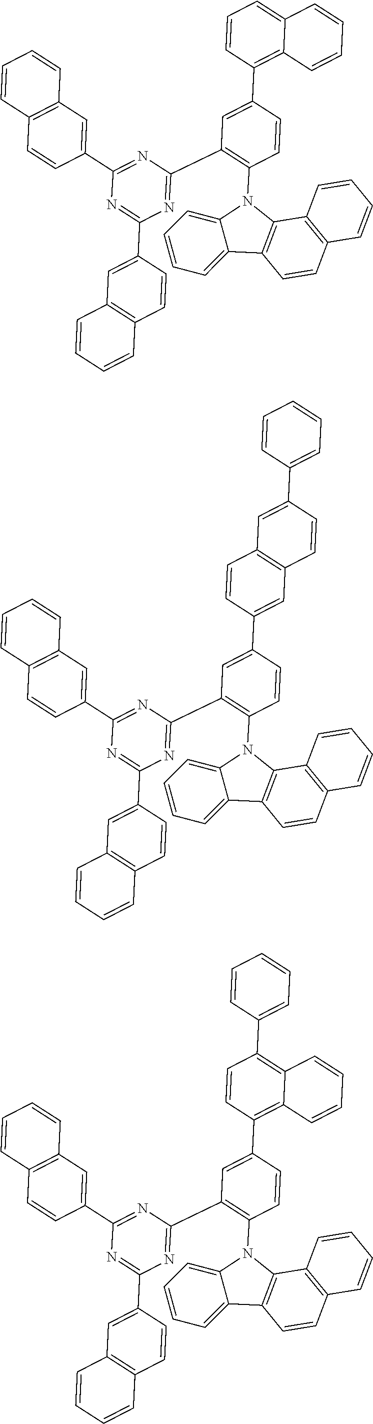 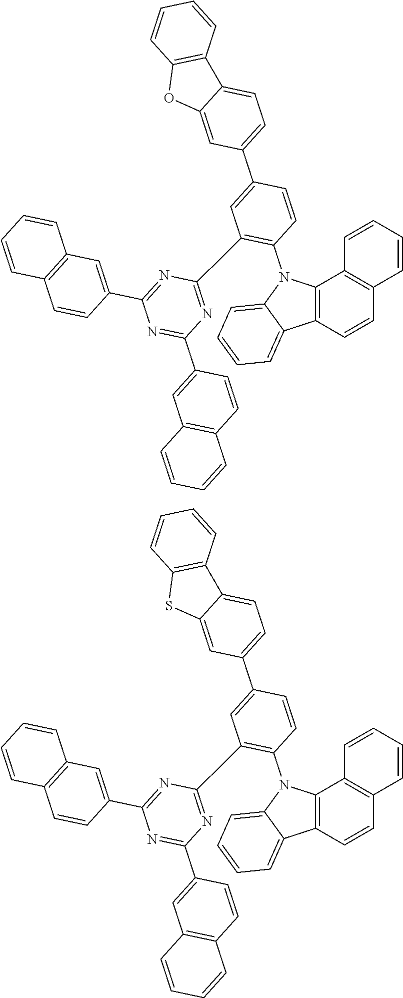 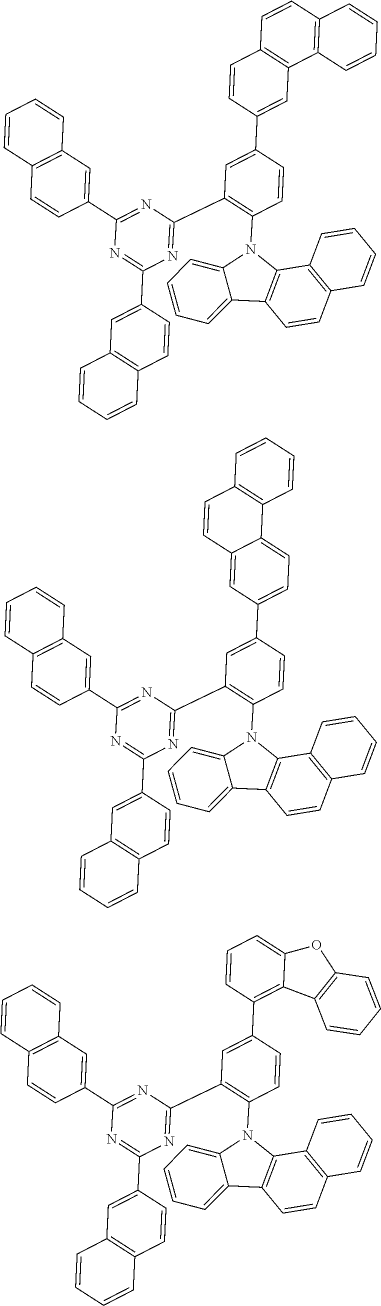 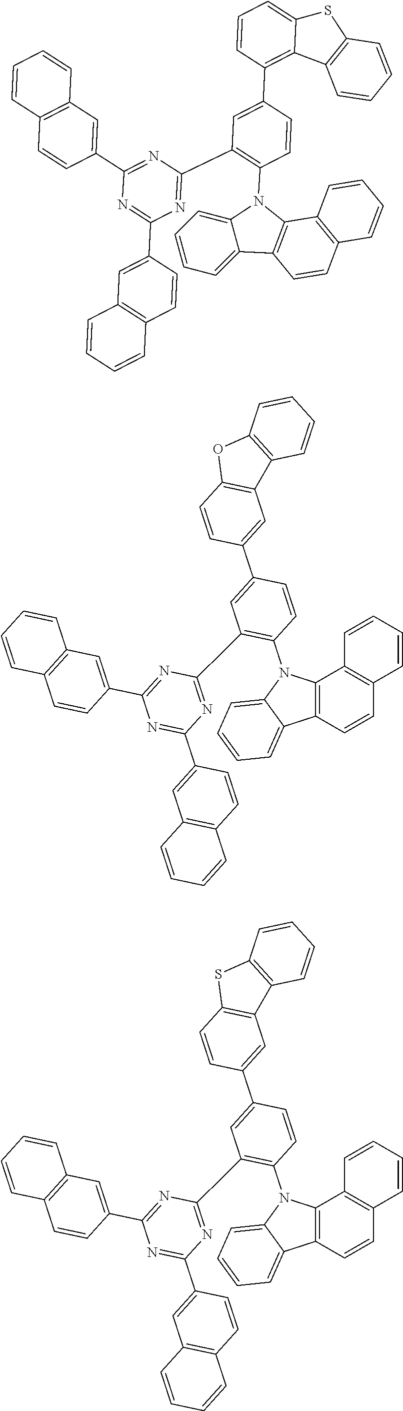 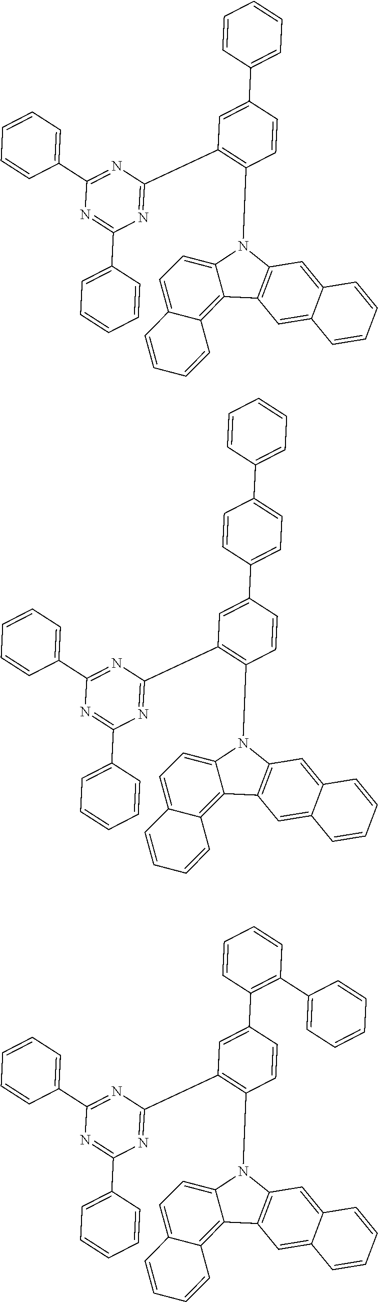 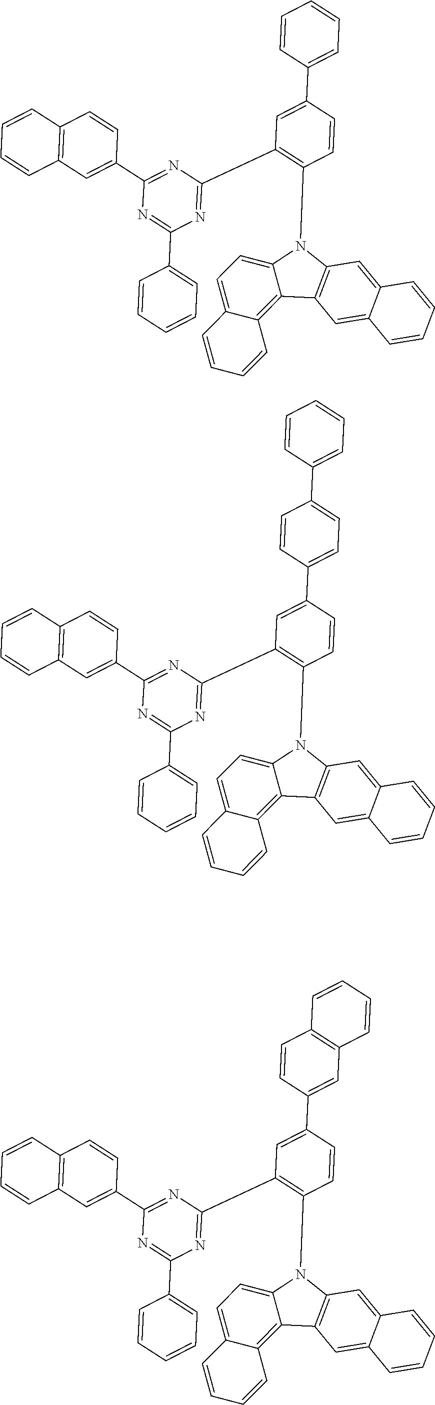 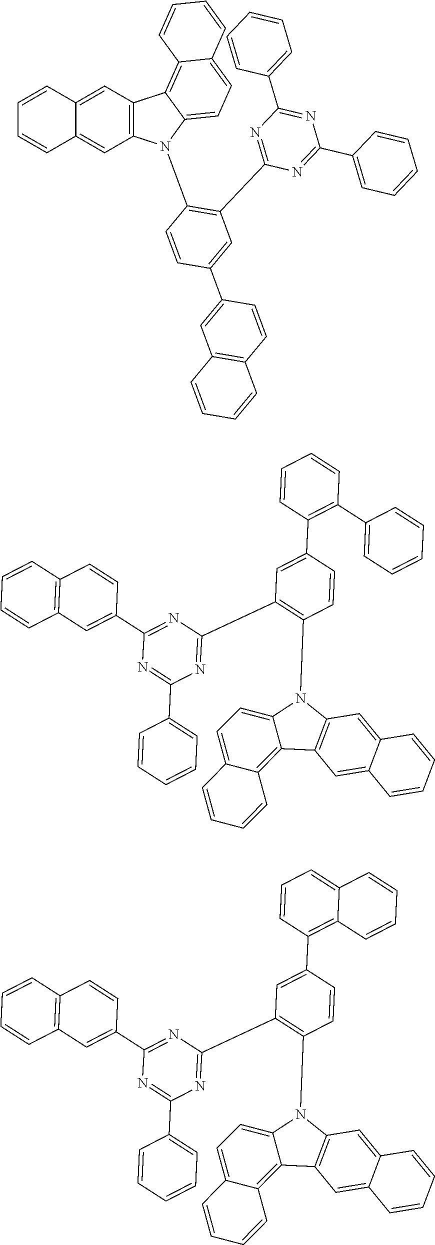 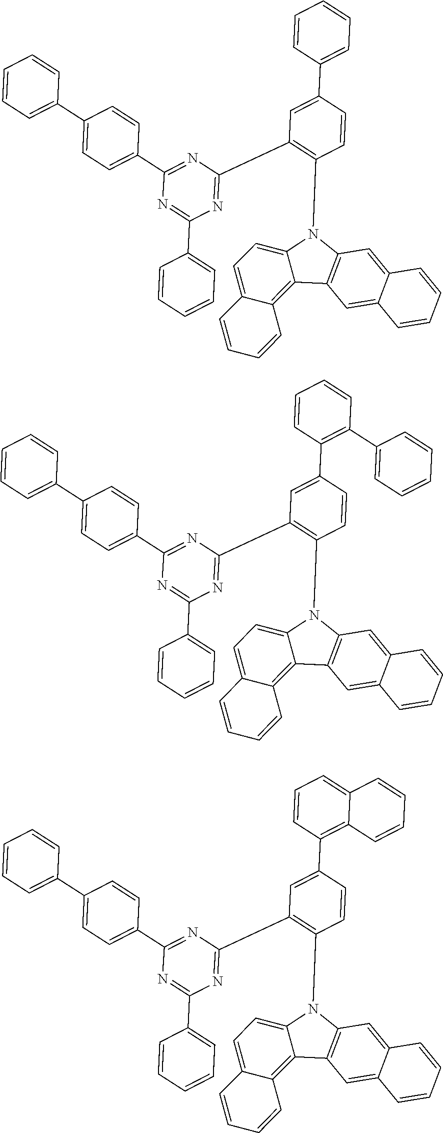 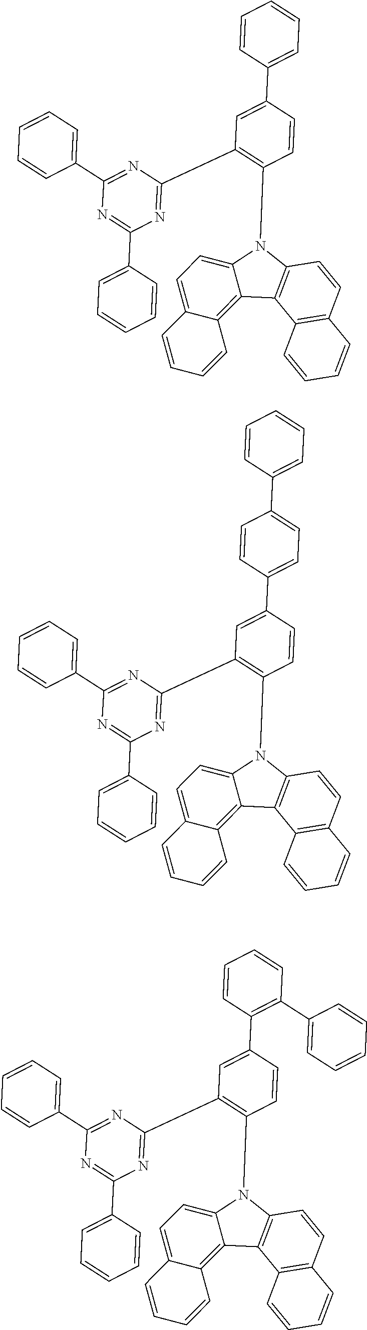 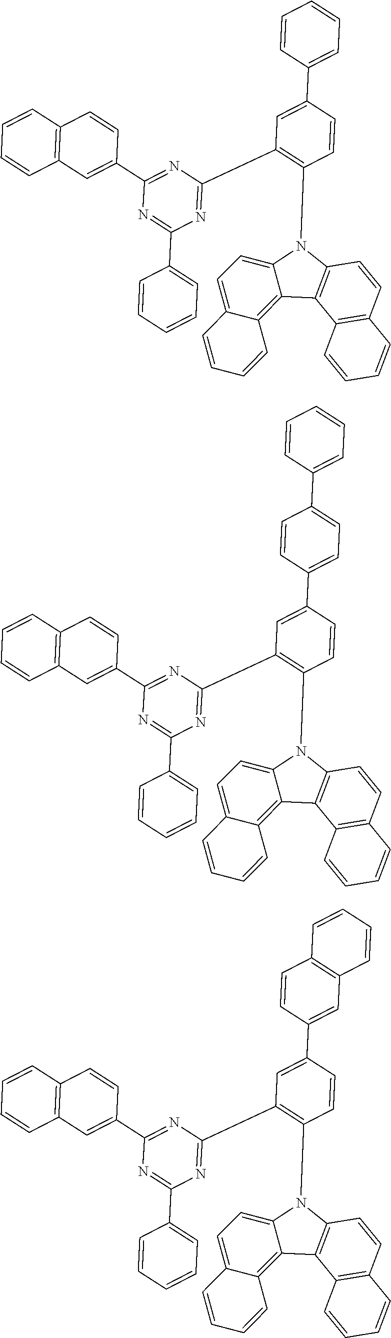 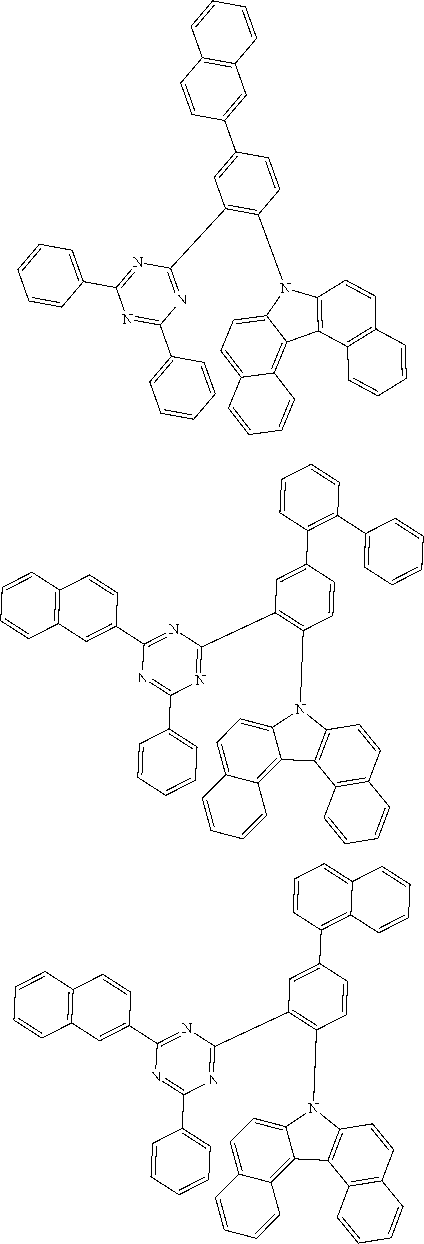 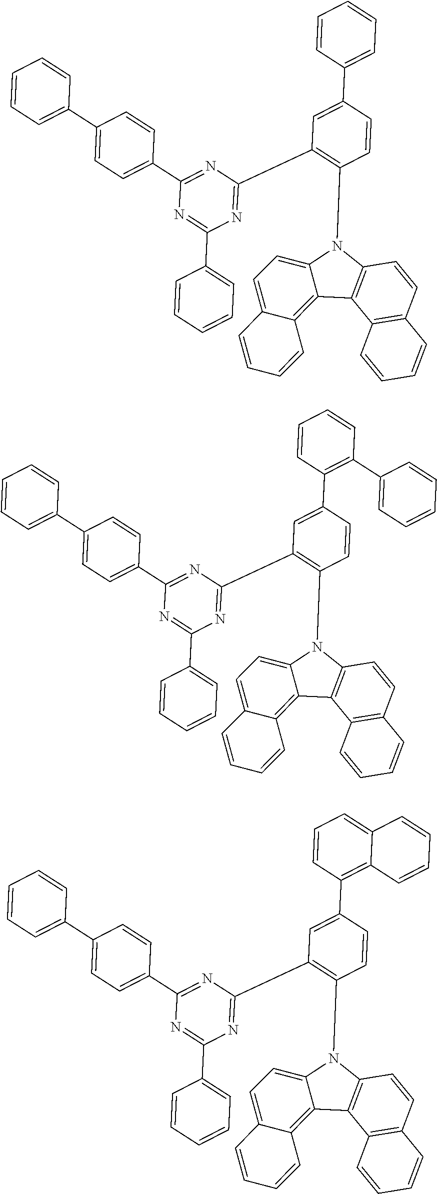 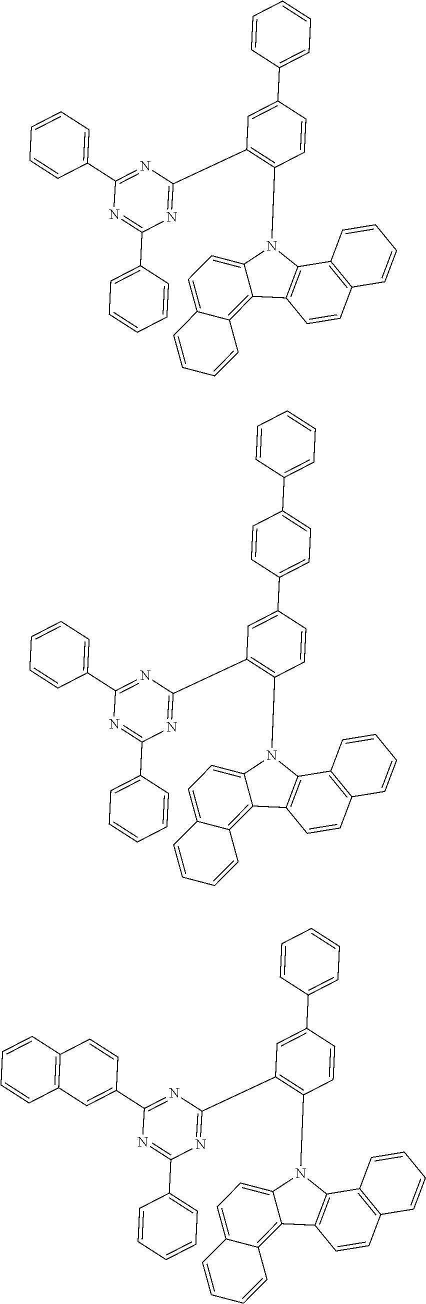 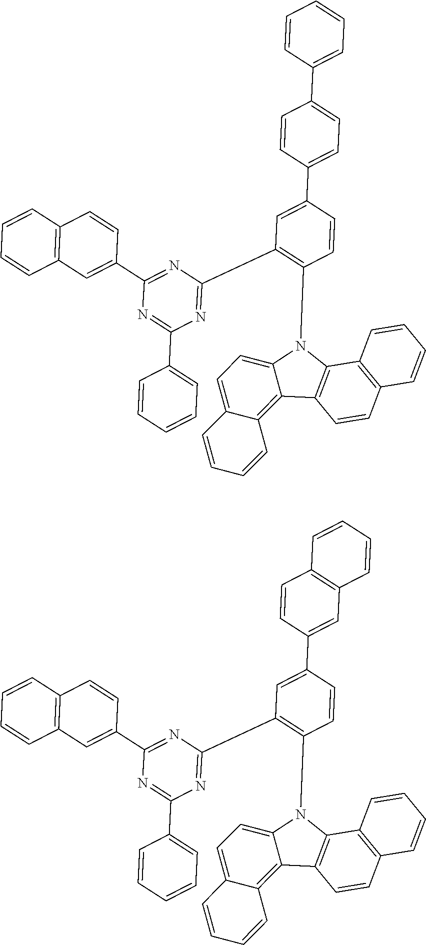 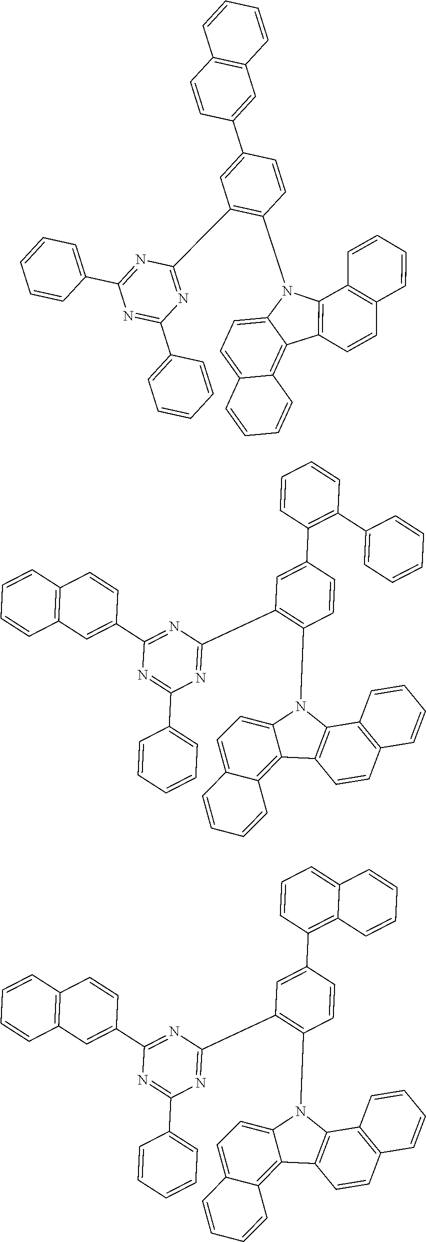 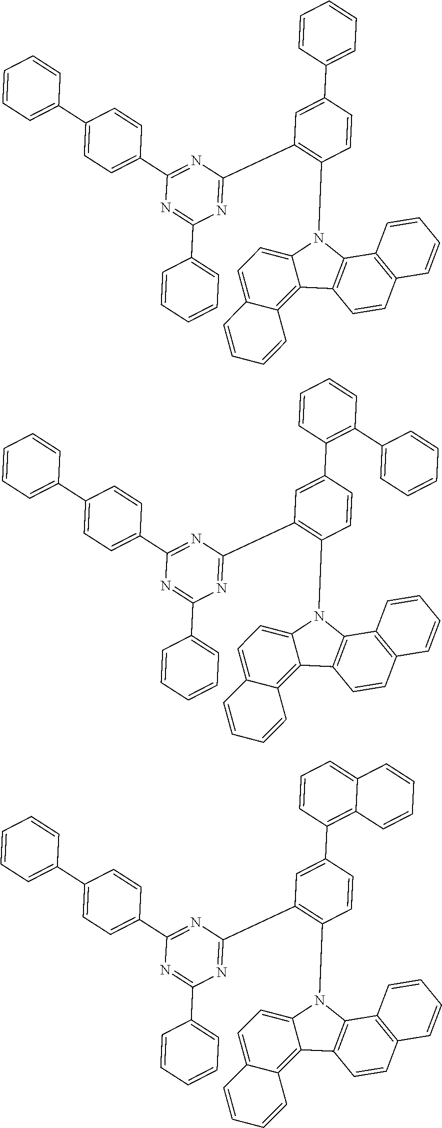 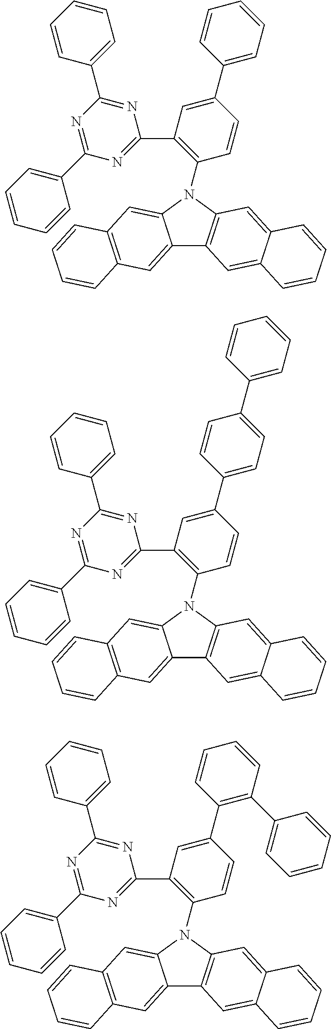 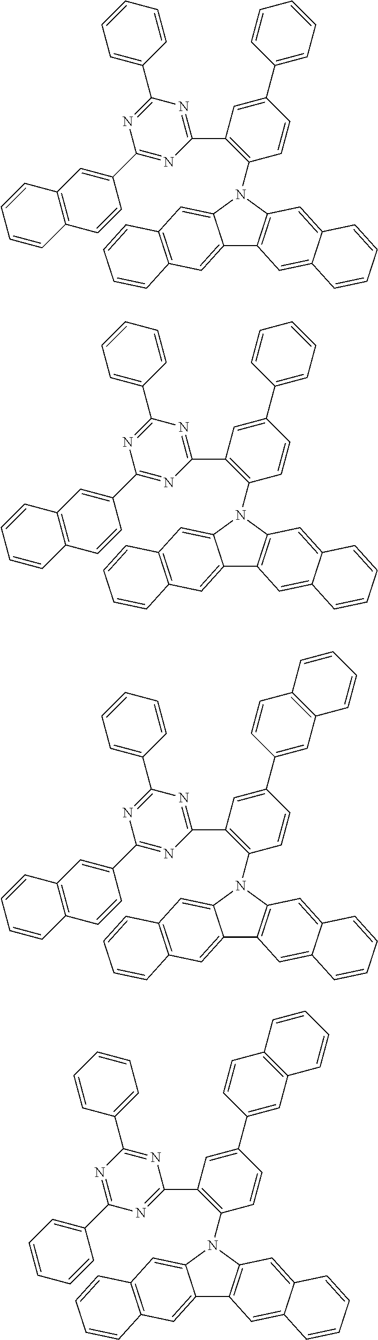 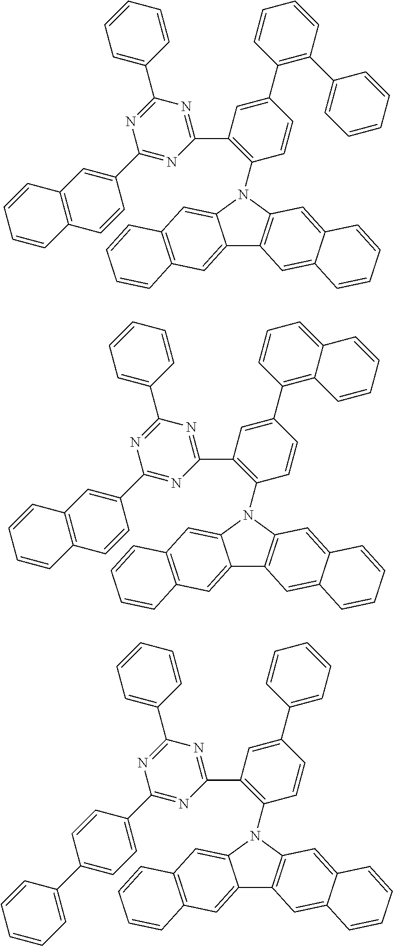 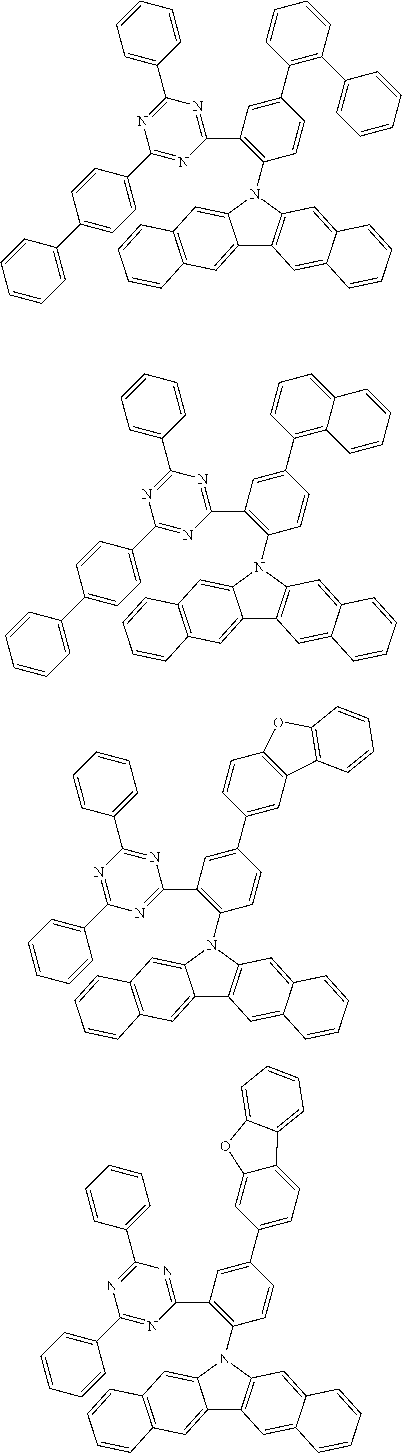 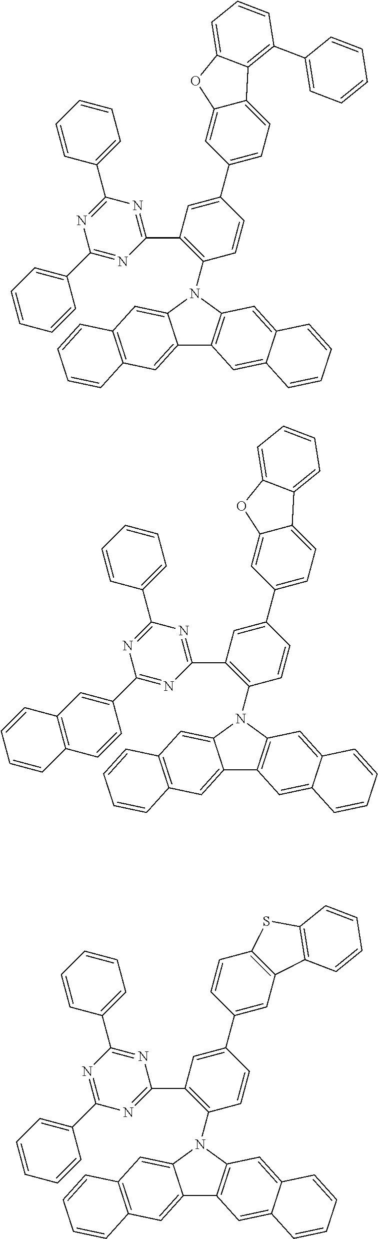 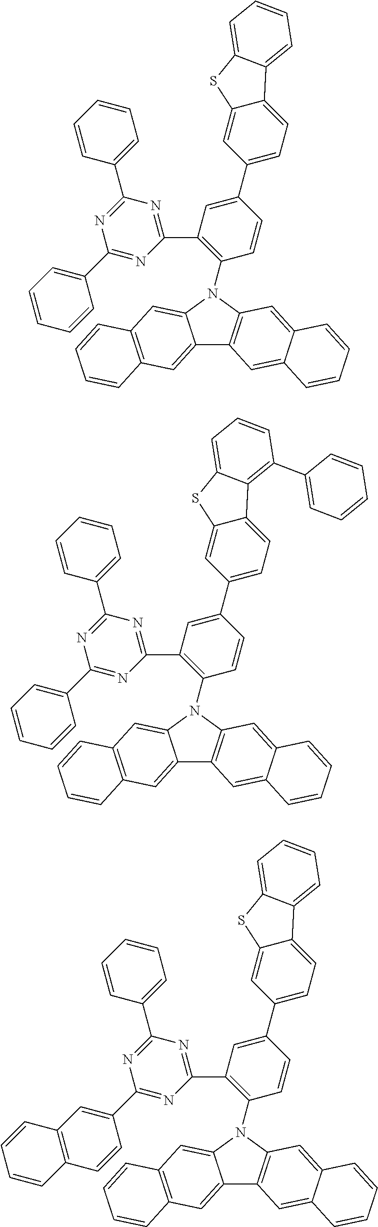 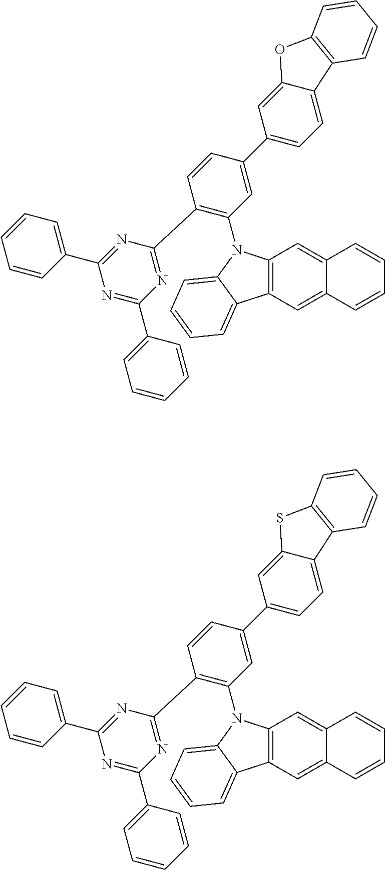 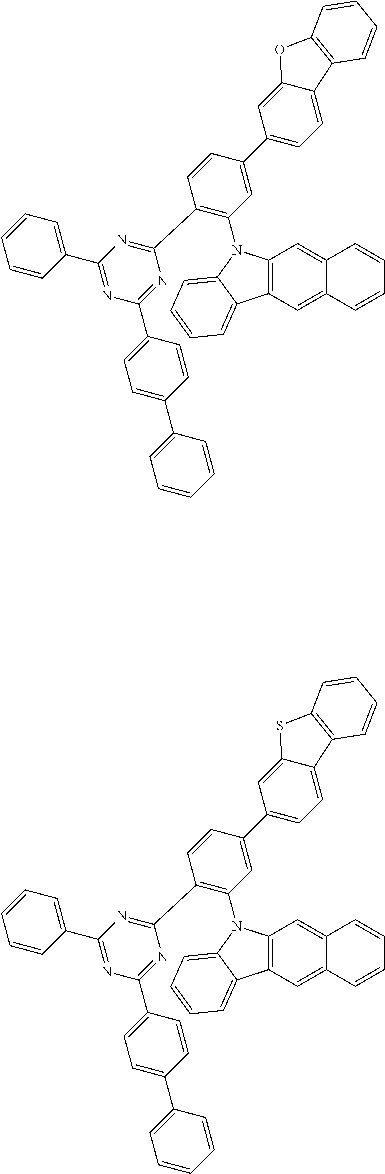 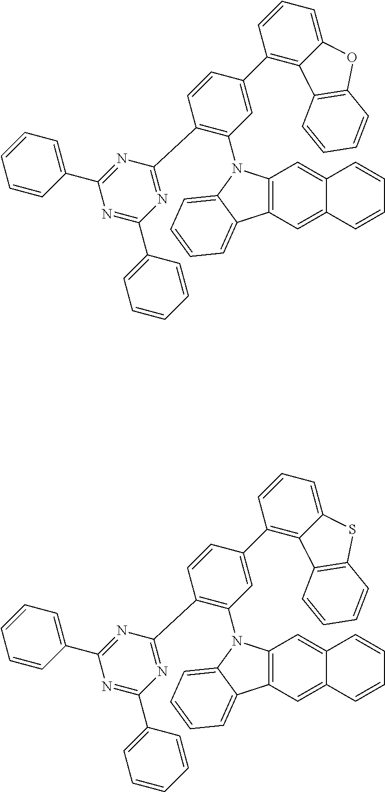 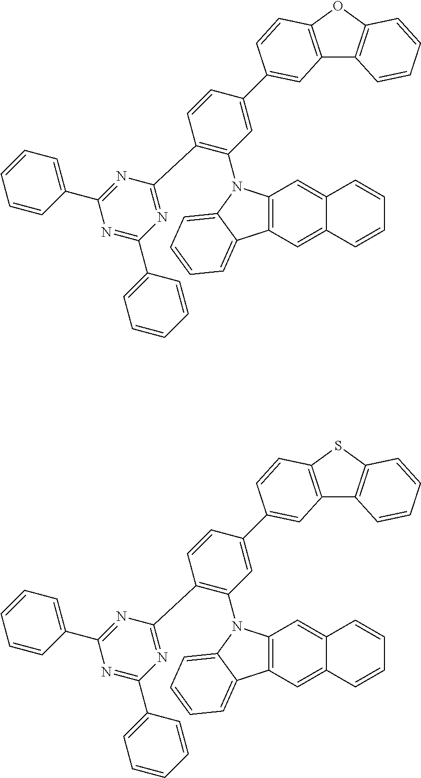 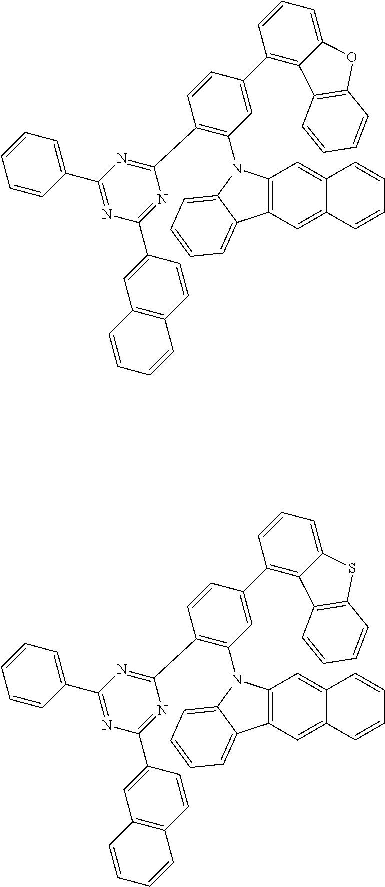 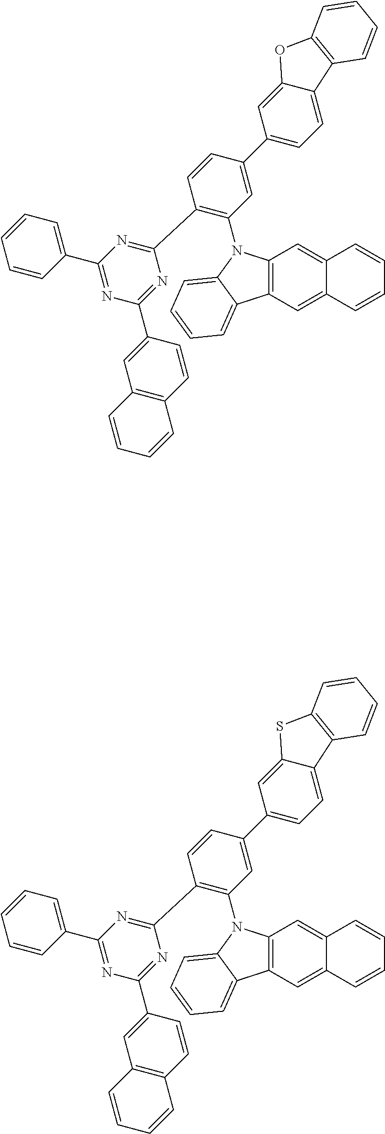 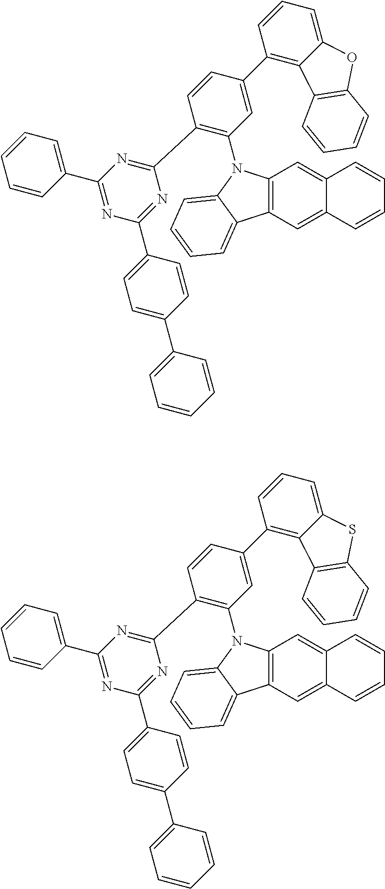 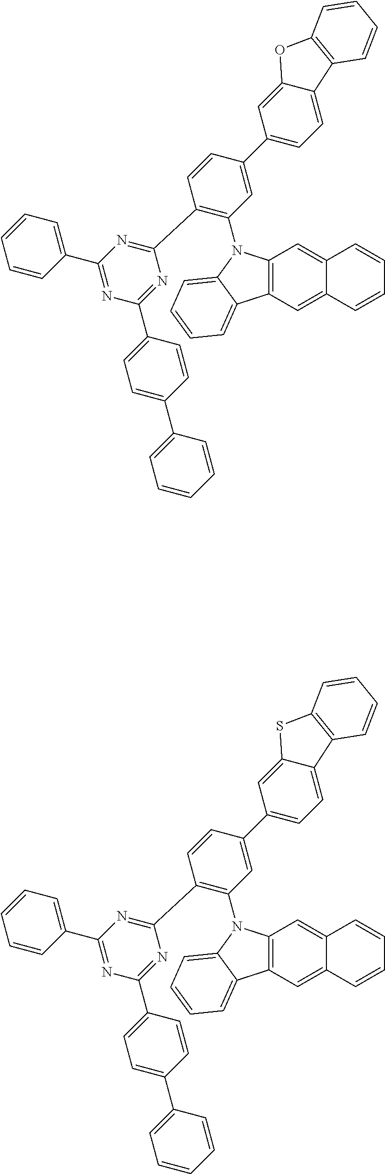 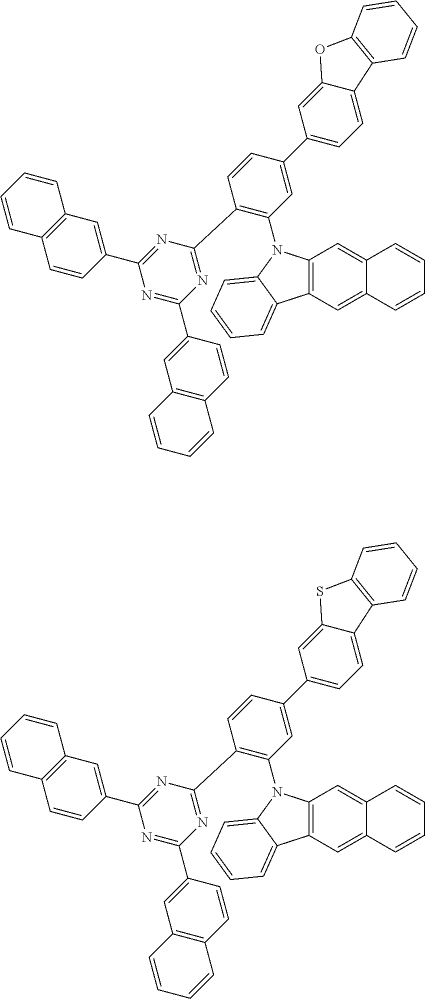 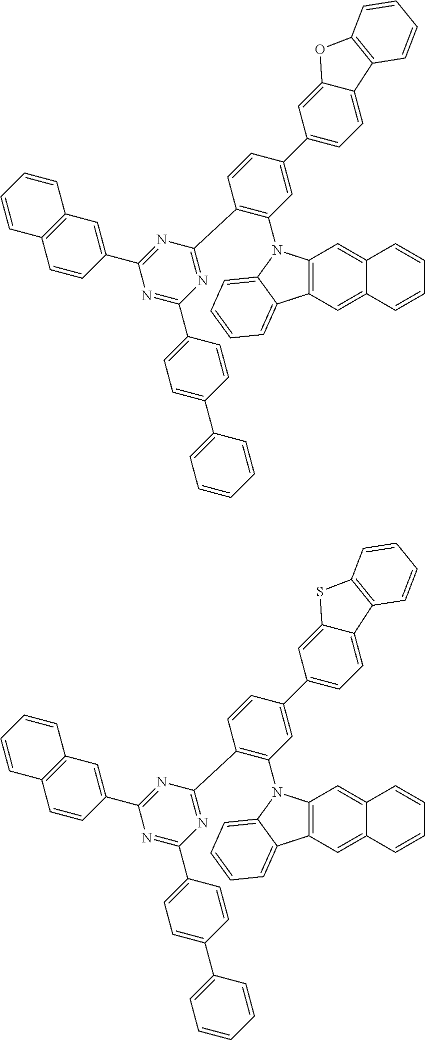 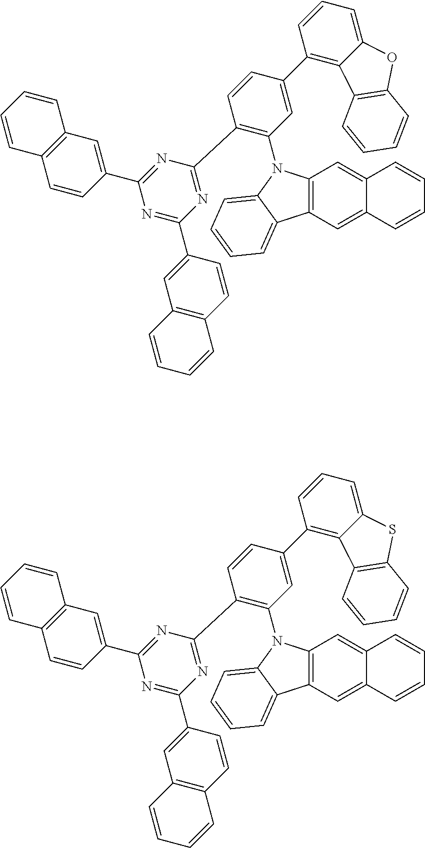 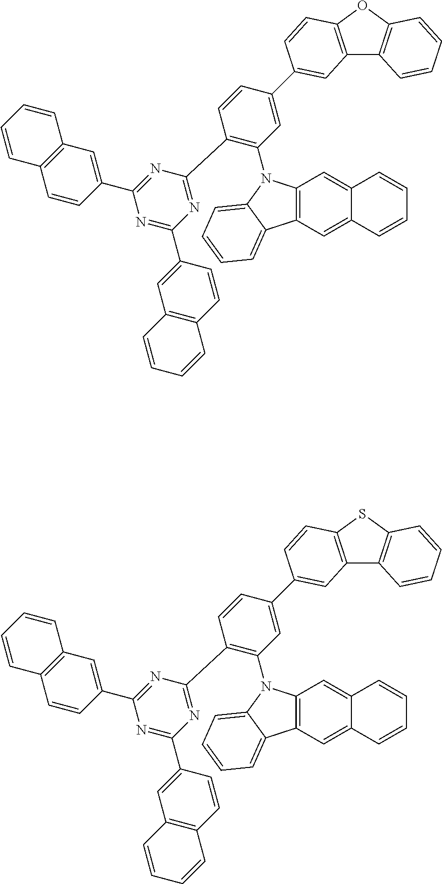 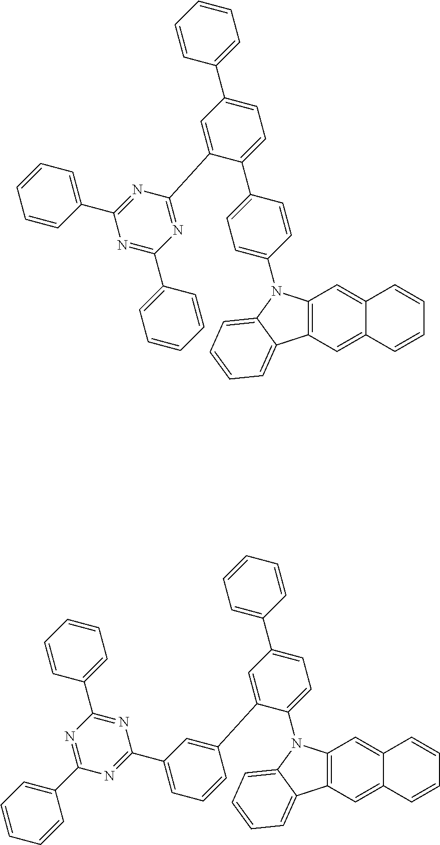 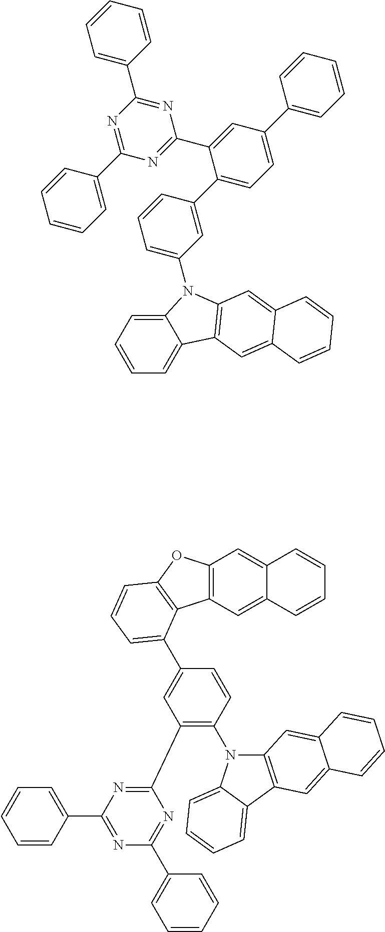 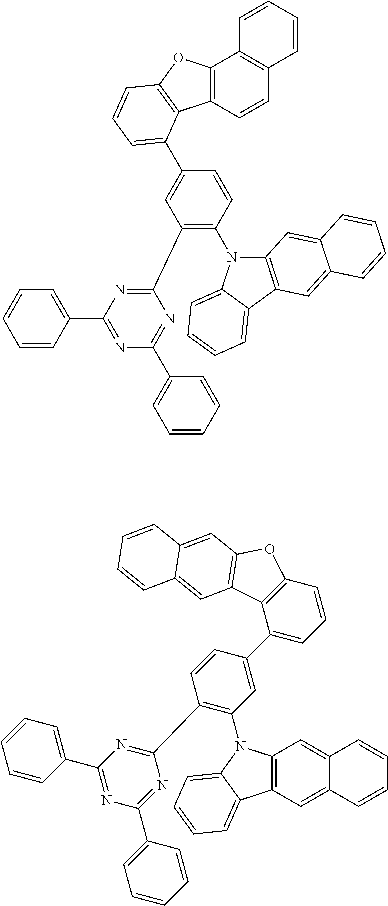 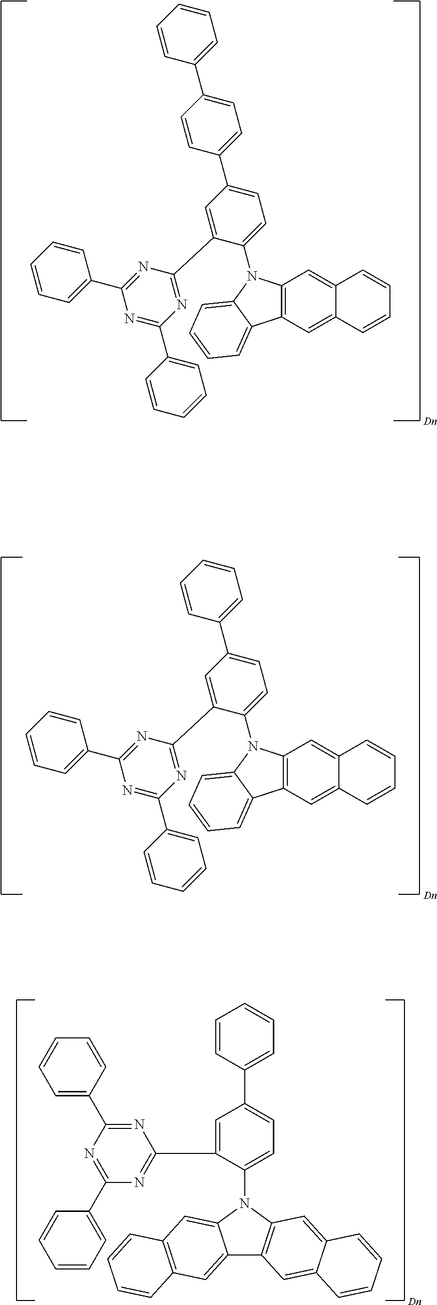 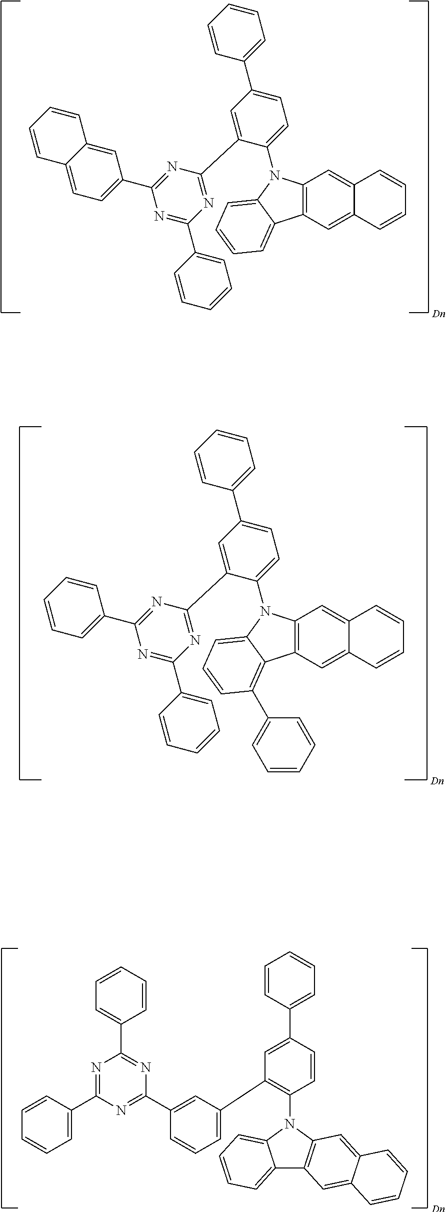 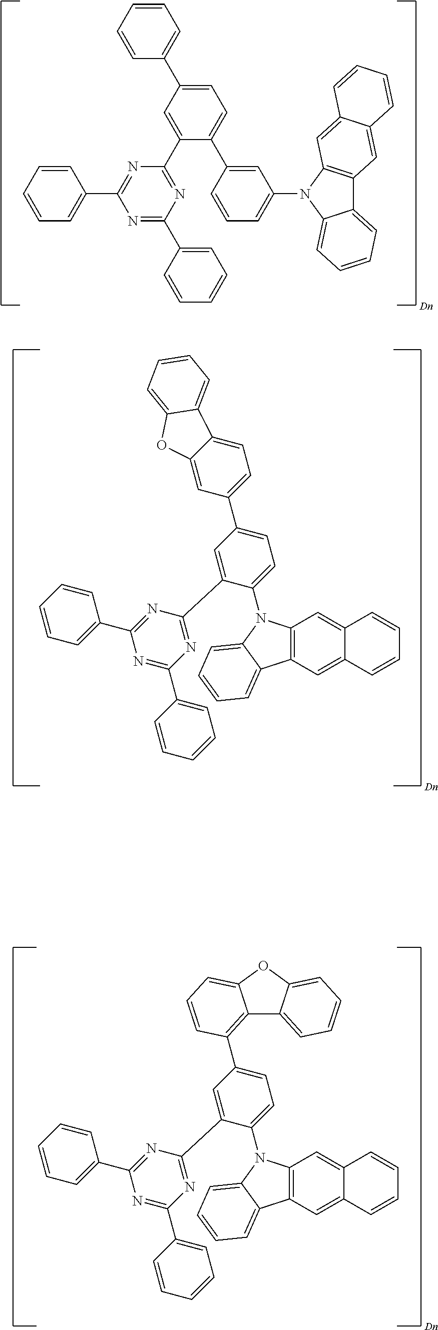 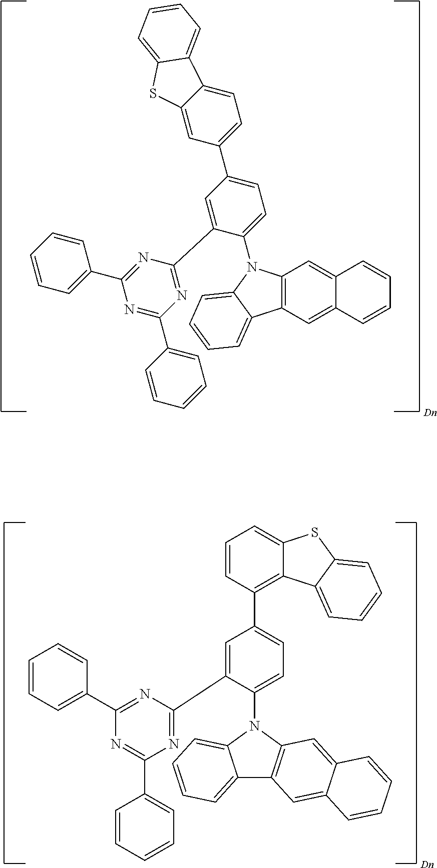
wherein,
Dn means that n hydrogens have been replaced by deuterium,
wherein n is an integer of 1 or more.

15. An organic light emitting device comprising:
a first electrode;
a second electrode that is provided opposite to the first electrode; and
one or more organic material layers that are provided between the first electrode and the second electrode, wherein one or more layers of the organic material layers comprises the compound of claim 1.

16. The organic light emitting device of claim 15, wherein:
the organic material layers comprising the compound are a light emitting layer.

17. A composition for an organic light emitting device, comprising a compound represented by the following Chemical Formula 1 and a compound represented by the following Chemical Formula 2: 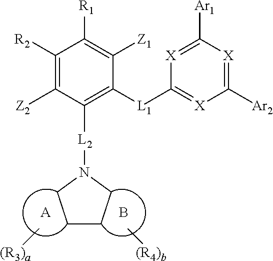
wherein in Chemical Formula 1,
A is a naphthalene ring fused with a neighboring pentagonal ring,
B is a C₆₋₂₀ aromatic ring fused with a neighboring pentagonal ring,
X are each independently N or CR, provided that at least one of X is N,
R is hydrogen, deuterium, or tritium,
R₁ is hydrogen, deuterium, tritium, substituted or unsubstituted C₆₋₆₀ aryl, or substituted or unsubstituted C₂₋₆₀ heteroaryl comprising O or S, but is not 4-dibenzofuranyl and 4-dibenzothiophenyl,
R₂ is hydrogen, deuterium, tritium, or substituted or unsubstituted C₂₋₆₀ heteroaryl comprising O or S, but is not 4-dibenzofuranyl and 4-dibenzothiophenyl,
provided that the case where R₁ and R₂ are simultaneously hydrogen, deuterium, or tritium is excluded,
Z₁ and Z₂ are each independently hydrogen, deuterium, or tritium,
L₁ and L₂ are each independently a single bond, substituted or unsubstituted C₁₋₆₀ alkylene, substituted or unsubstituted C₆₋₆₀ arylene, or substituted or unsubstituted C₂₋₆₀ heteroarylene comprising one or more heteroatoms among N, O and S,
Ar₁ and Ar₂ are each independently substituted or unsubstituted C₁₋₆₀ alkyl, substituted or unsubstituted C₆₋₆₀ aryl, or substituted or unsubstituted C₂₋₆₀ heteroaryl comprising one or more heteroatoms among N, O and S,
R₃ and R₄ are each independently hydrogen, deuterium, tritium, substituted or unsubstituted C₁₋₆₀ alkyl, or substituted or unsubstituted C₆₋₆₀ aryl,
a is an integer from 0 to 6, and
b is an integer from 0 to 10, and 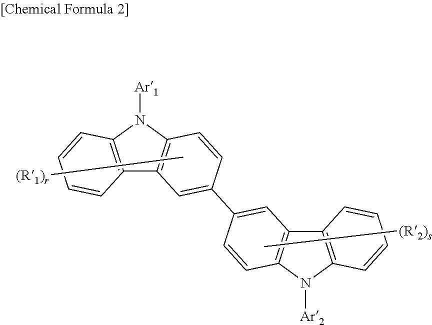
wherein in Chemical Formula 2,
Ar′₁ and Ar′₂ are each independently substituted or unsubstituted C₆₋₆₀ aryl, or substituted or unsubstituted C₂₋₆₀ heteroaryl comprising one or more heteroatoms among N, O and S,
R′₁ and R′₂ are each independently hydrogen, deuterium, C₁₋₆₀ alkyl, C₆₋₆₀ aryl, or C₂₋₆₀ heteroaryl comprising one or more heteroatoms among N, O and S, and
r and s are each independently an integer from 0 to 7, and
the “substituted or unsubstituted” means being unsubstituted or substituted with one or more substituents selected from the group consisting of deuterium, tritium, halogen, nitro, amino, alkyl, and aryl, or being unsubstituted or substituted with a substituent to which two or more substituents of the above-exemplified substituents are connected.

18. The composition for an organic light emitting device of claim 17, wherein:
the compound represented by the Chemical formula 2 is any one selected from the group consisting of the following compounds and compounds substituted with deuterium thereof:

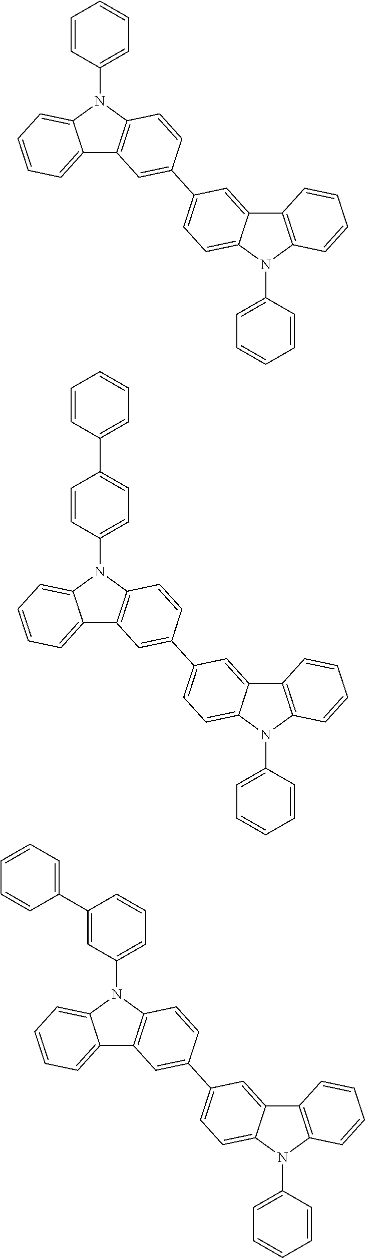 [SMILES: c1ccc(-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cccc(-n3c4ccccc4c4cc(-c5ccc6c(c5)c5ccccc5n6-c5ccccc5)ccc43)c2)cc1]

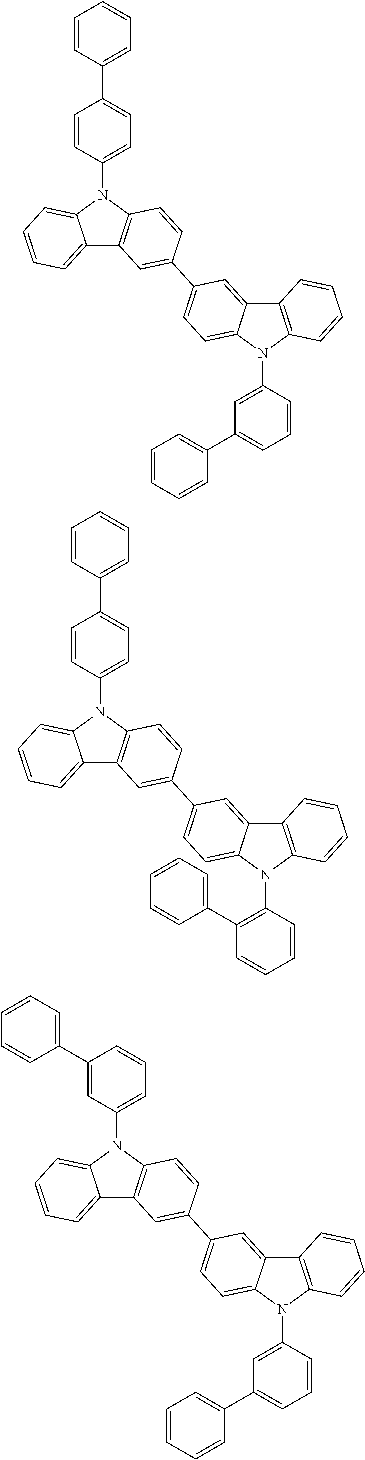 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5-c5ccccc5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1]

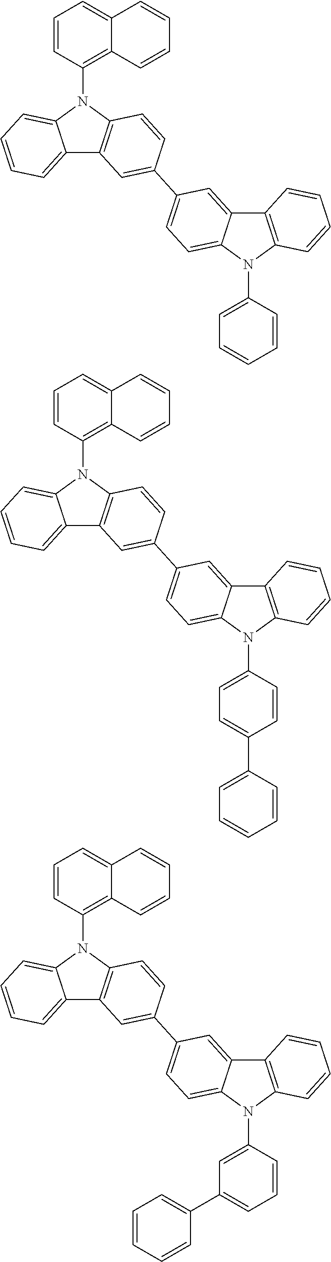 [SMILES: c1ccc(-n2c3ccccc3c3cc(-c4ccc5c(c4)c4ccccc4n5-c4cccc5ccccc45)ccc32)cc1 ; c1ccc(-c2ccc(-n3c4ccccc4c4cc(-c5ccc6c(c5)c5ccccc5n6-c5cccc6ccccc56)ccc43)cc2)cc1 ; c1ccc(-c2cccc(-n3c4ccccc4c4cc(-c5ccc6c(c5)c5ccccc5n6-c5cccc6ccccc56)ccc43)c2)cc1]

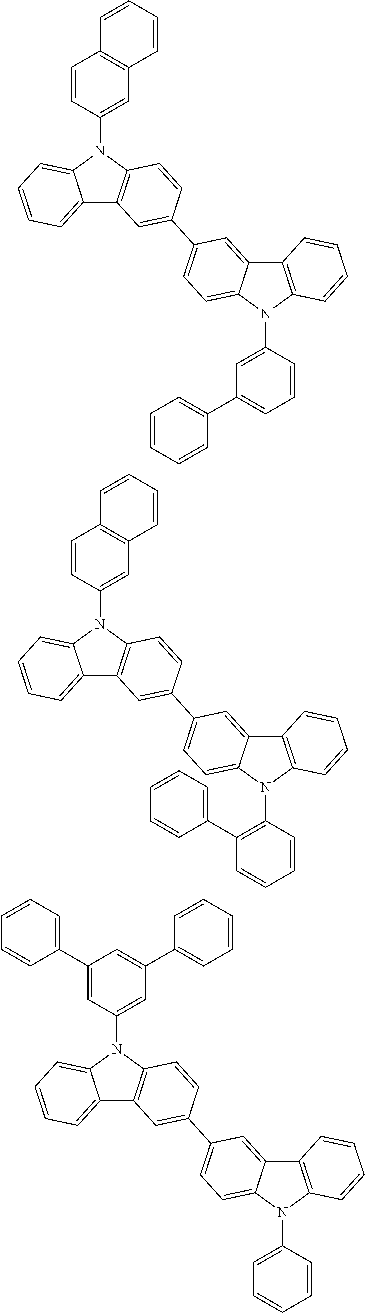

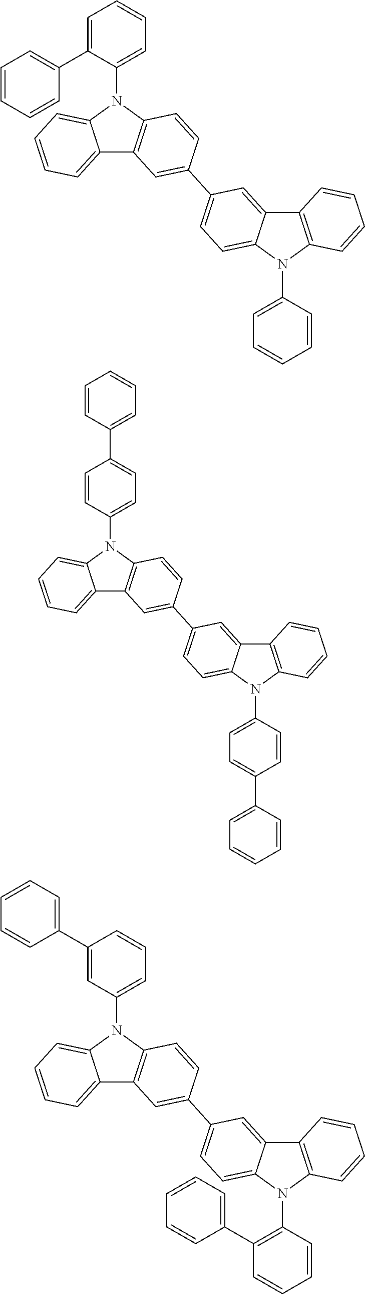 [SMILES: c1ccc(-c2ccccc2-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2cccc(-n3c4ccccc4c4cc(-c5ccc6c(c5)c5ccccc5n6-c5ccccc5-c5ccccc5)ccc43)c2)cc1]

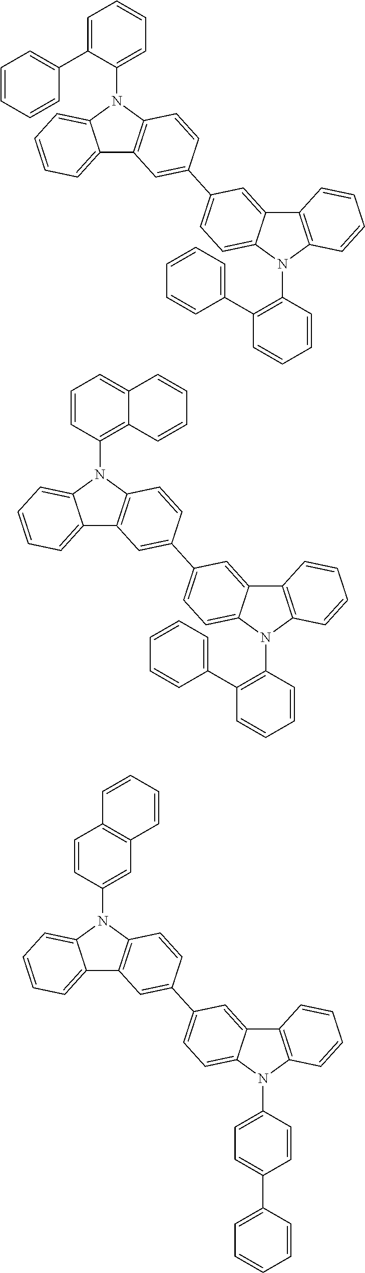 [SMILES: c1ccc(-c2ccccc2-n2c3ccccc3c3cc(-c4ccc5c(c4)c4ccccc4n5-c4ccccc4-c4ccccc4)ccc32)cc1 ; c1ccc(-c2ccccc2-n2c3ccccc3c3cc(-c4ccc5c(c4)c4ccccc4n5-c4cccc5ccccc45)ccc32)cc1 ; c1ccc(-c2ccc(-n3c4ccccc4c4cc(-c5ccc6c(c5)c5ccccc5n6-c5ccc6ccccc6c5)ccc43)cc2)cc1]

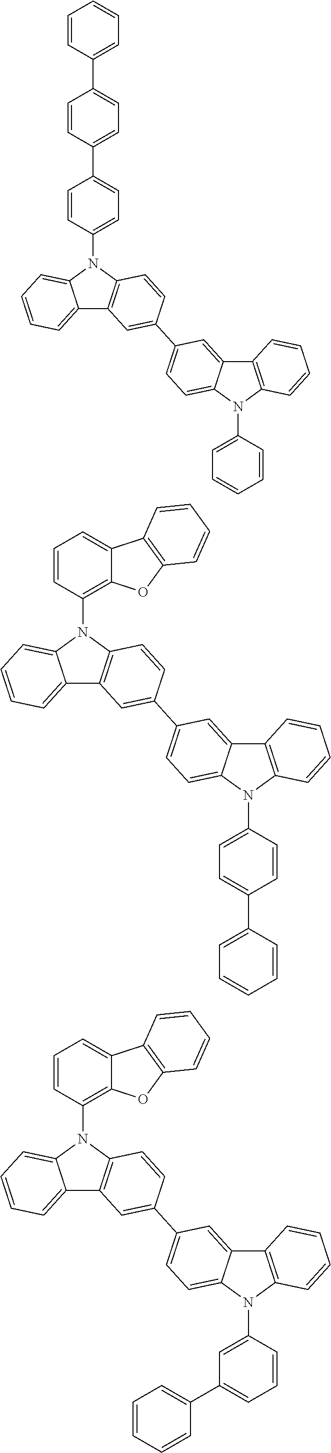

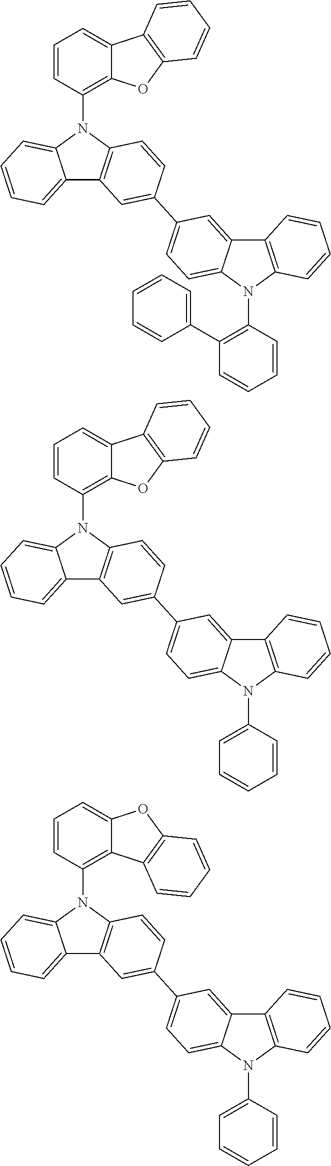

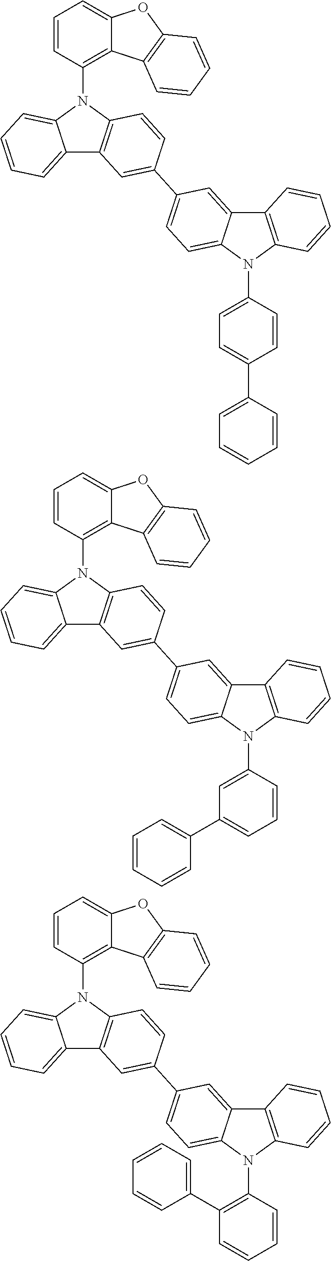

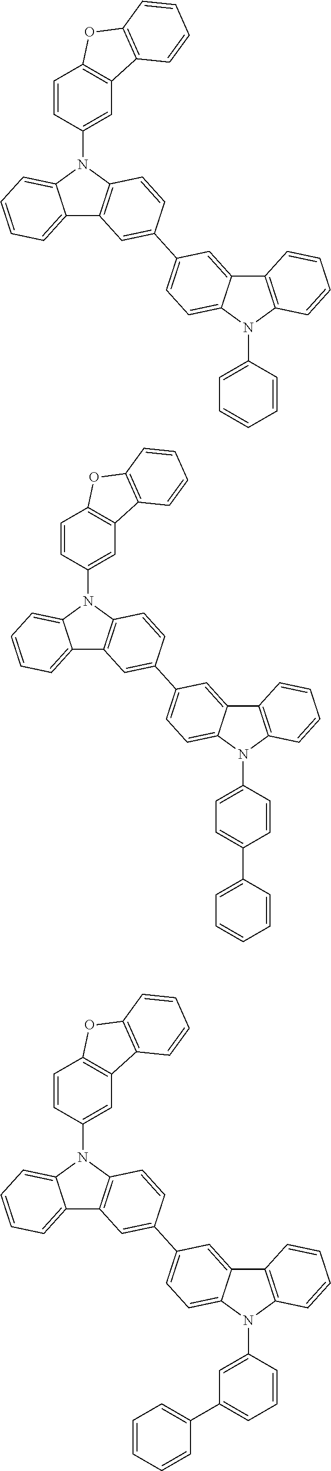 [SMILES: c1ccc(-n2c3ccccc3c3cc(-c4ccc5c(c4)c4ccccc4n5-c4ccc5oc6ccccc6c5c4)ccc32)cc1 ; c1ccc(-c2ccc(-n3c4ccccc4c4cc(-c5ccc6c(c5)c5ccccc5n6-c5ccc6oc7ccccc7c6c5)ccc43)cc2)cc1 ; c1ccc(-c2cccc(-n3c4ccccc4c4cc(-c5ccc6c(c5)c5ccccc5n6-c5ccc6oc7ccccc7c6c5)ccc43)c2)cc1]

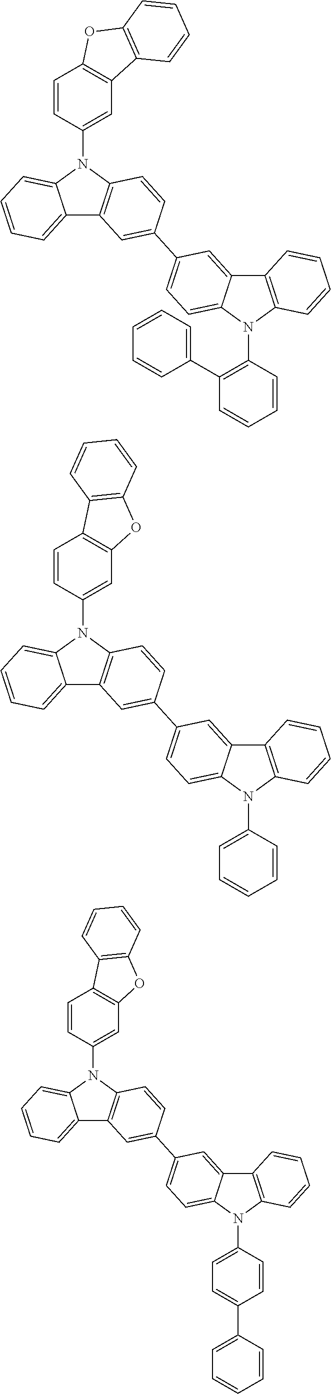 [SMILES: c1ccc(-c2ccccc2-n2c3ccccc3c3cc(-c4ccc5c(c4)c4ccccc4n5-c4ccc5oc6ccccc6c5c4)ccc32)cc1 ; c1ccc(-n2c3ccccc3c3cc(-c4ccc5c(c4)c4ccccc4n5-c4ccc5c(c4)oc4ccccc45)ccc32)cc1 ; c1ccc(-c2ccc(-n3c4ccccc4c4cc(-c5ccc6c(c5)c5ccccc5n6-c5ccc6c(c5)oc5ccccc56)ccc43)cc2)cc1]

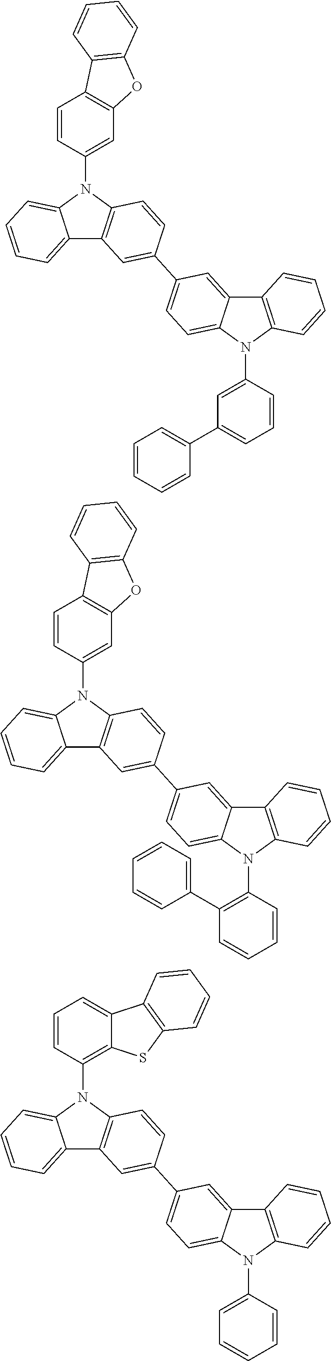

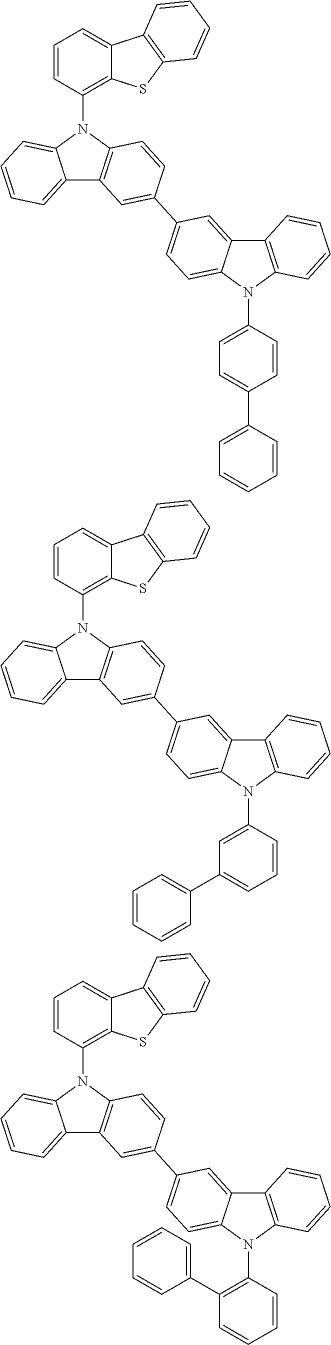

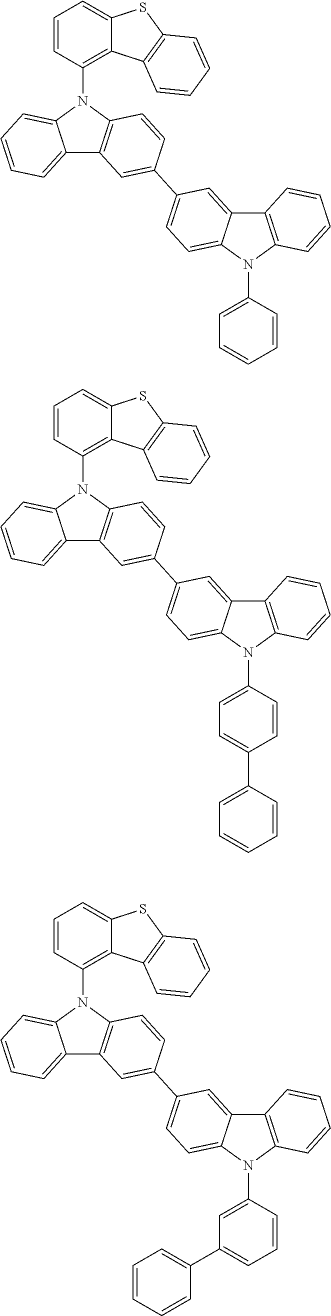 [SMILES: c1ccc(-n2c3ccccc3c3cc(-c4ccc5c(c4)c4ccccc4n5-c4cccc5sc6ccccc6c45)ccc32)cc1 ; c1ccc(-c2ccc(-n3c4ccccc4c4cc(-c5ccc6c(c5)c5ccccc5n6-c5cccc6sc7ccccc7c56)ccc43)cc2)cc1 ; c1ccc(-c2cccc(-n3c4ccccc4c4cc(-c5ccc6c(c5)c5ccccc5n6-c5cccc6sc7ccccc7c56)ccc43)c2)cc1]

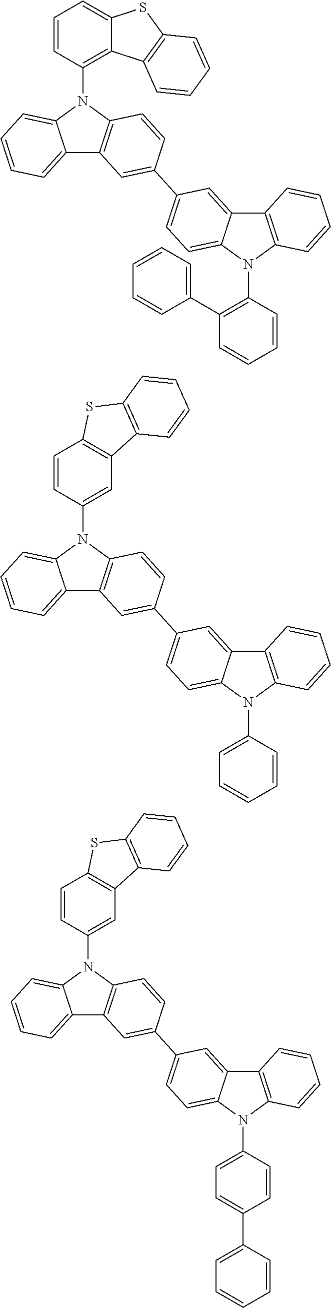 [SMILES: c1ccc(-c2ccccc2-n2c3ccccc3c3cc(-c4ccc5c(c4)c4ccccc4n5-c4cccc5sc6ccccc6c45)ccc32)cc1 ; c1ccc(-n2c3ccccc3c3cc(-c4ccc5c(c4)c4ccccc4n5-c4ccc5sc6ccccc6c5c4)ccc32)cc1 ; c1ccc(-c2ccc(-n3c4ccccc4c4cc(-c5ccc6c(c5)c5ccccc5n6-c5ccc6sc7ccccc7c6c5)ccc43)cc2)cc1]

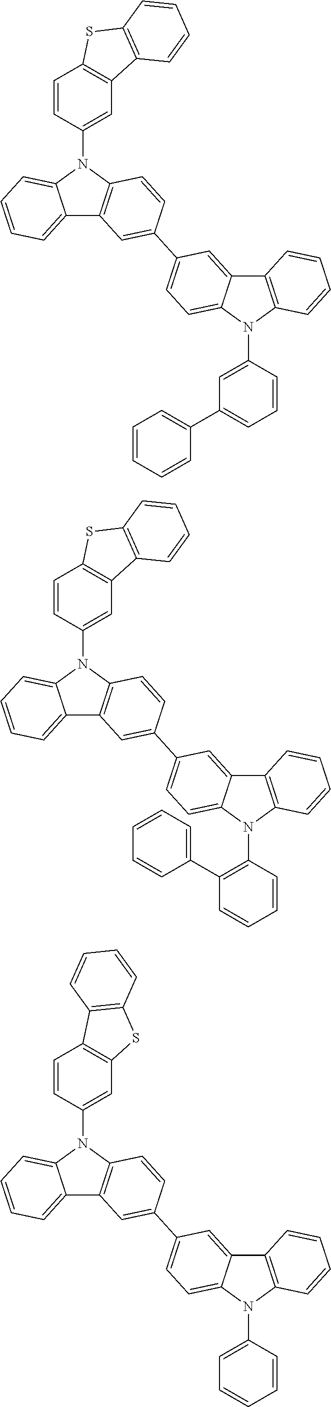

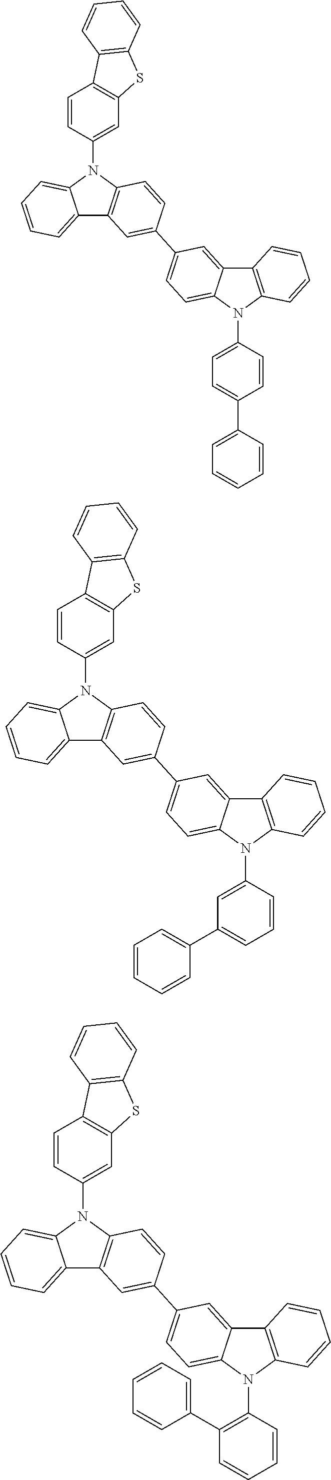 [SMILES: c1ccc(-c2ccc(-n3c4ccccc4c4cc(-c5ccc6c(c5)c5ccccc5n6-c5ccc6c(c5)sc5ccccc56)ccc43)cc2)cc1 ; c1ccc(-c2cccc(-n3c4ccccc4c4cc(-c5ccc6c(c5)c5ccccc5n6-c5ccc6c(c5)sc5ccccc56)ccc43)c2)cc1 ; c1ccc(-c2ccccc2-n2c3ccccc3c3cc(-c4ccc5c(c4)c4ccccc4n5-c4ccc5c(c4)sc4ccccc45)ccc32)cc1]

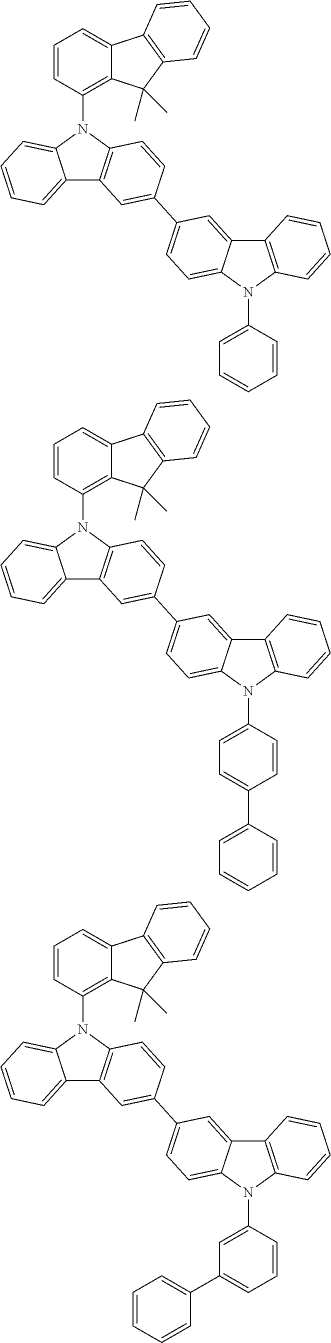

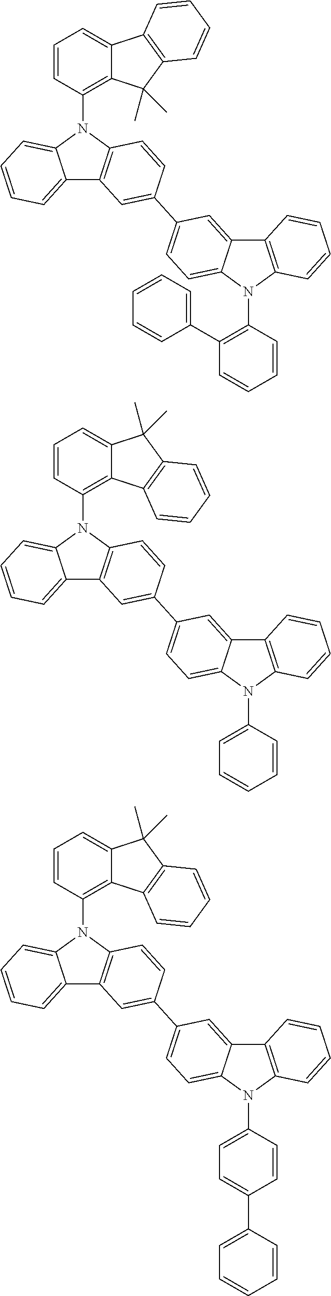 [SMILES: CC1(C)c2ccccc2-c2cccc(-n3c4ccccc4c4cc(-c5ccc6c(c5)c5ccccc5n6-c5ccccc5-c5ccccc5)ccc43)c21 ; CC1(C)c2ccccc2-c2c(-n3c4ccccc4c4cc(-c5ccc6c(c5)c5ccccc5n6-c5ccccc5)ccc43)cccc21 ; CC1(C)c2ccccc2-c2c(-n3c4ccccc4c4cc(-c5ccc6c(c5)c5ccccc5n6-c5ccc(-c6ccccc6)cc5)ccc43)cccc21]

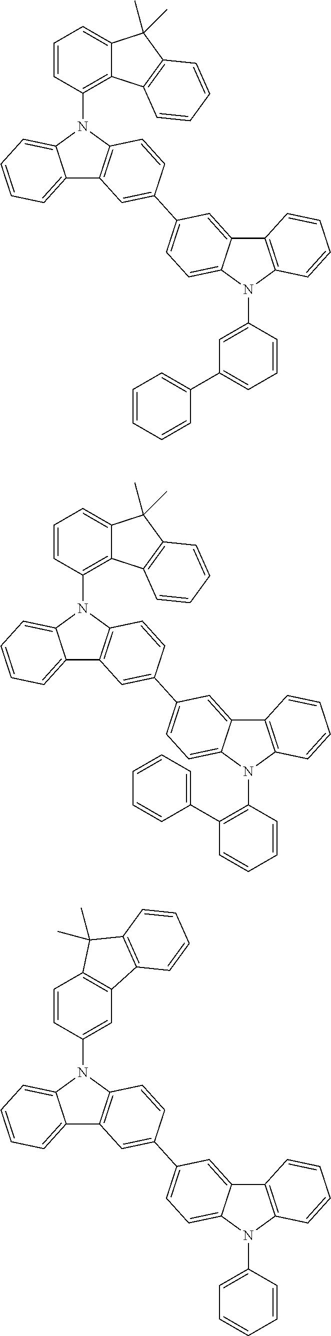 [SMILES: CC1(C)c2ccccc2-c2c(-n3c4ccccc4c4cc(-c5ccc6c(c5)c5ccccc5n6-c5cccc(-c6ccccc6)c5)ccc43)cccc21 ; CC1(C)c2ccccc2-c2c(-n3c4ccccc4c4cc(-c5ccc6c(c5)c5ccccc5n6-c5ccccc5-c5ccccc5)ccc43)cccc21 ; CC1(C)c2ccccc2-c2cc(-n3c4ccccc4c4cc(-c5ccc6c(c5)c5ccccc5n6-c5ccccc5)ccc43)ccc21]

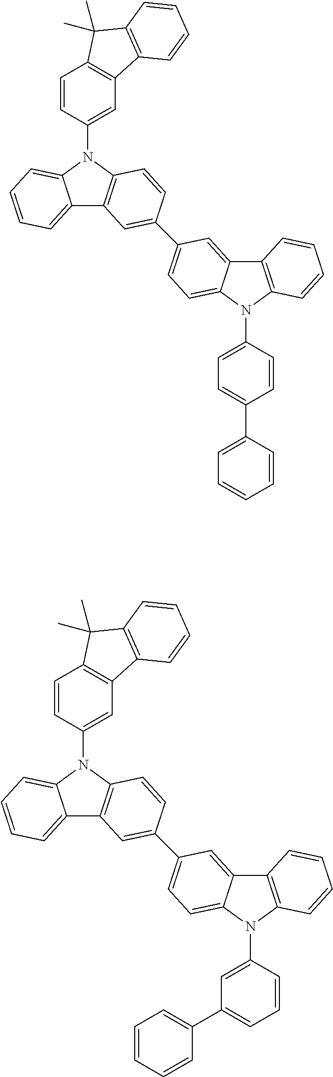 [SMILES: CC1(C)c2ccccc2-c2cc(-n3c4ccccc4c4cc(-c5ccc6c(c5)c5ccccc5n6-c5ccc(-c6ccccc6)cc5)ccc43)ccc21 ; CC1(C)c2ccccc2-c2cc(-n3c4ccccc4c4cc(-c5ccc6c(c5)c5ccccc5n6-c5cccc(-c6ccccc6)c5)ccc43)ccc21]

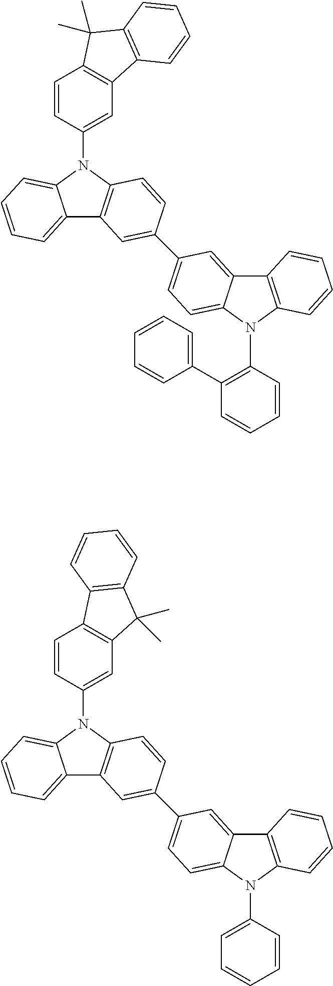 [SMILES: CC1(C)c2ccccc2-c2cc(-n3c4ccccc4c4cc(-c5ccc6c(c5)c5ccccc5n6-c5ccccc5-c5ccccc5)ccc43)ccc21 ; CC1(C)c2ccccc2-c2ccc(-n3c4ccccc4c4cc(-c5ccc6c(c5)c5ccccc5n6-c5ccccc5)ccc43)cc21]

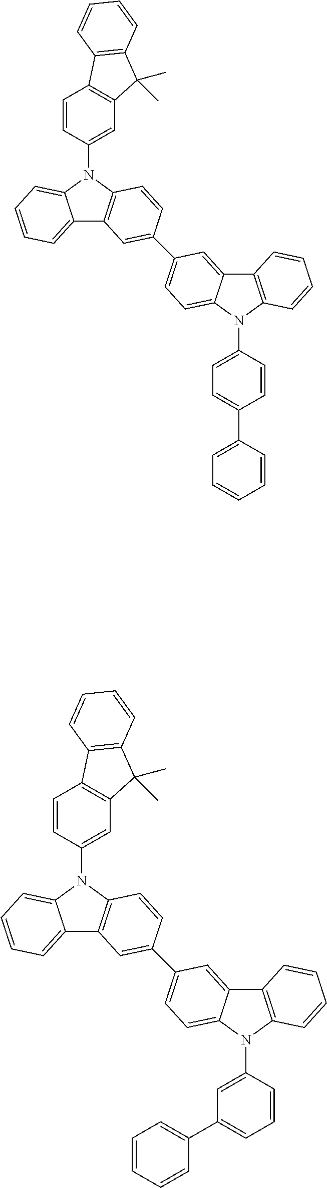 [SMILES: CC1(C)c2ccccc2-c2ccc(-n3c4ccccc4c4cc(-c5ccc6c(c5)c5ccccc5n6-c5ccc(-c6ccccc6)cc5)ccc43)cc21 ; CC1(C)c2ccccc2-c2ccc(-n3c4ccccc4c4cc(-c5ccc6c(c5)c5ccccc5n6-c5cccc(-c6ccccc6)c5)ccc43)cc21]

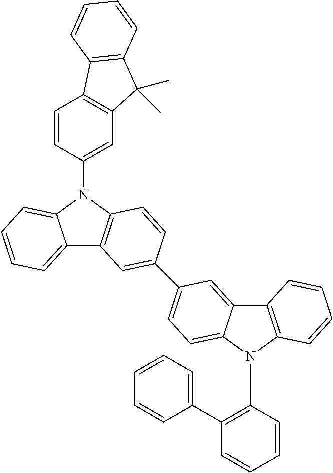 [SMILES: CC1(C)c2ccccc2-c2ccc(-n3c4ccccc4c4cc(-c5ccc6c(c5)c5ccccc5n6-c5ccccc5-c5ccccc5)ccc43)cc21]

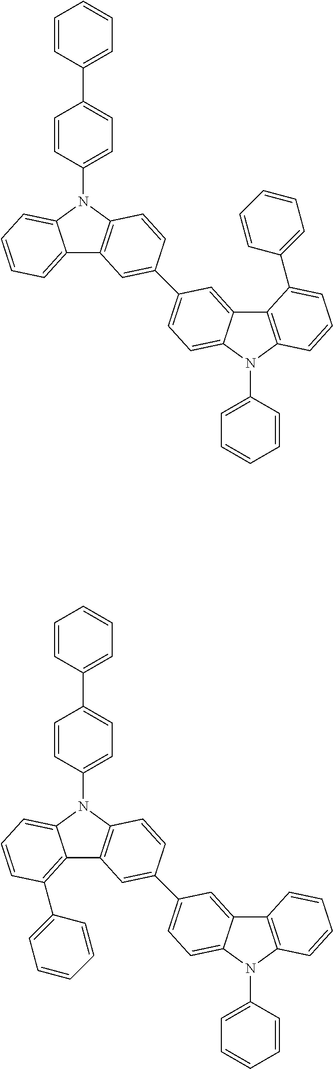 [SMILES: c1ccc(-c2ccc(-n3c4ccccc4c4cc(-c5ccc6c(c5)c5c(-c7ccccc7)cccc5n6-c5ccccc5)ccc43)cc2)cc1 ; c1ccc(-c2ccc(-n3c4ccc(-c5ccc6c(c5)c5ccccc5n6-c5ccccc5)cc4c4c(-c5ccccc5)cccc43)cc2)cc1]

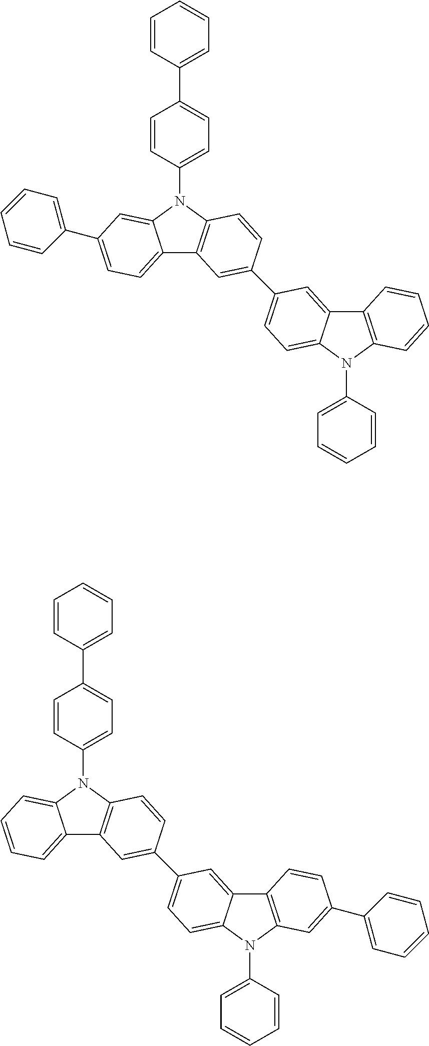 [SMILES: c1ccc(-c2ccc(-n3c4ccc(-c5ccc6c(c5)c5ccccc5n6-c5ccccc5)cc4c4ccc(-c5ccccc5)cc43)cc2)cc1 ; c1ccc(-c2ccc(-n3c4ccccc4c4cc(-c5ccc6c(c5)c5ccc(-c7ccccc7)cc5n6-c5ccccc5)ccc43)cc2)cc1]

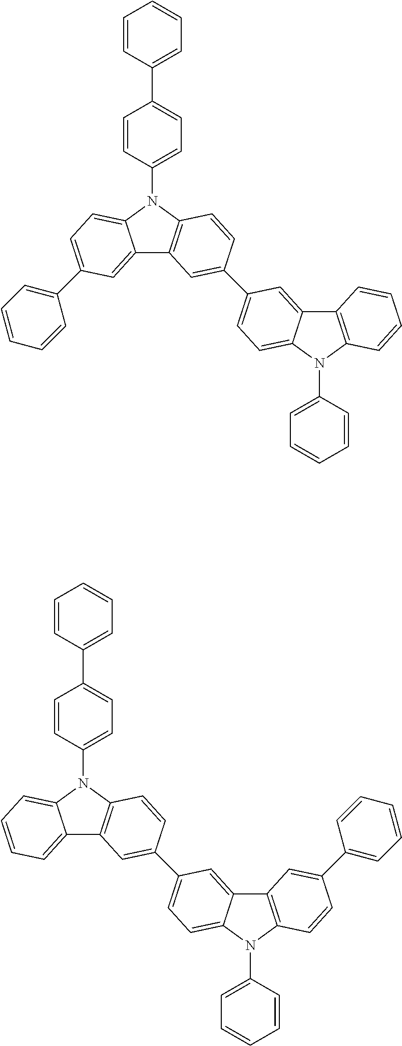 [SMILES: c1ccc(-c2ccc(-n3c4ccc(-c5ccccc5)cc4c4cc(-c5ccc6c(c5)c5ccccc5n6-c5ccccc5)ccc43)cc2)cc1 ; c1ccc(-c2ccc(-n3c4ccccc4c4cc(-c5ccc6c(c5)c5cc(-c7ccccc7)ccc5n6-c5ccccc5)ccc43)cc2)cc1]

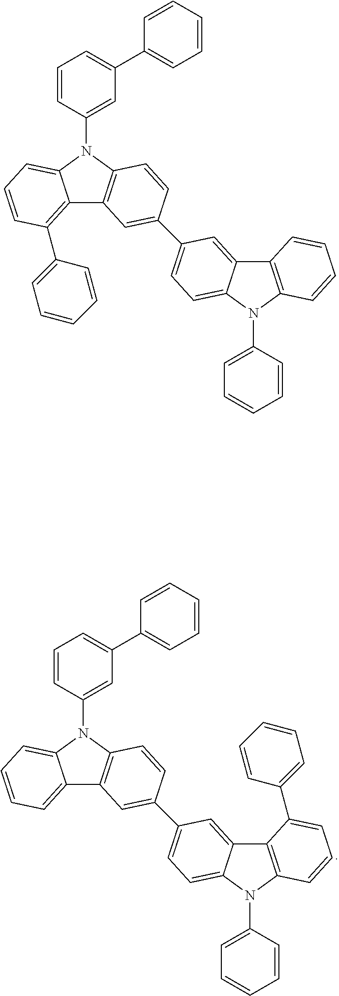 [SMILES: c1ccc(-c2cccc(-n3c4ccc(-c5ccc6c(c5)c5ccccc5n6-c5ccccc5)cc4c4c(-c5ccccc5)cccc43)c2)cc1 ; c1ccc(-c2cccc(-n3c4ccccc4c4cc(-c5ccc6c(c5)c5c(-c7ccccc7)cccc5n6-c5ccccc5)ccc43)c2)cc1]

19. An organic light emitting device comprising:
a first electrode;
a second electrode that is provided opposite to the first electrode; and
one or more organic material layers that are provided between the first electrode and the second electrode, wherein one or more layers of the organic material layers comprises the composition of claim 17.

20. The organic light emitting device of claim 19, wherein:
the organic material layers comprising the composition are a light emitting layer.
